SEQUENCE LISTING (55 sequences)

SEQ ID NO: 1 - DNA, 267966 bp, Homo sapiens
gcaaacaaacgacgtccgtgattggctccgttcgggcttcggctcccagccgaagcgggcgagcgtggggctcggccggcgattcccagacgcctgttacgcgggcggcggggcgctgggcggtgtaaggctgggtgggggaggaaggaggtggaggacgagtaggaggggggaggaggagtggggaagtgcaaggcggctgcgcagacagcgctcctcacacagagcagcycctgacccgggcgaatgcgggcttgtgccgccgccgccgccgccgccgcccgggccaagtgacaaaggaaggaaggaagcgaggaggagccggccccgcagccgctgacagggctctgggctggggcaaagcgcggacacttcctgagcgggcaccgagcagagccgaggggcgggagggcggccgagctgttgccgcggacgggggagggggccccgagggacggaagcggttgccgggttcccatgtccccggcgaatggggaacagtcgaggagccgctgcctggggtctgaagggagctgcctccgccaccgccatggccgctggatccagccgccgcctgcagctgctcctggcgcaatgaggagaggagccgccgccaccgccaccgcccgcctctgactgactcgcgactccgccgccctctagttcgccgggcccctgccgtcagcccgccggatcccgcggcttgccggagctgcagcgtttcccgtcgcatctccgagccaccccctccctccctctccctccctcctacccatccccctttctcttcaagcgtgagactcgtgatccttccgccgcttcccttcttcattgactcggaaaaaaaatccccgaggaaaatataatattcgaagtactcattttcaatcaagtatttgcccccgtttcacgtgatacatatttttttaggatttgccctctcttttctctcctcccaggaaagggaggggaaagaattgtattttttcccaagtcctaaatcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagtgagtattggttttattttacaccccttttccacctcgcccttaaaataagaaacctcgaaataccaggatctgaaattattagacctcctgataaacaggcaatggaaaagggcaaaacctatcttcagtcaaggatgaagcagctccctttttactcctcccataccccctcccccggtctcattaaccttgaattgagataatatgatatttatttggatgattcgatttcaaactgctttcttgtacttttcttgctgttggtagtcagtttcatcaagtgctcgagtgtttccacgttaaagtagtgatgacagtgtttacttgcccatcctttagaataaagagaaggacaagatttgattactaaaccattatgcacatttgcagaatttttggaatttggaatgagagaatattttaggtctcaggggttatcatagtttgatatacttaatagagacgttaaatgtcccaccctcccaacatttttttcttgtttgtatgactacacataatatttgttcttaatctgtggatgaacttcctcagtaaccattcacatttggaggctgccattgatttctctctttagaagggactagtgggagccaataaatacctagcagcagtaacatctctgtatgcatttgtgttcaattggataagtggtttgatgtgttatctcttaaccttcctgttttgacattaccaacctgccaccgtataatttatgtttaatcaaaagttaacttggaagtattaataggtccaagttttgattgagatttgttttgtcatagtgccaaatactaagttaaatgttgtcactggctcattacatttaagttcaattttggagtgctactgcattcattacctgttaaatgttctggcgggttccttaaaaaggacgatttaaaacactctagcgttagtttccatcatatttagataactgtggatttagttagaagaatgtggatttagtcctttccattttggcttttgcagatggctatggtcctagaaataatagggatatttgggctgtgttcagggttgtggatagactactcttgtcctgggtttgtgctgaaaagtatgctcttgaagtgtttaatatatactaagtatctaatgttccagtgtatctctatttatggagaagttcatgtggtatttaatgatagcttgtttaaacatacttgcagaatttaacaaaaatatctatattgtttccttattgtgggaaagaggggcatcatgagggcaaagacaaattaatttgttgcctagaagaactgagagaactaaagaactgagattttaagaacccacacacacgcaaaacatgatgttatgtgaaattgattatattcttaggcaacataaaatgatcacttttagttgaactcaagatttcaattcatgttggctttgggaactagcctgaaacagggtagtctaggtaatagctgaatgggagataatttttgaaagttaattttttttttttcttgtggcttgcaagtagagaatgcccgggtaggtaaagaggaaagctgattatgcatctttcagggaagggtcgtgctttctaagcacttcatggagtgatttgaaaaatattttaaaattccggggtaattttcagctatgatgatacaaatcttataatagctacttaagagcctcttgtggtactggagtgtttgtatgttttacatcttggtaatatagtttgaaaatcagtcagtctatttcagctaattgatttattagctttatttcagagatctgtaatgctagattataatgctacattttgggacttttcattttcttttcaggattttctttgaaaatgactcttgaattcttggtaatacagatactaactaaggatggagtaactcttgtaaaatatatagatgaaaatgtttaattccgattaccatataggtcaatttagaattttaaaagttggagtttagtgttaattatgtgtttttgacagctttgactttaaaagcatttaaaagaaaggggagtctttaagcatttgttaggatgctagcattctaaggaacccctgttgaaaacagacataagaaattgtagtccttgctctcaaaagcttatcctttagttgtagagggaggctatggggaaaaaaaaattaaaacaatagcgtgatgactgcaagattgtaaaatacaaattgaatcatttgttcagagtggcaaagagtaaaggggtgagactgatggttgggagaggaaagacttcctgcggacaaggtgtattaggactgatgttaaaggaggtggtaacttgaactggtagaaaagcatgaaaggtctaggtggaaggagtgtaccaaggaacaatgatggtgagttttgcatgagagacaataatcaagttgacaaatctgaattaaaggggcctaaataagaacataagaacagatggaatttgagaaataagagaaagttagctgatggagggcagaggggttctgtacaagtttgattgagattcagaagagatctggatttattgttggttgctgaataagacgtaagatgatttttgcagttgaattagtgaattatagtagagagatgctagagcctttttagggagctagtagggtatgttgtgaatcatgagggaataaagtaaaatgtgaaagtggggtgaaaggaacaggtttgagagacagtgtgaatgaagaactggcagaacttcatactggactgaagtggaatatttgtgaaggagtcaagagttaaaaataaattctgatgttactggcctggaagacttgactgctaagttatattgttaattgtaatgggaaaattgagaaagtgctgaggtgagagggtaggtgagttcactttcttaaaatttaatatacaataagttcccttgaaattcagacagcagttagttactggcaatgatatttgacttcctagtctattaattgcacagtttctaatttttctacacctaaagatgttggatttatattcgggagcagtaaactaagggcttttatttaaatgcttctcatcaaagtcctaattttttttttttttatatacagcttcaaaagcctcacacttgaaacctggttataatagtttcatgatggcttatttcagatcaatgaaaatacttttgggactaaactgtgactgtaacattttagatatgtagccattaatctttgatatatgcttaaaacttgttttttataaataatgattattactattattttaaatgcaaaatttatctaagaataaagtattgagagccttcactttgtgaagactccagaagaaattaaaagttgattttataacaagtattttttgccctttttgttgagtttaggcattaatcccaaatttaaatcagtatatttgaaatactactttttacttgctatagtgaaaccaatgtttcatttttgttcacttccaagtttacatgtaattaaataaatgtttataaagtttttctttagaatcacatgatattttactccatagggaaagtaagggactgtttgtggcacttttggttaaaatattagtaatccttttggtatatacaattggagaaaatttagctagttgtttaggtttaatacttaaatataagatttagaaatatgaaaataagtctgtatcacttaagttgtttcctcatataaaacttttttagatcaaaatttctattagcaaagagagaataatagtagctgcatattattctgcagtattacgtcgtatggtgattaaagccaaagagtaggtagcaacttagagtttccgtattcccctgggaagtttaatctctttatgacatctaaaaattaggttattagagtttagtccatttcttgataatcctgtgttaatgtggaaaagagttcatgtgtgagactgaaaaccagaactttattttcagtcatttcccaacccgacttccccagagttgttaaaacgtttaagatatttaagaattgcatgctttcttgttgcatgcacctgtagaatgctaattgtgaagtgacagtaagtcacaggcaaaaggaaaaacttcaagggaacccagtgattcacaaatcaccttatggctgattgggaactgcatttagaaaatgaggtgacatcttctcatttcactggcaaagttcagctgaaaatcagtacttgcacgctttactgtgtagcccttatattagaattgagttgcaggtgtgagataatattttggagaaacagcaaggctgggaaagtcagagattttattccagcagcagagtctaaagagtgggagagctctatttgtgtgtgcactttatgcatagagaaggctgtgaaacatgcatatgttgaccctattgtcacagttccatacttttaaataagacccatgaagttgagacagggttggagatgacagacaggatgtgagattgtggtgcttcttgtgatgctccaccctataatggagaatggaggggcagaacgcttgtgtgggtgagacaagagtgtgtgttggtactaacaggaccaaccgggttccccggatattgttcttctaagaacataatcaccataagtaaattaatttgttggaatgtttatcattttctagctgattttttgtgtgtgtgtgtggggtggtggtggtggaaggggagttttccccgatttcaagaataaaagcattttcattgttcagaagaagagcattttcattaaagcggaggttgcactcttggcattttatcttgagttatacaaatcatccttggttatacaaatcaaattgcttttgaaatttgttgagtgtaatgtataaatgtatatttatataaaaacctatgtgaatagaacagcaaggaaaaactgagtagagttaaagaagtttgttttccctgagattttctgcctttaaatttatataaaaaaggaatattccattttacgttattgagttttctatttttttaaatgagctaatactcagtacataatggacatttttagtaaattattccaatatacttgtgtctgtaatgcatttgatgtggtataagatttagaaacacaatttagttcggctggcacggtggctcacgcctgtaatcccagcactttgggaggccgaggcgggtggatcacgaggtcaggagatcaagaccatcctggccaacgtggggaaaccccgtctctactaaaaatacaaaaattagcgggggcgtggtggtgtgcgtctgtagtcccagctactcaggaggctgaggcaggagaatcgtttgaacccatgaggcggagattgcagtgagctgagatcgtgccactgcactccagcctgggcgacagagagagactctgtctcaaaaaaaaaacaaaacaaaagacaatttagtcgatttgggttcttagtttataatcccccttaccatttttgcttatattttgggaagagaggtcaacttccaaacaatgtgcaggttccaattttataagtgattaaatcttatattttttgtattgaatagttttcttagcatgtttggtttgatcacttgaagtaatgatgtatttgtcttgaaactagttttgtcaaaccaactttttatttccagagtaccatggttaacaacagcacaaagccaagtccttaacgtgttcccttaataacatggtctaaggttaatttcttgattcttatttcaaatcaattcagtaataaaaagtacactttttttttttttgagatggagtctcgcactgtttcctgggctggagtgccatggcgtgatctgggcttactgcaacctctgccacccaggttcaagcgattttcctgcctcagcctcccaagtagttgggattacaggtgcctgccaccacacctggctaattttttgtatttttagtagagacggagtttcactgtgttggccaggctggtctcaaaatcctgaccttgtgatcttcccacctcggcctcccaaagtgctgggattacaggcgtgaaccaccacatgtggccgaactaaattgtgtttctaatttagttttcctgaagaataacagctatgcatcaatgtggaattgtgttatttggaatctcaccttattggaattggctggtaattgacagtaaaccagaagtgctcctgagcagctaatatctgcagacaaatggagtgtaaactttgctcataggttagttttcaggcttttattaacggttccttgttattatttaacatatagttgtaattcattgagttgtctgggtttttttgaaatcacagatcaggggaatggataggtactgattaagcattatgctactgaaaagaatggatgattaggggaaacaagcatattgggattacttagtgtatgtagggtatgtaggttcatatagttccagggtaaatcattctgataattgttgaaaatgttcacccagagaaatttttttttttttttggagacagggtctcgctgtgttgcctatgcctagcctggagtgcagtggcacagtcttggctcactgcagcctctgcctcctgggctcgggtgatcttttaacctcaacctctggagtaactgggactataggcatgcgccaacatgcctggctagtttttgtagtttttatagagagggggtttcgccatcttgcccaggctggtcttgaactcctggaatcaagcaatcctgcctcagcctcccacagtgctgggattacagtcatgagccactgcacccatctgagaatattaattctcttttaaagaggcaggaggcagaaaaatctcttgtggaaacctgggcttctgcataaactacataaaatatctgtaatttgttgatcactgaagtgatgaagagaactgggtcacctctgtctgattcaccatcttctagttggatttcagcagtatggataaaggtactaaagtctttagtgtgctagcccagggtattgtttccaactctttcttccattgtccatctacatttattaactcagcagcagagttttatctgatttaagatttgttgtcttactatttctgtttttttttttttcctccttagtatatatggaattgctgcttggattttggctggcttgtaccttttgtttgtggactgcttagaaggcactgcatggagtcatttaagctttcttatgaattatgaatagctatgtagagaggaacttttctagatacatttatggcctatgagaattgaaaagctgggtatcttaattgttaggctgaagaaaaagggtgatagtggcaaagtagtttttcttttggaggtctgagaaattcttaggggcttaattggctaatataactagaaaggttgggtgatttaggtttttttccaaggaaaggtatcttatttaaatgcaacactgtctgcttcctgttattttataaaattagttaatacttttttttttttctcaagacggaatgtcactgtgtcgcccaggctggggtgcagtggcacaatcttggctcactgcaaccgccacctcgaggcctggttcaaggaattcccctgcctcagcctcctgagtagctgggattacaggcatatgccaccattccccgctaatttttttgtatttgtagtagagatggggtttcaccatgttggccagactagtcctgaactcctgacctcaggcaatctgctggcctcagcctcgcaaagtacagcaaaatcagttaatatttttaattgattaacactttcttcatgacctgtgttgaagttattgagttgagtcaattgaatttctaatgctagattttgagtatgtggtgtgagcacctgaagagttggaggtactggaattatgtctgacatcagactgatagtatttgcagggacatttgccctagtggaattcagggttttcacattctatacattacttaaattatgaaggaaagggggaagctttgagttggtagacttttaataataagatataattacatgtatatagaagagcaacgtactgtatatcatattctcataatatatggtaataacggcagtaattattgagtgcggtgtgccagataattgcatgttttatttacctattttgctttaaattttaaaaaatgtaatttactcacatggttcaaaattttaaagatattcagtgaagtcttctctcctgctctgcaacaaccagttctctttcctggagaccactggcattacaatttcttatgtgtcctttcataggtattctgaatctttacggattaatgatttctatagtagacttgcattttgtgtatacatacataaattaacaatagctttagcagaagtgttagaattaaaacaaatttaaatcagtaactctagaaaattgctattttttaacctattaaattagtaaatacttatttctctaaatataagttagttggagaataagtgcaaagatgacagagctcctctcgtggcaattagaggtaaaatgttaatagtcttaaccaatgaacaattaatagcacatttaaaatagatgtttatttcataataaaaagacaggataattttttaactttctatttttctaacatcttagatgagaaaacctgttctcttgagagatcagaggcgcagagtgcgtcagtgtcaatgaagcctgtgccttttacttctttaagagcgtacggtttcattctgctcctaaatcaagcaccccttagttgttgaataaaaattacttttataaaggaatatatgatttaaagaaatcaaatagtgcagaagggcttatgatggaaagtaatcttctgtcctccagtcctacttcttagaagcaacattggctatgacagctgtttcctctagtagtttttgtcatatttttaattatatgctttttatctatttcttaaaattaatttttttgtcttattgtggtagacatcatttattgtcatgcccatctcccttttgctttcatactgatataattatgccactatttaaaaactattatttatttaaattattattcaaattattctcagtatgtagacatggaacagtattctatgtagaaacaagtagtatacagtgatcacatttccttatgagaatagctttattgagatataattcacataccgtaacattcattttttcattactcaggtgttcatagacttgtgtaaccatcccaaccatctaattttagagcacttttatcatttcagagataaagcttatacccattagcagccattccctatgttccccttccccacagctctaggcaaccactaatttactctctgtctttatagatttgcctgttctggacatttcatataaatggatttatgtaatacatggccatttatgactgttttctttctcccatgttatagcatgtattagtacttcattcctttttattgctgaataacatttcattgtatggaaatcctgcgatttgtttattagttgatggacatttgggttgtttctgctttttgactagtatgaatatgctgctgtgaacatttgtgtgcaagtttttgtgtggacatatgttttcagatcttttggggtatatacctaagtagtggaatggctgggtgatatggtaactctctgtttaaagtgttaaggagctattaagctgtttcaaagtgacagtcacattttttcctttgcttttaatctttttagactttattgttgccattttctttctttttttttaaatactgtttgtaataattataatctcaattcttccccccaaatgtaccaatttatcagacattctctggatttctacctgcctcctgcccctttcttttcctggagtgtcctttttcctggagccctcgtccaactgctgggatctgatctagttgttctccaggccccgttgtacagctgtcatcctctgacttctttcgctgctcttcagggttggctctccagtttcctggatcccatatctctaaataacatttcattgtatggaaatcccatgatttgtttgttagttgaatagttcttatctgataactgtttgataaatatacctggaaaagttgttcattgtttttttccatatttgttccaaaagggcagttaagaagctatatactttttgggcagataccctcacccttgcagtatctagatactgagaaatagatgttggaaggttactttgtatcattttattaagtgattaccatgcaaacataaggcaagtacaaaaatctgaaaaatattaatattaagtaattgtttagactagagctattcagaagaaatattatataagccaaaaaggtgaattatatatgtaattttaattttctatgaaccactttgaaaaatagacacaagtgaaattaattctagtaatatattttatttaacctagtgtatctaaactaataatatatcaagatgtaatcaatataaaaattattgagatattttacattctttttttgggtagtaaatctttataacctagtgagtgttttacacttaaagaacatactaatttgggggccgggcgcggtggctcatgcctgtaatcccagcacattgggagaccgaggcgggtggatcacgaggtcaggagttcaagaccaacctggccaagatggtgaaaccccgtctctactaaaaatacgaaaattagctgggcgtggtggcagacgcctataatcccagctacttgggaggctgaggcagagaattgcttgaacctgggaggcagaggttgcagtgagccgagattgtgccactgcactccagcctgggtgacacagcaagactctttctcaaaaaaaaaaaaaaaaaaaaaaaaaaaaagaacatactaatttgggctagccacatttcaagggttcattagctacatgttgctagtggttattgtattggacagtgcaggcttagaattttgtccaggaattatattatttttgttatttttaagtaatagaaaaggtgtgtgtggtttaaaaacacttcattgatttatgcctttgtttttaatcatgttttcagcttattttgagatttaaaagaggttgaaaattgttacgtttttcagaagggggcttgctgtagggtgggtagtgattctttccacccttattttagtaaaactgttaaaataggtatttaaatttttgctgttaagataattaggtgttgtccttaccatgattgccttggcctccgtggaattttttcgatgcaaatattctttgtcatatattatttccccttgctgtttcaaagaccttagctggctttcagtctcaaatgccacctctttagtcattcatgcttatctcaaggcatgaaattcactttcatataactccttcagcactctctgtgacccttctaatgttcttccttgtattcttgttaatgtgtcttgtttctccgtaggagatcataagttccaacatgacataaaaatattttatgcatctttgtattttctgcagtgtggagcacagtcctttttgtattgggtcacttagtacatattctagttgaaggcagtttgtcatgtgatatggtgtctagtggtatgatagtttacaggtcaccaagctattgctgatgtttctaggaacatttaagttttgatgtaagaaattaagttaaatttgtgttttgtttgtttgtttttttgagatggagtctcgctctgttgccaggctggagtgcagtggcgcgatcttggctcactgcaacctccacctcctgggttcaagcgattctcctgcctcagcctccccagtagctgggactacaggcatgcaccaccacgcccagctagtttttgtatttttagtagagacagggtttcaccatgttgaccaggatggtctctatctcctgatcttgtgatccgcccgcctcccaaagtgttgggattacaggtgtgagccaccgtgcccgaccctgttttttttttttttcttaaatgtgaaggaatgatagtagggaaaggaaagagacatagagtatacagacacttctgttgcactgtgtgcttagcactgagtggctctgaaatctttggatggatgactgaaagaaaggacttgggtgctaggttgcactggttggtaagcacagtacctttgaaacattgagcatatgacgctactacggtatccccttacattattttatttggagtgggtaaggactgaagagagatttataacagtgaaacctgacagttcaatttctttcctttcaccaagtagcttactctgcaaaagaataaagtaaacagacagtatttaaatatgagaagtcttttcatcatggatgtacttaggtatgtaattgtattgtgggggagttgtgaaactgtaattttttttctttttttttttgagactgagtcttgctgtattgcccaggatggagtgcagtggcgtgatttggctcactgcaatctctgcctcccaggttcaagcaattctcatgcttcagcctcccgagtagctgggattataggtatccaccaccacgcctggctaatttttgtatttttagtagaagcagggtttcataatgctggccaggctggtctcaaactcctgtcctcaaatgatctggatgctttggcctcccaaggtgctgggattacaggcatgagccactgcacctggccgaaactaatttttacaagacctctttttgggtctttaagatgatgtttgtttgtttgatatgttagtatgtcagttctagagctttcccagagagggtgagaaatattaataaacaatagggtttctattgctaaataatacaacttcttttttttttaattagaggaaagagtgtcgtcgtcttctccttcttccttctaccttcttccttcttccttcgtcttccttcttccctccccctcctcccaccttttttggagacagaatcttgctctgtttatcagattgtagtacagtggtgtgatcttggctcactgcagcctcaacctcctgggctcaagcaatcctcccacctcagcatccacattagttgggtccacaggagtgcgctaccatgcctggctcatttttgtattttttgtagagacgagattttgctatattacccaggctggtctcgaattcctgagctcagtccagccaccttggtctctcaaagtgctgggattgcaggtgtgtgccacctcacctggccataatacaaattcttaaatagtggtcgaatatgactttctgtatttggttttgaaaaataaattgctttccacatatcagcatggtatttccttctgtgattaaatgtgtgcattgtgagatgtttggctggtatagggtacaattcttagacatggaagaaatttactttctctgtaagtagcaaatacttgttatgcccttcttgaaccaagaggatttactaaagaacaaggtataattctatttaaattcagttgaacatcaggaataacttttactgagagactctgaggcctttaaaaaatatgtttatataggccgggtgcggtggctcactcctgtaatcccagtactttggaaggttgagatgggtggatcacttgaggtcaggagttggagaccagcctgagcagcatggtgaaacctcatatctactaaaaatacaaaaattagccgggtacggtggcaggtgcctgtaattctagctactagggaggctaaggcaggagaatcgtttggacctgggaagtggaggttgcaatgagccgatactgcactactgcgctccagcctgggcaagagagcaagactgtctcaaaaagaaaaaaatgtttaatatgtatctacttgtggcagtagtctacttattaaaaaaaaaaatgtgtgtgtgtgtgtgtgtgtgtgtgtgtgtgtgtgtgtgtgtgtaatttcctgtcaaggttgtagaaaattaaaggagagttgtgctttttcaaaggatagaggagacaggccaggtgcagtggctcatgtctgtaatccctcccagagggagactgaggaggggcggatcgctgaggtcaggagttcgagaccaccctggccaacatggtgaaaccctgtctctaccaaaaatacaaaaattagctgggcatggtggtgcatgcttgtaatcccagctacacgggaggctgaggcaggagaatcgtttgaacctggcaaagcagaggttgcagtgagctgataccgtgccactgtactccagcctgggcgagacagcgagactgtctcaaaaagaaaaaaaagtgtgtgtgtgtgtgtgtgtgtgtgtgtgtgtaatttcctgtcaaattgtagaaaattaaagaagagctgtgctttttgaaaggatagaggagacaggccaggtgcggtggctcacgtctgtaatccctcccagagggaggctgaggctggcagatccctgaggtcaggagtttgagaccaccctggctaacatggagaaaccctgtctctactaaaaatacaaaaattagctgggcatggtggtgcacgcttgtaatcccagctacacgggaggctgaggcagaagaatcgcttgaaccccagaggcagaggttgtagtgagccgagatcgtgccacttgctctccagcctgggaaacagcaagactccatctcagaaaaaaaagaaaaaaaaaaaaaaagaggagaagttctagtatgatatttctgatatttctgctctacattggagaaggagaacttagttagaaacaaagggtgtgtttgtcgtatttgtcatgacttccaaaatgggagcaggtgggccaattcctgggtcgttagaaaagaagacccagagacaggagattgtcttccggcactgtgtgggctatggtatcaatataagttgtatagcttgttggctttcagtatttatttaggagtaggaaattgttctaaaatgcatttcacattatatctaattaaaaacacatctttttttgtgttaatttcttacatatagctgagaaagcctagactttctaaagtctaggaaagaggtgagaaatgctgctgtggaccataggagttggggtgggggaagaagctaattagaaaaaagtttgtggtcatgatagattttgagtttctctggatgctgttaatgatggccggaattttagaattcagattcagctactctttgaatgtttattgtgtgccaagcactctcattccatttaactttcataatgctctgtgacaatatttgccccatttaatgagtgaagaatctgaggttaaaaaaaatatatttttttaattgatttattccggggccttacagttagtagggaataggttccagatgttgatcccaggccctccagacttgaagtaaaggaagaaaattttttgagtacttaccatgtcattgtacaatagtaagcaattaatttaaaataaatgttactatgtatatttaagatatacaacatgatgttaatatgatacatattataataaaatggctactgtagtaaaagaaattaacatctccatctcacatagttacccaccccctctccctcgcccctaccaagagcaactgtaatctactcaactcttaacaaaagcctagatacaatacactattattaactatagtcttcatgttgtacattagatcttttgacatgtttatcccaggtatttgctacttcctatactttgacctacatctccccatttcctcccctgctcaacctctgctaaccagtttactctctatctctgtatatttgacctttttttgaaaaaagatttcacatataattgagatcatgatatatatttttttctgtgtatagtttatttcagttaatgtccttcaggtccatccatatgtgtgtgtacacacacacatatatatatacacacactgaagtttaaccattcgtccattgatggacacctagggttgtttccatatgttggctgttgtgaataatgctgtagtgaacgtgggaatgcagatatctttacgaagtgatgatttcataatattttgtatatacccaaagaataattactgggtcatatgatagttctatttttagtttctttagaaacctccatactgtttccagagtggctgcactaatctgcattctcaccaacagtgtacagaggttctcttttctctacacctctaccaacacttaatctcttattttttatttttatttttttgataatagatatcctaacaggggtgaggtggtatctcatcatggttttgatttccatttctctgatgataagtgatgttgagcaccttttcatatacctgttggccgttttttaggtcacctttagggaaatgtctattcacctcctttgcccattttttttttttttttttttttttttgagacggagtttcgctctgtcgcccaggctggagtgcagtggcgcgatctcgactcactgcaagctccgcctcccgggttcacgccattctcctgcctcagcctcccgtgtagctgggactacaggcgtgcgccaccatgcccggctaatttttgtatttttagtagagacggggtttcaccgtgttagccaggatggtctcgatctcctgacctcgtgatccgcccgtctcggcctcccaaagtgctgggattacaggcgtgagccaccgcgcccagccgcccatttttaaatagagttatatgttttcttgctgttgagttgtatgatgtttttataaatcttgaatattaaccccttatcagatatatggtttgccaaaatgttttcccagtctgtaggttgtctttttattttgttgtttcctttgctgtgcagaagctttttagtttgatgtagtctcatctatttttacatttgtttcctgtgttttggtgtcatggaaaaagtcattgccaagaccaatgtcaaggagatttctccctatgttttcttctaggaattttgtggcttcaagtcttatgtttaggtttttatccattttgagttggtttttgtgtctggtgtaagatgaggattcagtttcattattttgcatgtggaaatccatttttcccaacaccatttattgaagaattgtgttgtcttggtgcctttgttgaaacatagttgacggtctatgtttggatttgttgctgggctctctattctgttgcactggtctgtgtgtctgtttttatggcagtactaaactattttgattatagttgctttgtaatttaattttaactcaggaagcgtgaagcccccaacttttttttttcctcagaattgccttggctgttcagagtctggtggttccatgtgaattttaggattgctttttctactcttgtgaagaatgccattgggattttgataggaattgcgttgaatctgttttgttgctttgggtagtatgataattttaacaatattcttttgatccatgagcatggaattctttccatttatttgtgtcctcttcattttctttcatcagtagttttagttttcagtgtacaggtctttcaactttttggttaaatttattcctaagtattttgtttgttgatactaacacaaatggggctgttttcttaatttctttttcagcttggtcattatttgtgtatggaaatgccatgaattttttatgttgattttatatcctacaactttactaaattaatttattcaatctaacagtttttcatgtggtctttggagttttctatatatgggatcatgccatctgcaaatagagataattttctgatttggttaactttttttttcctttttcttgtctaaatactcttgatttagacaagctcttccagcacttctagtattctgttgaatagaagtggtcagagtgggcatctttgccttgtatcaggtcttagaggaaaagttctcagtttctcgccattgattatgtagttagctgtgggtttttcatagatggccttattatactgaggaacttgccttctatatttaaactgttaaagagtttttatcaagaaagtttaactttgttgaatgctttttctgtcagttgatatgattgtgtcatttttctttcagtctcttaatatatcacattgattcatttgtgtatgttaaaccagctttggatgcccatataaatcccacttgatcatgatgtgtaattttttgatatgttgaatttgacttgctaatactttattgaggatttttgtatcatcagtgttcatcagagatactggcctgtagttttattttcttgtaatgtttttgtctagcttaggtatcaaggtgatgctggccttatcaaatgtgttacagctctattatttggaagagtttaagtagtattggtattcttaaaaagtttggtagaattaagctgtgaagctgtctggtcctgaccttttcttttttgggaggattttaattaccactggttcaatctttttgtttgttatgggtctatttaggttttctattttttttctgattcaaccttggcaggttgtacttttttaggtattcttttcttctaagttatcacatttgttggcatgtaattgttcgtaacagtttctcagcgctgggtgtggtggctcacacctgtaatcccagcactttgggaggctgagacgggcagatcatgaggtcaggagttcgagaccagcctgaccaaaattagccgggtgtggtggtgcgcacctgtaatcccagccactcaggaggctgaggcaggagaatcgcttgaacccgggaggtggaagttgcagtaagccaagatcgtgccactgcactccagcctgggtgacagagtggaactctgtctcaaaaaaaaaaaaaaaaagtttctcatgacccttcttatttcttttctttctttctttctttttttttttagagacagggcctcactctgtgacccaagctgcactgcggtggtgcgatgtacattcactgcagtctcaattcctgggctccagtgatcctcccacctcagcctcctgagtggtactgcagatatgtgctacaacgcccagctaatttttgtattttttgtagagagagtgtttcaccatgttgcccaagctggtcttgaactcctagactcaagtgatctatccacctcggcctcccatagtgctgggattacagtcgtgagcagcggcacctggccaactgtttttatttctgaggcttctattgtaatgtctccactttcatttctcattttgtgtattttggactcctctctttttgtgtagtctaagggtttgttgattttatttttttaaaaaaccaactctttgttttattgtttttctccatgggttgtctattctctattttaagcaacttgtttgtatcttatttaattttcatcacagtcttctaaggcaaattgaatttataaatgaggaaactgacgcttacagagccttacacagccagcaagatgcatagccatacttcgaacctgttctcttaattgctccactgtatggcctttataatttggagagggaataagataattgcccgaaattggaagaattaaggttttttagggtacggcttaggaacttctactttttgtagtgttacttatggaggagaaagtaattgtcagtcatatataagtacagtttattttattttaaaataaaaattttaagagcaaataccttgcttttaaaactgatctggctagctataaaacagtggctgtgagagatgggtaagaagtgtagttgaaatccatattgcaaaatcagttttgagcctgagctcctatccttttcttttcttctttgagatggggtcttgctctcttgcccaggctgaagtgctgtggtgtggttgtggctcactgcagcctacctcctgggctcaggtgatactcttgcatcagcctttcaattagctaggactacaggcccgcaccaccatgccaggctttttttttttttttttttttttaattttttttgtagagatgaggtctccctatgttgtccaggctgggcttaagcaattcttctgccttgtcctctcagagtgcagggattatagatgtgagccacagagctcagtcaggatcactttaacagatgaaactgaatcttagagaagttaagtaactcttgccaaggtttcatagccagcaggtggcagagcctggattccaacccaggtaatttagtctagagctcttattcttaactactttactgtcctaagagtatgcgaatgtacagaaatggagccttgctacctctggtaacttgaaggcaccaattttgcgttcagttaacatatcatagtttgatccaaggcaagaacagaagtgttaacacttcacctgcctctgtccttattatttcccatggttattggtctcaataatttttgttgttgttttatattattcttgcactggaaaatcctttgtacttagttgtcatttaataaatgatgaatgttgaaaaataagccagaaattgaaaactttgaatcttaaatatagagaccaaattgtaatacatagttgattgtcatgtttttctttttaagaagttatttttatagtgtttgaagataattattattattatttttttgagactgagtcttgctcaggctgaagtgcagtggcatgatctcggctcatggcaacctctgcctctcaggttcaagcgattctcctgcctcagcctctcgagtagttgggattacaggcacgtaccaccatgccctgccaatttttgtgtttttggtagagactggttgttctctaccatggcgaactaccatggtgttcgctgtgttggccagagtggccttgaactcctgacttcaagtgatcctcctgcctcagcctcccaaggtgctgggattacaggcgtaagctaccgtgcccggccgaagataattaaatattaatagaaaaggttgatttatcattttcatgagttggagaaaattttctgtgcctctgaatcgaacttttcttagttgatgagtgttttaagtgtcagtttacagtgaaatcctgtgatgatacaaaatttagatatagtataattgactctcatcctctgccatgcctattgtctgcaggtgagggccaggctgacatttcatttttaatcattgacttaatattcagtaagtaaacctgatatgtgaacttttctctccctttcctttctattctgtaactggaagactaatggaaaaatacagttttctgttaactttgcaataagccctgtgtttcatgtattaatagttggattttttagaccccacttaaaagagtatatctgggttcttaatgtggtggcagtggtcccaaaccttcttggcaccaggactgcttttgtggaagacaatttttccatggacgagcgtggatggttttagcatgattcaagtgcattacatttaatgtgcactttattattattacattgtaatgtataatgaaatagttatacaactcatcatagtgtagaatcagtaggagccctgagcttgttttcctgtgactagacagtcccatctgggggtgctggggagacagtgatagattatcaggcagtagattctcataaggaatgtgcacctagatccctcgcatgtgtgctttacaatagagttcacgctcctatgagaatctgatgctgctgctgatcttacaggcggcagagctcaggtggtaatgtgagcaatgaggagctgctgtaaatacggatgaagccttgctcccttgcctgctgctcacctcctgctgtgcggtctggttcctaacaggccacagaaacccctgatatatagcgtttaatatataggatactcatataaccagaggtacttatttcctagctcaactatatggaattaagatgggaactagatagtagataaaaatgagcactgaggagctaaaagagtgatcacagaatctgaagtaaagttgttgtttttttatttaatcgatgtgaaagtcctttggccagtgtctcccacagtagttactctaaacgatgttgtgctgtaataaatagtctgtggccaaagtttaggaaatgctgcttatagcatccctcaaaggactgccattctttggatcagactttggaagctctgcattaggtgtatttagcccttgtttttatttgagaaggggaggaaacaaacatttaatgaggatttgctccactgggtctaagtggtatctccattttgcagacaaggagtctgagtccctgctggtgggtaaaggacacaaaactaataatgaggcagaactcggaatggatccctaatcttgccgattcctaagtttttgcccttttgaactacgttggcactgtgtggttataatgaactgctgtctattagactttactccttaggaacagcaatttggcaatatatacaaggaaaagactaagctgccctacttagggaagaagaaaaggataataagttgactattcaacacaggttccacatcatttgctttgtatattaatagatgaactctgtgtgtttctatagtgaaagatttcagacacgactgtaaagtgctttttgagtgagatttgataccatctttaggtattccatacatttttttttttttctgacattgtaggaccattttagcctcatctctgatcaggtatagtacagcttaggtaagatattaactttttatagcttcattgtccttatttggaagtggtgatagtaactacctttctgagtgattgtgaagcaggtaattatgcatgtaagatgcttagtacagtgtctggcacatagtaatcttagctgctgttattattagcagtaataacaataataaggcttaggttgggttgttacctttctatatcctctttttttttttatttaaactgttatatatacaggatgttttttttttttgttttttgttttgttttgttttgttttttgagatggagtcttgctctgttgccaggctggagtgcagtggtgcagtctcggctcactgcagcttccacctcctgggttcaagtgattcttctgcctcagcctcccaagtaggtgggactacaggcacacaccaccacacccagctaatttttgtatttttagtatagacggggtttcgccattttggccaggatggtctcgatctcttgacctcatgatccacccacctcagcctcacaaagtactgggattacaggcatgagccaccgcgcctggccttcccctatatttatttaaggaaaaatcatcaccagatgcatttttaaagtaatagaaattccatcttaaatatattttatagggcattaagaaagttgcagttgtcactagggatctggaagtttttaagtgagtattcgtaatcaggaaatgttggcctagtaggtacctttaaagttttgtttgtgtttaatgtgaaatgtgtattttcaatagatgtgatgtttgtatagttaatatttttagactgcattttttttttttttccaaaaactgttttcaggctagtctgtatgcactggcagtctggtttgtattgaccgttaggtattgagttttaataaaatgttcaaatatgatggacataccacattatggtgagatgtgaatgaagattgtcccccacacccccaactgggttgtccacagctgtattcagtagaattaacttaaatggtccaagatactcttcaaaaatttgaataactatttgggaccattcagtaccgtgaaggctattaactgtgaattgagttaagcagaatactgtatgttttagttttttctttagtttttattcttctcaattttgaggaagttggggagagaaacctttcccctactctgaaaaattttctcttccttcctttttatttcctactttgaaatagccaagatcattgggacctctaagaatatcagataatatttacagcttgaggtagagtgattttcatgggaaaccttttctcataaagtaaaatactcttcgtccctgtcaggacatttttagaggagcagcagacagcccctcactactttgtcattacccagggcaagggaagggaagaatgaatgacaggtttcttcttttttcttttccaccacttgttttcctttccctttccttcctttcttgttacccttaggtgtctgtgggttctgaatttggatttcagcagaatggagtaatttttattaaactttttagggaacctggtactcccaaacagatagtgaatacttttttccattttggggtttttatttttatgtcacttttaaattattaagaagttttcaattatataatactagatagactagtttaataaaccactgtatatccatcatcaatttctataattacatggtcaatcatactttttctataccgttactactaacccccaatttattattattattatttatttatttatttattttttgagacagggcctcactctgtcacccaggctggagtgcagtggcgtgatctcagctcactgcaacctccgcctcccggattcaagtgattcttctgcctcagcctcccgagtagctgggattacaggtgtgcgccaacatactggttaatttttggatttttagtagggacggggtttcaccatgttgcccaggcgggtcttgaactcctgacctcaagtgatctgccctctttgacctcccagagtgctgggaatacaggcatgagccaccgctcctggcgtcccaaatttttttttttttttttttttgagacggagtctcactctgttgcccaggctggagtgcagtggcgcgatctcagctcactgcaacctccgcctcccaggttcaaggtattctcctgcctcagcctcctgagtagctggtattacaggcgcgtgccaccatgtctggctaatttttgtatttttagtagagatgaagtttcaccatgttgatcaggctggtctcgaacacctgaccttgtgatccacccacctccgcctcccaaagtgctgagattacaggcatgagccaccgcgcctggccccaaatatttttaaagcaaatttcagacatcatatgatgtttttggaaaatactatttgtttctaaaagagaaggatgcataagacaaagaaatacaaatacagcaccatgattccgccttttaaaaattaaagaattctgtaacatcagatccagtcagtattcacatttctgtgattgtatctttttttcccttaaaaacatggtttgtttttttactaaagattcaaaacaaggtccgtagcatttggttgctatttttaactctcctttcaattggtaatattttttgtttagctcttccttgccatttatttattgaggaaatcagatcatttgttctttggaatttcccacattcaaggttttgctgattgcatccctgtggttatttaacaagctcctttatttcttgtatgttctgcaaatagttggttagactttttagtcagattactttattgcatggtactgaatacaggtgatcccgaattcatgatggttcaacttatgattttttgattttatgatactgcaaaagtgatatgcattcagtagaagctgtactttgaattttgatgcaaaattctttataataactttgttataaagttttatgtcagggctttgtgttaggtgattttgcccaactataggctaaattaagtgttggatcatgtttaaggtaggctaggctaagctgtgatgtttggtaggttaggtgtattaagtgcattttcaacttacaatatttccagtttacagtggatttagtgggcgtactccattttatgtcagggagcatttgtatttcctattgcataacatcaggtgtcatgtgatgcctagttacctctcttttttttttttttttgatgttacagttgattactgcgtctattcttttgaagagatcctttatgtattataaagttccccatcagtctttcacctaatctctttaacagtcatttatgatcattgtctagaaaagcttgtctaattcccgggccacatgtggcccaggacagctttgattgtggcccaacacaaatttgtaaactttcttaaaacattatgagattttttacaatttttttttcttcatcagctatcgttagtattcatgtatttgtgtggcccaagataatacttcttccagtgtcgcccaaggaacccaaaagaatagacaccgctggtctagatcaaggtttctgcaaactacagcttataggccaaatacgatccactgcttattttgtaaataaagttttattaggatgtagccacacttgttcctttatgtattatctgtagctgattttcacatacagtggcagagttaagtagttaagacagactgtgtgtcctgcaaagcctaaaatatttactatctggccctttatggaaagtgtgctgaccccaatctagatcattatttctttagagatagccaatggtcatattctgtcattcattttgtatttgtagctggaatttttctataaagaagaactttacctcaattttattattatcctaagaaaaagcagggtaagtcttgattctttagcatttgacagaaacagtgaggtgttttcttctttttaagtacgtatttatatgaacccccagttttgagtatatttgatgttttaatctattctctttaattgtcaaattgttccatttttgccaatgtgagccccgtcatgttggctctttttatctttccatataacctcagtagtctttggtaatcctgttttctagtatgacaagatgttccaggctcatcttgtacatcaactgtcttagatatctagaaccagtcattactctaaggggttctgattccttttagtggggagtagtatttagagagcacagtttgagtgtttgttgctctgggttggtggttgtttttaggatcttttagtggacagagctagggtgtggttgtatgtctgtgtgattttttaaaaaattgtgtgaaaatacatatagcgttgtattgactacttgaacgattttaaagtgtacaatcattagcattaagtaccttcaccacgttgtgcatctattaccaattagttccagagcttttccctaccctgcaaggaaaccctatattcattaaggagtcactccccatttcccccttgccccagacctggcattaatttgctttccaactctgtggatttgcttattctggacatttgatataaatggaatcatactttatgtgaccttttgtgtttggcttttttccacttagaataatgttttcaaggtttatccctgttttagcatgtatcagtacttcgttcctgtttatggctgaataatattccattgtatgcatatattacattttgtttattcagttgatggacattttcattttttatggcttttggctattgtgaatagtgttgctgtgaacatttgtgtacaagtttttgtttgaacacctgtcatttcttttaggtctgtacctaataatgaaattgcagggtcatatggtgtttctatgtttaatttactggactgccaaactgttttccataatggctgcaccatttcacatttcctccagcagtgtagtagggttcccatttctccgcatcttcttcaagccttatttctattttttgattattgctatcctggtgggtatgaactggtatttcatggtggttttgatttgcatttccttaatgactatcgatgtagagcatctttttaggtgcaaaccaccattgttggtggtttgtgtattttctttggagaaatgtttatgtaagttctttgtcttttaaaaactggatagtctttttgttgttgagttttaagtgttctttatatattcttgatacttcacctttgtcagatattataacttctccattctgtgggttttcttttcattctctttttagtgccctttgatgcacaaaagttttaaatttagatgaagttgaatgttcctgtttttttctcttcttgcttatgcttttggcatcatatctaaaaagagcattgtcaaatctaaggttatgatttacccatatgttttcttctaaggggtttgtagtcttatttcatatatttaggtctctgatccattttgagttaattttagtatatggagtaaggtaagggtctagttgtacaaactccatttgttgaagagcttatttttttccgcattgtgtggtgttggcactcttattactcatcagttgaccatagatacatgggtttatttttggatgcacagttcttattcccttgatctgtatgtttgtctttatgccagtactacactgtcttgattactgtagctttgtcacaaattttgaaatcagtaagtcctccaacttcgttctttttcaagattgctttaggtattcagggtcccttacaatttcatatatattttaggattgcctttttcatttctgcagaaaaggcctttgggattttggtagggattgagttgaatttgtagatcaatttggatattgacatcttgacagtattatgtcttcctatccatgaacacaggatttcttttcatttgtttaggttttctttaattttctttagcaatgttttgtagttttcaagttttgtaaatcttcagttaagtttattcctagttttttttatttttctttttggatactgctataacttgaattgcttattagtatccttttcagattgtttattgttagtgtataaaaattctgatttttgtgtgttgatcttatattctgcaactttgctaaattttggaatttttttaaatgggaaaacacactgtgattcatatcaatacttccaaatccgattttgtttcaagattaagggtttagggttataaggttttctctctttcttagctttatactttttttctcttatgctggaaattttgagaataatgaattgctttattatttatatttatacatatatattagtatttttgaagttattgtgaaaaatggttgttgaaaaacaatttaataattctttgcaattagtataaatactagggaggtacagttaaattgctttttcattaattaattatttatttaattttttttgagacagagtctcactctgttgcccaggctggagtgcagtggcacaatatcggctcactgtgacctctgtctcctgggttcaagtgattctcatgcctcagcctcctaagtagctgtgattacagatgtgcaccaccatgccagctaatttttgtatttttggtagaggtggggtttcgccacgttgaccaggctgggatcaagtgatctgcccaccttggcctcccaaagtgctgcgattttttttttttttttttttgagacagagtctcgctctgtcacccaggctgcagtgtaatggcacgatctcggctcactgcaacctctgcctcctgggttcaagcgattctcctgcctcagcctcccaagtagctggtattacaggtgcctgccaccgcatcctgctaattttttgtatttttagtagagacagggtttcaccatgttggccaggctggtctctacctcctgacctcaggtgatccgcccaccttggcttcccaaagtgctgggattacaggtgtgagccactgcacccggccagcactgatttttgtatgtaaacagttccatattgatggacattagggtattaaatagggaaagtttgatgggaccagatacttggttcttcttgtggcacttaaactttaaaatattttgttttcatttttatcataaagatattttaccacggatactcaaaacagtatagaagtgcacataataaaaagtttaaaatgttctctgattcccatctgcccacttttcctctattcatatctccagaagttactactggttagttcaagactttttattaaaggcatgtgcagcatttatacactgtgaatgcttttttaaaaaaattgggatcatatcatgaatattctgcaacttaattttttaaacatagcattattagttaaccttttaagtttggctaatgagaaaagagtcctgaatataaataagattttacaaaatgattttgggcctttttttctgtgtcattgatactacatacatattttttgcatgtttgtatgcttcctaaatcttacagtattgtaatctgctctaaagtccagaaagtagggtatatattcctctttactttctctgtaggctcaaacttatgtttcgaaagaaattgagaaaacttttattgttgtaatcaattatggtttaatactaaaagtaaatccagatacagttgagatgcctaccagcagagttgaatgtaagttccataaaagtaggggaaaagccttctgatcttgttcatcactgggtccctagcacctaggacagcgactacctcctcattcttcccattctctgtatttgttttacgttttactatataaattataactggatcacgccattcaaactgttctgagttcttttttcacttgtcagtatatcatagataggttttcatgtcaattcttaagtatctgttcttttaaatgagtgtattaatggtgatactatggattacataattaggcaattttaagacttaatttgaactcctttttttccccttagactttggtattagtgttttatctttaaatattagtgggagatactaagttcagagttaggatggagtgagagtgtataacttgctgtatatttttgaagcaaacatgtaatgaaaggtggtacatgtaacatactttaaacaagtctaatatgtgattattctgactatgaatattggagtagttcactttaaaatatttttggcaatgaattcacatgaagaggaatacaaacagtcaacatgtaaaagagtgtttagtatagttaaagaaacaaaaataaaataagataaaattatactctgtgttccccaaattgttaaaaattagaaatgcaatacttagttgctgacaagggtttcaggaggtatgttgctaaaggataataaattcatatatggccggaaatggtggcttatgcctgtaatcccaacactttgggaggccaaggcgggcagatcacctgaggtggggagttcgagaccaccgtgaccaacatggagaaaccccgtctctactaaaagtacaaaattagctgcgcgtggtagcacatgcctgtaatgccagctactggggaggctgaggcaggacagttgcttgaactcaggaccccgaggttgcggtgagccgagattgcaccattgcactccagcctgggcaacaagagtgaaactctgtaaaaataaataaataaataaataaataaattcatatattctttctggaggacagtttggaagagtttcagaaattctgcttctaggacttttaagtaaaataaattaagggtatgcccacaaacttagatatacctgactgtttatgaaaattttaatcctggaactaataacaataatagtgtctaaacagtgtttcttttgtgccagacattacatttatttaatcttcttaacactcctgtggtattcacacagtaagattacaaatgatgatgatgtagaacattttacattgaaataaattgataatagtgaaaatagcacattgcaaaggagtatgtgcagtgtattttcagaaattgactgcaacaaaatctagagaatttgtgagaatatgctctagaatatttagtaaattataatcccgttattcaaatagattttgctatctttattttcgaatttttctacagtaaacatgtattatctaacaagaaacctaagttattttttaaaatgtcctctgattttataaaataatttatttctggatgcctataaacagtaaattcctcacgcatctttaaaatagagatttaaagtgttactaatggaattctaagcaacgcttggtcagcacacaaaataggaattgaaatgtccttgtacagcctttgatttctgccaatttgatggtgtttttttgatgacagaaatggaaagagtagaaagttaaggttaagcatctaaagtttgaagcctgacaacagaaaagtgacaaaatcttttaaatgtttatttgtggttgctggcaatgaagcattaccataaataaacaaataaataaataggtggacagattgactgactgacttgctacattttcatggtgttgcaatcaggtattttctttttcccagttgaaacttagtaactcatatttattgtttaaccttcctaagaattgatttctttcaaatcatctgagtctttctgtcccaagattttctcttttttgtttttttgaagcagggtctcgctctgtcacccaggctggagtgaagtggcgtgatcagggatcactgcattctttatctcccaggcccaagcaatccttccacctcagccttccaagtagctgagaccaccagtgtgtgccaccatgcctggctaatttttttatttttgtagagacggggtctccctatgttgcccaagttgttcttgacctcctgggctccagtgattctcccgcctcggctccctaagtgctgagattacaggggtgagccactgtacctggccaagactttctgtcttgctcctacccttttcaagaattgaagaaatttgttcccagattgctgctaatgtatctggtctgtctaggtctaatacctgtgttagtttaatgtagtatcgtctttaagaccttgttaaaatgtttttcttaggccttcttttacacctttttaaaaaaatcggcagctgttgtccttgaccctgcactttccatttcccttctgtgctatattttttccttctaacacttatcaccttgtaatgtataatagattttgtttatctgtgtctattcttctcccctccctctttccgattttaagatataagcaccattgatgcaaggagttttgtttttgtgtggttcattgtgtattcccagagttcagggtaatgcctgtgtagttcattgtgtattcccagagttcagggtaatgcctggcatataacaagatttcagtaaaattttttttgaataaatgaatagtgggcactcagtatttgtggaatgattagatgttctatttggttgatgaaagtgaaatttattctaagtgtttacagaagaaggttagggttctatagctgttttgtagtatggctagttcattgtagctccagtaatagagttaaatttataggcaggttggaatttcagaactttgttccctgatacatggtaggtgcttattaagtattgctaaatgaatgtcaaaagaagctaataaagtgagtggggttgggggaaacctagcttgctgtgagattctggcctaactggcatagccttttgggtgaggaagttgaactaagatttcagagctctcttgctttctcactaagattcacctccagggactttgtgtttgtaagtatattcctttggaattcggaagttgtaaaaaagtgaattggatgaaacttgctgtaggattgcttttttggaatcaaaactgttgttgaatgttaattagcaccacagctttgtgtgaaggcttttagacttgagtgataatgagattcattattagagacagtgtgtgttaggtgttccaaggcttccacttttattcatccagcacttgagaccgactatatgctaggcattctgctagactcctgaagatacaaaagtcaaatagggcagtaacgaagagaagaagatataaatgtttgttccagagtgctgctttttgaggtttggggtttcccaaaaggcaggggaatttattttattgagtatttttggttttgatttaatttaaattaaaagtgaatatgagccattaattactgagaatatgtttaatcagaacataatttaatatcaatttgctattgagtcagtatgtatgtatgtatgtcttttttaaggaattagcatttattgagtattatatcttacactcttgacatgcattctttttttttttttttttgagacagagtcttgctctgccactcaggctggagtgcaatggcgtgatcttgactcactgcaacgtgtgcttcctgggttcaagcaatcctcctgcctcagcctctggagtagctgggactacaggcgcatgctgccatgcccggctaatttttgtatttttagtagagacagggttttgccatgttgcccagtctggtcttgaactcctgagctcaggtaatctgcccaccttggcctcccaaagtgctgggattacaggcatgagccaccatgcctagccacattattcttttgacacagtcctcataataatcctgagggagaagcattattacctgtattttacaaataagaaaattgagtttagagaagttaagtaatttgtttaaagtttacagctttataagttgtggatttcaaaccaggtttttttttttgatactaaagtacattaacagcaagacatgctaaagattactgcatcttgagatgtaagaatttctgtatatttactctcaggagactctataactccaccatccaaaattagcaactaattaccgctagtgacatgtggctgttgagcccctgaaagttggctagtccaaattgagactggctgtacgaataaaatacacaacagattttgaatacttagtttggaggggaaaaattcattaataagttttacattgattactgaaatggtatgatttatattatatttttacatagtagttaaataaaatatattatcagtataaaaaagtacattagctttttgtgatgcttctcaaaggaagttattatagtttttttctttgtctgtagataatgttaaataacaagatgtattgactttgaatactgtactgtaactgttccccccaaattgaatctttggctttttaaccagagtatttaaaagtacctttttccttactaaataaataaaatatttttgttgtagcaagcaatacaagattggtggaaaagaataaagagaaagaagaaaatatagctgggatagggagagagagattttgattgatcccttttaatcctttgatgtacaggtggtatttaacataatacagtttgggcttctgactctctgagagctggagtcaaatctgggctccactatttaatgggttaatgattggtgttttcatggttttggggtaagttacttattccggttttgcaaatgagagaactgaggtttagagatgtcagcctatgtcatagggttttgaagattaaatgtgacaaggcatgtaaaatgcttggaacatacctaaaatttctaggcacatcctagttctttgcacatagctatattatcttttactaaaatgagtcaacaaaaattgtaactgcttattttgaacttttaatatatatctatctttccacaggaatcaaaacatttaaaattatcagatgataactaatatatacaatggaatgctgtaatatattcagtatcctcttcttgctagacatttaattatttccagttttactttttgcagttaatgaaatgcataggtgaatattttcgtagcagaactttacctatgttcttatctgattaggatagagttggttggttgaagagtatgtatactttaaagatttttgacccattttgctgaattgccttctagaagttcctagtcacgttactaggatttacatgtaatcctagaatttatctggcgacctgtttttctaaattcttattaaatgagatattataatttaaaaaaaattgttgatttaaccacttttaatttttacattgctttgattactagtaagttcgaaggttctttttctttttcttttttcttttttgagacagaatcttgctctgttgcccaggcgggagtgcagtggcacgatcttggctcaccgcaacctctgcctcctgggttcaagcgattctcatgcctcagcctcctgagtagctgggaccacaggcatgagccaccacgcattgctaatttttgtatttttttagtagagacaaggttttgccattatggccaggctggtcttgagctactggcctccagtgacccgcctgcctcggcctcccaaagtgctgggattataggcatgagccactgtgcccagcttctgaaggttatttttctacctgtatattggccccttgtgtattatgctgttttcccattagtgtttcatgtcctcattcccattttgggggtatctgtcactcttctggctttggggtcatgctcagagaaacttgctcttcctcaaactataaattggcagtgtttaaacttcttgctctcaggactccttagtactcttaaaaactattgagaacctctggtcaggcacagtggctcatgtctgtataccagcactttgggaggccaaggtggggcaatcacttgaggtcaggagttcgagaccagcctggccaacatggtgaaaccccgtctctacgaaaaatacaaaaattagctgggtgtggtggtgcacgcctgtaatcccagctacgtgggaggctgaggcaggagaatcacttgaacctggaaggtggaggttgcagtcagccaagatcgtactagtgcatctccagcctggacaacagatcgagactgtgtctcaaaaaacaaaactattgagaacctcaaagacttttgtttactccatctcaaaaaacgaaactgttgagaacctcaaagacatctgtagatctttgccatcttagaaattaaaagagacatttaaaaaatactactttattaagatatagtaagccatttacatgttaacatttttaatgaaaattacattttccagaataaaaattaccaagaaatttttcatttttgccagtctgttgcttaatagaagatacctggattctgtatatgcctctgcatttaatctgttgcagtatcacatgatgtgtaccctctggaaaagttgattgtataatcaggagagaatgagaattaaaaaggcaaatgatgttattatgaaaatggttttggctttgtgggcttctgaatgggttcaaagatcccaagactacagtttgagaaccactgatgtaaataatcacttatatattttccttttgtgtttttatggctgaatttaaattccgaataaattctagtggaaattttctaatttctcccctctcctgcctaccctctccaattggctaatattctttgttgaaagatccattttcatgtaaataattgactgactaggatgaaacagtagacatatctgctttttcccagttgttactgagaagctcactgttcattgaaaacagtagtctttgcagtctttccttaatgttgtatctctattttggtatattcccttattttcttgatgtgttctcccctcctcactctaactcccagttttacaacaaattgccctaaaatcttaaagatttttcattcatactgattgtggcattttgctctaaatattgtttcctccctgatttagaaagtaatttaaaatgtacatagaaacataaaccaaaaaagaatattttgtcgctttgaatatgaaaagtgatattgatatactattattgctgtactgtgatggagtatatcacaggttatggaggttatttcaatattgaaaaaatgctagttgagtatacatacattcctgcaccattgttaataaattatattctttttgatgtgttgcttagcttcttgggaccctttactcactagacttttattgacagctgtgttccaaaatacagttagataaaaagaacaagacacatttttttttctaactttgcaggttggtagagaaatacacatgttctgtgataagtattattatagccacaaacaattaagaatattatgggagggcctaggcaagatatcgttaagaagtgagggaggaatgaaggaaagggaattgatttgaaaagattaaggttgtacaatcttcctttagtagttgatgatttctttaggtgtctgagtccaaaggagagagaaaggaggagtcaagaaagacacccagatatttgtctgggaagaccagatggataccagtgtttatcaaaatgtggaataagccctacttccaacaaaacaaaactcattacatgtgctacactttcaaagacactgtgttattgttttccaagatgagttcaattgtgggcatgttagtttgaggggcctagggtggagatttgtggaagccatttggagagctatcaggagacttgtatgagctgatgagcagtgtagcataagtaggtgaaggaaatgcttaagatcatcctttgagaatttttagtaggagaagagaagatggtcagagaccctctgataattgagggacaggcagagggaaataaattaagcaaaacataaaaaagaaaaaagggtcaggttgtgttgaaggcaagtgaccagtttaaagaaaaaaggttaggggagagagggatctgaaatgttaaatcctgcctaaaggacaaataaaatctgaaaagtgtttattagatttaggaataagaaggcattgatgactttggaaaaagcaactaaactgggaagttgggtgtagaagctggagaagtggaggatatagaggtgaggaagtggaagacagtgagtattgactaccgtttgaaaagtttgcctatgaagctggtggagtacaggttaaaagtgagcaagcagggatttgaaagtgttaatgttctagggaaggagagagaggagggaatgagagattgaactaaggataaaataccatcatcttgaactttctagagaaatgttttttatgcttcttgtttgctctttttgtaaaaattgttctaaggtattttttagataatgctgaatatcttttttgatacctttatgatatttcagctaccatgaatatcagttattgtactgccggtatcaaagaatatattaatagcaacagccctgttctaaaatacaacagttcagggctgtattagttcattttcatgctgctgataaagacatacgcgagactggatacttcatacaggaaagaggtttaatggactcacagttccacgtgactggaaggcctcgcaatcatggtggatagcaaggaggagcaagtcacggcttacatggatggcagcaggcaaagagagagcttgtgcagggaaactcccccttataaaaccatcagatctcataagacttattcactatcataagaacagtatgggaaagatgtgccccatgatacaatgtgggtccctcccacaacacgtaggaattttggcagctacaattcaaaatgagatttgggtgggacacagccaaaccattcaggggccgtttaatgtaattatatagtagctttttaatagccagttttatcttttccttgtgggtgcacatgtgttttgcccactaactcattgatgtggttggtcctagcaatagtagtccctattactacaaatgtgctaagtaactgtgaatgggaaatccacattaccaaattgttagaattgttctttgaatgaaattactgattttaatcattagattgtaaggaaggtctggggattacatctgtaaagtgtactctgtgatagatgtagttttctttttcttttgagccagtgtctcactctgtcgcccaggctggagtgtagtggcgcgatctgggctcaccacaacttccgcctcccagatttgagtgattcttctgcctcagcctcccgagtagctgggattacaggctcccgccaacacacccggctaatttttgtatttttagtagagaagaggtttcaccatgttggccaggctggtctcgaactcctgacctcaggtgatccacccacctcggcctcccaaagtgctgagattacaggcatgagcaccatgcccggcctatagttttctactgctgttttacttagaatgtagagctctatagtagagctctgtagttagactgctttagaatcccagctctaacacttacgaactctgaccttgaacaaatgacttacctaagagctttcttcatctttaagttggtgataataatagtacctatctcacacggctattgaggttaaatgatgtaatgcatgaaaaacatagcttggtacaaactaggtacttactcagtaaacagctgttaataattattttctccctgcctccaggattttgattttctaagcttgatgtttttggaagtcttcttgattagaaagatgttactatatttgaaaagggaatagagtcaactagagtcatattgcaaattttatgtgtctgagagtgtaatgagaatttgcagcatatgtaaacaagtagatttatttttctgaggtaggtccatggattgttaacagtggtttagggaaggttaatgtgtttaaaaatgagaggtaaaaaataacctagaattaccatatgggatattttacttggggtgcgttgggggttactgtggactagctattttaggacaggaaattaacataacctggtgaggctcatgaatctttcagccttagtatcagaactactcggtaacctgtaatttaacccatggcagtacttggagggcaggaagttagggtttgtgcctggtctgagaaagcctattttcacctttttatgagacttgtgctcttaaaagacagagaatgaaattatctaagacatttgacaaccatcaatttagtcattttcttccttctgtcaaataaattgtactattcttttaaacattttagagccctgttgaaaatttgcaccttttttatatagtaaaacatcaatggatggaaaatcctgttttctggggtagccctctttattgaacttctctaacacccacagatcccgtttataaagaagaggcacatttagtatgtttaaaaatagtgaactctttccttctgtgaatttctcaggatgtgttaatggtgtttaaagtttaatgctgctaaacccacacaggaattttcactggttttaaatgccacaaaaacagtttttacttttgttgatagctggtttagtaaaaagaccttgttgttaaattcaggaatgatttaacaaattattaaaggtttcattgcttttttcaaaatacttcctatttgcaagtctcttcatatctgcagtcataccaaattactcatatcttttttttttttttttgagatagagtctcactctgtcgcccgggctggagtgcagtggcatgatctctgctcactgtgacctccgcctcttgggttcaagtgattctcgtgcctcagcctcctgagtagctgggattacagatgcccaccaccacacctggctaatttttgtatttttcgtagagatggggttttgccatgttggccaggctggtctcaaagtcctgacctcaggtgatacacccgcctcagccttccaaattgctgggattgcaggcgtgagccaccgcgcctgacccagtatagtgctttttataattaattgtgtacttttaaaaaacctttattcatgataaagtaaatccagtctactgagtggctgtgctatatctttaacagctattttatctttatttccttcagtgttacagcttccctacatagtacagattatgttacctaattaaatagttgagctatgtcccattctagttaagaaaactgtcctgttgcctattttcatctgattaaaattttctgactttctttcaatatctgtttttttgtaaaccagcctattcagttttatgtcccaacgtcctgttctcttgcttgcccactgatttttttctccatcattaaaaaaaaaaaaaaagaaattgatagacagcatctggagtgtcctagaccctactagacctggtgtttggcccactgatttcttctcaaaatgattgtatcctgtagtttaatttagctacagtgacccataacacttcactcaaactgcttttctgcctctcagattgtttcctcagtcttgcctgcagagcacttctctacctctccagccacctaccccgtttaagtgtttatattttgacctcagccatttgttcatactctgcctaccttccttctaccctagtgatctggtttatgaaaaactagatattaattctactgctttgaccagatctagtcctctaacccagatcttttcagaatcctagacctgcaggaattaccacctacatgttccacagctactttaaactgaacatgttaaaactgaactcattttctctcctgaccttctctcctcccctaaacagaatgaaacctctatattcttcttcttcctctctatcttggtaaatggcaccattatccatagacgcccctggcttccttcccaggaaccaaagttgtttcctccttacacctagttctccaccagtagtcattcttcaagtctaccccctaaatatctttggaacctattcctcttcatttactagagccttcactttagtttaggaccttatcactctgcctagaccagaggtttgcaaactatagcccataaagccaaatacggctagccgtctgtctttgtaaatcaagttttattagaatgcaacttctgtggtttagatatggtttgtttttcagcattaaaacttgtttcgatcatcaatgtggcagtgttaagagatgaggcctggtgggagatgttttgtttcatggggggtggatccctcatgaatggctttgtacctttttgtagcagtgagtgagttcttgctctggtggtactagattagttctcatgggaatgggttaattctgagataatggattgttataaagccaggatgccgccagattttttgtcttcacatctgaccacttcccctttgagcttctttgtcattttgtgatgtagcatgaaagtcctcaccagaagccagggccatgccctttaaatttcctagcctgtagaactgtgaggtgaataaaccttttctttataaattacccagtctcatgtattctgtcatagcactacaaaatggattaaagcagctgccttgctcattagtttatgtattaattgtctattgctatttttatgctacgaaggcagaattttgtagttgtgaccgagactgtatggcttgcaaagcccgaaatgtttactgtcgggccttttacagaaaaggtttgcctacccttgggctgttacttattttctgtctccagccttgtctattcttctgtcctaccccaggtaaacattcagaagtctgatcatatatccccccatatgaatcttttatttactactcatttgttacagaataaagcttaagtcctttatcatggtgtaagatctttggctacctttctgggtttgtcttctgttcagaaggcatgtggatttgtctctagtatatgcttttacctctgccttaaatgttctctcttctctttctcgtcaacttacctgtctctcaacatctaatacagatgctaccttcccaggaagtcttctgacattccagatttttctttttcttgcttccttcataccctctaccgcttttgttgtatttaacattgtgcagcttttatccatttgaccgtatttcctttgtaaattttaaaaccacccaggccaggcctggagccacattcactgaatattccctcaagaagtgtctgttgactgaataaatggattgcttttgtgttagactcttcctaactagtcagtcagagcaaggattatgtctcatacaatttttacattctctgattgtatctagggacatgctagattcatagtaggtacagatactgattaaattgttgatttgaattgtcatgtagataaagtgaagagacagattcattttggagggttggacattcatcaaattgagactgtctagcaaaacccattaaaaaggaactagatttgccttagctactaacttttcccttgacatgtatagggattttttcccttttctgtacttgggaatttgattgactttcattgaatgttttgtgtcattggttaggcatcatatgatctgttttatgtagtgcaaatataagtgacaatttagatggaatgtgctaagttgatgtattaacatagggcagagtcatactaaaagtactgttattctgggtataaatgggagcagaaatggagtgttaccaatgacattagctgttaggatccataccttttcttaactggattaatttgcctgtttgtggtacctaggtgccaattctggtaggactcctagtagcagcagtagcagcagtaataaacacctatctagtgcattttttatgtgccaactactgttctaagcacagtacataaattaactcatttagtcttcataacaacccaatgagataagattctattatccctgcttaacagatgaggagcctgaggcacagggaactgaaataaataaacttggctaaagtaatacagttagtaagtggcagagccagtatttaacccaggctgtctggctccagagtttatgctcttagtgactatgctagactttgtatgagtcttggaacaatttgaatgcacagttcttgcattccttttgttcccctctaagagaaactctggagttttggaacagaggaggaaatgaggaaatactaaatggttaaaggtaggtttatttttaaaaggcacttatatcacattctatatttattatgagtagttgtaggactcctttttagcatcattctgtttttatcttttcctataagttgaggtaagagtttggttttcttttaccttatctgtagccagttgatgcttactaaatgattggctttaacggttctagacattgtaattgtatttacatagttctgtgaagaactgtgaagatacgtagttcatatatttggaagtgtattaacaattgtggtaatcaagaggaaatcatcattagagctggacagcattgatattctaggtgttgcttttaacaggcttaaaaaacacttggaggtagaacatatacagcagattgcataaagcctaagtgtactgctcagtgaatacaacacccagatcaagttaagtacagaacatcttcatcattattccagaaggctttcatgtgtcctttcccagtcaacaaataccctaccagttatgttgctttgtttttaaaatattgtatttattaccacttgacatatattagtttgtcttccctaatggagtttaaacttggttttgctcatcattctgttccattgctcagcatagtgcttgacatatagtaaatgttacacaactatttgttgaataaattaattggaaaacacttatttgttatgtaccatattctatagcacacagatgctattttatgtactagttgcttgaggatagatcataaatcttagttttctaggatgaggcttttgatatttagtgtgagacttacactacttttaagacaaggaaattgacttctcctggcattgggtttagtgtgtgttcgtctttggttacgtgaatgttagggaatatgagaaagttgctaccagtgagagatactggtagcctatctatctggattgccttcttgagagtatctgattccttatagttatctcagattcttaacttacactcttgatgatctttttgaatacttcaaacagttgcaaaagactaaatgctcttgatcttgtggagagaaaattgtgataggaaatacattacttatcattaatttacttataagacagtttggttatcaccttacattgaaaataatgaaaattctgtttgcctatcgtgaaaaggtttaagataaataaaggagtctgtgctttagaatttgttttagttcaaacttaggttcatgtagaaaagaaaataaattttgagctgaaagtgactctagtattctgattcatgttcctgtctgtcaaatttagctctctcattttacatttgaggaaactggagtgcaaagaagtaatgtgatggtctagtctaaaattaaaagtttaggctgggagcggtggctcacacccataatcccagcactttgggaggccgaggcgggcagatcacttgaggttaggagttcgagaacggcctggccaacaaggtgaaaccctgtctctactaaatatacaaaaaaattagctgggcatggtggcgcatgcctgtagtcctagctacatgggtggctgagacgtaagagttgcttgaacctaggaggcagaggttgcagtgagccaagattgtacccctgcactccagcctgggcagacagagtgagacgctgtctcaaaataaataaataaataaaattaaattaaaattttaataatgaataaactttccaagtgatacatactctagtggcttagaaccattgtgctgtcatgcctttttctttcttactttttaagaaaagagactgttctttaaagcagttttaggtttacataataagtgaacagaaagtagagtgagcctatataatcccttacccccacccatatttccccatttcccctgttattaacatcttgcattggtgtggtacatttttcccggttactaacatcttgcaatgctgtggtacatttgttacaattgatgaactaatattgatactaaattattaactgaagtatataatttaccttttataatttccattagggcccacactttttattgtatagttctgtgggtattgacaaatgcataatgtcatgtatccacgattacagtgtcatgcagaatagttttactgcctccagaatcccaacatccacctatttagccaccttctcatccccctcctcccatcccctcctcccatccacctcctcccatcccctggccaccactgaactttttactatctctatagttttgtctttttcagaatgtcctatagttggaatcatacagtatgtagcctttttagacgtggttctttcacttagcaatatatttaacattcctttatgtctttttctggctggatacctaatttctttttgtccctgaataatacctcattgtatggatataccacaatttttttagctgcttacctattgaaagacatcttggttgcctctacttttgggcaaatatgagtaaaactactgtaaacatttgtgttcacttttttgtgtagacatgttggaagtaaatgcttggtgctgcaaagtgaaaccagcactcaggcaaaagttttcttagcaaggcagtttatttttgcagaagggcgtgtctcgtggatggagcaatggcgagagcacagggaacaaaggagagaaggggtttttatttctgatgtgtagtccctacctcagtgtcactcccctgtgggctggggttgaaccgcacaatttaagctgacttgattggctacttgtgaatttttttttttaaataaggaagagggaaggggaaggtgagttacagtggtgggatgtgtggtttcgaagggtggacgtgtggtttcaaaaaggtggaatgggtgcagagtgggtaaccaagggaacagatgtgagttattgattagagctgatgggaaggttgtttacagtaactagaggcaaggaggcatggaggacaagaaagttgagtttgagaacaaagaacaaggaagttaacaggctgaacctttgaagagaaattcattatatcttacagttccctgcttttaatttttataatcttttctctttaaacctttttaacatgtctgggctttgctgtccgacttgatcctttaaaaggaaaagcttacttgcataaggtggaggagagctaagggagattttagtaagtgctgtttttataagcgtttgcactaacctacaatgcatggtatgacacagcacccaataagaatgagtacaagtattacaactgcaagcaaagtaagaattgaggccacgatttcttttcatttattgaaccacctttttagccatcttgaaaaagagttgttgattctagttaattcattggataaagctgtaatcccttgcaagaccttcactatgctctcattgggggcagtattgtttgggatgaaggtacaatattgagttttcattataatacaaactccacctttttcagctaatatcatgtctagggccattctgtttttccaagccattcttgctagtaggacctaattggtcagctattcctttgacagcatctctggtgtaattaataaatcgctgctgattataatagatgtaatctatccaatctacatttttgttgatacttacccatggaggcaatgattcaaatcctgcagctatttggtcttgggctttgaatttatcaggtactccctgtgagattccaattgcgtctaaataaacttaagagtcaaaagacccataaggggcttctcttacttttcagtgttgtgattttttttttttctctgcttgatgaaatgccagggtgaaagggatagccaactggacaagagagcaagtgctgctccagttacttggtggagtgcccagtaagggtccaccacaataccatcatatacctgctcaaggatggctaagggcagactgatgggtaagctcttggaagggcctaagctcactgtgttccattaagcttcccaggaacgccaagtttttcccattccgtgagagacacaaggtgaaattgacattgagagatggaagctggatggcccttgggggctgacccacagggtgttggactttgggatagagcagggagagagcttggcatgatttattactcctagctgtagaatcctggaagagagttaccatgcagcccatgcccggttgactggagcaccattcgagtggaaaggggacaatctgggcctctggccggccatgcacacaagcataacaattgcttttgtttaatgtgcggacagaatttttgatgcatttcaaccagacatttgtatcttgatatcctattttaattgctagagtttgccttagatcatctgcttctacaatgtttacttcagcattgttgtagggcagggtagaaggtttagatggagaaggaggagggaggttaataaaacgcattttaaagaagcatatagggttgtatccattggtctttactcttaagccgtaaaagtgctctgaagggggtgtagggttggtggaggtaggagtattaatggaaatagttactgggttacaatggtgagatggacaattagagggggtggttcctttggtaaaagtggaggtaagattttaggttggtacagccatctggggaggtccagcccggatctctggtagtccagatgatatctgcccattgggaacatatctctcaatttggggtgtcttggtacccctgccatgagcaagatgcagggtcagtggcttttctggaggggcagaggtatttttctaaagtagagatttgtctttgtcattgttcgtcctcacaaggtgtaacaagacaagcatcaaaggtaatgatttggggtgaatctgacctagttacattgataacaaggtggtcatcaatagaatgagaaaagaagaaagtaatagaatagatggaaagatgaaagagagttaaactttttgtagctttagtttgagaggtttttttctttggataatggcccatgactctggaggtggtggtgttttcttgactcgggtgtgatgggtccatcctttttttgctgtctggactgcggttttagtggttagaagccgcaggtaaggtccttcccaggccagctcaagtttttcctccttgatgaggacgtgatccccaggctgttattaatggactgagagctccagggttagcgcctgtgctaagagacctttggttttaagaaaaagagaaagtaaaggagagaccaagtatataattcttgaggaattggtcttttgttttaaatgtaggaatgtcagcagtggagtgtaaataaggcagtctgtagagccatctcataaagggaaaggcctgtatctttttgaggagcagtttggattttcatcagggcaattggaagacatttggtccatggcaactgagtctttataactaatttggttagatggttctttaagctctgatttgtctttttaaccttccttaatgacggtgggtgccaaggagtatgatattttcatttaatgtttagggcttgggatagctttttaatgatatgagctgtgaaatgggttccattgtctgagtcaatattttttattagcctgaacttgagcactatatttttaattaatgccttaactatattattggctgttgcatttgacccagtgagtgaggtgatctattgtcattaataagtattttagacaacccgttagaggcatctctgattaatttgggtactttggaatggtcttaagcctggactcctttccccaagggataatctcttttatagtttgtttatcagttttcatatatttacatataaagcaactatctataacccgttttggctagggtataagttccatgaagactgcatcacacatggcctggggtccttaatgagtcctttgatgcaggttggacaagacttggtctgtcaatatctatttttattctgagctctctttagtgcctgttcttactagcttttctttctcagtggaagagaaaacagagatcacagtaggaggaggaaggtaaggagttaagtggagaacaggcatttcagaagaaatggcagcctgtttggcttatctgatctgttaggttattttcctgactcttaaaagacaggcttttttggtgtctggggacatagacaatagcgattttctctggcaactgaaggttattcagtgctcgggtgattaattccttgtgaacaaggttttgaccttactgttaataagacctcgttcagcctaaaatctttccaaatgtatgagccaccccaaaggtgtacttagaattggtatagatggttcctcctggttctgcaagtactttaaggcttggctgagtgcaaacaattcacaagtttgggcagaccaattattgggctattttcttgactctatttctttaagagcgtctccatcaattactgaatacccattgtgtctttttccttcaatcacttgggaggagccatctataaagaactgtcgcccagtcctgaagtgagtttctcctaactttggccggacctttgtatggtaatcaattaaatctaaacatgtgtgctttctttttagatttagaactcttgttaagaaacctcctgggttaagcaaattatcagtggttaaatcatcctcttgttttaacagagtagcctcatactcatacacatgtaatacttttccagtggtatattttaatataagtgactctaaagaatcaaagttcttttctggtctggggtagccaacaatgatcacattgaggatttccttatagaagtcttttttgaaggtatgcatgatatgtgtttccatgggcttctttgtattcttgtagtatgggttccatcctgtgctcaccaccatcttatggacatctctacttccagcactggcccaactacaataaatgccggtggatatatcagctggaaggttatctaccacttactcaggaaaattagctgcagggatgcccagctgcttggagccgcagccaaatccctgcaccacttggccccagcagaagtaaggcagatgtcccataatgtagcctgttcggggtttggcccgtggcctggtctgcatgttttgcagagccaccctttacatggtgcctgaaccctggaccagcaagggggcaggagcgtgggctctgcctactgtggaggcacaccagggcccggacgatgccagtgacacagtgtggcagatccttgatgctgcccaccctacaaaagctgcctggcgacttaggtgcatggagtccctttaaggagggatacttaggctgggagggaataggttcatagggtgttgattcccaagagtgagaattgtaaggaggaggagtaacatgagtaggggaaggatatgaagcaggatctaggatggcaacagctgcctgagaggaaagggttggggtcactgagcaggggaagacagtgtaggggatcccatgtgatggtctttaggcatgggaactggcttttctaaccttttattatctttccaatattttaacatgagacctagggggctaggtcttttaacaagagattggggggactatctttgttgctgtttttatcctttttacttcttgtcttgcttggcgtatttcccatgttgggtcctagttaggctgaatcccttgtactaaagacttcttgcctattcttctctgggggcttgttaaggctcaatcctttgtatcagtgatttcttgcctattcttccttggggagcttgttaaggctcaataccttgtgttagagatttcttgcctatcctttagccccacctactggaggtttcttgcacccttttttcgctgcatctgctctggccacttccctcgcgggaatatttcaggtccctcttagcattgatggcgggtcagtataaactcctgacaggatccccaaagggccgccctaagctgtatgaggtgaccacggaactgccgataggacccactcactctgcacagcagtagtgcttagtaccattcacacaagcagcactgcaagcagtagtgcttgtgatcatttacacacttttaacctccagaatcccaaccaccaaggaaatactttgtcgcccctgcgaccttctgactttggtctgtgcacagagttacctagtcgctgtggtatgtgagcctcctttccccgagctgccggcctgtttctttcccatgttgctgagagtctgggtttatttgtcacactgggtgggcctcaattcctttatccctgaggctgctgcaacgaggcagtggggcgcacctcttcacgggagaggaccaagaccctttcccagaggagaatgggaatcccggatgagcccgcagatttgttggagataaatgctcagtgccaaaagttttctcagcaaggcaatttacttctgcagaagggtgtatcttgcagatggagcaatggcaaaagcacagggagcacaggagagaaggggtttttatctctaatgcatagtccctacctctgtgtcactcccgcatgggctgggttcggactacacaatctaagcagacccgattggctacttgtgaatattttttccaaataagaaaaggggaaggtgagttacagtgatgggatgtgtggtttcgaagggtgatgtgtggtttctaatgggtggaatgggtgcagagtgtttaaccaagggaacagttgtaagctattgataagagctgacaggaagattggttttttttttacagtaactagaggcaaggaggcatggaaaacaagaaagttgagtttgagaacaaaaaacgaggaagttaacaacctgaacctttgaagagaaattcagaaaaattcattgtatcttacagacataagttttcaactcacttgtgtaaatatctatgagtgtgattgctgggattgtgtggtaagactacatttagctttgcaggaaactgctgatgtgtcttccagaatggctgtactattttacatttctgcaaacagtgagtgagagttcctgttgcctcacattttcatcagcttttggtgttgtcagtgtttgggattttagccattctgttccagtggatgtataatggtatggcattgttttaatttacaattccctaatgacatatgatgttgagcatcttttcatatgcttatttgctacctgtatatcttcttttgtgaggtatcagttcagattttttgtacactttttaattcatttgttttattattgagttttaagagttctttgtatattttggatactagccctttatcagatatgtgtattgcaaatattttctcccagtctatgtcttgtcttttcatcctgtttcatagtgtctttctttcaaagagcagaagtttttaattttaacgaagcccacttttccattttttttttttttatggactgtgcttttggtgttgtatgtaaaaagtcctcgtcaaaccaaaggttacttggatttttcctgttatcttttagaagttttatagctttgcattttactttaggtctatgatgcagtttgagctaattttttaatttttgtgaatttttgtgcattttgagttaatttttgtgtaagttgatgtgtgtttatttgcatgtagatggtcgcttattctagtaccatttttcagaaaaagactgccatccctttttgatgtcctctttcaatgatgcacatgtttttttgagacagagtctcactcttgccttggctggagtgcagtggcttgatcacagctgactgtagcctcaacctcccaggctcaatcagtgctgccatctcagcctcccgcgtagttggtatcataggtgtggaccagggtttcaccatgttgcccaggttggtcttgaacgcttgggttcaaggagcacacccaccttggcctcccaaagcactgggattacaggcatgaaccaccatgcctggcctcagtggcacagttttgagggtgaattatgataactaaaattgaagaagaaacctgagattgttgtaattaataatttgacttcatattatgcctttaaaaatagcttaggaggaagtaacatgttgagtaagcgcctgggctctgggataaattccgaatttgctatttacttgctgtgacattggacaagttactgaactgtgcctcagtttccttgtatgtaaatgtatttttaggttaaatgaacaatatttaatataaattgtttagtacagtatcttgcatatgttatagctatggaagattattagtaataaagacttctttagatgttttatatattttttattcatcagttcctcttattttattctctatagtgtattacaggaaaatataatagtgttaacctatggactgtttgtgtagttattgatgtcatctggcagaagcagatttacataagggagtttaatgactgacctagaataaccatcataaagagcataaaatgtaaaaaatggagttctaaatgcatgcttatatgctcagtattttctgaaaccactaaataccattaaatacctcaagactaaaggtttgatgtgtaagatggtaaaaaaaaaaagagctgtagtgctttatttttattttttatttacttctttttttgaaatagggtctctccctgtcacccagtctggagttcactttataaaacatatttccttcctttgcatatgacgtattacacactaccggcttgcttaatatatttaggattgtttcttattttagattttgagttatctctccaataagaatacaagttcttcgggataatgtgtcttcagtttttcagtctgtcaccatttctggatggagaagctcatgtttcttaattgcttaagaagttaagcaatttggataattggataagctattacttttaaattattgctttattttatcttgccatttgattcttccttctagagtggatagagggaccatttacaatataatagcgaaatgcatatctgtttttaaagttagaactggaaggccagtaagataaacaagggtcctttagggcaataccttgagtaaaatagcaaagcaatttttttgtttgtttcaagtaattgctagtgaataaggagtatttctatacttctgtatattcagcatttttctttgtgttaggaaacaagatgataattgtttaatgcagtgataggaactgctgttctgctgagttatcacagactttgggttttgtcagttaaaacaaattttgagtgttaggttttgttttttaacaggtgttgacattaattttatttctatgtttgggaacttgtaaatttgtctgacattgattctaaaacttttattgtagcatattattttgatatctgtgtatccaagcttttttcctaaaactgctagatggcatagatctgctttttttttttttttttttttgagtcggagtcttactctgtcgcccaggctggagtgcagtggcgtgatcttggctcaccgcaacttctgcctcccgggttcaagcaattctcttgcctcagcctcctgatgagctgggatttcaggtgcccgccaccatgcccacctaatttttgtatttttagtagatacggggttttgccatgttggcccggctggtcttgaactcctgacctcaagtgatctgcccaccttggcttcccaaagtgctgagattacaggcatgagccactgcccagccagatttgctttttaaattaaatttttgtctctgagtcatttagtaattaagttagtcatggtacccccttctcttagatactttaattggcgatactctgggggtattgcaacaacaaaactgatgatcagaattagtttaacaacataaatattcttaatgaggatatcttctgaattatcataataaagtagtgtttatgtttttctgttacataaaaaaatcgaggactttctttggtaagttatacttcataccagttttagtcatttagcacattagagagtaccagtgagctatacattatgtagactatagatatgttaaaaagtacattctattcatgataaaactaataaactaggaatagaaggaaacttgctccatctgataacatctatgaaaaaacccggagctaatacatagttaatggcaaaatattggatgctttctgcctaatattaggagcaacataagggtgtcctctgctgccacttttttttaacattgtactagagtttctagccagagcaattaggcaggaaaagaagtaaaaggcatcagagtgataaggaagaagtatagctatctctatttgcagatatttatagaaacatcctaagaaatttgctaaaaatctattagagttaataaatgagttcagcaaagttgaactcattttatgtaggctcaatgtataaaaatcaactatgtttctgtacagtagaaatgagcaaactgaaactgaaattaaggaaacagttccacttataatgccatctaaaggaacaaaatacttatgaatacatttaacagaagtataaaacgtataatatggaaattacaaaacatcgttgaaagaaattaatgaatacctaaaaaaaaggatggacatcctatgttcatgaattggaagacttaatattgtttaaataatagtgctctcctagttgatctacagttccagtggtttatcaaaattccagttgccttttgcagaaggcaaagttccaattgccctttgcacaaattgataaactgatcctaaaattcatatggaaatacaagggatcctgtattagtttccgagggttgcagtaacaaagtaccaaaactaggtggcttaaaacaaccgaaatttttctcacagtttagaagctggaggtccaaaatcaaggtgttggctctgactgctctagggaattatccccgcttgctgcttctagtgtttgctagcaatccctgacatttcttggcttgtagatcactaaagtcacttagatttcctctacctgtgtgtcttcatattatttgctctctgtgtatgtttgtgtgtccaaaattctttttgtaaggacaccagtcatattggattagggcccatgggaatgacctcattttaattttaatttttttttttgagatggagtctcactctctcgcccaggctggagtgcagtggcacgatctcagctcactgcaacctccgcctcctgggttcaagcaattcttccaccttagccacccgagtagctgggactacaggtgcctgccaccacgcctagctaattttgtgtatttttagtagaggtgaggttttgccatgttgtggtcatgagctcctgacctcaagtgatctgccctacttggcctccccaaaatgctgggattacaggcatgagttacctcgacctctcctggccctcattttaatttgattacctttgtgaagaccctgtttctaaataaggtcacattctgagttactttgtgattaggagtttaacatatcttttttggggtacccagttcaacccataacagactctgaatagcccctcaaaaattgaaaaataaagaataaagttagaggaatcacatttctcaatttaaaaatttacatcaatccagacggtgtggcgcttggatatagattcatggagtatgatcattagtttaaaaataagcccctacctttatggtcaattggttttgacaaggataccaagataattaaatggtgaaataattttttatcccccacaaatggtctcagacagctggatatttgcatgcaaatgaatgaagtttaactcctacctcataccatctacaaaaattatgtcaaaatggattaaagacctaaatgctagagctaaaacaagtaggagctaaaactcctaggagaaaacataggagtaagtttttctgaccttgggtaggcagtggcttcttagatgtggtaccaagcacacaagcaacaaaagaaaaaatggataccttgaacattatcaaaaataaaaccatttgtgcttcaaaacaccatcaagaaagagacaacagaaagggaaaatacttgcaaattatatctgataaaggacttgtatccagaatacaagaagaactcttacagttcaataataaaataaagtccaattaaaaatttggcacaggttctgaatagacagaacctatgtatatctacttctacaaggaagatatacataatggttgataagtgcatgtattagtctattcttatactgctatacagaactacctgagactgggtaatttatgaagaaaagaggtttaattgactcacagttccaaaggctgtataggaagcatgggtgggaggcctcaggaaacttaaatcatggtggaaagtgaaaggggaagaaagcacatcttaccgtggtggagcaggagagagagagagagcgagcgagcaagcaaagggggaagtgccacacactttcaaacaactagatctcgtgagaactcactatcacaagaacagcaagggggaagtctgtccccatgattcattcaactcccaccaggcccctcctccaatacatgaggattacaatttgagatgagatttgggtggggacacagagcaaaaccatatcaatgcacgaaaagatgctcaacatcattagccattatggaaataaaaatcaaaaccatgagataccacttcagactcattaggatggctagaataaaaaagtcagattataaccaatgtttgttaggatatggagaaattggaaccctcgtacactgctgtgggaatgtaaaatggtacagctgttttggaaaacaatctggtagttcttaaaatacacacatagagttaccatataacctagcaattctatgcctagttatatatacccatatgttcatatatacacatgttcatagcattattcataatagctaaaagtagaaaaacaacccaaacatctatcagttgatgaatggagaaatcaaatacgggatttccataaatggattattactgaggaaccaaaagaaatggaccaaagcatactacaacatggatgaacctggaaaacattctaagtaaaagaagccaagcacaaaagaccaagtatcatatgattccacctatataaatgtccagaataggcaaatctgtagacagaaagtaggttactgtttccttagggctggaatggaaggattgggggatgacacctaagaggtacagtgtttctttttggggtaatgaaattgattggggtgatggttgcacaagcctgtgaaagtactaaaaatcgttcaatgtgcactttagttgggtgaattatttgttatgtgaattaaaacttaatgaagctggtttaaaaagtacattgaattattacaaggataagggaccctatgttctagggaaatgtacatattggaatatttaactaacatgagatgttataaagtagaatatatatgctgccaaatggatggtgtagatcagtgctgttcagaaagtatattgcaagccacgtatataattaaatttttctaatagccacatttttattttttagagaaggggtttcactattttgcccaggctggacttgaactcttgggcgccagtgatcctcctgcctcagtcttctgagtagctgggactacaggaacatgccactgctcccgccttataagagccactttttaaaaacttaaatggatgaaattaattttttttttttttccgagacggagtctcactctgttgcccaggctggagtgcagtggcacaatctcggctcactgcaacctccgcctcctgggttcaagcgattctcctgccttggcctcctgagtagctgggattacaggtgcctgccactacccttggctaatttttgtatttttagtagagacggggtttcaccatatcggccaggctgatattgaactcctaaccttgtgatccgcctgcctcggcctcccaaagtgttggggttacgggcatgagccaccacgcacaggctgaagttaattttaataatatattttatttaccccaatatataaaaatcttaacatttcaatatgtagtccttataaaaatcattattgagatattttacatccttcaaaaaaaattaaggccttaaaatctgttatgcatttcagaacgttcgattcaaactttgccactttcaggtgttcaatagctcatgtagtgactaggacaacataagtagagacattaagtgttaatccagagggaggagtaatcattgtagtctggaatgattaggaaaagtcacagaacttgagttattttgctcattcttttatttactgatttcagtcatttatccacaaatatttgagctcttactgtttataaaaaactagcaaaaaatgcagatatgtcttatctcctcatggacagtgcattaaacagctaattatatacttacttagggctggttgcttaagaagtagtggaaagagtataggcttagatttaaatattccctgtgaaacagaggatggtgataactgcattgtgggattggtgggagtaagtaaaattggtttgtcacatgcctaacactgtgcttgttgttaagtgttttctccttctcctacttcaccctctgtccctctacttccttgttgaatacgtgaggtgggccatgggaaaaaaaggcttaggaagtagcagagtcctgactacccaaggacttgagaatgagttcttaccctcatttcttatcctcagtcgcctttcacgggcctttccagggccctgcttcctggacccagactcaagtttgtattattagtactaaagcttattaaccagtattaaagggattttggcgggctaatttaggaatgtaattattgctttttctagagatttttatctacgtttcattccacacaactattttattaatgaaaatttgaaatgctatctgctaaattagtttgcctcttactcagctaagaatgtgttttagaaatatttcaagaagtgttgatataaaaattattttataccttttttgaaattctagacctaccattaaatacttagcactttggtccatgtaatttttgcattgtatgatatcttggaattttaacatactcttttacagtaagacttgttaggtctagttacattatgtttgagacaataatagatgagtttgttgggaacagttatagtgtattgaacagagtttgttaggagcagtttttatagtgtattgaatactggatttagattgggcagatcttggttttccatttgttagctatgtggttgtaagtacatttgttcactggtttttgattcatacagaagatatagcttgactaatatgctcaggacattattgtgtgtttacttatttaaaaattgtgattaagtgctttgaaggaaaagaactggttactgtgaatatagtaacaggtgaaaactattttagattggattgtcctagagaaggcatctctgaagaagtgaggtttaagctgagacataaaggataaaaaggaagggattagggatgggggtggtaaaagaatattgtaaggaaagggaacatcatgtgaaaaagtcctgaggtggcaaaatgctttgtgtattcaataaactgaaagaagcctagtgtgactgaaggattgtgagcaagggaataatggcatgatatgagaatgaaactagatcttacagggttttgtaaaagatgctgaaggctttaaagagttttaatcaggggaatgagacacaagtgtaactccaaagtggggagttgaatataggagtctctaaaggttgaaagagtagtctggtctacagaaatacatttgagaaccaacagctctcaaatggtaataatttggataaataccatgtatttaaagatctgggaatgcatgagataatctggggagagtatggagacagaagatagaagagaaagggagtctaggaccaagcctagaagaaccccaacgtttagaagctgatagggaaggaactagcataggaggacctcaagtgagcagagttttttggatgactgaggaaatataatggttctgaaatagcaatagggaacaaggaggatgtgattttttttttccaactagatataaacctttatttcacaactttgtcataattcaccttttaaaagacagactgggggacacagctgaaaacactgggaggccagatgctggtgtctttcaagccagagcatagctgtggtcactctagggcccatgtgttcctgtggctctcaggcatcatggaccagatacaccagtactgtccaatcccagttttacttagagccacctccttttttgggggccattagtcctcatttcgtgccagattttcactagaagctccctgttcttccaaaacagttaatgaccataagtaacataccatattccaaagagagctcccccaggatgcactgcatgataaaaaaatttccatcccatgaagatttctgctgtatccatggccataatggcttttagggcattctctgctatgaatgtgaacatctgaagggaaataatggcaagcctcccttctgggatcttagtgcagacagctgcaaagaccatcatgatggtgccagatgcaccaagtgatggtccctatcttcctgtggcaactttacacacataactgacacaattggaaataacacctgcagataataacactgctcttgatccagaatgttcactgtgctggaggcgaagctccacaaaacataagatatttgctgccatgtgaaataaggagaaatgactgaatgttgacagcaacattggaaaagaaagggcttttgaggctggattagatgcgaaatatctgatcattgtccgctgcagagaaggtactctctataaacagaatacaaagacattcatttgcagctgtgataacctgtcatagtctggtggccatcgcttaggttattctaccacttgttaatctcctttctgaagtctcttttttgtgatcttatgctatcccaaccgtcagcttttatatcatcaaaataactctggaccctggatttcagtgattcatattgccaaatagtagctgatccaaatgcacagcctgtaaacccaacagtaaaaaataaagattttatgagagtccctataggatacaaagaaggataaaaggccgtttcttccacaggaggaaccaaagcacttctcttgtatgcttcaccacttgtgcctgtgtctgatctttgaggttcagccttcctgggtgcttttctgaatccacatttttgcgaagaagccaaacctgcgtctgagcagccgtggcggggctagggcctcagtgagttccttgcagctgcggccgcccactggcggagaccacgcctggtcgctgccccaacctctctctgcccagcctcaccaggccatcttcccaacctctgccccaccatggcctgcaaggacatggttttgagacgtgtggaggaactgaataacatcagcctctgtgggatttattaaagtagcatatttgggggagttatgttttccataaggctagaaagtggagtttgctgatcacagaagaggtagagctgaggagctcattcagatgagagggatgtgtagagtagtaggggcatgagagttaacagtatttctggtggagacttacgtaaacaaggatatgaggtgttgatagatttaggccttttaatctcttgtaggctagagggatctatggccttagtggcatagtctggaaggtttcaaatagtttgttgggtaaaggactagtttctcgtgaggtaaatctgtctcagcaggttctagttatggtatgatgaggctccaggctggcctcttcatttcttttatctattaagtagtaataatacctcattaattgttatttaaatggacagtcacatagtgattaataaaagttaagactagataaacatacagccttttacgaaatcttttaaacacacatcccagtcagtcatgctctagccaagttattttcttgtcactcttttctgtgctgaatataacaatgcctaattgaagaataaacattgactatgaatgaggcactcaattcatctctttgctgattatactcaattttagaactaaggataaattattcaagaaatagttgctgattttttagtttgaagtgttctataagcatattggatttttttttagaacccagtgacagtaatacttctttggaaagaatataattgaatatctttttagaactttaagcaaatttatctttaaaacattttatatgctcagtgtagataatttggaaattacagagagatacaaaaaagaaaataaaaatttgcctgtattctcactatctagagaaaactgtttactttttggtgtgtttgtgtagcatttgttttcaaaattgggatcacaatatatagcaatttgtagctatatactctgctttttcatgtaatcttatgtcatgaatattttcctaaaccattaaatattcttggaaaatgtaattttaatgagtgcttaatagtctgttcattgtgagtgtatcatagtttacctaatgattctctgctattggacatctggattggctctaatttattgatatggtaaataatgccttaatgaatatctgtgtaaatcaattattttcttccagtttctttttactttcttttgccatattcctagaaagggaagtactgggtcaaagaatagggtttcatttttgttgctctgctttttttttttttttcctgagacagagtctcgctcagtcacccaggctggagtgctgtggcgtgatcttggctcactgcgacctccacctcctgggttcaagtgattctcctgcctcagcctcctgagtagctgggattacaggcacgcaccaccacacctggctaattttttggtatttttagtagagacagggcttcaccatgttggccacactggtcttgaactcctgacttcaagtggtctgcctgtcttggcctcccaaagtgctgggatcacaggcgtgagccaccgtgcctggccgttcctgctgctttttttttttttttggaagtcttgccctgtggctcagactggactgcaatggtgcgatctcagctcactgcaacctccgcctcctgggttcaagtgattctcctgcctcagcctcccaagtagctgggattacaggcgcatgacaccacgcctggctaattttttgtcctgctgcttttaaggaaaattatacttcaagatttcatatttaaaaaccaacgtgggtcgggctcggtggctcacgcctgtaatcctagcactttaggaggccgaggtgggtggatcactcgaggtcaggagttcaagaccagtgtgactaacatggtgaaggcccgtctctactaaaaatacaaaaattagccagatgtggtggtgcgcgcctgtaatcccagctacttgagaggctgaggcacgagaatcacttgaacctgggaggtggaggttgcagtgagccgagattgccctctagcctgggcaacagagtgagactccgtctcaaagaaaaaaaaaaagagtgtgtgtgtgtttgtgtgtgtgtgtgtgtgtgtgtgtgtgtgtgtgtgtgtgtatttggcggtatagcattgcttatttctctgtttccagcagtggtttatttttgtgtggatttgcagacccccttgcggtaataaatccttacaggttctctttggcttatatgttgagaacaactactatagattataaactacctcagttctctcccgtatcctttgtttcgctgctcttgctccagcagataccttctcattgcctctaacaaataactctttctcctttattcagtgattgttttttcctagcatcccttgcttgatatttggggaaagttttaagacttttagaaaatcgtgtttgagagcacctggttttattgtatcgcttgttagccttgagtgtttattttttaatatttgctgatttgtgagatgaaatatgataggtatatttgggggtaggaggaatgtctgaaatacattttctttcccaacttatgatttaataagagctggaaggagaaaagtaagctataaaacccagtatttgacattttatcctcttaatctggctaccgtccatcatactttcagagctgaaagggctttaaatttcatatttatccagtcctttcagtgtacagatagggaaactaaggcctaaaaaggacattgcttgacttcctcaaggtcataagccaaacagtggtagaagtttggcctgttttttcataacttagtgtgttggtttttctacaaacatcttctgcctctctgccgcatgtgtcccccaacctgccttttaggttgactaacttttaggtgactaatttttttctgtttggatgcctggtagactcttttttatccttgagtttaaccacagtactttgatcatcctgagtcagttcttgcatacattgtacctttttgtcctgcagatatagtgctctcaatttcaaggaaagtttcaacgaaattttctattcatgtgtggcatctacttatggaacaataattatccttatattggatcatctttgttcttcatatttgctatcttctccagaattacttcaatctcttaagtgattccctctgcattcactattatgatttcctttcttccctgccattaatttttcactgtgtttttttctgttctttgctgtttctagtttattagatctctgatgttttgttctgctaagaacaaactgagaactgttgtaggctaaaaaacagccctccagaagatatctgtcacccaggctcattgcaacttccgcctcctgagttcaagtgattctcatgcctcaacctcccaagtagctggggttacaggcacccaccaccatgcccagctaaatttttatatttttagtagagatggccaggctggtctcaaactactggtgtcaagtgatctgcccgcctccgtctcccaaagtactgggattacagacatgagccaccacacctggcccccagaagaatcttactatccattagcagtcactccccatctcctgacacacactgcctgctttagataaccaggtagattagtgagtctatcttctttctatagatttgcttattctgaacgtttcatgtgaacaggatcatacaatgtgcagtcttctgtgactgtccacttattttcagtttgttaaactccttcaagatctttctttttacatgccttctgccattaatgttatcgcttcccactccattgcttccctcctccccctaccctctcacacatttcagaatttcagctgaaataccactttttcggagggacctaccctgattggattctagatactccattatggacttaattgtaccccaccattctcttttgtacctcttacttcagttattatctaatgcagcagttatttgtataaatagtatctgcttttctcatgaggtagggagtttgttgttgttttgataaatacttagatgaacatattcaggcactttacccagtgggaggggaaggaggttttcagagaagagagatatgtgtacgtatgttaactattaatgtagcaatgaaaatgtaagctgaaaaaatagaaaattagtagttaactaggctgtatgtgtgtagactggacaggtaccataaagaatgtcaaagaatgtctattatactctcccagaaataccatgaatctggaattgttttttttgagacagggtcttgctgtgttgcccaggctggagtacagtagtggctccagtagtggagagcatacatggctcactgtggccttgacctcctgggctcaagcagttctcccacctcagccttccaagtagctgggaccactggtgtgtgccaccgtgcctggctaattaaaaaaaaattttttttgtagagacagtatctcactatattgcccaggctggtcttgaactcctgggctcaaacagcagtcctctcacttggcctcccaaagtgctgggattataggtgtgagccattaaaacctaagactgttaagtgccagcttaatttttggagtccatttatgttgttggttagaatatttggaacatttaccctacctgaaaagttgaataaaactgatatattctgttttaaatttggattcatcagttcagtttttcaagctcatttgtaaataatggggcatattgcctacgcgggtcacaatttgtttcaggaaggtgctgcatcatctgctttgatggagatcttaatgaaattggctgctcttgacctctgagattgaccatttcttcatcttttcatggtacctagattgtcaggtacactgttctcttctcccttagtgtttgctttactctttagtggatattggagatacctaggacaaaggtttgtgacgagggtgtgtgtatgagggtcttaagataagggaaatggtatttttcttcttgattaaatttaaggggcatcatgaaatttaagtaacaccttaagagtaataggtcagatggctttttgttgatattattgatctataaagttattgctagcactgtgtttaccatattaagtattaaaaccttatattctgtgggactcttcacatgggtatttagtttcagtttttataatggaaattggtgagaaaaaaggatgactctacaaatttttgttttgtagttaacttttattacaatttcatctaggatgcatgaaagtacatggaatttaggggcagtgttggacataaccttggagggacccagctcaggaaagtcttcagtggaacctttgtcttattatttgcatatattcacatcaaacccttttaaagtttccactgattttttttttttttaaatgagtatgtgttcaatgcttctgcaggttgtagaggtatggtataaaaaagaaattcaccagcagtcacagctagcagacttgggctagtcctatcctggtatatcattaagtcaggtagttaacagtaggagatgacaaagaccatttaaaaaagggcagagttaggcatgtacatatttcccaaggatgaaagttctgtttcaagggaaattgtttcctactttaatgtccttatctacttttgacactgcgaataatgagtaagcactgtgggagacactctgctttggcatcggatgtcctaagacattcttccatgacccaccaataagaggattagtctcattttatttctcaggcagtgataaaggaaccaagtcccagttgtagacccactgtgagtgcaggattattccagtgtcttttatttaatagagccagttagcatctcttctagtagagctttgtggggagaaagaaatgctaagtggactgctttgtgaattgacccacttaatctgacagtactcttccgccttgaagaggtggacttggacttgccctatttgaatggcattgccaggttctctaaaggccggtctttttttctttcatttctttcttttcctctctctctctcttttgtggggagaggattgggaggtgttaagcaagtttttagagggaggaagataaggcaaatacaaaaaccaatatggatttgattataagtaactgtaatcagttaggacaatgttatttaatatgtgcataccttgacaatattgtgggtttggttccagaccaccacagtaaagcacatcacacaatttttttggtttcttagtgcatataaaagttacgtttgcactatactgtagtttataaagtgcagtagcattatgtctaaacagccttagttaaaacatcctttattgtttaaaaatgctaataatctgagccttcagcgaatagtcatctttttgctgctcagtgttgatggctattgactgattagggtggtggttgctgaaggctagggtggctgtgccaattgtttaaaaggagacaaccatgaagtttgctggattaattgactcttcctttcacggaagatttctctgttgcatgagatgctgtttgatagcgttatatttgtttttagtttttaatagagatgggtgtctcactgtgttgtccaggcaggactcaaactcttagactcaagtgatcatcttacttctgcctcctgattagctgggactacagtagccagcaccccaggcattatagcattttacccatagtagaactttcaaaattggaatcagtcctctcaaaccctgctgctgctttatcagctgagattatatcctaaatcctttgttctcatttctgcagtagtcacagcatattcaccaggagtacattccatctcaagaaatcactttctttgctcattcataagaagcagctcctcatctgttcaagtttgatcacaagattgcagcagttcagtcacatcttcaggctctgtttctaattctggttctctttctatttctaccacatctgcagtgacttcctccactgaagttttgaactcctcagaatcatccaggattggtatcatcttccaaactcctgttcctgttcctatgaatcatgaatattctttttttttttttggagatggagtctcactctgttgcccaggctggagtgccgtggcgcaatctcggctcactgcaacctctgccctccgagttcaagtgattctcctgcctcagcctcccgagtagctgggattacaggtgcccgccaccatgcccggctaatttttttttttttttttgtatttttagtagagacggggtttcaccatcttggccaggctggtcttgaactcctgatctcgtgatccacctgcctcggcctcccaaagtgctgggattacaggcatgagccaccgcacccgctgaatcatgaatattcttaatggcatctagaagttgtttagtgtagttatcttcatcagtggtcttagctaaatcttctggataacttgctacagcttctaaatcagcacttactgctttaccttgtacttttatggtatggagagggcatctttcctctgctagcttcaaacttggcagccttccttgaaggcttctgtcggccttcagagaattgaagagggttccggccttgctctggtttaggctttggcttaaggaaatgttgtggttggtttggtcttctatccagaccactcagactttctccgtatcagcaataaggctgttttactttcttgtcattcatctgtttactggagtagcacttttaatttccttcaagaacttttcctttgtattcataacttggctgtttggcacaagaagcatagctttcagcctatctgggcttttgacatattttcctcactaagcttaattacttctagcttttgatttaaagtgagagataggtgactcttctctcacttgaacacttagaggccactgtaggcttattaattggcctaatttcaatatcgttgtgtctggaataaggaggctgaggagagggagggagatgaggaaacaactgattggtggagcagtcagggcatacacaatattaatctattaagtttgccttcattgatcacagatcaccataatgggtataataatagtgaaaagtttgaagtactgcaaaaattaccaaaatgtgacacagagacataaagtgagcatatgctgttggaaaagtgacactgatagacttggttaacatggggttactataaaccttcaatttgtaaaaaaacataatttctgcaaagtgcaataaaatgaggtatgcctatatatttttctcttaggcatgttccttattatattaactataaacagcatcattactaatgaagttgtaaactagaatttaatcagtatttgaaaatgtatggtgtcccttgcagaaatggaattaatgacaaaactcgtagttattacctgctattttattttccatagttaattttaagaggttcttataaagttgtttggaggtactctatcttcgcaacagttaaaattgtaatattttaggcatatctcctcctcctattttgttatacagaaaagctttagctgataaacagattgcattctaaaagttcataaactattttggaacttagaaagtattttttcatagtaagtttagtaaatatgtaaatgcgaataggttcccaggctatcctacaaaagcctagtgaagccatagttagctcaaatatctacatatttacagtgacacataatagaaaataatatggcaagaatagtagttaacaccagaacactgttaagaattagttttgaaaaattttcttctatgtgtgattgatacagggatttaatcagagggaggtcaggcgtagagacttgcgggagtctggcaggcctttctgtgtcctttcaggtactttataggttgctggcatttctttgttttgatattggtatacagcagaaatgctatctttttgcttaccggctatggttaagattgtattttaggttctgagtggaatgaaaatggtagaaaagaaattgtttcctcaggtaaaaaagataggaaagaacaatggaagacagagtaacctaggatgtgtgtttgaaaaagaaattgtttgagatagatttcattgacacacaaataatgcaggtttaagggaataggggtagagtataacatgcaaatgattcctgatggaggagagcaaaggtgttgataaagtgttttgggttagcaatccttatgctggtcagtaagtggttttggatatctttggaagctatttagaactgggaatggggagtagaatgctactatggtatttttatatgaactgtttcagtattttttttttcataacatggtacatcagctcaccaaaaatatttaaatagcttgatatacatttggagtgttttttgatgtattaaactattattaagaaaatatattttttgaatgtatgaaaagtaaaagtaaaatattgtactttagctaattcaacagatgcctaaaactattatgtgatgagagagatttggtttttacattccttttttttgtttgtttattgattagaaaaaccatgcttcctttttcaatttccaaaatgtttcatatttagaagaaagcagtccaaaaagaaaatcaggaaatgttccttctattcctacaaaagtagcacctgttgttaggctatgtattattacttagtattgtgataaatctgttgaagtgtttaaatgtttgctatctgttcactgatgtgatttgcctcgggaatttctggctctctttttactcgttttagatgatttgtctctcaggtagctttgtatatcagcaaaatgacacagttttatatttaaaggtatttttgcagtttatattagaagatgtaaagactcttactcaaaatgtgtggttatattggaccgagttatttttatggatttattttagaatgtaaattattcaattcactttcacttttataagtaatggaaagtatactaaacttaggaattggatgaggcaatttggatgagtactataagactatagtatagtttttaatactacacagtctaaactgtagtgttatactctagtttttaatactatagagtcattgctgttatacatggggcattggttccaggaccactgggcacatcaaaatctttgcatattcaagtcctgcagttggccctatggaaccaccgtatgtggaaagttggccctatggaaccaccgtatgtagaaagttggccctctgtatatgtgggttttacaccctgtgaatactgtacttttaatctgtgtataagtggacctgcgcagttcaaacccatattgttcaggggttacctgtatataaccttgagcaattttacttattttttgagtctcagttcccttctccctgatttagtactggttcttcctcctttgtaggactgttgcttggatcaaatgagataatgggtctaactgtgcttttaaactgtaaagtgcttcacaaatttatttgtgatgatgagtgatgatggtttggtgatcatgattggtcctagggaaaactgtcaaatatatgaattttaactgcattatcttctcaatctgttgaattgacttcactcttttaagagggtgattgcagggtccgaggtcatctctgaggaggaagtagaggtatttattttggtgactttcagactttagtttccaaagtatagtagaagcctgtagaaatttgtcacttataaatgaacaaatattttttcagactgctatctgcatagtcataaaatgggacgcccccagtgtgtgataactgtaagtgttgccctcatggattgggtattagttgttaaatcgtaattgataaagtatgtaggactgacttagagtaagcagtagtcttctgggagaaagcaaatcctgtcctctctcctcccctccctactgttttaattcaacagcaatttttgatttaataacctccactttaaagtgattaaaaatgtgtgtaaatttgtatgctgtgctatggaaaaagcagtttggattaaggagtgcagatatctgcatatttcaggtgagggcagggttggataagctgttatattgggggcatttgggttgtcagaatgcaatattgaggaccaaagggatgtctagtaggccaagaacagacctaattgatttaaaaaatatatttagagcactgatctggtattactgtcactaaagattacagtaaaatataccagctttttgagatctattttctggctttcttctggctgctcaaggttgatgtttctagaatgtgaccactgactggatcagtgagtgagtaattgtatttttaaaagtaattatttacttctttagtttaagtacttaccattttctcttttcatccatgtatagcgtactaattctttgataatctggtaaccttccttcttaagaaaaatttaaatatagtgtctacccttgccctccaaattgggcagcttgtaaatcaaacggataagcgtgtttttaagagttaaaaattctaatgtaaattcctaaaagccaaagatttttatgcacatgctgtaagccttttagacgtgtatggaaaataaacctgtttctaaattacgtcttgaaattatttcaagtgaggagttaagaccaagtgaaatatttattcactttgggtttggtttgattttattccagttcagtgaagttagagaaaaggaaaatacacttgattccagtttatggcttacaagtaaaataatatttaagttcagactgttgaaaattagcatgtttctgattttaggtttattgtttggtttaaagtaaaatttggttttgaggtaaaaccaagaagataaaaaattgagtcaattaaaattggctcagaagaagtatggatatttcaaagaaaaaattggagtggctaatagatattggcctcagcaccttttgtaagatatttggagttttaggttttgctgaacacaaaaagttggtttcagggcctaaatacccatctcttatatttatgacatggtccttagcaaagtaatgtagtaggacacgagtttttcagtaaatggacaggtctgaatcctgtggatatgagttgttgaagggattactttgagcaaattatcaaatctttttgaacaccgtaaattttcccatttgttaaaatgagtgttctgagtatgtcacaggctttttgtaggaattaaatggtatattgtggctcttcaagtgtggtagctggacctgcattaagaagcatttgaggtgcttattaaaaatacagtttctggagccccacttcagagctactgaccttttgggggatatattctagggtcttcattttagtaaactcccaggtgattcttatgcccactgtgagtggtgcttgcaaactgtaaacagttctcaaggaatatgcttacataaattgaacttaagatctcagttttgtctcctgtccagtttttgccaattgctgcatattgatgctctaaagaaaccagaattatttggaaaagggttaggaacagagtaaatttcccacccatacctaaatacacatgtgggacattgagtcatttgttttggctgcttgttgtcaccatgttttcagtaagccctccattctgttttactacttgtaaggcttttctatatatcttctccccaggaatttatatctgaccaatggcaagcataggaagacacaaaagcactacttaggtttaacatgaattagtcagttctcaaatcctaacaattctgcttgaataattcttacaaagtctgtggcaaacctttgctgagcatttgcattgtgctgaacctagtgcacatgggaaatgtaaatgattccaataatgtctagttttttttttttttttttttttttttttttgagacagagtctcaccctgtctcccaggctggagtgcaatggcgtgatcttggcttattgcaacctccacctcctgggttcaagcgattctcttgcctcagcctcccgagtagctgggattacaggcgtgcgccaccatgcccagttaattttttgtatctttggtagagatggggtttcaccatgttggccaggctggtctcgaactcctgacctcatgatccacccgcctcggcctcccagagtgctgggattacaggcgtgagccactgcgcctggccaatgtctagtttttctaagagctcttagtgaagtaaggggttcagacttttaattatagggcattgtgataagtaccgtgatagggcacatgtgagtggtaaaagagcatagtggagcagaataatatattctttcctatttccactgctaataatttgaagttcaaagaaactgtaatttattgagcacccactaagtggcatgcatttaaaaaacaacaactgtgtaacctgtgctattcaagtgtcacaaccctaagtagcagctatttccattttatagatattaagtaggttgaacttgtgatataaacctttgtatgacttcaaagcccatattcttttcattaggcctctaaggacaaaatttgatttttttctttttaaagaaggggatgttatgattcttcagaattgatatctgttaatttcagtagcatgatgtcttcctgtgattgaaaagaactctggagtctttctgtcatctcttgtcctctgccaccctatgttttccttttcatttctaaaagtgataaacaagtaaacatctccggaagccagcctatgtgacatctgtccattttgttttctctgcctctccttttatttactcttcctatccccaaaggaaatttagtaattgttttttttgttttttctaatctcttaggttagaatcatgtgaagatgaatggtatctaagggaaaagaccttgttagaaagatttttatcacatgaagtttacatagaaattgccctggaatttttatcctgctgttgatcattagggttgatttgattgattcaactgcttggttgatatttcctttctttcttcatagtttcatttgttccaacatatataagcagttgtgcactctggctagagcttccgcatcataaataaagcccctattttaatcacaagaataagttcatttttgaattttctaactctgatcccatggggctcagttttctactttttattccctttgctaattaatttttatgttgttttcttgtttctagcagtttgtttattttggggagaacatctttgctttttaaaattaaatttattttatttaaaaataaatagagacaaagtttcactatggtgcctgccatggcctcccaaagtgctgggattgcagatgtgagccactgtgtctagcccaacatctttgttgttgttgttgtttgttttgtttttgctttttttttttttttttgacagagtctcactctgtctcccaggctggagtgcagtggcatgatcttggctcactgcaacctccgcctcccgggttcaagcaattctcttgcctcagccttccaagtagctttttgtacttttagtagagacggggtttcgccatgttggccaggctggtctcgaactcctgacctcaggtgatccactcaccttggcctcccaaagtgctggaattataggcttgagccactgcgcccggccaacatctttgttttaatagttttttttttttttttaaacgtggtttctttggataaagagttgaacttcaacttaacctgaaaaatttacagtggccagagtttcctcttctgacagtcttatgtaaacatcctttaaaaatagctgtgtgttatttctctacatgatagagaaataattggcagtgcctttccctctgctaatactattgttagacagaaaatgtcatgaaaatgctttgtatcttctgtgtattcatgggcggtgtgctgatttctctaggcttttgttgcagacttttaattttctgttatttctacgtggagacttcattcttgaggtgaaggaaaatttttatagaggcagtcattgcagacttaactctccacagcaaggtcaccagagtatttattagactgtggcatgacttttctcagtttgtagtacaccttattcatattctctaaaagcacctcagttacaaagtgtctcagttgttgctttttctcagcactggattttaagaactagtctccttaaaacaagaaacatcaaattaaatgcctttgttaaatgaacaaagtatgttcagctttgagatttatcatttgaataaaattagagtgtcagtgccagacacctacctacgaaatatttaaacatccttgaagtggacttttggcttttggatactccctccataccttggatgggtccatgtcaccgtattcatcgtggagtggattggtctgaggagtgacttgagaagacttcaaaaaatatgactacaaaatgttgtttacaggtacaatcctgtttttaggagattgagaactaggctgtgtgtattattttgggatatgcctaaaccgccaaggcttttgtaaaaattgtattctagactggatacagtggttcacacctgtaatcccagcactttggggaggctgaggtaggatgattttagacttaaggccaggagttagagaccaacctgggcaacaaagtgagaccctgtctctccaaaaaataaaaaaataaaaaatattagtgaggcatggtggtttgtgcctctagtgttaactacttgggaagctgaggcaggaggattgcttgagcccaggagttcagtgttacagtgagtgagctatgatcacgtcattgcactctaacctggacgaccctgtctcttaaaaaagaaaaaaattattctgtatttaacagccttcaatagaaagtgattgttctgttgtataccgtcttttcattgcgtcatgaggggtctattctggtttatgtatttaagatttatctattatggtaaagagtagggatttaatgctgtgttttaattatgtagggaatttgtacaatgtgagtgaagtcctctgacgggagagagggcctgaagctgaactagttcctgctgctgtgctgttgtaccttcaaggcggtttcgaagaccaattttattataatcgctagtgccatataattagtttggaaattactgtttcatgtatgctaggaaatttagtactgggcaaagtggttattttttgtttaagcagatttgttacagtttctagtttttcttgaggattttggtctcttggaagcagtgctatggtctgaaatagcccaatttgttgtagcaaatattaagttgtttggatgtcacagaatcaactgttgtacagttttatttttgatatggttttgctttggtataaatttcaaaacctaatgttgccaaagtgtctctgggaatagatgttgatattaaggagtacatggttgaggccagaaaactataaaagcctgttgatttattctcttacttcctattactctggagctgatccttttacattatgttattggaaacattatcttctttttttaaggggaaatggagaaaagaatatgaagttagtggtagtgggaaggttttaaaatacagcagaggcagcacgaggcatgcgatgagttctgctacctaaagctttatgataaattaatctattataggtattaatgtagatgaaacaatgaatgggattgttctttgtaaacagtacaattttatacagttgttatcttattgccaagttggctaattccaatgccaaatggtaatgcgcttttccttggcacgctagttgctgaccgacccatggcactactaatgttcatgccaagtcatcccccatattgccctgccattcttttcagctctgctatttatcttttaattactgctaaggtagtaagtaccttgtctctagatgtgcaaaagtgtgaaagaattggtccttgtctacatcaaatctcagcttaaaattaagaggccttaagagtctctttcagctatagtatctgtaggacctgttttacctttccactctgcttttaatactttagcagaattgtcttatttgatgctttttcccatttgcaaattatgatgctgggatagacattcctaggctcagtggctggagtgaagagatgctaaatcccagggttctaatggatatctatcatagctagttttcaaacccttgaaatgttaccttttttaaaaagaagtagaacctgtggaggcaaatagggaacgtcatcacaaatgcttatattgcttaagttctcttactagagcaaggaaaatacaaaggtggggctcctgtttcctttattgaaatatggttatgcagtagaaaaacataatttttggttggaccccagacagtttttgttttccgtttttcagttaattaaaattgtggagtacagtaggctttttgtgttgctgttattggaaatggtcttgctgtgttgcccaggctggagtacagtggcatgatctcagctcactgcagcctcccgcctcctgggttcaagggattcttgtgcctcagcctcccttgtagctgggattacatgtgtgcatcactgtgtttggctaatttttgtatttttagtagagacaatgttttgccatcttggccaggctggtcttgaactgctggccttgagtgatccgcccaccttggcctcccaaagtgctgggattacaggtgtgaactaccatgcctggccatttatttatttatttatttatttatttagagacagggtctccctttgtcacccaggccaaaatgcagtggcgcagtcttggctcacttacaggcatgtgccaccacactcagctactgttcttaaaatctatattagtttattaatgatcctttgttttgtgagttagatgtgagattttgcaagcaacttgttcagtggaatttttaattcagagtcctcgaaatctttttttggtgttacagttttgacttggataacatagggaagagtcagactatgtttgaaataacgtatgctttttatgttctcttgagacaattggattgtaagaatgcattcaataccttggattttaacctttctggatctcttcatttagagtttaagttcagaagactgtgattctgtttttaggactgctaagatacaggtaaagaaaagttattttacaaatgagggagagaataacttatgataaggtttatagtcagcttgaaaagttgtagttaagattagcatagaattttggggggtggggcatgataatttagaactcagattaaactaggtttttagcaaatatacctaaggtttattttactgagggatgagacttgaagggagagtttgaaagctttgaaaactgctttttttatattggcttatgctggggtttgtgtgcatttaaagcatatataatactgctgtaaataaaaatggtggtgctgacaagatttgagttctttcttttcacaagtggttttggatttggggtgattaaatcatgccagtttgagtgttttaccttggacctaaagacataaatattcaagatggctggccatgataaatatagactgcctgtatttcacctggctatcttatgaaggaatccctcattgcttttgaatctgtcatgagtgccatctttttttttaattattattatactttaagttgtgggttacatgtacagaacgtgcagttttgttatataggtatacatgtgccatggtggtttgctgcacccatcaacccgtcatctacattaggtatttctcctaatgttatgtctcccctagccccccaccccccacagaccctggtgtgtgatgttcacctccctgtgtccatgtgttctcattgttcaactcccacttatgagtgagaacatgcggtgtttggttttctgagcttgtgatagtttgctgagaatgatggaatgatggtttccagcttcatccatgtccctgcaaaggacatgaactctccttttttatggcggcatagtattccatgatgtatatgtgccaaattttcttaatccagtctatcactgatggacatttgggttggttccaactctttgctattgtgaatagtgccgcagtaaatgtacgtgtgcatgtgtttcatagtagaatgatttataatcctttgggtatatgctgagtaatgggatggctgggtcaaacggtatttctagttctagatccttgaggaatcgctacactgtctttcacaatggttgaactaatttacactgccaccaacagtgtaaaagtgttcctatttttccacaacctctccagcatctgttgtttcctgactttttaatgatcgctattctaactggcatgagatggtatctcattgtggttctgatttgcatttctctaataaccagtgacgatgagcattttttcatatgtctgttggctgcataaatgtcttcttttgagacgtgtctgttcatatcctttgcccattttttgatggggtcgtttgcttttttcttgtgaatttgtgtaagttctttgtagattctttatattagccctttgtcagatggatagattgcaaaaatttttctcccattctgtttcttttgctgtgaagaagctctttagtttaattagatcctatttatcaattttggcttttgttgccattgcttttggtgttttagacatgaagtctttgcccatgcctatgtcctgaatggtattgcccaggttttcttctaggatttttatggacctaggtcttaacgtttaagtctttgatccatcttgagttgatttttctataaggtgtaaggaaggggtccagtttagttttctgcacatggctagccagtttttccagcaccatttattaaatagggaatcttttccccattgcctgtgcgtgtcagatttgtcagagatcagatggtggtagatgtgtggtggcatttctgaggcctctgttctatccctttggtctatatatctgttttggtaccagtaccatgctgtttggattactgtagccttgtagtaaagtttgaagtcaggttacatgatgcctccagctttgttcttcttgcccaggattgtcttgggtatgcaggctcttttgtgtttccatatgaaatttaaagtagttttttccaattctgtgaagaaagtcagtggtagctataaattactttgggcagtaaggccattttcacgatattgattcttcctatgcatgagcatggaatgtttttccatttgtttgtgtcctcttttatttccttgagcagtggtttgtagttctccttgaagaagtccttcacatcccttgtaagttgtattcctaggtattttattctcttagtagccattgtgaatgggagttcactcatgatttggctgtttgtctgttattggtgtataagaatgcttgtgatttttgcacattgattttgtgtcctgagactttgctgaagttgcttatcagcttaaggagtgtttggcctgagacgtcaaacagagacaatttgacttcctctcctcctatttgaatatgctttatgctttttcttgcctgattgccctggccagaacttccaatactatgttgactaggagggatgagagagggcatctttgtcttgtgccggttttcaaagggaatgcttccagcttttgcccattcagtatgatattggctgttggtttgtcataaatagctcttgttatgttgagatacgttccatcgataccaagtttattgaaagtttttagtatgaaaggctgttgaattttgttgaatgccttttctgcatctattgagataatcatgtggtttttgtcattggttctgtttatgtgatggattacatttattgatttgcatatgttgaaccagcctgtcatcccagggatgaagccgacttgatcgtggtggataagctttttgatgtgctgctggtttcgatttgccagtatttcattgaggattttcgcatcagtgttcatcagggatattggtctaaatttctttttttttgttgttgtgtctctgccagatatcatttggtatcaggatgatgctagcctcataaaatgagttagggaggattccccttttttctattgattggaacagtttcagaaggaatgataccagctcctctttgtacctctggtagaattcggctgtgaatttgtctggtcctggactttttttggttggtaggctattaattattgcctaaatttcagaacctgttattgatctattcagagattcgacttcctggtttagtcttgggagggtgtatgtgtccaggaatttatccatttcttgtagattttctagtttatttgcgtagaggtgtttatagtattctctgatagtagtttgtatttctgtgggatcagtggtgatcccctttatcattttttattgcatctatttgattcttctctcgtttcttctttattagtgttgctaatggtctattttgttgatctttcaaaaaatcagctcctggattcatttatttttttttttgaaggttttttttctgtctctatctctttcacttctgctctgatcttagttatttcttgtcttctgcgaggttttgaatttgtttgctcttgcttgtctagttcttttaattgtgatgttaggatgttgatttagatctctcctgctttctcttgtgggcatttagtgctctaagtttccctctaaacactgctttaaatgtgttccagagattctggtatgttgtgtctttgttctcattagtttcaaataacttatttctttctgtcttaatttcgttatttaccaagtagtcattcaggagcaggttgttcagtttccatgcagttgtgtggttttgaatgagattcttaatgctgagttctaatttgattgcactgtggtctgagagacagtttgttgtgatttctgttcttttacatttgctgaggagtgctttacttccaattatgtggtcaattttagaataagtgggatgtggtgctgagaagaatgtatattctgtttatttggggtgcagagttctgtaggtgtctattaggtccgcttggtccaaagctgagttcaagtcctgagtatccttgttaattttctgtctccttgatctgtctaatattgacagtggggtgttaaagtctcccattattattgtgtgggagtctcagtctctttgtaggtctccaaggacttgctttatgaatctgggtgctcttgtattgggtgcatatattttaggatagttagctcttcttgttgaattgatcactttaccattatgtaatggccctctttgtctcttttgatcttcgttggtttaaagtctattttatcagagaccaggatttttttttgctttccatttgcttggtagatctttctccatccctttattttgagcctatgtgtgtctttgcacgtgagatgggtctcctgaatacagcacattgatgggtcttgagtctttatccagtttgccagcctgtgtcttttaattggggaatttagcccatttacatttaaggttaatattgttatatgtgaatttgatcctgtcattatgatgctagctggttattttgcccattaattgatgcaatttcttcttagcatcgatggtctttataatttggcatgtttttgcaatggctgctaccggttgttgctatccatgtttagtgcttccttcaggagctcttgtaaggcaggcctggtggtgacaaaatctctcagcatttgcttgtccgtaaaggattttatttctccttcacttatgaagcttagtttggcgggatatgaaattctgggttgaaaattcttttctttaagaatgttgaatattttccccctactctcttctggcttgtagagtttctgccgagagatctgctgttagtctgatggccttccctttgtgggtaacccaacctttctctggctgcccttaacattttttccttcatttaaaccttggtgaatctgacaattatgtgtcttggggttgctcttcttgaggagtatctttgtggtggtctctgtatttcctgaatttgaatgttggcctgccttgctatgttggggaagttctcctggtgaatatccttaagagtggtttctaacttgattccattctccccatcactttcaggtacaccattcaaacgtagatttggtcttttcacataatgccgtatttcttgaaggctttgttcatttcttttcactcttttttttctaatcttgtcttctcgctttatttcattaatttgatcttcagtcactgatatcctttcttctgcttgattgaatcagctactgaagcttgtgtatgcttcaccatgttctcatactctggttttcagcttcatcaggtcatttaagctcttctctacactgtttattctagttagccattcgtctaaccttttgtcaaggtttttagcttccttgcaatgggttagaacatacttctttaacttggagaagtttgttattactgaccttctgaagcctacttctgtcatcttgtcaaactcattctgcatccagttttgttcccttgctggcgagtagttgtgtttctttggaggagaagaggcgttatggtttttggaattttcaacctttctgctgtggtttctccccatctttgtggttttatctatctttggtctttgatgttggtggcctacgtatggggttttggtgtggatgtcctttgtgttgatgttgatgctgttcctttctgtttgttagttttccttctaacagtcaggtaccttagctgcaggtctattggagtttgctagaggtccactccagaccctgtttgcctgggtatcaccagaggaggctgcagaacagcagatattgctgcctgatccttcctctggatgcttcgtcccagaggggcacccacctgtatgaggtgtctgttggccattactgggagatatctcccagtcaggctacacaggggtcagggaaccacttggataacagtctgtcccttatcgggagctgaaacgccatgctgggagaaccattgctgtcttcagagctgtcaggcagggacgtttaagtctgcagaagctgtctgctgccttttgttcagatatgccctgcccccagaggtggaatctagagaggcagtaggccttgctcagctgtggtggtctctgccgagtttgagcttccctgctgctttgtttacactgtgagcatagaactgcctactcaagcctcagcaatggcaaacgcccctccccccaccaagctcccacatcttaggttgatctcagactgctgtgctggcagcgaacaaggctgtgtgggcgtgggacccgccgagtcaggcatgggaggggatcttctggtctgcctgttgcgaagagcattgggaaagtgcagtatttgggcaggagtgtatcgctcctccaggtacagccactcatgccttcccttgggtaggaaagggaaatcccccaaccctttgcacttcccaggtgaggcaatgccccaccctgcttcggctcgccctccatgggctgcacctgctgtccaaccagtcccagtgagatgaaccaggtacctaagttggaaatgcagaaatcacctgtgttctgcaaagatcttcctgggagttgtagaccagaactgttcctattcagccatcttggaagcgaccatgaatgccatcttacaaatgaaagtgttaattggtcacagacattcactggcttttgtgtatattaatacttctgtgaatttaaatggatataaatattggttataatattccactagtgcatttggatattcatttatctagcaatttatatttcacagagaaaaatactcataaatattatattacttttcttttattaggttaattggaattcagttagtattttagaaaggaaattgtaatggaagaataacaggaaaaaataaaagtccaataagtgagcctaaggacttatattgtactgagaattggttttagatttgtcagggaagagagaagaaacagtagacatttttgtgtacgttgaagtcactaaaagaacaatgtaaagctcttttagatcagatactaggtagcataatacacagaatcgattaagaattcagaaaacaagatggtgactgtggactatccctagtgaagtaggaggtggctcttgtgcctgaattgaagagagccgttggattgctactggggtgagagaaggcaaaaaaggaaagaagaggtctgtgagtgaatagagaccctggagccagtgatatgcttatataattgggcctcagttcagaatgggttggattatttaaatttttatatctgttaaccccaaattaacattcttttctccacctgagctttctctgagaggtcttctactttgctaaaaagaagattcagggaagcaaacactcaaagccttactttcctaaatgcagaaggatctgaagagagaagttttaaacatttttatttgcacatattttattttcaagtaaaataaggtcttgaagaaagccctaaggccttttagaattggatggataaggtcagactcttatgggttcaccttttgctatttgaccatgggcaaatttaacattcttcctattacctataatatgggaacagtactgtctacctcatggggttgaaactaagcattgtgtctggtctatagaatgactgaatacatgggagtttaaaaaatgacttataattgaaacaagtaattgaagtaattacatgtaataaactaaaaataatttgtagtaaagaatatagtttactttgaccaggcgtggtggctgatgcctgtaatcccagcactttggaggccaaggagggcggatcacctgaggtcaggagttcaagaccagcctggccaacatggtgaaaccgcgtctctgctagaaatacaaaaattagccagacgtggtggcaggtgcctgtaatcctagctactcgggaggctgaggcaggagaattgattgaacccggcagacggaggttgcagtgagccgagattgtgtgactgcacaacagcctgggtgacaagagtgagactccatctcaaaaaaaaaaataaaataaaaaataaagaatatagtttaccttgtatattttggcagttttctctatttaattccttacagttctcgtgaaaaaaaaatttcctttcttcctttaggagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaaggtaaaatgtccagtatttcatatattttgttgctgtcgtaatacatataccataaatgattatttttttagttgcttctttttgcttcaaatttcttctattgtgcgcatttttgttcagctcaaccattgtagtgattttatttctagtcatgtattttgtagagataacattcactgaagattgcagtattacttttgtagtcattacaaatgacatatgtaatatatgtattttttaatcctgtgaaaggtaagttaaaatttcttaatttatactataaaaagtgacctgtcctcaccgttgtactgtgttgatgtagaccagaggaacactgcctgtagcatataagcattttatgtatttgactatgcttccttttttcaaaaaatttttttaatccatgttgcaacttgtgttagtctgtttttgcatctctataaaggaatacctgatattgagtaatttataaagaaaagaggtttaattggctcacaattctgcaaactgtacaggaagtgtggtgcagacatctgcttctagtaattgtctcaggaaacttctaatcatgggcagaaggtgaagggcgagtaggtgcatcacatggcaagagctgagcgagagcatgaagggggagatccagactttattattattattattgttattactattttgtagaaacggggttttgccatgttgtccaagctagtctcaatctcctgagctcaggcaagtcgcctgtctcagcctcccaaagtgctgggattacaggcgtgagccaccatgcccgtccccagactctcttaaataaccagatctcctgtgaactaactgagtgggaactcactcatcaacaaggggatggtgctaaccccattcatgagtggttgacccccttgatccaatacctcccaccaggctctaccccgtctctactaaaaatacaaaaaattagccgggtatggtggcgggcgcttgtagtcccagctacttgggaggctgaagtaggagaatggcgtgaacccgggaggcagagcttgtagtgagccgagatcgcgccactgcactccagcttgggggacagagtgagactctgtctcaaaaaaaaaaaaaaaaaaaaaaaaggagattacatttcaacatgaggtttggaaagggataaacatccaaaccatattacaacactatccatttttctcgtaactttttttttttttttttgctatcttcccttttttttcttgtccttctgcctttagacactagcaatcccaaaattcagttctcgtcatgtattttcattttctgtacctcattaggggagcctatgcatggaagctcagatgtcatctctatgttgtggtatagcagagagagtcagaagacaccgggtgtaattaagcctctgtcactatgcagcgttgctccctttactactcttccaacctctgcaacattttagtgggagcctccaatgtaccaagatctatggtaaacatgaagggatataacaagaagaagtgatcactgttctggtataacactagttataccacattctagtgtccatctgctgtaaatatctggaacattctaaagaatttttaaaaattcagttgtttttgttttcagttctcacctctcccccaagtactagtctcatgttgtcaaatgcccactggatcttgatatctgaatattgtactgttatctcaaattgtcaagccatcttatctaactatgacttaacttttaagactcaatggaaatctctcctgctagttcccagaactttggatttgccttttctttttaatggttttataattcactgttattcagactgttccatttgctcatccctaatatccagtcattgtttaacttgattttcttttttcctcatactgccttgtggctgtgtctcttctttttctttctcttcactactattctagtcagaacctgtcattatttggcttaaccacttataaacgtgtaccttagtcttctcctttctaatctcttctgtgtactaagactagaataatcccagagttctcattggtcatgttgtttctcttgcccataaatcttctatggttcttcatagtctagaaagtaaaacctgcctagtttacttgtgtacgcagaaacttccatagtcttccattttctccagatacttaactttccaaactttttatgttactctccttgaaatatgttcttctctatgaccactctgcttttattcagctcttctatcactcccattctcctgctgtctaaattctgtgcatttatgaaaactcaggtcaaattcttcctgtatgaaacttcccagatgattcaagtcagctatggagatcttgctattctaaattcctcgagcccatattgtccataacctaggaaattccatattttttatgacagggtctccattttttaaatatttgtctcctaaatacccacacattccattttttgggattggtagttaatgatgatagcagatacttactatatgtattttttgacatgagcagttttacataattcttaccattagtactgtgtgattgatattgttattatttggataaggaaacctaagtacaaagaaattttaaattaacttgtcagggtcacacagttggcaagtggatggatgagcaaggattcgaatctagacagactctccagtgccttaccacattttatcctcgctgtatcaagcccctctaccccttctctctttctctcacagtattcatcctctttctatatcatagtatcagattctcacagtgtcataacctctttctatcaaaatatttaaaccacaccctctttctccttctcaagagagattggttttatttcattctctaatggttgcattatattttattgtgtggatataccctaattagccattgttcaattgatagctattaaaatttttttccctgccaaacattgtatatatgtgctttatcagaatacagtttataaaagtagaattctagggtttaaactttaaaccttgatataggagagcactttaaaccttgatataggctgtcttccagttagattttatccatttaaatgtacgttatttgaatatgagtaatgcccatttcctgtgtagcctttagatattaggagtatttttatgtttttcagttctgtaggtgaaaaattatttttatattttgttactttcttttctgttttaaattttttgtagagacgcatctcactacatggcccaggttggtcttgagctcctggcctaaactggttctccctcttggcttcctaaagtggtgggattatggtctgaatcatcatgcctggccctgaaaaattattttaatttacaacaccattttacatgttgtgaccctttttaccccttttagagtgtgtgtcttccttactcatttgttaaaacccatttaaaatgtaatcttgtagagcaacaaagattttattatctcagtgcttctgtgcgttggaaattcaggcatggcttagctaggaacctctggcccagggcctctcaccagactgaatccaggcatcagctggagctgcagtctcatctgaaggtttgtctgaaggaggaaatatctgtccccaagtgtactatgtgattgttggcagactttggtcccttgccacatgggcctgttcactgggctttctgaggtcttaccagttagcttcctacatggcaagggagccactggaaaaacagtaagcccaagacagaaaccacagttgtttttctaacctaatattggtgagttgattaaacaagggtgtgaataccaggaggcaggtatcatttagggccatcctagaagctgcctccacaataatatgaaccatctgtcatcatctcaaagtgagaaaaaataatttcagtttgtgttgactttgcttgtgatatcttttgcttttcagatattgttgatttgggctttggtataatgctgagaaagggcctgttctatcttgcttataaataatgtcgttttctctaggaattttattttcacatatttctttaatttttctggggttatattttgggatgtagtgtaaagtagtaatataacttaacctttccccaagtagtaggtcattgtctcagaaaccatttttataaagtttatccttttcccactggtgggcaaatgccattttaaaaaccatgtattatgttaccatatatacattggattttcttctgggtttccttttcttttccgctgatttgtccattttggcttatgtaacacttctctgatgatgagaactaacagcatattttgagtgttccttgttttttctcttaaaaaaatctgtgtatcaaatatcatcagcctgtgatgtttctcctgcaaccccaacaaaacaaaaacaaactttagatttgattaaaattgtgtaaaatttgcagattagttgggcagaatgtgcagatttacaatattcagccttcccatccagaaatgtgataattctcagcatttaatccatttttgatataaaactttatagttatgtacgttttaaacttttcttacttttttaatctagatatgtgtagtaatttttctgttgctcttgggaatgggagtcttcctccatgtaaatacatcttccatgtaaatgcaagtcttcctccatgtaaatacatcttccatgtaaatgcaagtcttcctccatgtaaatatgtcttgttttaggcatttgaacactgcctcctccgttggttttgaacttgggcatcttctagatgcatttaatgtgctgaggccctttagtagggtactttactatgttgcttttctaaatattattacatgttcattataattttctagcttacctagaaacctgagtttaaaatgttactggtgaactagagtgtttttgttgttttgttttctgttgttagatgaacagtttttattatgattagacactgattttcatatagggtttatggtttttttccctatgatctaaatgtaatatataaacatgataatattcagtcggttttgctttctaataatcctgtttgctccaggtgttttaacattgtgattttttttggaggtattttatgtattagtgctttcttttattttttgtttttctcagtcagacttgctatatagctttgctttagttatcatctcttttcaaggaaccagctgttctacctttcttctaatcattgtatctttcatcactattcctttttaagaaggttctaaagaaagaatatgtcaaatactttgattaaagattgaaaatatgtccctatgtaaatgaactgtagaactctgaaaacaaacatttaaataaaactattaataaacagtgagaccctttctttttttattaaaaaaaaaaacactaaataaaactatttatattgggtagcatagggtaaaacacagtaatacttctgattgtcctcactgggaatttgaatacttttcatgtattacagagaggaacaagtaacctggctgaaaataaaaggatgtgagtcaggaaaataaaataataaagcaagagagcacccaaagccttggcttgcaagaaattgtgtattagatactatggtttttatatgtacttattgacagagaaagtggggatctattgcttaggtaacaaaatagtagatctaatgttttattatgatttttgtccttttttcatgttttgatggagaagatgtacagacagtggtttaagaataacaaacacttgaaacttgcaacttagcctatgctaggattgtgtgggtatttcaccaattaatttaataaatacagagttttgtctaatgagcggtttgaaagtaataatgtgcacacatagcattttgtagtttaaaaattagtttcatatacataaatcatttgatttttgctactttttattttgatataatttcaggcttaggccaggagtggtggctcacgcttgtaatcccagcacttcaggaggctgaggcaggaggatcacttgaggtcaggagtttgagaccagcctggccaacatggcaaaatcccgtctctactaaaaatacaaatattagcaaagtgtggtggcacgtgcctgtagtcccagctatttggaaggctgaggcaggagaattacttgaacctgggaggcagaggttgctgtgagccaagattgcgccactgcactctagcctgggggacagagtgagactctgtctcaaaaaaaaaaaaaaaaaattatttcaggcttaggccgggtgcggtggctcatgcctgtaatcccagcactttgggaggccgaggcgggtggatcacgatgtgaggagatcctgaccaatatggtgaaaccctgtctctactaaaaaatacagaaattagctgggcacggtgactgaggcaggagaatcacttgaacccgggaagcggaggttgcagtgagctgagatcgcgccactgcactccagcctggtgacagagtgagactccgtctcaaaaaaaaaaaataattattgtttcaggcttaacattgccattactgtaaagaacttccttaaaccttttatccagatttccaaatattaatatttatcacactcacttttgcagtttcttcctctctatatatattatttttatttttttcaggtgagatcttgccgtataacccaggctggagagcggtggtaaagtcacggctactgcagccttcaactcctaggctttagcgattctcctacctcagcctcccaagtagataaccataggtgtgcgccaccacacctggctagtttttaatttttttttttttttttgtagagacagtgtctccctgttttgcccaggctggtctcaaactcctgggcttaagtgatcctcctgccttggccttctaaagtactgggattatagacgtgtgtgccaggctacacatattatttcctgaaatgtttgagtgcaacttttaagtatgaggctcttttatccctaaattattcagtgtctgtttcccaaactcaaaagacattcgcttatgtaaccacataataattataaaaaaatcaagaaattaacaattatacaatactattatctaatatgcaggctttattgaaatatctccagttgttccaaaaatgtccattgtggcacaagaaaatcctgggtcatgtgtttagttgtgatatctctagtcttctataccaggaagagttcctcacttattgtctttcatgacattcatgtttttgaacagtataggacagctatttgggtttgtgtaatgtttcttcataattaggttcagcatattcatttttggaaagaatgtcattgaaatgatttttttcttcttaatgcattatatcaggagacccatgatgtctgtctagttaatgatgatattcacttctatcacttagggtagtgtttgctaggtttctttactgtagaattacggtttttctcttcataattataaggaatatgtaggaaaatacttttagactacataaataacctttttctcatccagcttttaccaactagttttagtatgcattaatgagtttttgttagaatcaattattataataatggctacaaatggtgattttctaattctttttttctttattaattagattctactctaaggattatgaggaaagcttttcccttctctcatttaattttatatatcatacggactcattgactctcatttaattgtatatatcatatggactcattgactctcattttattaaatgggttataatctttggtcatttattttgatactcagattgcttcaggccaggtgcagtggctcacacctgtaatcccagcacttagggcggccgaggcgggcggatcacgaggtcaggagattgagaccatcctggctaacatggtgaaaccccgtctctactaaaaaatagaaaaaaatagctgggcatggtggcgggtgcctgtaagggaggctgaggcaagagaatggcccaggaggtggagcttgcagtgagctgagattgcatcactgcactccagcctgggtgacagagcaagactctgtctcaaaaaaaattgcttcagatttggtcagcgaaagccctttcaagctggatctagatccccttagccagtagaactagagaataggtgtatgtgtgtgcacatgtgtgcaagcatattcacacgtttttttctgtatttctttctattaaaaattaatacttttaattgcagagcaacatcacagagttcactctagccttctttatttctatatttgtaactcctctttctgaactgagaaaacctagctgctgttatccacaatatatttacttatttgctcaatcctagaatttacaagctagtttcagagctgctaactcatatccctgggggcagagttggacttactaagagttcactgtttatgttttttatctttagcttgaaaggcatatggattttatattatattcaaaaagtatttgggtttttctttttttttaattttaattttttcctgtagcatagactattaatttgaagtagttaggttcatctgtttctgtatgtatcgcttcttagaacttcaccccctttctttttgattaaaaaaattatttatgttttcattctgtgaaacactaacatggttccaaaagtcagaaatctagtgtcatttcttttcgattctttctattttatttccactcactccctgtagatggccaacctcattatgttctggcttacttttcctcagttttgttttacacatacctgctcgtttgtgcattttctttttttttttttcttccttacacaggaggtagcatattggagacactcttttgtactttgctttttctttaagctatatttcctggcaatcattgcatatcagttcagagatttttttttttttcatcctttttacagctgctaaatgcccctccataaaggttataccaatttgcattctctctagttaagtatgagtgcctgttggtccatagttttggctataaaagttgccttactttttaaatttttgctaatctcataggtgagaaatggtatctcattatagttttaatttacaatcttcctattttgagtacactttaacatcttttcatgtattcaaggtccattttatttgtttttgtgagtctgtatgaattgtcctttttttggtccattttaaaaatgtgattttgatttttcccaatcaaccctgaagagtactttattatggaatttatattaggtgtgatatgtgtaatttttactttgtgtatggtagttttttttttctgatatgtaagagtccttccttccctctatgcagttgaatttattgctcttttatctggattttgtgttatggttataaagctttccagctatcctcaggttaaagaggaagtcatctatattttcctctagtagttatcagggttcagtttttgcatttagatccctgatccatttggagtttattcctattatggtatgaattattgatctaattttatcttttctaaatcattaactacttgtctgggtacaatttagtagaagactatctttgttccagttatttgagatgttactgttattatatgctaaatttctacatgtacttggctctatttctggactttccattttattccactaagctgtttgcatattcatgtgtcactactataccgttttgattatagagactttatagtatgttttaatgtctgatagggttaatatgcccttgtagcttttctttttggtgttttcctacctatttttacatgtttatttttctatatgaactttggcatcagtttatctaactttataaaaaaagtgttgatcttttaaattgggattttatttataaattaatataggaagaactgatatatttgtgagcttgaattatcttagccaggaataagaaatatttttccatttgttcaaatctacttttatgtctttcagaagttttggcttagtcaggctttagaaatttcttaagtttattcttaagtattttattgtttttgttgctattgtatagtttttcttgttttttattctacatgttaaaaagtgatggactgctttataccaagctcggtttttttccttgtatcacccttcctttgtggaatttaagtattctttgttgtggccaaaaaataaaatttgatatattgacctatttgtttctcttgattatcataaaataggaatttctctgacaacttaaaagtctgtagttttaaaatctgtgatttacatagctgctgtttgggattgccttaaattattaggtgtttccagcagaaggttgaaataggatcttctccttgttcaattaaataaaccaacctgcgaaatctcctaatggcagtgagaggctttttttttttttttttttttggtgaatagtaaagatgagagataatggtcaaagataaaaagaaaaccaagaataattatccctgtgtgccaggagtcacataaactgtacctaagacactgggctttttaaaagaatttctggtataatcttcatttcagtggtattttatttttttcccactgttgtccatcatactgtgacagagtacttaaactatcttaacttgggcagtttggaagtgcttgtgataggaaatatttgtgttgtggggataaagattgggtaggtacctaattaatgtaggagacctgtactgttgtaacagggagaacatcagtcaatcccaaacctccttatggtttgttgaggctgactgttccaacttgaaaatcagtaagttgcatgtttattagtgatcttggcttcatttactattcatttctcctttgaagtgagtcataggtttcaagtctcctgcctagtttcacttgttctgtaattgggatatagagggatagggatgtgaggtatcactattgttaggttgtgaatggagagcatagtctacttttaataccattttatttatttatttattggagacagaatttcactcttgttgcccaggctggagtgcaatggtgcaatcttgtctcactgcaacctccgcctcctgggttcaagcaattctcctgcctcagcttcccaagtagctgggcttgcaggcatgcgccactactcctggataattttgtatttttagtagagatggggtttcaccatgttggtcaggctggtctcgaactcctgacctcaggtgatccacccacctcagtctcccaaagtgctgggattacaattgtgggccaccgcgcttggcttaataccattttaatacctgcttttatagcattttggagaatgaggaaagatgatgtctgtttttgatccttaaggatatgagtattctctgaatatattctggagacagtgccagaggaagattttggtcttacttaataattttgaaagcttgagcatatctattgtaagagttaaagaaagaggaaagaaacacgaaatgtggctggcagttaaagacaagttcactttagacaaaacctgagaggtgctcctggctgattttgatcaggagcgctttctcttacagactaagagtatatattggttttagggtgagggggcttatcacaagattggaatgtttatgtgtgtggagaagtttatggtggggttagaatctctctgggaggaggggaggttatcttggggcagacatctttccggccaggaggggtgttatctcggggctagcatctttccaactggagcggggtttatttcggggctagcatgtctctggtcagggaggagtttggaatgtttctggttggagatgttatttgtggtttatggtcatgctgaccttagccattaggctgatgccctttgaatttaggcagttttttattaaggtgaattttagaatgaggggcttgtccaagatggcgatgctcctactctgtcaatccagaccctataatttgaggagggacggcgtgttcttctggctacttcctgctgactaggggatggagagttttctggtctcaggttgactgtaggagcaatgacatctgtagatgtttttgggtagttgtctgtgaaatggccatgatcctgtcagataaaaatctttgaaaaaggttaattgggcagggtaagaacattagtcccaggcatattattagaagggggctcaggaatgggatgacacatgctatgattttgtttccaaaccaagaatctgtttggttgtttagatatttccttagctttttatttccttttttttttttttggagtcagactcttgctccgtcacccagactggagtacagtggcgcaatctcagctcaatgcaacctccaactcccaggttcaagcagttctcctgtctcagcccccgagtagcttgggactacaggcgcctgcctgtacgcccggctaatttttgtattttttaagtagagatggggtttcaccttgttggtcagggtggtcttgaactcctggcttcaggtgatacatgtgcctcagcctcccaaaatgctgggattacaggcgtgagctacttcacctagcaagatttcttatttctttacattgatatttatgacttttgggggctgtcaggggttgcttccttagctttccaggctttgacttgagtgtgatgtatttaggagttgattcctgtaacaggtaccattttaattaccctgggttccgtggattcactagacatgggggtgaaggattctgggcaccctcagttattagttgtcagcaccagcagtgaagagatttccttctgtgatggtcggggagcttagaggcagcgtctgctgaaacatctagttttcagtttatagggctttaagaaagcccgacttattttggaaacttgtagccagaaaaattagaatttaatttaagcagtagaaaataataaaaactgaaaaatgttaggcaacactagaatttaacaacaggtgtgctatggttttttaaatataattttctttttccagtttcccatttttattaaaagacaaatcatggtaggaatggtttgctttattatacttggcttaattatttgcatacagtgcagcaagaataattatttgttacataggccttttaaattggctttgatggaactttgttctgtagaaggaatctgagagaaaaccgttttagccaagcccagccctggatttgtactatcaaatatctatgagttggtgaatttctttcctcttgaggtcccaagataacttggtattcctggtctgctagaaagtgacattctttacttaccatagaccagaaaccctgtacagggactgtgtacacaaaatatgaggccagttttccaagggcatttttgcctttgtaagtatggttccttaaaggaaagcatattattccagttaaagcctttgtaaaaataaccagtttttccaattctgtcctgttacaaaagaaaacagatttttagtgcacttatgcaagtactgtaacttaagaatactcacagatagtttcaaaattatggagaaaatcaggtagagagaaacaagtatgttccaaattttgttcatggtagtatagtaaattgttaaaagctgttaatagctcaaaagaaaaatttctttgactttgaaaagcaaaacaaaggattagcaatatgttaaaacattaaaaagattggtctcctgttagtttagttcatggaattaattcctgtcatgctcaatattaacattttatctcttcaaaagtcctgaacgtttttcctctattctgatgttacagtctccagtgttatcagaaacctgcatttaagagcacctgttagagctttatagctgattataaaaccaccttttaaagaggaccaaaacaaggcaacaattgtttatggatgacaaaaagttttagggtagccataaagacacaattgacaaggatgacacacaataattttaacataacgattgtaattattactgaaaatgtacactaatatatatcagaattataggagtcttccataactttggaacacatatgagtaacatacaaatatagaccaaagaaagccaaataccgttttatatttgacaatgcttcttgtatgattttataccagataagctaaatttcacctttctattagtgtgctattaatgttaaactcaattttaataagaccttgtaggcatatttatccaattttaatatctgaccataaggtaagatttttatagactttttttaatgctttataagttttgttaaagaacaggttagtgctttaagaaaaacccattgtgcttttgttttaatgcccaatttacagaaaaactggatgatacccctttaactttaccaatatgtttacacacagaatttcctttataattaacgtttcaaaacttgcttaaaccttcaaaacaattttttaactttttaatgtaggtaaaaatccacattcttatgcctccttataatccttttacgaaaagtatattttactttccttacacaccttgcatataaactgttttttcaatagctttaaatacatgttacaccattaacttttattattatactttaagttctagggtacatgtgcacaacgtacaggtttgatacataggtatacatgtgccatgttggtttgctgcacccatcaactcatcatttacattaggtatttctcctaatgctatccctccccgcagtccctcatccccttacagtccccagtgtgtgatgttccctgccctgtgtccaagtgatctcattgttcaattcccacctgtgagtgagaacatgcggtctttggttttaggtccttgtgatagtttgctgagaaggatggtttctagcttcatctgtgtccctgcaaaggacaggaactcatccttttttatggctgcatagtattccatgctgtatatgtgccacattttcttaatccagtctatcattgatggacatttgcgttggttccaagtcttgtgaatagtgccacaataaacaaacgtgtgcatgtgtctttatagtagcatgatttataatcctttgggtatatacccagtaatgggattgctgggtcaaatggtaattctagttctagatccttgaggaatccccatactgtcttccacaatggttaaaccaatttacactgccaccaacagtgtataagcattccagtttctccacatcctctccagcatctgttgtttcctgactttttaacgaccgccattctaactggcgtgagatggtatctcattgtggttttgatttgtgtttctctgatgaccagtgatgatgagcattttttcgtgtgtctgttggctgcatagatgtcttcttttgacaagtttctgttcatatctgttgcccactttatgatgggggttgtttttttttttgtaaatttgtttgagttatttgtagattctggatattagccctttgtcagatggatagattgcaaaaattttctcccattctgtaggttgcctgttcattctgatggtagtttctttcaccgtgcagaagttcttcagtttaattgatcccatttgtctattttggcttttgttgccattgcttttggtattttagtcatgaagtccttgcccataactatgtcctgaatgatattgcctaggttttcttctagggtttttatggttttaggtctaatatttaagtcttttatccatcatgaattaatttttctataaggtgtaaggaaggggtccagtttcagttttctgcatatggctagctagttttcccagcaccatttattaaatagggaatcctttccccatttcttgtttttctcaggtttgtcaaagatcagatggttgtagatgtgtggtattatttctgagggctctcttctgttccattggtctatatatctgttttggtaccagtactatgctgttttggtgactgtagccttgtagtatagtttgaagtcaggtagcatgatgcctctagctttgttctttttgcttaggattgtattggcaattcaggctcttttttgattcaatatgaagtttaaagtagttttttccaattctgtgaagaaagtcattggtagcttcatggggatgacattgaatctataaattactttgggcagtatggccgttttcaccatattgatttttcctatccatgagcatggaatattcttccatttgtttatgtcctcttttatttcgttgagcagtggtttgtagttattcttgaagaggtccttcacattctttgtaagttgtattcctaggtatttaattctctttgtagcaattgtgaatgggagttcactcatgatttggctctctgttggtctgttattggtgtctaggaatgcttgtgatttttgcacgttgattttgtatcctgagactttgctgaagttggttatcagcttgagattttgggctgagacggtggagttttccaaatatacaatcatgtcatctgcaaacagggacaatttgacttccttgtttcctaattgaatgccctttatttctttctcttgcctgattgccctgaccagaacttccaacactatgttgaataggactggtgagagagggcatccttgtcttgtgttggttttcaaagggaatgcttccagtttttgcccattcagtatgatattgattgtgagtttgtcataaatagctcttattttgagatacgttccatcaatacctcgtttattgagaattttagcatgaggggctgttgaattttgtcgaaggccttttctgcatctattgagataatcatgtggtttttgtctttggttctgtttatgtgatggattacgtttgttgatttgcatatgttgaaccagccttgcattccagggatgaagcctgctggatcgtggtggataagctttttgatgtgctgctggattcaatttgccaatattttattgaggatttttgcatcaatgttcatcagggatattggtctaaaattctcctttttttgttgtgtctctgccaggttttggtatcaggatgatgttggcctcataaaatgagtcagggtggatcccctctttttctattgattgaaatagtttcagaaggaatggtgccagctcctctttgtacctctggtcctggactttttttggttggtagactattaatcattgcctcaatttcagagcctgttattggtctattcagagattcaacttcttcctggtttattcttgggagggtgtatgtgtccaggaatttatctgtttcttcttgattttctagtttatttgcatagaggtgtttatagtattctctgatggtagtttgtatttctgtgggatcggtggtgatatcccctttatcattttttattgcgtatatttgattcttctctcttttcttctttattaatcttgctaccggtctatcaattttgttgatcttttcaaaaaaccagctcctggattcattgatttttttttttttttaaggtttttctgtgtctctatctccttcagttctgctctgatcttagttatttcttgccttttgctagcttttgaatttgtttgcttttgcttgtctagttcttttaattgtgatgttagggtgtcaattttagatctttgctgctttatcttgtggccatttagtgctataaatttccctctacacactgctttaaatgtgttccagagattctggtatgttgtgtctttgttctcattggtttcagagaatatctttatttctgccttcattttattatgtacccagtagtcattcaggagcaagttgttcagtttccatgcagttgtgcagttttgagtgagtttcttaatcctgagttctaattttattgcactgtggtctatgagacagtttgttgtgctttctgtccttttacatttgctgaggagtgctttacttccaattatgtggtcagttgtagaataagtgcagtgtggtgctgagaagaatgtatattctgttgattttgggtggagagttctgtagatgtctattaggtctgcttgttgcagagctgagttcaggtcctggatatgcttgttaaccttttgtagttgatctttctaatgttgacagtggggtgttaaagtctcccattattattgtgtaggagtctaagtctctttgtaggtctccaaggacttgctttatgaatctgggtgctcctgtattgggtgcatatatctttaggatagttagctcttcttgttgaattgactcctttaccatcatgtaatggctttctttgtctctttgatctttgttggtttaaagtctgttttatcagagactaggattgcaacccctgcttttttttttttctctccatttgcttggtaaatcttcctccatctctttattttgagtctgtgtgcgtctttgcatatgagatgggtctcctgaatacagcacactgatgggtcttgactctttatccaatttgccaatctgtgtcttttaattggggcatttagcctgtttacatttaaggttaatattgctatatatgaatttgttcctgtcattatgatgttcgctggttattttgcccattaattgatgcagtttcttcatagcatcagtggtctttacaatttggcatgtttttgcagtagctggtaccagttgttcctttccatgtttagttcctccttcaggagctcttgtaaggcaggcctggtggtgacaaagtctctcagcatttgcttgtctgtaaaggattttatttctccttcacttatgaagcttagtttggctggatatgagattctgggttgaaaattcttctttaagaattttgaatattggcctccactttcttctggcttgtagggtttctgccgagagatccactgttagtctgatggccttccgattgtgattaactcgacctttctctatggctgcccttaacagtttttccttcatttcaaccttggtgaatctgtcaattatgtgtcttggggttgctcttctcttggagtatctttgtggtgttctctctatttcctgaatttggatgttggcctgccttcctaggttagggaagttctcctggataatatcctgcagagtgttttccaacttggttcccttctccccatcactttctacaccaatcaaacgtagatttggtcttttcacatagtcccatatttcttggaggctttattcatttcttttcagtcttttttctctaaccttctcgctttatttcattaatatgatcttcagtgactgataccctttcttccacttgatcgaatcagctattgaagcttgtgcatatgtcatgacgttcttgtgccatgattttcagctccatcagtttatttaatgtcttctctacactgtttattctagttagccattagtctaatcttttttcaaggttattagcttccttgcgatgggtttgaacattctcctttaactcggagaagtctgttattaccgaccttatgaagcctacttctgtcaactcgtcaaagtcattctccttccagctttttttctgttgctggcgaggagctgccatcctttggaggtgaagaggtgctctgattttagaattttcagcttttctgctctggttctcctcatctttgtggttttatctaccttcgttctttgatgttggtgacctacagatggggttttggtgtagatgtcctttttgttgatgttgatgctattcctttctgtttgtttgttttccttctaacagatccctcagctgcaggtctgttggagtttgctggagttccactccagaccctggttgcctgtgtatcaccagcagaggctgcagaacaacaaatactgcagaacagcaaatattgctgcctgatcttcctctggaagcttcgtcacagcggggcagctgcctatatgaggtgtctgttggcccctactgggaggtatctcccagttaggctatacgggggtcagggacccacttgtggaggcagtctgtctgttctcagagctcaaatgccgtgctgggaggaaccactgctctcttcagagctgtcagatagggacgtttatgtctgcagaagttgtctgctgccttttattcagctatgccttgcccacagaggtggagtctagaggctgtaggtgttgttgagctgtggtgggctctgcccagttcgagcttcccggccactttttttacctactcaagcctcagcaatggcagacacccttccccagccaggctgccgcctgacagatcgatctcagactactgcgctagcagtgagcaaggctccgtgggcgtgggacccgccgagccaggcacgggagagaatcatcttgtcagcgggtgctaagaccttgggaaaagtgcagtatttgggtgggtagtttcccatttttccaggtagtctgtcatggcttcccttggctaggaaagggaaacccccgactccttgcgctttgtgggtgatgcaacgccccaccctgcttcagcttgccctccatgggctgcacccactgtccaaccattcccaatgagatgaaccaggtacctcagttggaaatacagaaattagccgtcttctgcgtcgatcatgctgggagctgcagacaggagctgttcctattcggccatcttggctacgctgttaacttttagtaaactttacttttgttgaaaaccttgtaagtttgggattttaattatgtgctaggtgtagagcccaggacctagaaagaagtgcagttaaatgttctagcatttaactccacgtgtcgtaggttttacctagctgcaaagcaggccagttgtccagctaagagttacagtgacattttataaagcattcaggaggcctaattacttttaaattatacaacatttcttgtataaattcccttttataacatttttcatgactttcacagacaatccttgacatgcctcaactttctgacttgtaaacatctctttctttaaacaaccagttaacttaggacaagaatttaccatataagattctttttccataacttctcctttctttaatgtcacagatgataaccattctttcccaaagcaaacttccttcatgtttgtggactagactgcctaagccacaagattagaagctaagatattttactaaatagtttaagatacagctatcttcattaaaccaatattaatatttcatttattaaaaaattacacaaggctgggtgcggtgtctcacgcctgtaatgccagcactttgggaggccaagacgggtgggttacctgaggttgggagtttgagactagcctgaccaacttggagaaaccccatctctactaaaaatacaaaaaattccccgggcgtggtggcacatgcctgtaatcccagctacttgggaggcagaggcaggagaatggcttgaacccatgaggcagaggttgtggtgagctgagattgtgctattgtacttcagcctgggcaacaagagcgaaactctgtcttaaaaaaaaaaatgacacaagcaaagatgattttgtttgggctgagttagttttgcagcctctatgcaaaatgttgacaccttacagtatttggccgagaaaagtatgaatttgcctgattaacaaatgcaaacaaaaaggtatgctggcaacttttaagatacttctaactttagtgtaccagtaagatttaaagattaaggtcacgtgaactgaaaggtcccacagcttttacttttcccttaaaaatatttgatttaagtgcttagttttcttaggccaattaattagagctcttttaatagacattgcacagacaacacatatatagccacacagacaaccagaagaagattcagtagctataacgttttttttttctctctgctaatttcccaattggattattgaccttcaggtgaggcccttgaagaacaggactaggaaaacagtttccagggtctaataaacaggtatagctggaagacaaatacaaagattttgagaggtacttatttacctctaattccacggtttccataaggaaaacagatttttccccaaaatgggatttgtggtgccttttctcttttccaaaggaatcccagggcaccagaagtcattttagggttttttacatatgcaccaagagttgcaagacacagtgaagaaaagtaattgagttaactgggggaaaaccttttccaggaaaataagattgatgaagagaaaactataaagaccttttgaatatactcatagttttgatctgtgacaagtcagacatggagaaagagacatgaacatgagcagtgcctttaactccagccacttcccagagagggagaatggcagaggtggtttttgaatgggtaacaggtggaggaggaggaggagtggggtttggggctgaaggcttggaggcaggtagggccaccagggtggagggtttggacaggctaggagcagatgcagggggataagaatacagagagggttcatctgcagggtcaaaaggagtttcggaggaaggagagacccggagagggtttttaataagaagaaagattttataagaggtacaagcttgacacaggtagggttgggatttaaggtagaagtagaagaaggcctgaatatagagaacctcttgccatttgccattcctggtcataaagttgtgaagttcctgagaatttgcaagttaaagatgccattttcggaccatgggctgccattatctaatgtgttttggggccaggctgtgttacagtaaaagactaaacacttaagccaactttggcgaggtcatgaaggagacagcacagtggagagaccgtaggaatgtggaattgtttggcacccatgtggactggtgagaagaagccgagggtgtctgtttttgttctaggcatccccagacaaaagacagaagtccggaatcgtctttctgaagaggacagtcatcaagctgagaagaaactgggcatccccaagatttcctctagcttagtcctgctggtcctctgaggaccgggatggctgacctgacttttcctgggtactgcaagaaagccaggggacggcagatcttaccagttggctggattagtgtccaatgttggatgttccggttggatttggcaaagggcctcctggactgcagccgcacgagggagagagagagaggaagaaaaaaaaagggaggatgagggaaaagtgaagtgagagaaaagatggggcatgtggccagagaccctcaggatccaggaattaactcaagatgagctgctgttgcccactgcttcctgggttgcaagagagcctctgcccccagcactcatcccgggtttgggcaccaaatgtaagagttaaagaaaggaaagaaacacaaaacgcggctggcagttaaagacaggtttattttagacaaaacctgagaggcactcctggccgatttcgttaagaagtgtttccttttacagactaagaatatatattggttttagggcgagggggcttatcacaagcttggaatatttatgtgtgtggagaattttatggcggggttggaatctctttgggaggaggggaggttatcttggggcagacatctttccggcccagagaggggtttattttgaggctagtatgtctctgatctgggaggagtttggaatgtttcaggttggtgatgttgtttgtggtttatggtcatgctgaccttagccattagggggatgccctttggatttaggcagtttcttattaaggtgaattttagaatgaggggcttgttcaagatggtgatgttcctgctctgtcatctgtgaaatttatgaccctcaatttcactgcctgtaaaatggggacggtactaatacagcaatgcccagcccagtgtttggtacataaaggaactcagtaaacattattgtgctgccttttttctcttcttcagtgttttgtgaatcaaatggaacaaagttttatgacaacattttctaagttttaaaactccttataactaaactctttgtttttattcttaaggttgtaagttactacacactgctatacgtcaaattttttccccctattggttttatactctcttaaatttggtaattctgttttcaggcacctaattattagtattttatatagccatttaagtcacttgtaaaaaatctatttgctgataaaaccgatagaaacttgaaagaaatagtaatctttgtgctatgtgtagcactgttctttaagtcatggaaatttgtagttttcaagtggaggctataattaggtggaagaaaagggaaggacagctacaagttggataggaaaagtgttacattgccagtacagctctaagaaagtaacagcatggcaatgggagtcctcaaaccaaagttaccaagtcagaggagttcagtttttcctgggaaaaggtctccattaagtatgcctgctgttctcagtcatataggctgggaatctttgcctgtccccagcaagagaatggatttgataggtcagcagttgggaccattgataatgatgttccttgcagtgggaaacctgagaggcaggttttcatacaaagtttcattaaaactttcatacagagtttcttaaggatacagactgttttatgctgcttttatagtttctgaagagcctaggtaggttaaatacatttttaaaattttaatggaattttcagggaattaacaaagattgatgtggaagaactgtattgagtttgctgttaaaattatatatagttttattgcctccttgattgttccactgagaaaaaatggaggcacgatatttgatatttgtggacttgaatttcttctttctggctttcagtgtcttgagtggtttgagagaaggagaaataaaacttgatataaggcataatgtgctttttaagttttctcactttttttgagtttactgttattttgtgtttatactgtttcaaattcccatatatatctgaggtaaggccaaaagcagtattgaatatcaggagttaaagttactcttttgactagtttcactaaaacctttctattttttttttttttttttttgtggggagtggtcagctgcttgatatatttatttataacaaccttattgagatataattaatgtgatgtatatttacccttttaaaggatataactcattggttttagtatagtcacagagttaggcaactatcactactatctaattccagaatattttcatctcaaaatgaaactgtgtacccattagcagtcactccctatcctttccttcccccaggccctggaaactactgatctactacctgtctatggatttgcccatcctggaatcacacaatgtacgatcttttgtgtctggcttctttcacttagaatagtatttttgaagttgattcctgttgtagcatgtctcagtacttcattccttttatggctgaataatattccattgtatggacatgccacatatggtttatctgttcatcagctgatgaacatttagattgtttatactttttggctgttatgaatgatgctactatgaatatttgtgtcaagtttttgtggggaaataggttttcatttctctttagtgtgttcctaggaatggaatcacaggcccataaagtgattaaatgattaacagtttgaagaaatattaacctattttctaaagcagctgtacctattttctaaagcagcaatcccccaacaatgtatgagcattgcaatttctctgcaactttgatagcacttgttattatctagttttttattatacccatcctcgtgggtgtgaagtggtatctcattgacatattaatttgcattttctgctgttctatatcttttcatgtgcttattggtggtttttatgtcttcttggggagaaatgtctatttgaacagttgtagttttacagaaaagtgagtggaaagtacagagaattgccatatatacactcattccaaccccatctctagagtttccaatattactggtgccttggtgtagtacatttgtgacaatcaatataccattatttatacattattattaactaaagtttagagtttgccttatggttcactctttgtgttgtacagtctgtgggttttgacaattgtatagggacatgtatctactattagagtatcatacaaaatactgcactaaaaatttcctgaataacacctactcatttttcccttcttcaagacacttgataactgttgatctttttactatttccatagttttgccttttcttggaatcatacagtatatagccttttcagattagctttttttctcagcaatatgcatttaaggtgcctgcataactttgtgatatgatagctcatatctttttttttgagttggagctttgctcttgtgccaaggctggagtgcagtggcacaatcttagctcactgcaacttccgcctcccaggttcaagcgattctcctgccccagcctcccaagtatttgagattacaggcgtgtaccaccacacccagctaatttttgtatttttagtagagactgggtttcaccatgttggccaggctggtctcgaactcctgacctcaggtgatccacctgcctcggccttccaaagttctgggattacaggcttcagccaccgtgcccagccgatagctcatttctttctagtgctgaataatattttatttatggatatatcacaatttgttcatccatttacctgttgcgggacatcttggtttgggtagctgaagtttagacaattatgaataaaggtgctgtaaacattcatgtgcaggtttttatgtggtcatattttcagtccatttgagtaaataccaggtagtacaattgctgtggtaagagtaagtttagttttgtaaacaagtgctaacctgtcttctaaagtggctgtgtaaaataatttcgtattcccaccagcaatgagtgagtgttcttgttgctccacatcctcaccgtcctttggtgatgtcagtgttttggattttagccattctaataggtgtgtagtagtagtatgccagtgtattaatttgcaattccctgatgacatatgatgttgaacatcttcttatatgctttattttcccatctgtttacctcttttgatgaggtatctgttcaggtcttgtcttagtccattgaagtagctaaaataccataaacatgggtgggttataagtaacagaaatttatttcttatggttatggagactagggaagtgtaagatcactggcagattcagtgtctggtgagggcttgttttctggttcatagatagtgccttcttgctgtgtccttccgtgggggcatcttttataagggtattaatctcagagtcctggggcttctggggcatcttttataagggtattaatctcagagtcctcatagcatgatcacctcccaacagccctacctcctaataccatcacattgatgcttaggtttcagtgtatgagtttgataggctagtctactaacattcagaccatagcaagccttttgtccaattttaattgagttgttggttttcttattgttgagttttaagaattctttgtatgttttgggtaacagtcatttatcagatacgtattttgcaaagacttttcctagagtgtgtgacttttcattcatgtcttcctcagagcagacatttctaattttaacgaagttcaacttaccaaattttttctttcatggatgtgttttttatgttgtattttaaaagtcaccaccaaacccaaggacacctagatttttttcctgttatcttctaggagtttaatagcttttcattttacagttaagtctatgatccatttcaagttaatttttgtgaaaggtataaggtctatgcttagattcatttctttaattgtgtctgtccagtttttccagcactacagttatttccccttactttcgggggtatgttccaagacccccagtggatgcctaaaaccatgaatagtaccgaatcctatatgtaccatttttttcttatacatgtgttctgtgataaaatttaatttacaaattaggcacagtaagtgattaaaacaaaataataaaatagaacaattatataattacagtatactgtaaaaaagttatatgaatgtggattatctccctctctctcaaaatatttattgtgctgtaaccacccttcttcttgtgatgatgtgagatgataaaatgcctacatgatgagatagtgaggtgagtgacataggccttgtgacatagccttaggctactactgaccttctaaccatatgccagtaggagaataatctgctgtgaatgatcctagatcagtgagccataataatgtccatggttagacgtcaagagcagacaatgccgatgactaacgggcaggtagtatatacagtgtggatgtgctggacaaatagaggatttatattcctagtgggatagagcaggacagcgtgagatttcatcacactactcagaacagtgtgcaatttaaaatttatgaatagtttattactggaattttacatttaatattttggaactgtggttgactgtaggtaactgaaactgtggaaagcgaaagtgcgggtaaggagagactaatgtagtagtagtagtagtaataatgcttactacttagggttaatataatgattaaatgtggtaatgaatgtgaaatgcttaatccaatgactggcacaaagtaaggtctcacttagtggcagcttttacgattagcagtagttcctcaatattggaaagtgtagaattctgaggagccaggcatccactgatctttttctaaattataccacagacaagcatcagtcaaattggaagcctaggaaaactattatttagacaaacaattgtaacaaacgtggctcttttaacttctatagaactgttttttgttattgttttttaataacaaaatattatggtacaaaacaattgaaagcttctagtttttcataggtcctacaattgcaaaaaggtataattgcttaaaaaatccatatcacagaacagtaaaaagaaaaaaataggtgggcgcggtggctcacacctgtaattccagcgctttgggaggccgaggcaggtggatcacgaggtcaggagatcgagaacatcccggctaacttggtgaaaccccatctctactaaaaatacaaaaaattagctgggcttcgtgacaggcacctgtagtcccagctactcgggaggctgaggcaggagggtggcgtgaacctgggaggcgaagcttgcagtgagccgagatcgcaccactgtaccccaggctggactacagagcgagactccgtctcaaaaaaacaaacaaacaaacaaacaaaatagataaaaattatcttttttcccctccccttaattctgtttttcattatagaatttgtaggtccttctaaatctttttctgtggataggaatcataagaaatgaagcccatcttgatttcttatgatgcctatcatataccttatttgaatctcatttattatgatgcctatcttataccttatttgaatctcattgtgttgtaccttgaaatttcttacataaagtttctgttagaggtaaaatgttattagctcttacctgtataacagctatctatatagacacctaaattggttaattattgctaagagaactaaacatttagtaaagttgttttataaaagtagtcttagactactaagtgacttgaccataagccagaaggagggtcatctcctttatttcttacagttctggaggctaggaagaaattagggaacttagttaagaaaataagtttaatttttaaagaaattagggaactttaattgccttataaaaattttattgcaggccgggcgcggtggctcacgcctgtaatcccagcactttgggaggccgaggcgggcggatcacgaggtcaggagatcgagaccatcccggctaaaacggtgaaaccctgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaaaaaaaaaaaaattattgcaaatagtttaagtatcaatatgttttatatcagactttttaggaagagatgtatacccaaatatataaggccagttactaaaactatggaagctataatgaaattctgttagcaagctgtggggcttttactaattctgccctaaaactcttccagtcaaaagcaatgatcaaattattactagagcataagtaaatgtttatggtataaatgaaactgtacctgtaggagttttaatacatttaaaatttaaaaattctacaaaatggttggtttcttttaaaaattctagttttgtgatgttttaatccagaaattgaatcttgactgaagttggtcttcccatacttgaaatttatgttttaaaatttggatggggatggggagaaactataagtaaaacctacagcattctgaggtccaaaacttcctagattgcttcaacttcaatccatttggggaattttggagacatttgtcatttatataagaaagttaatgattaaactttgagaagtcagtatatcatggaaacttttaccttaatgatattattctgaaaagaatataatcatacagtggaatctatgcagttattttaaaaatatgcttcttaatgtgggagatattctaattgtttaagaaaagtctacaaaacagtgaagtgatattttcatttagacaacaaaaaagaatatagcctatcagagtatttacagaaatagttttcatgatgctccagaacagtaagtgatgattgtttttgttttttattttttgttttttttgtttgtttgtttgtttttttgaaacggagtctcacactgtcgcccgggctggagtgcagtggcgcgatctcagctcactgcaacctctacctcccagattcaagcgattctcctgcctcagcctcctgagtagctgggattacaggcatctgccaccatgcccggctaattttttttttttttttttttttttaattttagtagagacggggtttcactgtgttggccaggctggtctcaaactcctgaccttgtgatccaccggccttggcctctcaaagtgttgggattacaggcgtgagccaccgcgcccggctgatcgtttttgttttttattctcaccagcagggtatcccaaggcttggtttagtcagtctctttaattttagctattctaataggtgtatagtggtatttcattgtagttttaatttgcatttttaaaatgactaatagtgttgaatatcttttcgcatttatttgccatccgtatattgtctttggtgaagtttctcttcaaatcttttacccattttaaaagattgaattgtttcccccgccccttatctgagaaggatacattccaagactcccagtgaattccagaaactgcagatggcactaaaacctgtatatactgcatttttgtatacatacatacctatagtaaagtttataaatcaacacagtaagagattaacaacaataatatgacagtgtcactactcttgtgctttggcaccattatgataagtaacacaagggttactggaacccctgcagttgatctgctaactgtgagagggctgctatgtgatgaatgggcaggtaacatgttcagtatggacaagctagacaaagggatgattcatgacctggtaggagggactgggatggcatgagatttcaccacgctactcagaacgatggcaatttaaaattcattcattattcctggaattttccatttattatttttggactgtggttaactgaaattgtggaaaatgtatccacggaaaatgaaaccgtggataaatgtttagactactctacttgttgaaaagactatcctttctccattgaaattgctttgttcctttgtgaaagtttagttgaccatatttgtttgctagttctgggccatctgttactgttccagtaatctcttctttcaccagtaccatgctgtctttattcctgtagctttatagaaagccttgaagtcaggtactgtcattcggctgactgttgttcaatactgtgatgactattctgaatcttttgcctttccatataaattttagaatcagtttgttgatatctgcaatgtaacttgctgggattctgattgatactgcattgaatttgcagatcaagttgggaagaactggcatcttaatattgagtgttcttatccatgtacatgatgtaagtttccatttatttagaccttccttgacttttttggtaacagttttatagttttcatcatatagatttttttttttttttgagatggagtcttgctttgtcacccaggctggagtacgttggcgcaatctcggctcactgtaacctccgcctcctgggttcaagcaattctcctggctcagtctcccaagtagctgggattacaggcgtgtgccaccacacctggctaatttttgtatttttagtagaaacggggtttcacggtgttggccaggtttgtcttgaactcctgacctcaggtgatccacccacctccgcctccccaagtgctgggattacaggcatgagccatcactcccggcctcatgatatagatcttgtacatattttgttagatttatacctaagcatttcatttttgcgggtgcttattatgttttaattttaatgccaattgttcctgatatatgagaatacagttgacttttgtacattaattttgtatcctgcaactttactataattgcttattagttctaactgtttatttgtggttgattccttgaggttttcttcacaggcagtaatgttattggcgagcaaagacagttttattttttctgtaccaatctgtatacttgctattcacatcccttatttttttttggcatattgcatcttacacaattttgctttgtagcatctattttctcaccattaaatgtgatgttagctgtagagttttttattgtactttatcaagctgaagaagtaaatttgaattttaccaaatgctttttcagcatctatatatatgatcagtttgtttttcttccttagcttgttgatgtgatggattacattaattgacaaatgttaaaccagtcttgcatacctggaataaatgtcacttgctcgtgatgtataagtatttttatacattgttggatcagtttgctaatgttttgttgcaaatttttgaatctgtgtttgtaagagattggtcagtagttttccttttttataatgtttttgtccagttttgggttaatttctttgtgtgatgtaaagaagtggtccaggctgggtgtggtggctcacacttgtaatcccagcactttggccaaggcaggtggatcacttgatgccaggagtttgagaccaccctgggcaacatagggagcccctatctttatgaaacattaaaaaataaaaaatgggctggacgtggtggctcacgcctgtaatccagcactttgggaggcccaggtgggaggatcacagggtcaagagatggagaccatcctggctaacacggtgaaaccccgtccctactaaaaatacaaaaaattagccgggtgtggtggcacgtgcctgtaatcccagctactcaggaggctgaggcaggagaattgcttgaatccgggaggcagaggttctagtgagtggagattgtgccactgcactccagcctgggtgagagcgagactctttcaagaaagaaaggagagagagagagagagagagagagagagagaaagagaaagagagaaagggagggagggagagagggagagagagagagagaaaagaaagagaaagaaaagaaaaggaggggaggggaggggacgggacgggagaaaggaaaagaaaagaagtggtacagcttcagtcctttacagctgaatatccagttgtccaaagcttcatttgttgaaaaatctattcttttgttatagaattgtgttgacaaccttgtcaaaatctgttggctataaacatttttggactctgaattctctttcattgattgataacatatgtccttatgccagtaccacactgtcttgattactttgtctttgtaaaaagatatgaaatatgagtcctccaacttcttctttacaagattattttgactatctgagtgcacacaaatttccatatgaattttaggattatcttatcaatatcttttttttaaaaaaaagatagctggccaggcgcagtggctcacgccggtaatcccagcaccttgggaggccgaggcggacatatcatgagatcaggagtttgagaccagcctggccaacatagtgaaactccgtctctactaaaaatacagaaattagccaggcatggtggcacacgcctgtagtcccagctactcagaggctgaggcaggagaatcgcttgaacccgggaggtggaggttgcagtgagctgagttcaggccactgcactccagcctgggcaacagagcaagactccgtctaaaaaaaaaaaaggtagctgtaatttcaacaggattgtagtggatctgtagatcaattttgggaatattgccatcttaacattattaagtcttccaatccatgattatagattgtctttatttatatctttctatgagattttagagttttcaatgtataagtcttacattcatttgttaaatttattcctgaatgttctttttgatgttattatatttggattttttttcctcttaagttcttttgcagtgtgttctttgctagtacttcattattttgacatcttttcagtatgtaatgttaaactctgtcctgagtttattttaatattaagataggttacattttataaagatcaccctgagataaaaaatctatctagatagggaatggtggtgaagtaatagctattcctgaactctagggtttaatttctatttttctgtaaattattggaaaaggactttgttgttaacccccagccagtctctctgattccttaatgtggttggcgattataatacctgaaggtacttgtacttgcctgtgcctcttttccatcagtactagggtttcttattttcctaatcctcccaagttactgaatagagagccctttaagaaattggagaagtcattttacctctctaaatttgtttcttcttggtaagatgtaggatgaaatatttaataatcattttaagttttttcatacttaagtgagagtgctcacataaatagcttctttaatctgtaaagaaatattcaaatattagggttcctttctcttcaaattagaattttcctttcttagcttgggataatctatcaagatttatctgaataagctacttcacctcacctttctgttaaaaaacctccactgagtttttgcattacaagatcaagtgtgaactaaggctggccaactcaggttgtatatactgtgtgcaaaggactacattggatgttgtgtaggttacaaggagacatatgaacagtctgtgcaatcaaggagtttatagtctatgtgaagtgttaagtgaattgtctagtttggtgccagctgtgggagttcatgatttatatgatagactatcttcctgtagattccccggaaaattgtgctcacatgaaatcttagagaacctacaagatttataaacatagtaactgtttatgatagataacaaccaaactgaatttctagggctttattgaggagagtgtttatctaggcactgaccatgttactacccaactcttggtaatgcctttaaaatttttttaaattatttttggcagacactctgtgtgaggtacagattttcagtcctttgaacacaataaatgaatgaaaattgatctgaagagaatattaaaacattttcacttgaatcttcacaatctaccagtactagggtttcttatttcctaggtagatggaaagtgttttcgatgaatttggaccagaattggtacaaaaatcaaagaattcaaaatattaaattattttgtctctctttaagattgttataatttgattaagtattcttttttagtgtcatcagaaaaataaagcataggctgggcatggtggctgatgcctgtagtcccagcactttgggaggccaaagcgggaggattgcttgagctcaggagttcaagactagcctgggcaacctagtctcttttatagagaccttgtctctataaaaataaaaaaaaaaaatggctgggcacggtggctaacacctgtagtctcagcacttcgggaggctgaggcaggtggatcatttgaggtcaggagttcaagaccagcctggccaacatggtgaaaccccgtctctactaaaaaaaaaaaaaaaatacaaaaattagtcggacttggtggcaggtgcctgtaagcccagctacttgggaggctgaggcaggagaatcacttaaacccaggaggtggaggttgcagtgagctgaggtcacaccactgcactccagcctgggcaacagagcaagactttctgtctcaaaaaaaaattaaaaaaaaaaaaaaaaaaagaaaacataatattacaaaaacctataattcaattattttgcttttgatttgtttgctgtgttaaaaatgagcttttagatcggccttccctagctttattgagatataattggtatatttaaaaactgcatataattaatgtgtgcaatttaatgcatttggacaaatatgtgtgctcatcttaccatcaccacagccaagataataaacatatatctattgcctccaaaagtttcattatgcccctttcttactgtttttcatttttttgagagggtaggtaagaatatttgacatgacatttaccctcttagcaaattttcaaaagttcaccttgttaattataggccctatgttggaaagcatatctctgtaacttacttatcttgtataactaactttataccgttgaacaacaactctccatatcttcctcccctgtctcctagtaaccaccattctattgtctacttatataggtttgattccttgttatgaaaaataatgcagtatttctccttctgtgactaacttattttgcttagcatagcgtattttccaggttcatctgtgttgtcacaagtggtaagatttccttcttattaaaaaaacttgtaatatttattctattgtatgtatataccacatttttcttatcaattcatctgggttttcccatctttgctattgtgaataatgctgcaatgaatatgggagtgcaggtatctttttgagaccctaattgcaattcttttggatgtttacccagaagtgggattgttggatcatatggtctttttgcatatgcctgttggtcgtatgtatgtcttctttgtctattccagtcctttgcttattttttattcatctcattttttgctgttgagttataggagtttgttatattttctgaaattaactccttgtcagacatttggtttgcaaatattttcttccattccataggctgccttcttactctgttgatggtttcttttgctgtgtagaagctttttagtttgatatagtcccacttgtctatttttggcttttgttgcctgttatccaagaaattattgacaagaacagtgtcaagaaggttttctctgatgatttcttcaaagggttctacgttttcagatcttatgtttaaatctttaatccattttaagttaatttttgtgtatggtgtaagataaggttccaatttcattcttttggatatgggtatccaggttttccaacaccatttattgaaaaaactgtcctttctgcattgtaaattattggcacctttgttgaagatccattgactttatttctggattttctgctctgttccatttgtccatgtgttttttttctttttctttcttttttctttttttctttctttttttttttttttttttgtggggaagaagacagtctctactctgttgcctaggctggagtgccgtggcataatttcagctcactgcaatctctgcctcccaggttcaggcgatctcccacctcagccttccaagtagctgggattcatatgtctgtttttatatgcttgtatcatactgttttgattactgtagttttataaaatattttgaaatgaggagtgtgatgcctccaacattgtcatacttgctcaaaattgctttgaatatttgaggtcttttgtgattctatgtaaattttaagattaaaataaattatgtaaaaaatgtctttaggattttgataggggttgtgctgaatctatagatggctttggataatatggacattttaacaatattaatttaatccatgaacacaggatgtttttccatctatttgtatctgccttaatttctttcattagcgttttataatttttagtgtacaagtctttcacctccttggttaagttaattcctaagtattctgtttgttgctgttgtaaatgtgattgctttcttaaacttttttttggatagtttgttgttggtgtataacaggggcccccaaccctcaggctgcggaccagtgtggtgcatggcctgttaggaaccaggccacacagcaacagatgagcaccaggccagtgagcattactgcctgagctccacctcctgtcagatcagcagcagcattagattcccataggaacatgaaccctattgtgacatccgcatgcgagggatttaggttgcacactccttatgaggtagaacagtttcatcctgaaaccttcccaccagcccctcaccggtccgtggaaaaatagtcttctatggaactggtcccttgtgccaaaaaggttgaggactgctggtatatagaattgcaaccgattttttaaatgttgattttgtgtcctgcaacttgactgaatttattctaacagttttgtgtgtgtgcatggtctaggattttctacctatttcatatgcaaacaaataattttcttttttttttccaatttagatgccttttatttctttttcttgcctaattactctggctaggacttacagtactatgttgaatagatggcaagagtgggcattcttgcctaattgttcataatcttagaggaaaagttttctgtttttcactgttgtgtatgatgtgttagttataggctttttttgtttttttgtttttgtttttttttttttttgagacagagtttcactcttgttgcccaggctggagtgcaatggcacaatcttggctcattgcaacctctgcctcccaggttcaagcgattctcctgcctcagcctcctgagtagctgggattacaggtatgcaccaccacgcttggctaattttgtatttttactagagacagggtttctccatgttggtcaggctggtctcgaactcccgacctcaggtgatccacctgcctcagcctcccaaagtgctgagattacaggggtgggccaccatgcccagcagttatgggcttttcatatacacatgctccatgacctaaaatgaagttacatccagataaactcattgtaagttaaaaatgtcattaagtcaaaaatacatttaatgcacctcatctgtagtacattatagcttagcctagcttaccttaaacgtgctcagaacacctatgttggcctgcaggtggacaaagtcatgtatcgcatagcctattctataataaagtgttgaatatctcatgtaatttattgaatactatatgggtactcatcattaatgtacacagctgaaaacaccattgtaaggttgaaaatttttgaggtgaaccatcaagtcagtgactgattttataacttttattttgttgaggtaagttccttctatatctgatttgttgatggtttttgtaatgaaaggggggtgaattttgtcaagtgctttttccttttttttttttttttttgtttgtgagacagagtcttgctctgtcacccaggctgagtgcagtggcccaatctcagctcactgcaacttctgccttttgggttcaagcgattcttgtgcctcagcctcccgagtaactgggactataggtgtgtgccaccacccctggctaatttttgtatttttagtagagacggagtttcaccatgttggccaggctggtcttgaactcctggccttaagtgatctgcctgccttggcctcccaaagtgctgggattacaggtgtgagccactgcgcccagcctgtttttctcatcacttttaatgcaccaacaatgaggaagagattagtgctgttgttatatatacacaatctttggtgagccagagcttggctgaagaatggtattttcaagcctggccctgtgaggatagggctcttgatcagtttttaacctttggagagattagctggagagggattagagagagtggtacatcacagctctattctcaggaatgggaagtagtcctttacatgtgttttgaggggtgggggctcgtgacttgggaataacttgtttttctaactcctttatttttctgactggtagtgacacttcttacaatgaaatgtattgatatgtattaacgtatctctttttgtcagtaggggtgttattttatatcatcagtgtaaatacatgttcgaccatagattattgagttgatatgggctgggcattgttctagattctagacatacaacaataaacaaaacagacaacaaaactatgtcattttaaagcttatattatagtggggagggatggacaataatgaataaatatgcaaaatagatataatgtattagatgattgttactctggagaagattgaggtaatgaaggggtttaaggattgctgagagtaaatgttgaggttgcaattttaaatagtgtgagaaagtatgtagcatttgaggaaatacctgaagtatgtgaggggggctgatattcagatatttatgcagttgtaaagcaaagtagagaaatcccacatgggtaaaactctttacccactttccctcaatgattacatcttgcaaaattatagtacattatcacaattaggatattggcattgatagaatccactgattttattcaaatttcctcagattctcttgtactactttggagagctgtatgtgtatatatttctttctattttttttttattacatgcataggtttgtgtatagaccatgatagtaaagatacagaacatcacaagtatcctcttactgcccttttataaccacattgacttctctccctgccattactccacccctaacacccagtgtaacccctggcagtaaccactaatctgtttaccatttccaaactttttttctttcaagaaggttataaaaatggaatcatatccagtatgtaaccttttgggctagctttttttttttttttttttttttgtctcagcataggtttctggggatgcaaccatgttgttgcatggatcaataattgtttcttttttattgctgagtagtattccatggtatggatatactacagtttgtttattcacccagtgagtgacatctgggggctattctaaataaagctgctttgaacattcatatgccggttttaatgtgaacgtaaatttctatttctataaaacatatgcccaagagtgcaattgctgggatcctgggtaagttttgaaggttgagccaacaggatttcttgacagattaatgtgaggtcaagccgggcgcagtgtctcatgcctgtaatcctagcactttgggaacaataattgttattgctaataatagcacttagggaggctgaggttggaggatcatttgaggtcacgagttcaagaccagcctggccaacatggtgaaaccccatctctactaaaaatacaaaaaaattagctgggcatggtggtacacgcgtgtaatcccagctactcaggaggctgaggcaggagaatcacttgaacctgggagacagaggttgcagtgagctgagatcatgccactgcactccagcctgggcgacagagtgaggctctgtctttaaaaaaaaaaaaaaaaaaaaaaaaagtcagagagaaagaggaatcaagacttcatggtttttgacctgggcaactagaggagtggaattgccaacagctgacatggggaaagctatgggtagagcatgtttaaaggagatgaaaaattttacttataacatgtcaagttgatgtatattagacacagaaaaattgtcacataagcagttggttaaaagattttggaattcacaggggtggtctggtctagagatatacaattgggagtctgatcagcctttaaagccctgagactggaagaggtgatagaggcagctgaaagaggtgagagactaagttctggctacttcagtgtttaaaaaatagggagattatgaagaactagcaaagaaaactgcaaatttggccaatgaggaaggattaaaaagcaagataatttagtgtcctggaaagtaattggagaaaacatttcaaggagagagggagtcaaccaatcatttttgtgtaaaccttagttgtagcacaaaggcatattctgcacagtaaccagaggttttagttgtgttgtattccaggggatatataggctgatgcccccgtttctagtttctcctgagtaaggagaattttcgagttttagtttctgtattcagaaagcaaatggtctaagagacaagttgaattctacctgttaagctccaacttaacactcttttcatgaaaaaagtgaacactcttttcatgaaaaaagtgaacactcttttcattaaaaatctttttaatcttcctagttggaagtattctttctgtttttagaattctccatcctttttttttcttccagggtcgagggagagacagggtctcaccctgttacccaggctgaagtgcaatgtcacaatcatagcgcactgtagccttgacctccctggttaaagtgatcctcccacctcagtctcccgagtagctaggaacacaggtgtgcatcaccatgcccagctaatttatttttattttttgtagagatgcaatctccctgtgttgtcaggctggtcttgaactcctgggcccaagtgatcctccttccttagcctcccaaagtgctgggattacaggcatgagctgccacacccacccacccacccacccctttttcgataactctagtggcacttaacattttctgttgtatggcatctgtgttagtccattctcacactgctgtaaggatactccttgagacggggtaatttataaaggaaagatgtttaattggttcacagttccacatggctggggaggccttaggaaatttacagtcatggcgaaagggaaagaaagcaggcaccttcttcacaaggtggtgggagagagagtcatcgtgcaggagaaactaacatttataaaaccatcaaatcttgtgagaattcactcactattaggagagcagcatgggggaaaccatctctcccagtcacttccctcccttgacacttgggggggtataggtcctcgcctcagcacgtggggattacagttcgagatgagatttgggtggggacgtggagccaaaccgtatcagtatctttatttgcctacttgtatacttgtcttattttgaattataagctctttcaatactgaagtcctcctttattgtcattgtattttcaagcacatagtaataaatggaattgagttacatttcattgtgatagagcatggaatctgacacacctggtttaaatcttgcctgtgctgtttgctaccaaatttcttaacctctctatgcctacacccgctcttaagtggggatcataatgttgcttacttgataggtttgtggagaggattgaagttgtgaagatacagctcttaacagagtggtcagctcatagtaaattctttgtgttctgtagttgttggtgccatcattgctattgttaataataatatgctgttgtgttattggctctcaaagatacagggttaaaggaatacacacttaagctggcattgacttacatgctagggcttccgaaaactaagtattcaagaacctggtttatatctactttagtagtcatattacctgtcaggatataaaagtccgttattcccttagttcatgcaggcatcctttgtgttgtttctcttccatttccctttctgtgctgggcaagtagtctgtgtaactgattgggaaaagtagcatacatgagtcctttttattgtttctataaaattggggttttgttacagaacatgttaagcctacctaggcagtatgttattttataactataatttaaaattttaacttacacactaattatagttatgtaaaaggaaaatatcataaatttccagaacattagtccctaaatcaatatagaaagtccactatgtatatgtactaagtacaaaattgagattgacggtaaggaactttcctttcttgccttgtggtgcagggattgggaaacagtgaaccagccacctgtgtttgtaaataaaatttataacaaacagccacgttaattcttacacatattgttgtaaacagacagccacattcattcttataaatattgtctgactgcttttcccctacaactacagagttgaataaacctgaagaatttagtgtctggtcctttatagaaaatgtttatttgcccctgttcaagggtattatttttgtggctcatctcttagagcttttctcactctgtctcttgtattgattccttttcttctgtctgcttaaatgatgatattctccagaatcttttcttgaatagctcacctttcctaaggcttacgttactccactgtctaggttctactttctgactaatttctttttagtgtcctttatagactttctgctgctggggagtttgatcactttttttcttattttataggttgagcatcttgaaaccaaaaatccaaaatttgaaatgctctgaaatttaaaactttttgagcaccaacacgatgtttaaagggagtgctcattggagcattttgaattttggattttcagattaggaaggcttaatgggtaaagataatactaaatccaaaaaaagtctgaagtctgaaacacttttggtcccaagcatttcggataatgtgtattcaacctgtataggcaataatatatactctcctatggcttctgaaccacccacacctccttgaggataagaagaacatacggtattgctttctggaccttttcatctgcttcagtgtccctgagtcgtttcaaaatgaatgtgtcccaaattgaactcacttctttctgcttccaaaacagcttccccggccactgaactgtaccgtgttcatagtcatgtgagtgagaagcctgatagctgtcacctccctccttctatttactaattcaatagcagtaggtgatcactactattgtagacccattttcttccatttgtttttctctgtttttactagtgtagatcctagttttatccgccgccacctttgtaacattttttttcatagcaatcttcctaaaacaaatatcttgtgtaaaacacattagtgggctgggtgaggtggctcacacctgtaatcccagcactttgggaggccgaggtaggcggatcaccagaggtcaggagttcgagaccagcctggccaacatggtgaaaacccatctctactaaaaatacaaaattaggcaggcgtggtggcaggcgcctgtaatctcagctattcaggagggtgaggcaggagaatcgcttgaacccaggaggcggaggttgcagtgatttgagatcatgccactgcactccagcttgggtgacagagtgagactctgtctcaaaacaacaacaacaacaacaaaaacaaaagcaaaaaaaaccaatcagtggttctttatgccttgtaatatttagcgctgtatttaagactttccacctgaccccattccagaattttacaaattaataagcaaatttttcttagctcctgtccccaagtatctatagcactttttaaatgagctcaacttaatcacattgtgttatagttgttggtttgctcacccattagactgtaaaacatttaaagattgtaaatttttgtattctcagggcccccagttctgacacatagttggcttcaaaatatgatcattacatgttctatgcatggaacaaaatatccatgtacctcataaatagtaaaatatcatgtatcaattaaaaatatacttgtgcaaatggggttaagtttttttgtgtttttttttttttttagctgttggttcttttagggcaactgaatttactgtgaaatcaaagatttagagatgaattaaatgctttttgagtcttatgatgactaaggatggataatgcaactttggataaccttcattttttttgcagaaggtgtactttgggaaaatattctttaagcaaaacattggaaatgagtcctattttactagattttatgaaagtagaactttctatcatgggggaagatctcccaacagaacaaactttgatgaagaagaaaagatgataaccacatgctgcccatcatacacccacaatattaagcttgctggttgtttcttcacctaaaagtttacttttcctttattaaactttatgtaatggtacaaaaatatagccttctggaactttttcagtctgtgcttttagcttccagaagtttttctttctcttttctctctctttttaaagcatgagatgtcagatattgtaaaaatgaataaaagcctccatgtctactctagaggtggatatatgttaatgagtattggttaaaacaaaagaatcagttttcctttaaatgtgccatttagactttttaaatactgatattatgcttattccatcttgggtagtcattttgttttataaataagttgagaatgattagtaagaatcttgtttacatgtattcttttgtcttttctatttgaaaaaagatgcctttacaggcaagtcagttaccacaatttaaaatttctaactatgaccttaaagtaagacatgcattttaacattgcagcccgatgcacagaaaaataattaacaagtttcatgaaaaaaacccttactgtatgtaaagtgatattctattccattctattctattcttattcctccattctattttatgctatgctgttctgttctgttctgtttcttttctattctattctattctattctattctattctattctattctattctattctattctattctattctattctattctattctccttccacacttccttctcagtgctgattggctggtgcccatagtttggaaaataacaccaagttggaaagtagaagaaattacagaagctgggaaattactttgttaaagacaaagtaacctgctctgagaactctgagagtaatacagaaagatgttcttaaaacacttatagtatgttggttgggcagggtttgtgagtgtgtgtctgtgtctgtgtctgtatagtgactataatatctgatttctataagctacttttgaaaagtatccttttatataacatcaatgaactgtagttgtaaaaataaaaccaagagaacatatagtaaatttcaaaatatttgtaagtgctgcagggagagattggtgatggcctcgttaaaaaggtaaaagttaaataggaacttaaaggatgggcttagaatgtgtgtaggttggagagagagtcagcacattccactgatggtaatgtgatggtaaatgtaatagaattgggaatataattggtttagaattgggaatgaaacaatatattcaggaagagtgagaagtagcatctagcgataatatagaattcacattggtcatgagtggaagactaaactgaaaaggagaggccagattataaagtgatttagactttatcttgtaggcagtgggtctctctctctgtctgtctctgccaacatgtgccgctccatctctcttggttactgtttgtattgtatatcatttttcatccttttacttttaagttgtttgtgtctttgaatctaaagtattaattcctagggatagattatagttgcagctttctttttatatagccttttgattgtatgcttagctctttcacattttatgtagtgtgtgatgtagctggaattacatgtgccattttgctctttgttttctatatctctcatgttatatttgttcttccgttcttcgttactatcttctgtgttgaacaaatattttttagtgtatcgtcttaagtctgctgttgatttttaacattaaattattttcttagtggttgctctaagaattacagtttacattccaatttatcaccattaactttaggttaatactgacctaatttctgtaaaatatagcacttgtgctccattataactgttttctccccctcctttttgctgttattgccttatatattacatttatttacgttataaacccaactgttatcatattctgtcttttaatgaaattaagaggaaataaaaatatatctatagtcttttatattttctcatttatatttaatatatctggtggtcctcacttgtttttgtgaattccagttaacatgtggtgtcattttcttttagctcaaggtacttgttcaagtatttcaacttattctccatctttttttttaagtgtgaggatgtctttattcttcctttcttgcttgaagaatacttttgcagtaaatagaattcttagttgatagttttttttaattttttgttactgccttctagcttccgttttttcctgatgattagtcggctattaattgtattgtttttcccatgtatgtgatgagtgatttctctgttgctcctctcatgtttttttttttggtggcttcaaactgtttaaatgtgaaatgcttagatgttgatccctgtgtttatcatattttgggttcattgataatttttttttcttttctttcttttcttttttttttttttttgagacagagtctctctctgtcagccaggctggagtgcagtggcacgatctcgactcactgcaacctctgcctcccgggctcaagcaattctcctgcctccgcctcccgagtcgctgggattacaggggtgtgccaccacacccggctaatttttgtatttttagtagagatggggtttcaccatgttggccaggctggtcttgaactcctgacctcaggtaatccacccgcctcggcctcccaaagtgctgggattatagacgtgagccaccgtgcctggccggttcattgatcttcatggtttgatagattagtggttttcagcaaatttggggaatgttcagccattatttcttctaacatttttttctcctttctctttttccagttctgcttttacacatatgttgatatgtgtgatgttatcccagaagtatctgaggctctgtttatttttctgcagtattttttccctctttgttctttcttttggacaatctctatttcttcaagttcactgggtttttttttttaatgccatctcaaatattttcttgaggacatagtgaatctcatttttgttataatgtgtttcaactctagttaattctcatgttttaaaagttagcatttctttattgagattctctgtgttgtgcaattgtcatttttctttaatttttaaaatatggtttcccttaattttttgaacttaattataaattataatagctgctttgtattctttatctgctaaatccaacagctttgcccaccaggagttaatttctgttgactgcttttttttaacctgattataggtcaacttttcctgtagtttgcatgtctcacaatgtttttttccaattgaacatattatataatatattgtagcaatgctaggttctgaatttttttgagacagattctggcttgttttctaggctggagtgcactggcatgatctcagcttactgcaggctccacctcctgggttcaagcagttctcctgcttcagcctcccgagtatctgggattacaggcatgcgccaccaagcccggctaattttatatttttagtagagactgggtttcaccatgctggtcaggctggtcttgaactcctgtcctcaggtgatctgcccgcctgaacctcccaaagtggtgggattacaggcgtgagccactgcaccctgccactttcagcattttttgatgatcctatatgtgagttgggaagtatattcaaaattctgacatttttcacattagccttggcttttactttctccggggctctgttgagcctccctccacccccatatgtgcactgcctgtcagacagttatgtgtggggagctaatttaggccctctgtgcctctctcatttccagtatccccttattacatttctgggtgtccttctagtctgtcattttctctaactgggactacattctcattttagcagagctatgggcctttcccctttctgtccttccaaatttgctatttttattgccagtgctactaggcatgggtttttgctctctatcctaaatgaccatagccctctggttttcactgccaacactgccctgggaaaactgcctcactgattgagttgggtggttgtagggggatggtagcatcgcttggcaaagaaggccattttcctctactgttctgtcatatctagcagcttttcaagaataaatgcttctcaatttgttgttgcctttgctcagtttccagagccctgaagtgattgttttcttcagtgtacttcagttttataagtattgtgggggagtagatttgctactgtcatcattctgctgtacctggaaatctctcctcaaactttctaaccccttaggaccctgtgtcccctacacccactttaattcaatctagtcagtttttttcaccattgttctactgaaaccattcttgtaagagtcaccaacatcttgcattttcatagcaattgttattttcctgtgtttatctagccattatccttctgcctatttcttgaatatgccaagcccaccactgagtttttacatttgcagttccctgtaaccatcttccctagatcttcacatggttcttacctttatatcatccaggttgatgttcatatcttaacctccttcataaggttttctccggcttccttatgtaaaatgtagatatttctaaagtaacgcaataggaacagaagagtaaacatagatgaatatttaggtccttatacttgcttcacttaatgcatctttcagtagcttttgacataaatcatcactcagtttttctaaaaattttttaacgtggcttctaagataacattctttcttatttttcttcctgctttattaactactccactctgtcctctatttttcatttcatgaattttttttctgtatgaacttcttccgtaggtgatatcattaggtacccttgctttaaatcttctttatctagatgactcccaaatgcatttttccagccttagcttctaccctgacctcacatgaatgtcgtataaggatttcaaacttaatagataaaaacttaataggttaaaaaaaacttaatagataaaaaacgaaactcttgatctactttttccttcaaatttattctcttaaatttttagaatgctttttttttttttttttttttgagacggagtcttgctctgttgcccagtgctgtggcgtgatcacagctcactgcagcctccacctcctgggttcaaatgatagaatgcttttctgaaaggtaaatctgattgtatcactttcctggtcaaaacctccattagcttccctttacatttagagtgaaattcagtttcttaccatgtttgatgaaggtttatatgatcttgtgcctgcctgcctctctgaacataactcctacctctctgaacttaactcccacctttctctccttactcagaatgatgcagccgtattatctttctggctatttcttgaatatgccaagcccaccactactaggtttttacatttgcagttccctataaccacagtgctgtttcctcagatcttcacatggttcttaccttcatatcattcaggcttgtgttcatatcttacctgtttcatgagattttctctaacttccttatctaaaataagacatttttaaagcaacacaataggaagagtaaatgtagatgaatacttagatccttgttcttgcttcacttatgcattctttttgatcaaaagtattatgcacaatcttatcaacttctgttatctgggaatttaatactctgttaccttggttagtgagtctggaaaaataaataacttattttaacatagttggctatgttaaaaaaatcagtcttaatagctgtatgttatgagaggaatgagtattgatttactagcagttctccctcaaagccagctacctaaatgtgaattaacaaagtatagaggctgggcgctgtggctcacgcctgtaatcccagcactttgggaggctgaggcaggtggattgtctgaggtcaggagttcgagaccagcctggccaacatggtgaaaccctgtctctactaaaaatattaaaataattagctgggcgtggtggcaggcgcctgtaatcccagctactcaggaggctgaggcaggagaatcgcttgaacccaggaggcaaaggttgcaatgagccgagatcacgccattgcactccagcctgggtaacaagagtgaaactctgtctcaaacaaacaaacaaaaaaacaaagtatagaaatacttccagaaatagcttcaatttggaatggaaatggacacaattttgaagttggtagttttctatgttcctctctctgtactagtcaacacttaagagtcttttgttagttatttgttaaagtaattgtactgtactttctgacttattttctgtttactttcggactttctgtttccgttacttttactaacaagtaatggaatgaaagattgatatcttataaaaggattttaaataccattgtacacataagcccaactccgaaaggtcagtttaaaggaacattcacttctgctctcatcttctaagaactagaagccatcctacccttcctattctaggatagctatctttatctggttctttggtcccaacgcatcctcatccccatccttcttggcctcttttctgaccgtaacaccatttcctttccagattctcaggtgtatactttatgttttctagctcttctaaatcattactttaactaaagctttatttttttggctagaaagctggcattttggtattgcttctgtgtgttatgtaatttgtccttcattaagtggatttctgtcagtcaagtatattgttggactataataacctctttgttaatgtacagtctagtcagatttactgctgcgaagtctgtggtattaagaaatcatcatattatttagtgatttctttttttgaagaatgaacataactttgaaacttcccaaagggaaaatagttttgtgtggtctacaaatctatcaggactgtataatttcttttcttttctttctttttttttttttttgagatggagtttcgctctgcattcactttttctaaaacttagcaactgagctcttttcatggagttgatgctcagttttttttttcactgcatcttagaaaacaataaggtcaagctatttgtggtaaagtaacagttcttacagttttgaattcatgggagactgttgtttttgtgaaatatacaaatgaattgctagaaagatgaaatttttaaaaatataaaatatggatcctaattcttattaccatattcttagatatcaatttattctctcattgttataaaagctctaaacactctcaatttctgtatttgtcttattggagagcagtaacagatagttcatggactagtaacaaatattttgcagatcagaaccagtctacagatcacactttttagtagcatatctatagcttgctaattttagtgcttaatgttatttcagaagcatcttgttcaatatgatgaatagctgttatcctttaaaaatatatttttatgtgtttccttttcagaatcgagagctccagatcatgagaaagctagatcactgtaacatagtccgattgcgttatttcttctactccagtggtgagaaggtaggtttgacacaaagtgtgtactgaatattgtgataaatttgttatttttctagaaattaatctccccaccgctcatagacagttttgctaatggaatggaaaaattccgaaacacttgccccagcaatactcagcccaaggtatggtcattgatatcattagttatatcaggaaagcattagtaagataaagggaatgcacttttaagaattttcttctttgttaaagtatgtcttcacttctcaagaaaatctatatactttttttttttttaaagacatctatgttccttatgtgcttacgcagccctctgatagtttacctatttattgcagggaggaaaattaagtcattttgttaagtgaagttgataagaggtaaagaggtagcaagaggactgataggaacagaatgagagttaaacatttggaagcaagttgagccatccttggccagtgaaaaagttggaaaagatgtaatattgcagagcttactttaagtgatccctatgtctgaccacattgaattctgcgaaaccaaaggataaagaagactttgtgctgttaataactccttccaacctctttctcttcagaagacacaataatcaatgaagttggagcaaagaaaataaatttcttctgtttttccttggtgtctttatttttctgcctgtcgctatcctttctttcttttcactggtttggagtgcatacgcagggcaggtaacaaatcacttgattcttttggctaaataaatatgactttggataacttcagttggtattttggacttcagatttcatgcatataatatctaatgttcttagatccataatacttgattcctagctcagtgttcttgttactttatgtaactgttttttagtataacagtttttagccattttctattaagctttttttttacagcaaatattgcctgacaacattttctattaaactttatttagattatttccatagaaagtctttttacattacagtgcctattatataggaagatgtcaatgtattaagcccgaattttgatataaatacctacagtcatataattgttgtgtatatacacatgtattacacaatacgttttaacaactatagttagcaaattggccctttattgacacttgcttttcagttgctttttttttttttcagtttaaatgatacctctggtttactagggattgtgaaccctcataagtattagagctttttaaaacctaccaaatggcctgttattatctttattctgatctgttttggcatgtgtttgctttttcctgtctcttcctaagtaaaattgtacagcctctgttaggttcacaagtacactctttgtaatgaaattttgcatcttcattttgttgttgttgttgttgttactagatacatcatgatactcctgggtaagaattatttgttccataaactgctggattacaatagattcatttgaaaaaaggcctcctcccagagttcttaaacattttctctttgattacttactttaatgtatatagcattccttatatgctaggcactgttttaatgctttataaatattaactcatttaactcttaagaagttgaagagcagaaaagaaagactatcaaaccgtttgaatgcaaggcatctgatggcttacttgtctggttttggtttggtggaaagctcctgtcatagtcagctcttgccctgacttaatcctgcattgggggtgacaactgactgcctcttatctgtatgccccagaggataatttcctctctgctgctgttctcagtttcttacttatcttaaaggttcttacagataaagagcaattagaagtcattaacagagattaacctctattataaaaagtaatagacctaatattttctttatgtcagaactttctcttgagctttgtaaattttccaaaaatttttttaaaaaaagatacctcctaattttgggatagcttcattaaaaacaaaaaggaagaagagaagaaaagttgtgatatattgctgaaaataatggtccttctttgtcaataaaggaaatttttctaagatgtgcagaaatacataggttcaagacttcccgtaatctcccagccaccaaagagagaatttctttttgccccattcatgatctccacttctgaattgttctggcaggactaagatatccttcaggagatcaagtagtaaactatttcttcccataggagaagatggattcttttctagggttgagtgctcaggttagttattttcagttctgactttcttggaataggttactgagagtatccactgctgtttagaaaggaacctaaatgtctgtgaaattcttgcaaatccttgaagatgatttatcttggaatgtggcagtcctatatttggtaggcatttttctagcagaagcattatgtattgcttatgtatattatatatgagaagagttttggaggaggaaataataaattaatcatttgagatgcttatgacacatgcaagtagagattcccagtagatggatagatgtgtgacttcgtagatggtggtggtggctctaggtggagaagtgacattgacaaagtaggataaagagggcgtgtagccatgtggagcttggtggggaggagacatggagtgctgtttgaggccaggacagtgaaaaatcagcggtttggcaatatttattgccttctttttctagaatttgtcaggaggtgaccagtcttcatttgaaagaactgcaaggaaaccagtgcctttaggtgagattcaggttttctctagagcaaaacggtaaaaagaaccctgcatgaagaaattgaggtcattagaaattttcttcttgaggtttcagttttctttttgaggtttcttttgacgtttaggtgagatccaggttttctctagggtaagacagtaaaaagaaaccctccatgaagagattgaggttattagaaattttctttttgaggtttttgagagaatgttatgggagggagatcagtgataagaacatgagcctacaggaagtttgaggaggcaattagggagattagttgaggacattgtgaagatgagaaagaataactcattgtttcaggattagagagattaaaggcttggttaggttctagaatagatgacttaaaatgcatttgggaatttaaaaaaattctctcattctctataacatctctgactttctttccctcttttttttctatcagtctctttctctagcccttttttcgcctttgtctttagtcttcacctttctggattcctcaacctttcactgtaggctccaagtctctatctttctgtctctgggcttttgtcccccctctcttggtctgtgtcaccccctttttggatttcttttccctccatataactctttctcttttctctgtctctttctttcttgtctctctcttctcccccacaccctttttctctctgtctctctccctctcttatttcctttttatgggtctccatcaacctctttctctgggtttctatacctgtctttcaatgggattttacccatcccactctctttctgattgcttctaaatctgtatatgggttctcatctgtcttaaatcttcttcgttgtctctcttagtgtctcttctctctgggcctctattactccctttttggaaaatgttaaggaaaacaactaaattattcactactactgtcctgacctttgacttcagttctgtgtatcttattccatacgtacatgtttatataggtatatgcataattatttatgatctcataaagatttttttctcttgcctcataatatgtataggcattttggttatctaatattccattgttatactatataatattttatctattcccacattgttatttttaattttagaattgtagctaagctaccctgtggccttgtcataatttctgtctgtaagtacttttgcagctctgggagaattaaagtaggatttctgtaataacaggcatgttttacatttcattgaccacagtttgactgaatgatatgtctattttggtaaccttctttttattgtattctgccttttgcattttaaagactttctttagggatagtggataatcagcttttatcctgtaacaggggaaaggggtagccaatgtaactagaagaaagaggaattaagttaggacagagattagagatgtgtactaattagccttagttttctggttggaattattaatgtttgctgtagtaactagagcatcatgtagtcaattcgtatactctatgttacccagtagtaactaactatgatttagtttagagtaagtagtacctcctctcttaccttcccattagagaaaaatgagatatttgagtgttttgaaaccctgggcttatagttttgtgagttgcaagtatcttacagaatagtctttctctttttttaatatttggcagtagaaactttctacatccccaatgaaattttctgaagacctctgatagaacatatgaaaagcagagttgccctagttgaagctaaggtgtgggatcagatgattttgccttttctgagttatagcttaccccaaagaactctaggactctgaagatcatatgataaaaaccatgttatgagctgggtgtggtggctcacgcctgtaatcccagcattttgggaggccgtggcggttgaatcgagaccagcttggccaacgtagtgaaaccccatctctactaaaaatacaaaaaattatctgggtgtggtggtgggtgcctgtaatcccagctactcgagaggctgaggcaggagaatcgcttgaacccaggaggaggaggttgcagtgagccgagattgccccattgtactccagcctgggcaacaacagtgaaactccgtctcaaaaaataaaaaataaaaataaaaactgtgttatgtcttcacaagatccttaaattttatttcaatcaattaatagttttctgtaagaaaaacttctctaagtaacttttcatttaaaagtttttttccttgtggttcatagattactttaataataaagcgaaaaacctctgccttctactttgtgagagactgttcaaataacaaagatgacagttgattttatttctcatgccacctttgttctattgatagtaatgggggcctgtatccagaaggggaggattgtaccactgcatctgggatcagggagcagaatgctgtgcttaattttgctggtctaccactagctctggagaggaaagggtaggggtgacatatatgccaaggattcattctgaaaagtaaagagcactggctttgtagctagaaatatttgagtttgcattccatttctgccacttgatagtagtatgactctgggcaagatatttacttttataagcctcctccccgccatctgcacagtaggggttttgtaaggaacaaatgtaataaggttatcacgtgctcattatagtacctggcatataaattaacacttaataaacattaatttactattttacctattattgtgtttattaatagcaaatatatattgcgctttttatatgtcaaacattgtcctgagctgtttacatattttataactcacttaaacctcatagcaaccaattaaagaatgagttttatagaagcactattttatagaagaaactgatacaacagccgttaagtaagttaccatacaactactaaatggcaatgttgggcgtcagctccaagtagtctgactccagagtctgtatagtatgctatagtgtttagcataggacgtacattcctccagaagcagtgtgctagaaatagaacattaaaaaatatcttagtaaattatcaaacaaggggagattataatattaatagtagtattacgccagtcaaggtggtttacgcctgtaatcccagcacttttgagaggccgaggcgggtgaatctcctggggtcaggaatttgagaccagcctggctaacatggtgaaaccccatttctactaaaaataaaaaaaattagccaggtgtgatggcaagtgcctgtaatcccagctactcaggaggctgaggcaggagaatcgcttgaacccaggaggcagaggttgcagtgagccgagatcgagccattgcactccagcttgggcaacaagagcgaagctccatctcaaaaaaaaaaaaaaaaaattagtagtaatagttgcagtactagaagtagtggtattgataataatagctaaaaggtatgagatactatatgccaggcattaatctaaacacattacttatatttactcatttaatcttcacaacggttctatgaagaggagatggagagaagttaagtgacgtgacctgcccatagagtgatttaaaccagtattttaaatttctagtttgcaggaagtatgtgactcttctaaaataaagattgtgcattttttcgagggagctagcttgggtgttttattaggttttcaaagaaatctctgacccccaaaagataaaacagaaccatcaagccattcacccaagagccaaggagatataattatagcaagctatgtaaatatttataaatatttattagaaatgttttgatgagatttgagcccccacagtctagctctggattccattaaccttccctaataccaccactcttaatttcagtatatgtttttcttttaataaagaacttttatcagaggtttctaaaacttgtatgtatgtatgtatatgtactcatttctaattcagttttaactttttcgagagcaggtatgtgtgcatatggttatattaagggatgaaagaaagaaagaaaaggccaagaacagttccttcttaattttgaagcagtatagtataaagaatcaatttgagtatgaatggtgactctgctgttcatgtgtctggccatgaggactggctatgtgtcctcaagcaaattgcttaatttttctccttatcgctaaaatgagggtaatatatgtatgtataatatatatacttgtgttttatatatatttttatatatatacttgtgttatatacaccatgtgtatacacacacacacacacacacacaaacacacacacacacttttgttactgtgaaaattgtggccatgtaagtaaaatgtttatagtatcatgtattagtttttctttcattacctctaaccattagttcttggtgttccctagggttttattctccatttttctttactttcatacttatcctgggccatattctcaatccttgtgacttcagtgactctctaatgtttatcttcagttgaggtctccctgatgaactctagagcctcaaattttagtaccaaccagaaatctctacctggaagcctgagccgtcaacatttagcatgttagaatttacgactccccttatccttgctatatatcaattattaaaataagaaacctggatgttatgctagacctttttgttgacttatttgaatctgttctttctatttcactgataatgctgccttagttcaggcccacattatttcttatctaaactcttggtgatttctcttcttctagtacatacctttacttcctccccacccctagcctaaacagtattttaaaacagggataaatcatagattattctaaacaattagaagagacaatatgggtcattgtcttaagaatttattgtctgggtttaccaaaagctttatatgatcaattagcattggagatgttggagatgggagttgtgaccatccctaatataacatggcagcaggcaatccttctgattgtatcacttcttcagttacctaaaaggtaaattccagatttcttagcatgacatacaaggtctttgaacatctatatcgtgaccttacttgactcatctctcctttctcacaggctatctatgttctagccttgcccaaagtatgtgcaattgtcagaatatgttgtattatccctgttaatgcaaataccttctggaatgcttctttcctaccctccacccctgctctatttttgttgagttctttttcattgtattgtctctgctcaaaagtagctccatctgatatctcactattttctctccccccagccccccttttagccccaaaagcttctctctccttggaatatcttgcacatacctctttcatgacatttttcttgtattttaattattttttttttaactagatctttttactaggttgtgtaattctcagcagcatggtgagattttattaattttgtttctttaatgcttagcccagtgcctgagacaatgtgaagttggctatgtagatttattgaaagccttctagatagtcgtagaaaactgagagctaaaataggctcagcccagatattaacttttgttttttgaagtatattaagatttattataggtattacaataactatttctatagattgtgttattgtaaaggaattattttcttaattttacttttgggatattcattgctgaatatcaatatataattgtatagaattcaagagcattaattacgtatatgatataatgcagctatcactgttatttttaaaacattttccgttttatcacgaaatggaattggagtttatcagatgcccttttggtgataattgaaatgatcgtgtggggttttttccatcattttattaacatgatatattacattgattttcttatgttgaaccactgttgtattcctgagataaaggattataccccaatcttggtcatggggtataatccttttaatatgctattagatttcatattctagtattttcatgaggatttttgcgtttatatttataagggatattggttggtaatttttcttttttgtgatgtgtttatctggctttggtatagggtaatgcttgccttgtagaatgaggtaagaagtgctccttccttttctattttttttagaagagtcttagaaagattattgttaatttcttctttagtaaactggcagaagttaccactgaaatcatcaggtcctggactttactttgttgggaggtttttttctttattactaattaaatctctacttgtgtgtatgtttgttaagatcgtctgtttcattttgagtcagtttgataatctgtgtatttgtaggaatttgcccatttcatccaggttgtctaatatgttaacatacgattatttgtagtatgctgttttcatcctttttgtctctgtgaggttagtagtaatggccccactttcatttctgattttaattctttttctttttatttgtcagtctatccctacatctaaactcttttaagggtttgtcagttttgttgattgaaaagaaaaaaaacttttagttttgttgattctgttttttttctgttctctgttttattcatctctattcatatatttcttatttccttccttctgttagctttgggttcagtttcttctttgtagttctccggttgtagagttagattaatgatttgagatcttctttcttaatgtaagcatttacagctgtaaatttccttctgagcactgtctttgctgcatcccgtaagtcttggtattatgtcttaactatgtaaaaatttttcttgtttaagagtattatcttagtcagtttagtgctgctataaaggaatacctgaggccaagtaatttgtaaaggaaagaggtttatgtggctcacaattctgctagctagaaagtgaaagtctcaggctgcctccacttgtggtggaaggtgaaggggagccaacatatgcagtggttacgtggccagatagcagagaggtgtcaggctctttttaccaaccagttgtagtgggagggaactcatagaatgagaacacattcattaccaagaggaaggcacaattccattcatgagggatccacctctgtggtccaaataccttccattagaccccatctccaacactgaggatcaagttttaccatgaggtttagggggacaaacagtccaaactatagcaagtgtgttgtctgttatcttctttaaaccattggttgtctacagtgaattgttcaatttccacacatttgtgaattttctggtttcccttcattactaatttttagtttcatttcattgtggtcagaaaagatatttcatatgatttcagtcttttaaaatttattaacattgttttgtggcctaatatatggtctatcctgcatagacctatgctcaggtgcaaaataatgtgttctctgctatttgggggtggctgtattctgtatatgtatgttgggtccagttggtttttagtgttgttcaagttatctgtttccttattgatctgtcttgatgttctgttcattattgagagtggtatattgaagtctccaactattgtacagctgcccatttctcccttctattctgttagtctttgtttcatgtattgggggctgtgttgattggtgcatttgtgtatataattgttatagctccttgatgacttgatttttttaatcaacatacgatacccaaggctaggtgcattggctcacacctgtaatcccagcactttgggaggccaaggcaggtggatcacctgaggtcaggagttcgagaccagcctggccaacatggcaaaaccccatctctactaaaaaatacaaaaattagccaggtatggtggtgtgcgcctgtaatcccagctactcaggaggccgaggcaggagaatcacttgaacctgggaggcagaggttgaagtgagctgagatcctaccactgcactccagcctgggtgacagagcgagactttgtctcaaaaaaaaaatatatatatatatatattttttatatatatttatatttttatatatatttatgttatttatatatataacctgaagccttttatttttttcttctggacaccgttctggctagagtctctagtacagaagagataaatgcagataaatgcttactagaagagataaatgcagacattcttgttcttgctcctgattttagtggcaaagcattcagtctttcatcttcaagtgtgatgttagtcttgggtttttcatagatatagtgttatcagttgaggaagttctcttctatttccagcttgttgaagcatttttatgaaagggtgttggattttggcaagtgctttttctctattgattatgtggtttttgtttttattaaaatggtgttttatataagttgatttttgaatgttaaacctaccttacatttctgagattaatctcacttgggtcatagtgtgtaatcatttttatatgttgctaaacagtactttgttaagagtttttgcatctatattcataagggatatcgatcagtagttttattttgtttttgttttccttgtcatgtctttggttttagtattgggtaatactggcttcatagaatgacattgagaagtattccctctgcttctgttttttgaaagagtctgtgaagaattggttttaataccaattaataggaactatttagataaacagtgtagaactgatgtatgccaaggagtacagagtattttaaagacttggtgatgtttaatagcacagtgtgtgtagaggagatacaagtagtttgaaattgctggtatataaagttggggtagtggggattgtagtggagatgaggctaggaaagtaaaagtgggttcatatatgattaggtcaaggtcagcctcctagtataaaaataattttgttgtcgttgttctttttcaattagatgaaaaccccaaattacagaaatggtaccttgatcaggttgacaatatgcaattctcccctcttttcctctctctttatcttactatattatgaaaattactgatagaagtgaattttattcagtgaaataaagtgattacctatctaagttcgcttggtctatcttccttaaagttttatgttgatgtcttattaaagtggtttattgattatctggatgaaacttgactgatgaaatgctaacattttcaatggagtatgacctaggaagtgaatatataagtaataataaagctcttgagtttgaattatcaggacctcctcataaagtataaatgttactgacagcaaatgaggtggtaattatcatctgctcaaaaataatctaggagtttcttttgaatatcagttgtcatctgtttttatgttattttaatatttcagcaaaactaaaatgttattacatgcatactatataaaaaattgaagtaatacagaatggagtaaattaaaatgaaatattttttaccctaatccctaaattccctaatcccactcttaacagatataaatttcttatatgcatgtatgagcacatatattatatgtaaattagtaataacatatgtattttggaacatgttccctttttaccaacaatacagtaatagggcttttatgtctatatggacagaccctgcaaagtttcccctattgttaagatattaatatggtacatttgttacagttagttaattattatagtttattcagatgtccttggtttttgtctaaggtcttttgtttatgatcccatttgagataccacattacatttacaaagtataagaaagaataccacattgaggatccctcattacaacataatgagatgttagtggtcttatcttacttacctgcatggcagtttcttagactttccttggttttgatgatctcgacagttttttggagtactggtcagatgttttgtaggatgcctcactattggagtttgtctgcttttttaatgataaaactggggttacacgtttttggcaagaagacaacagagaggtaaagtgtcattttcatcacatcatatcaaggacatatactgtcgacatgctttatgagtgctgttaactgactttcaacacctgactgatgtagtgtttagttttcttgactgtaaaattactcttttccccactctttccatactttattccttggaaggaagtcactatgcagagactacacctaaggtgggaattatgcccctatacttcttttccttaatggtggagtagctacacaaattatttcaatttattttgcgtaagagacatgtctcttttcccccatgtattaatctatttagtcatttatttctatcagtgtggatttgtggatatttattttattggccacattgtcccaggtttggccactgggagttctgtcaactacctcctgtgcttcttgtttgtttttttgttttatttattattatggttattatttttaagaaggagtctcgctctgcctatcaggctggagtgcagtggcttgatctctgctcactgcaacctctgcctcccaggctcaagtgattctcctgccttggcctcccaaatagctgggattacaggcgtctgccaccatgcctggctaatttttttattttcacaagagacggggtttcaccatgttggccaggcttggctcgaactcctgacctcaggtgatccacccacctcggcctcccaaagtgctgggattacaggcgtgagccaccgcgcttggcggctcctgtgcttctttaacatactatcatgtgggctttttgttttattttaaagcatgtttttgttttctggcattataagataatggcatgcagatgtgtttttaatttcttgctcttcacaaattttatgcatgcacgagcgtgtacacacacagaattgttttcatagatgtaatccctaaagtgtcattgctgagtctatttgggggggctctatgtcttcacttctgaattaataacccattgcctgcccaatagattgcatgagtttggactttcacaagaattgttgtagttcatattggatggtgatctatttcaatacttagttttctcagactgaattttcttttctagtttgatagatgaaaaattgtggcttattgctatctttattacatttctttaattatgagtgaggctgagcatttttctacagttttgttggccatttttatgaccatctgttaatgcgtatttccatatttcctttcttgtttttgtttgtttgttttgcaggggttggggtgggcaacggcatgatttcgatcactgcaacctccacctccggggttcaagcaattctcctgcctcagcctccagggtagctaagattacaggtgtctgccaccaaatccagctaatttttgtattttcagtagaggcagggtttcatcatgttggccaagctggtctcaaacacctgaactcagcctcagcctcccaaagtgctgggattacaagcgtgagccaccgtacccagcctatatttccatgttttctgtttgaatactggttttttcttgttgacttccaagtactatttgtgttttaaagaaaaacaaatcaggcttttatcatgtgtagcagaagtttatttatttgtttatttgccaaattgtcatggtttttttttactgggtttgtgtgagttttttttcttaaggtagtatagatgttttacagttctgtgtaatcaaattggcattatttttctccatggcttagctcttttgtcttccaagatttgcagaaatacccatgttttcttctggcacatttattatttttctaaaaaaaaaaaaaaaccctttggtaattttggtgtttattttgttttaaggtgtgagatagggatgaaatttgccttaatcgttgtatgctaggtgcctaccctttcccatttccttcccacagcctctcaagcaaaaaggtattctcttacctaatccacacactctttactattagtactatattctcatctctagccagcctggcgtacccatcagagaaaagatagctattttggagatttttgccagttggaatgcccagtccaattctgatgagaatcctgggttactgtgatgctgcatttgatgtcttattgcggggatctctaatatgttgtattgcattacatcagatttttaaaattatatcatgattctattaaaaatgtaacctcaagtcattttatttccttgacctcagaagttatctggagataacacatcatactaatgtgttttccaaaggtaggatggagttatctcagctgttgttgagttgttgttacatgtgatcaaaattttatcacttaaatttcttactcagtgtgtggttttaatgtggagttcatagaaaaatggttatcagtgattagagaatagtttattaataaaattaataactaggaaaactgagtcatagagagaaatactccctaaattatacattatgtaatttttttggaagtttctaagtagatccactttgtataatagcagattcctttcaaaggacttctggcattgactcttttgccagttttttggaaacctagtatcagttatttgaggttagcagactcagcgttttcagctcctgcctcccaaatgatactgtgtgttctatttaggttttttaaatttttattttatttttttatttctttttgttttgttttgtttttttgagacagtcttgctctgtcacctaggctggagtgcagtggcacagtcttgactactgcaacctccgcctcctgggttgaagcgattctcatgcctcagcctcctgagtagctgggactacaggtgcatgccaccacgcctggctaacttttgtatttttggtagagacagggtttcgccatgttgcccaggctggtgttaaacttccgggctcaagagatccacccacttcggcctcccaaggtgctgggactgctggcgtgagccactgtgcccggcctatttagtttttaaagtttacaatttgtgagtattctagcattgtgtctgagaatccaaaggagatagattacattaaggaaatagcaaacctgagatgtttcagaaaattacgagtgctagaaatgctgaatcctggtcttctcaaaatattagcatctttagccttcttaattatgttgtgcaagaagcttcaaacaaaaccgtactttggaactgcaaaaatactactgttttccctacatatggtgtttcctagggatgggcactgtcccctcggggttattccctgtggctatttattttacattagaacatttgtaaatttaacaagagcattttttggtgccttcatatttctcatagtgtagtaaattagattgttcagcctatagcagacatgctttcattagccttcctttgatgtattctgctgtgttaatattatgaataaaatatgtggccaataaagggatatcactacttgcaatgcagcatgagctatacaaagtagagtggaattatcttcattgatttgaacacaatacttttgtagcctaatgttgcatgatgttttttattggttgcatctcagtgttttttctttttgagttctgttcaattacacttctttaatttccagatgtattactgtcaagtttgatctggagttcctctgttctctcattctcaataatacattgacactttgattttagtcatagggcctttgttaaatttcatattgcaggtttggtctatcataccagcccaagttctttaaatttttattcaatgtttcattttaagccattcttctaggtctgtgtcaatcttaggtggctgtgttgaaatcagaggttataaattgacagccaaatctatcccatgatgtgttttatttgacaagcacatttttttttttaaactagaatgcttgtagttagttaagttttgtactgtgtgatttaccaactgttgccccttattgcgttacacaatttggcttcacacttttacatatgttgcttagctgttgatggcatttgcaacctgtaataaatgaaatcatttataaaaatattgagacaagggaaggacagtttatagtcctgtaactagaaatctctgttgaacttggcattcatcttttaatcttacatataatttaaatatataaaattaaataaatgtgtgtgtgcatgcgcacgtgtgtgtgtgtgtgtgtgtgtgtgtgtgtgtgtgtgtgtgtgtaggagacagaatctcactgtgtcgtccaggctgggttgcagtggtgccatcacggctcactagccttgacttttcgggctcaagtgatcctcccaccttagcctcccgagtcgctgtgaccacaggcgtgtgctgccaggcctggctaattattattattattttttttttttttttttgaggcagagtttcgctcttgttgcccagactggggtgcaatggcgtgatctcagctcagagcaacctctgcctcctggttcaagccattctcctgcctcagcgtccagagtagctgggattacaggcatgcgccaccatgcctggctaattttgtatttttagtagagacgggatttctccatgttggtcaggctggtctcgaactccagacctcaggtgatccgcccacctcggcctgccaaagtgctgggattacaggcgtgagccaccatgcctggccaattttttgtttttttgtagaaatggtgcctccctgtgttgcccaggctggttgtgaactcctggactcaagtgatccttctgccttggcctcctacagtgctgggattataggcatgagccactgcgcctggttcagcctttatattcttaactgttagtatgattgtttagcaaatgaagtaaattggaatgtagtggtatacacatctccaggttgttcataattgtataatgattaattttgtaaaatggatcattaaaatcaagaaactttgtgattgagtaatagttaaaaaaatggttttgacatctgtgttaatgacctttctaatacctgagactcaccattctttgaattctatttaatacagcaatccttttcacaccaattgagcattctgttaatttgttctctttattagacctcatttacaatgggaactgattgtttctgaatattgatctctgcatttttttcttttagccttcagcattactcttgttacccctagttttgcatatcatcttatctcttactttacaagaaataggatcatcacacataactcattcagcttacatttctttccttttccctaatacctaatcacttattagatttaaagaaaaaaagataactaattttttaaggatgaaaatagctaaaactcgagtctttattgtgtgttaggtaatgtatttaaagaatttcacattgatgatttcttataatatcatgacagttctttaaaatagatactgtcattagctctatattgcagatgaggaaactgatgcttagagaggttaaataacttgctcatagttacataactcctagtaacagaattgggacttctatatagttttatgtgactcatgctaatcttacttcttaattactatgctatatagtttgtctttgtccttgtttcttccttctcctgattttctagaatcttgcttcttatcccttttccctctttttttttttttttttttttttatttttttgatatggagtctcgctctatctcccaggctggagtgcagtggtgtgatctcagcttactgcaacctctgcctcctgggttcaagcgattcttctgcctcagcctcccaagtagctaggactacaggcacctgccaccatgcccggctaattttttgtatttttagtagagacggggtttcaccgtgttagccaggatggtctcgatctcctgacctcatgatccgcccgcctctgcctcccaaagtgctgggattacaggcgtgagccaccaagcccggcctttcctccttttttataactttattctacctatgtctacttagcttacaaataaatttgggtctcttttagccagtaaaaagtgcaaacaaaagcccagcaacaatgtttctttgacttttgctccccttgagttaccaactcccagttctctccttacttcatcaccagagactttgtgagaataaactgaattaactacctcctattcctcatctgccacctgctcacttgtgaacccaataaagcttgattgctactggcaccatggtctgaaacctctttttccaagagcatcactgaaccaagatacaccaagatattgggtatagatcgaataacctctttttagtcctcatctcgtctgctattgaaatgcttcacagtttcacctctcacccttatagaaatttccactttgaattgtcgatgctaatctttccttgctttcttttgcctcttagttttttcttcatgtttgctaagtcccctgtgttgtctgcttcttaaatttagtgtttttcaggattccatactcagtcctttccttactctccaactctgggtggtcttcttaggtgaacaagtctattctcatggcttcagctattgcctgaatctcaagctgtactccactcttgcttcctgagttttgccttgacctctgtttgcaaccacctatgtaatataactactcaaagtgttctgagattcattaagtaaaagttcaaataatttttccttcaaacctttctactgtttttgctttctgtgtctcatcacctatttggttgcccaagcttaaagacagttaaattaatcccaatctctttgtttcttggattctctattagtctcatgttcaactagccggtatacctaccctgccttcccgctgctttaaaaaaagtccctattgccttatgccctgattcagcctttggactcttgtagaaggtttccagctgctcaccctgcattcattctcactttcccaagtctcctctccatacccattttccataatactgtttgagttaaaacagacatgtcagtttgtaatctaatttttttcttcaagctttattgatataattgaaaaataaaaatgtatatatttacagtgtacatgatttttatatgtgtatacattgtgaaatatgtaatactacattttttaaaatgcccattgcccttacacttaatttagtcagcaaatatttactgagtacctactgtgtgctaggcactattctacatggagatagagcagtgaataaaacagacaaaagcctttgccatcatggagccttgtctagtcagaggaagcggataaaataaaataaactatgtggttatacaatggtaagagtcatttataaagtggggaaggcagatggggaatgtgaaggtggaggaatgggttgtaattttaaatggaatgattaaggaaagccatactaagaaggtaacacttttaccaaagattttgaaggagttgagacaaatgagctttgagcatgtcttgggggagaacattctaggttaaagaattagaagccctgaagtacagaatccctgaatctgaaaatgtaccatatgtgttcaagaaatagcagggaagtagcttggctagagaaaagtaagcagtgaggacagcagtggagtagaagaatagaggacagaaataatgagatgggtgtgtagtaccttatagaccaatgaggtagagtagaagagatacagtctaacttgacttgattttagttgatagtagactggaaaggaataagggtagaagcagaccatttaggaagatattacagtaatccacgtgaaaggtgatagtagcttggacgagggttctaccagtgaagatgatgagtggttggatccagataaattttaaagatggaaccattcatatttcctggcatttggatatgagggtgtatgtgtgggtataggggtgtgtgtgtgtgtgtgtgtgtgtgtggaagtgtaagagagaaggggctgggagttgattaaggatgattctaaggttttcagccagagcaataccagatacacagttggatatactagtgtggagttaagggcagaggtctagtatatgtgtatgtatttatatataatcagtgtatagttgtattgagtcattagactggatgagattatcaagggggagtgagtttaaagaacagaagagatctgaggactaagtccagggatcttccaatttttaagagatcaaggagaataggaggaagcagaaatggagaaaagaagagttgtctgtagggtaggatgaaaaccaaaaaagtatagtgtcctatgaagtgaagaaggtgcttcaatgaaaggaacgtgattgacttttccaaatggtagtgccaagtcaaatagatgagaagggtaacatgatgactgaaaattggctttggattcagcaacaaggagattgttcatctggaataattttggcatagtggtagtgaaattctgttggagaaagttttagaataggaggagagaggaactggagaaagccagcttaaggagaggaggcagaaatatggtggtgattagtgtggttagagatagttttttttttttaagatgggagaaataatagcaagtttatgtgctgattgtaatgtttccacacggaataattaatgcagaaagacaaaaaaaattctggagcaatgaccttgagtacaggaccatttttgtttttgctttaacatgtagtgatgtttgccttaggaaggagctgggttagcttatctctagttacagaagtaaaataagagtatatggagatagatgctggtaggttagtgcttgtagtgatgcaagcaaggtcatctgttgaggctgaggttagagaaggagtgttagaggttgaagaggaggaaagaagaaattaagtggtctggaaaagtgaatggaccaggaaagtgtataattattgctggacaaattgattttgagacaaggtgtggctctgtcacccagactggagtgcatggcatgatcttggcttattgcaacctttgcctcccaggctgaagccattctcccacctccttctcccaagtaactgggactacaggtgcacaccaccacgcctggctaatttttgtattttttgtagagatggggttttgccatgttgcccaggctggtctcaaactcatgaactcaagcgatctgcctgcctcagcctcccaaaatgctgggattacaggcgtaagccaccacgcctggctaacaaattaaattttgatgatgacttgaacatgacaccagtcagcataattagaggtttctttgcagccatgttgagctctttggtcgtagacatggactgggcagaaagctgaactcaaccagggttgtggaaaaaccacaagctttttcttagatatttttggctttgtgggctatctagtctctgtcacaattactcagttctgtcactgtagtccaaaagcagccatagacaatacttaactgaataggcatagctctgttccattaaaagtttatttttaaaaacaggcagcagactattcctgttgtttgttaactctcgtgttagagaggataatgatggaccgtggaatttaagcaggctaagaagggaagagaggagaacataagtgcgtgaagtgtagagagaaggtagtagggtcaatggcatgatgtttcactatttcctaatctctgctctactcttacatgaccttatgttttgaatatagttttctctctctgcgtgaaatgcttcttttatacacagtttagatgtcatctcttttatacttttatttcctctgctgtggccactgataatatggaaaatgctcagtaaatgctgaaaattgaaattctccacttcagtttgataggtaagattatttcaaattgggagattgtgaaagtttgtttatccacagtatttctcactcagtagccaagcctatgcttgaacatggtgattctcaactttgttttctgcttcaatttacctgatactggccacatgaacaaggtaagggcagtgccctgctcttccttcctacttggtgcttaagttagaatgcctttctttttctccactggtgttgttgagttggtgtctttatgctgcagagcatggatatttaagcataggacctgtaatgagcttgtgagtatggtaacacgttgccttataggttattttttaaatttcagttaattatagaagacaagtgattgttctatcagttatatactatcaggtaactataagatctttttgtgtgtgtgtaggttagagacattttggactgtttagtctgacttttcatattgtagttgaagaaactgaagcctagagatgcaagtgacttgcacacaccaactagcagagctagggctagaaatccatgtttcttagtttcagattcagtactctccctgccaccacgatacttatcttaataggatatgaggacattgattatcagcctagaaaatattatattttatgacaggtgatatcctctaaaatcatggtaaccaaaataatcgtttaaatgtaattgggaatcttaacaatattgaaatgactattttttccccattgaattattagtaaaaaggtgtggttgacttgttttattattatggttatattctgaagtaattttatataattaaaagtgacacattaatctttaaaaatccgaataagtaaatatctagagaatttactctaatttgaatatttaaagaccagtttctaatctgttttgttttttgtttttttaaataaactctttcagaaagatgaggtctatcttaatctggtgctggactatgttccggaaacagtatacagagttgccagacactatagtcgagccaaacagacgctccctgtgatttatgtcaaggtatgtaaaaacataacaattttccattttagaaaacattttttatgaaccaaggagagcctcttttatgttttaactatattattgtatttaatactcagggagaaaaactgtaaagtaggtattgttggaactttgttaaatccttttacagttttaaaatgtgaattgtgcaccttttgaagttttataactcagatagtaaagatattctggtaaattacagtgaatatttttcacagacatttatagattttatttctgctagccacagtacattttaatttaacttctgatatctttattgtgccacaaaccaaaataggagcgtatcatataaaattgtaaaatatagttataaatatgttcaaattcatttgttcacagatgccatttatatattttttagcatctgtagctgcagcttaaactgttggcaattttaaatatgcattgggtgttgtttattttttggtatctaatatcaatgccatcttgtttatttcctagaactcatggcagaatggctaggtatttatctagcataatttgtattaaagttgtggttagttattagtaacaaattattttattaactatggcattgtgaaaactagtagttaagtttgaaagttcacatatctaaatacaatttttaaatttaaccctttatggaaataactgtagaaaataaaatgtcattgactctgaatgtcttatggttagagtttttgaagtcttgttaaaaatttaagcttcacaataataaaagggttatcaaagtagaaccattaataatagaagttctttggatggattttgcataagctaataataataataataataatagaagttcttcaagcacttttgttagctagattactttggcaaggagtcatatatatatatagtatatataatatacactatatattgtatatataatatataaactatatatactatagtgtatattacatatttatatataaactatactatatgtagtgtatatcatatatttatatataaagtatatatatactttatgtatagataaggatgttcttttttattatttttccctcctcccttccttccttccttccttccttccttccttccttccttccttccttccctccttccttccttccttccctccttccctacctacctacctacctacctacctaacctacctccttacctacctacctccttccttccttccttccttcatttcactctgttgcccaggctggagtgcagtggcacaatctcggctcactgcaacctccaccctctgggttcaagcgattctcctgcctcagcctcccaagtagctgggattacaggcacacgcgaccatgcccagctaatttttgtgtttttagtggagatgtggtttcatcatgttggctgggctggtcttgaactcctgacctcaattgatccaccagctgggactccccaagtactgagattacaggcgcaagccaccacgcctggccaggatattattttttaaaatgtaattaaattaccattgtcatacctatgaaaataaatggtatttcattagtgtcattagatacccagtttatatttgcctatgacataatttttttaagtttatatttttgaattggcattcagttaaggtttatatactgtgattggctgatgtgcctctaatgtttcttttaatccataggttctctttttctctctcttgaactttgttgctaaagaaaccggatcatttgacctatactttcttggtctggattttgctgaatttctgtagtgtaattagtatgttctttggtcatctatatttcctgcaaattggtggttagataactgataattaaatatatttttggtaggagtcaggaccacttcatttgtagtattgagtgctttctgaaggaagaacataatgtctttcttttcctgacgttagtagccattgatgatcactgcctaaattcattaattcattaaaggttacaaaatgttgatagtctactgttattattccttcttaatttataagctggaatagtttcataaagagagattttcccatatcaactatttgtgccctgagatatagtttatataaaaagacaggaaatgtatttgatcccttttctttatttacgaaaatttataatagtctattccatagcatcctccataggtaccaatgaatgaggtgttttgttgttttaaagtatcttatgtgagtcttaataatatttgaccgtttccttgctttctaacatgacaaagtattccaagttcatcttacacatttcttcctcagacttagaatcaaccttttcttcaagaagaccttgttcttttagtgggaggtggtatttagagaccacagtctggatgctagaggtgctaattgtcacgggattggttattgtttctgggtctttttagcgaacagagctaggaaatatgtgggtaattttctcagatgtaatgcatcttctttttttccaaaaataaatacaccatgagttcatactgatactttcaaataaaatcgatgagtttttacttaactgtaataatcttatatttgtatttcctttctcttacattgaaaatttcatttctcaatgatagtagtataattaataacttattctacccagcaccctacataaccatcttagaataacaattccaacactaggaccaccagctaaaaataatttaagatctaagcacagctcagttttacccattagatcccattagggatgaacaatcaaatttctctgttctaaagaaacactgaggtcaggcacattggctcatgcctgtaatccagcattttgggaggctgatgtgtgcagatcaccagaggtcgtcaggagttcaagaccagcctggccaacatggcgagaccccacctctactaaaaaatataaaaattggctgggcatcctggagcacacctgtaatcctagctactcaggaggctgaggtaggagaatcacttgtacctgagaagcagaggttgcagtaagccaagatcatgccactgcacttcagcctgggtgacagagcaagacttcatctcaaaaaaattgaataattcctctctgtgtgatgatgttccattaagcatttgtttgtttcagtttacttttattatagattgtggttttctaaattttcaaataaaatttactttaaaattcatataaaatatttatacatagttcaaagcaagattgacaaaacaaggtatgttcaaagaagttcagcttttatctctgttccctctgctctgttcctttcctctaccagtgttaagtattttttaagtgttcataatttatcctaccatttttaaaataagcaaacatgcagttattcttactctttttatataaatggttacataaggtacacatttttctcttttttttcagttaaaaatatgtcctagagatcagtccatattttcgtgtgtgtgtgtgtgtgtgtcaccttctttatggctaccatattacttcatcatgtggctgtattctagttaattcacccagtgaccaataatgcacatattgttttcctcttgttttttgttgtggtttttttggtttttgctatagtgaacaaccctgtgcacttacggtatttttgccagcaaatagttgagctagatatctggcatcctgtattttaaaaaagaaaataaaattattgtacatactgaagttggcatacagagagaaatctagcactcctttctttctcccccttgtgggaaaagggatgatttttcttttttttttttttcttttttttttttcttatgtaaccacagcgctttcagaggccagatctattggctctgggcattattctttggggacatcgatcccttttatctgaaaagataaaaacagtgtctggaattgatgagttatgagttgatctccttcacgtagtcattctacatagttttattgtaatcctattgtatgaaaaatgttagattgtgagagcgatataaaagtagtaaatgcaagtaactcttgccttcatgaacctcatcatctgataaggacttgtttggcatacatgttaccagaatggcaactgcatttctttataattgcctttcactttttctgggagagttgagtattttctatgagtaatacacaggttaaaaactgagcacctttgcttttctgatgagtatatttcagaacttaagtaggttttaaaatgcaactctaaaacagatgactggcaaatcattgttgtgaacttaatttttttttaattgaaaatacatgtttaaggacagggtctgatcacacaagagagttggatgcattttttgaatcatgaacaaaattgttattacttgttttaacatgtattctattgttttattattttattacttcagatattatggcacagagaagagggcattggatttgagttaaatataactaactgttcaaattgtgctcaactgcaaaatacattcctttctgtgatattttgtcctgtaactgaagcatagtaattgttttatggaaatgttaacagttttgtaccaactttgaataaaatgaaaaataaaaaaaattgttttcattgaacctcaatatcttctgtaaaaacagggataatgtgtatctcagagtggttgcaagaattaactaaacaaataggcaaataggcagaaatgcttttcttggtttggcacttagtatatgttaaatctgttatattttaaagtgtgttaattatatttgagtttctaagaattaatcattctagtcttttatttagccaccaaactgctttttttttgttttgttttgtttttgtttttttgagagagagagaattccgctctgtggcccaggctggagtgcagtggtgcattctgggttcactgcaacctctatctcccgggtccaagcaattctcctgccccagccttccgagctgggattacaggcgtgcaccaccaagaccggctaattttttgcattttagtagagacggggtttcaccatgttgcccccagggtggtctcgaactcctgagctcaggcaacccgcctgccttggtctcccaaagtgctagggttataggtgtgagccaccgcgcccggcctccaccaagctatttaatgattatattttacacctttatttaataaaatatggcaaaatacataaatagtaattgaaagagaagcacttactcaaaccactacagaatattgtgtgtgtgtgatattgagcaagttgcttaatctctctgggcctcagttattttatatttaaaatggtaataatattagtacttattacataaggttattataaggagtaagtgaaataatacacataaagcagttagaacagtgcccagcccatgataagttgtttttctgttcttgtttttgctatcattctagtctagaaagcctctcagctttcttataaatacacttttacaaagtgaaataacaacaaatatataatattgcatatttcattctttatcttgacttttctagataaacttttcaaaacaatgaagatagagtagatctttcagatgaaagaatacagtaaaaagacatcttaaaattaacctcatttaaacgattaagctttttttttttaaaaaaaaaaaagaaaactcgttttcactgattcttgcagacctagacatcaataccctgacacagtaaaatctctttttttagtaaatttatgcctatgaactcagtttcttatgggaggttatctttttagtggtaatgaattaaactcctacagtaagctttaggacttagaaatggattctgtgaatgatggtacaataatagaactatgaagtttttaaaaaatgttacatggatacctaaatgccttaattttaactttgttggcaaaatgctgaccttataaaagatcttatctgtacagtttacatcttgcttcattttaaagaaaacataaacttttccagctaatagttcacattaacttcaaggatgtactaaaattcagctgtctgcagttttcaccatgcttcttagaatgaaagtaggtttcatgttaactttaattaacctaagccttagcagtagcttttaaagatggtgccatatgtcaatgcttcattagacttttaagtatctttttttttaattgtttttttaccaagatatttattatctttggtttgactcatttcaaaaattctgtccatggtgtaactttggggtagaggtcagaaggtaaacagccagaaaagtttaaaagtgaatcagaacactgaccttgagataaacagaaggtttaaattatactctcttcgtccatgaagtttctctctcctccagtcaagagtctgattctttactccatgcagacccagtttctcagtgtatctttttggtaagaaaactgagaaagtcttctcttttgaaagatgatgtcaatagtcaggcatctgtttttgaatgtcattttgaattatactttttctgaaggatattaacaggaagaatggtgatgctactaacagggttattgatgtcttgatagagcagtgattacagattttctcagtccacatgagttttcctagaccactgtatcaatcaccttggaggctgacatctcagaagaatattctaaagatcacactttggaaatacgtttacattcagcagcatgctctactagaaagaactataaacttaaaaatcaggagacctggtttctggcccaaagttactgattattcatgatttggggtgcctctctgagcttttcatttgtgttttaaataaaagcccatcttagcaggcttgtaatgtgagatgttagtaaaattaaaagttataaagcacattgtaaaattttcttcttttgagttattaaagaatgaagcgaatagtacatatggttaatgacagtttagctaattcatatagaaatagctaattaatacagaagtgacacaaaacttgtgaaaggaagattgagtgccagtaatccatagcatttcacctgttcttttctgagaatctgttactttattgactgcttaaggattctgatttgtttagaataggaagtatttaattaatttctactatctattccagttgtatatgtatcagctgttccgaagtttagcctatatccattcctttggaatctgccatcgggatattaaaccgcagaacctcttgttggatcctgatactgctgtattaaaactctgtgactttggaaggtaagctactgtccctctccctgccccttcccttttcccctcctccccctactatcttcttttaaatatattccttttgcaatatcagcctaaacacagtaattcttattcaagaagtataaactagtgcagctgtgagagtagttttgttagtttctgtcaaaattataaatgtatatactccttcatttaacagtttaacttttaggaattatttccttaggaatacatatttacaatacagtgtatatatagggatttttattatagcattgtttataagcatccattagtaggaaaatggctaaataaattacggtacatgcagagtaatacatgcaaccatgaaaaagaatgaggcaactctctgtgaactgaaagggaatgatgtctaggaaacattaaatgaagaaagcaagatcaagagtatatgtattgtatgccaccttttatgttttaaaaacatacagaatgtgactatatatatatatatgtacacacacacatatatacacaagtacatgtgtggcttatttctgaaatggtacgtaatccataatatttgattgcctgagaaaggatctttgggaatataatgtagttgggaaactcactttttcattgattgtattttagtattgtttgaatttacattaggtatatatactcctttttcagtaagaactaaattttttcaaaatattgattattatacattcctgtgtatctttttaacttcacaattaacctcaattcagggatgagataaaaaacaatgcagtgttttcttcattttcctctataatttaaccttttattccaccctttttcctgcaaatttcatcaaactttctgcttttaaaagctcatttcttggtaatatactgtgaatttgtaatacaactttattctttccattttcagttttcagtactttgtgcctctctgatctaaccttttaacatgtagtcaagcctacattcccatgtaactcaggcagttaccccgctacttcctcactggtgtaatcacctaatctctctcctcatggtccctaagacatctttacataatttggattctctccaatttaattttttcacccttgttacagattcttttcaccatagctctatagataacatattgccccacacttgttaatacctgtgttcctatccctttgtaatacttttacatcaattcacagtttaaatttggggtttaacataggatatgttttccacaggcttctcttgagacagatcctttaaaaaataagtaaatctgttctgtcatgagttaatttttcttagtctcacaatgtgtcctctttcagactgtcctcattttactgttttctaatttcagatgacctatcactttgcaggccatgactctgttccctgaactatttttaaaaagggtgaaagatagtaaaactcttacctggtgtttgaaacaaagcactactacttgtctataggaaaaactgcccaaatcagatatgttttattgttaatggggtgtcttcaccattgagagtgactgacttggtagggacagactgagacaccaaggttagaagagcttctgttctcatatagtgtcaaaacacttggtacagcccattctcttgggacataaaatagctttattctgataaatttggaagagggatttcgcttgtttgatagctcatttttataaaccctctgatttttttgtttttaacaaaattggtttggtcagatggagtttgggaaacactttggactgtacattatcagattggtaatttgaacaaaatactattactttcttcttatttttttactagatgaaaaggggccaaaaagtagctaaagaactagttgggctatttaggaacaaaaagctagatgtgatcacaatattgggaagtagttaaaaccagtctcctctgtaagagaggctgctccgtttttgtagctccatcaggtagtgaaaaaacttcaggctacaaagcttgggtattaacgttttgtctcgtaatgaattttctgaacataattagcatctatcttgttaattaaaaagcatcctttagaagatataacagtctcctatcaattatgctgaaaactaaatgtcataattttcatgcagcctaaaaacagggattagtatttattaatttatgaaatggttattgtatgtcagtacttggggagagggttaaaaaacaaacttcaaaactagtgatgaaggaaaaagaagttaaggttagagtcatcattgactagaaataaatggaagaaaagaaatcccagtcattgaaatccttgtgactactttcatctgtctttaccctggttttccagtacctttactttttttttttggcacttactgttttactgttttgattttgttgcattttgtccctttatttatctttattattaatgtatgtcagaatggctggcttctacagcagtttattttattctgtctttggattatcttgggcgacatctcaaaagaaattaattgtaatttattatacttcatatttaaaatacatgcaatgaagtattttagtaggaaaattggaataagaaaacatgcaattatacttactatagcagagttagaatttataggcagtgaatcatttctgccctgaagttgaagggcctgtttataatttattgggtaataaatgtttatgtggttaataaattgaccaagatatggaaaaatagattgagtctataagccttgaaatattcgccgtgagtcagaagtcaaaatgcaagacagtggccagtttctcaccctttttaaagaaatacattgggtgaggagtattcattcttagaatccattgtcatgctttttggtgggggaggtacctcttttctgcccttgcttctttgtattttagaggtggaatttggggaacagaggatcagaagaaattcaacacttatccttttttgcttttggtgccttcttaggtgacaaacgctttctttaaaaattgctattcttttttgcagagcaaggtgataaaattgtaaatttcttttatyagagatagttacagtttacattttctttttcatatatgattcaaatgatgtgaatcttataaaatcatttgatactgtgaaaggatagcagtctaagttctttaaggtttagatttaaagaatttctgctttttatttttattttttttctgttagtgcaaagcagctggtccgaggagaacccaatgtttcgtatatctgttctcggtactatagggcaccagagttgatctttggagccactgattatacctctagtataggtaagtacaaatctgaatataatgctcataattattaattgcttcttgattttggtaatttagtactactaatctgtacttttatttccatggaaagatggatatatcccataatgttttttttcttaaagcttgagatgcaaacatgtataggcatacctcattttattatactttccaggcactgcttttttttttttaacctctcaaaggtttgtgtcaaccctgagtcaagcaagtctgcccacactagttttataagagcatgtgctcacttcatgtctttgtgtcatcatgtctttgtgtcacattttggtaattattgcaacatttcaaacattctcattgtatctgctatggtgatctatgatcagtggtctttgatgttactattgtaattgttttggtttgccacaaaccacaacaatataagacagcaaacttaatcgatcggtgttgtgtatgttatgaatattccaccagctggccattccccatctctcttcctatcctcaggcctccctattccctgagagacaacaatattgaaattaggccaattaataactctgcagttgcctctaagtgttcaagtgaaaggtacatttttcacttttaatcaaaagctagaaatgattaagctgagcaaggaaggcatatcgaaattgtgccacacagttagccaagttgtgaccagaaaggaaaagttcttaaaggaaattaaagtgctactccaatgaacagatgaatgataagaaagtgaaacagccttattgctgttacagggaaagttttagtggtttggatagatcaaaccaaccacaacattcacttgagccaaagcctaacccagcaaggcctgaactcccttcaattctgtgaagacagagaggtgaggaagctacacaagaaaaatctgaaggtagcagaggttatttcatgaggattaagggtaaaaagctgcaccatgccataaaagtgcaaggcaagcagcaagtgctgatgtagaagctgcagcaagttatccagaagatctaactaagatcatcgatgaaggtggctacattaaacatcagagttttaatggagaccaaatagccttctactgaattggaagaagataccgtttaggactttcatctctagattttctagagatggaagacttcttctagagaggagaagtgtcattgcctggcttcaaagcttctaaggacaggttgactctcttgtgagggcctaatgtagctggtgacttttaagtgcaagccactgctcatttaccattccagaaatcctagggcccttaagaatgatgctgaatgtattctgcctgtgctgcataaatggagcaacaatgagtggatgatagcacatctgtttatagcatggttcactgaatatttgaagcccactgctgagacctactactccagagaagagtatttctttcaaaatattactgcttatggacagtgcaactggtcacccaaaggttgagaaatacatttgtaaggctatagctgccctagatagtgattagtctgatggatctgggcaaagtcaattttaaatcttctggaaaggattcaccattctaggtgtcactaagaacatttgtgactcaatcacatgaacaacattttggaagaagttgattccaagcctcatgggtgaccttgaggcattctagacttcagtgaaggaagtctctgcagatgttgtggaactagcaagagaactagaattagaagtggagcctgaagatgtgactgaactgctgcaatctcatgatgaaacataaacaagttgcttcttttatggatgagcaaagaaagtggtttcttgaggtggaatctactagtggttaagacgctgtgaatattgttgaaatgacaacaaaggatttagaatattacataattgtagttgataaagcagcagcaggggttgagaggactgactcccattttgaaagtggttctaatgtgggtaaaatgctatcaaagagcatctcctactagagaaaaatctttcatgaagggaagagtcagttgatgtggcaaacttcattgtcactttaagaaattactccagccacatcgtatagccttcagcaactgccaccctgatcagtcagcagccatcgacatcaaggcaagaccctctgccagcaaaaagattacaactcactgaaggcttagatgatcattagcatttttttttagtaataaagtgtgtttaagttaatgtattacatttatgtttttatacaattctattatacaagtaatagactacagtatagagtgaacataccttttatatgcactgggataccagaaaattggtgagacttgctttattgtgtcttattgttatggtctggaaccagttctgcaacatctctgacgtatgcctgtatagaatttttcaaattctactttttagaatttaaggtatacctactattctatttttcttccttggactgttagaatcataaagctaatagcttttaccttaagaatttaaattaattctaagtaacttttttttactttcttatttaaaatcctcctgaaatacttctgagcatgattatactagttggataatttattaattaatgttatttcattaagttaataatgaaagtgatagtatggccatattgctaacatttccttcatgccatctccattatttaatataaaatcagcttaaatcactagtagttttgctagatgcactgattctgcagaagtatgcacccaagatgtttagtcatagaaggaactgaatagagaccattagctttaatcatcattgaccttctcaagtcttcaccccagatgtttagtgataggaggaactgaatagagaccattagctttaatcatcattgaccttctcaagtcttcaccccagatgtttagtgataggaggaactgaatagagaccattagctttaatcatcattgaccttctcaagtcttcacccaagatgtttagtgatagaaggaactgaatagagaccattagctttaatcatcattgaccttctcaagtcgtcacccaagatgtttagtgatagaaggaactgaatagagaccattagctttaatcattattgaccttctcttttactacatcttagcccactagccaggagtgaacagagagagcatgatcagctggacatgaataggaagctaacaaacaaggagcaactttaagctggatgtgcaagtggaaaccaaaattattttactcttacatattgtttctgaagagaattcctttagtccagaaacaaacaaaagcatgcaaataataaaccaaaaacatccccacagtaacagaaaaccaaaacaaacaaaaaacagacctggcctggtgcagtggctcacacctgtaatcccagcactttgggaggctgaggtgggcagatcacttgaactcaggagttcgagaccagcctgggcaacatggtgaaaacctgtcactaccaaaaattagccgggcgtggtgacatgcccctgtagtccccgctacttgggaggatgaggtgggaggatggcttgaggccaggaggcagaggttgcagtgagctgagactccaccactgcactccagctcggtgacagaatgagaccccccctctcaaaacaacaacagcaacaacaaaaaatagaacagaccttgtgacagtttttttgtctttttgatataaatagaaattctctgatacaagcaaacaccctaatcagaaacacattatcattttttattttgccaaatggcaaaatgccaaagcacattttgatttctgccaagaggccagcagttttacccaccatttcctttgtaccctcagtacaaatgttaacccagtgaaggtgtattcttatgaaaagagttttgccctctgggacatctagaaatgtctagaggacccaggtgcaaagagtacaaagtgtcctctgtagaccacactttgacatctgctttaccagagggtagagagcataataatgtgtatatgtatgtgtatgtaattctcagagtcttagtatggctgtggtacatggcatatatgaatggacagatgaaaaatagtacaaagaatattttgcttgtggtcgacctgggtttgagtgtcaggtcacttgaagtaaagtggagattactgacagttcttttctgagtggctgtgaggcttaaataaaatgttagtgaaaatactgtagaaatgaaaggtggtattcttttgtggcatttttagacttgactctccccaagagcaaatttcagccccttgtctttgggtatgtcattggattttccagctgctttacattttattcaaatatatgatactgattatctgtatataagatactggtgtgagctgtgccagaaacacctaaaataaaggcagtttgattgttctcaaaaagttaacacatcatctcacacattaaggaacagttatataaacaggagtcttttgaaccctagcaagcctaggtttggcttcccattataaacttaggatacttgcctcttaagactgtaatgtttttaaacagaccctcccaaaaagtatactaatattgttacatttttaaatgtttttaacagtaatgtttgaaatgaaagagtaactgtttctgacaagattattgtcatatttagatacagccagaggtaaagctgatgtctctgattatattatcttactgacaaatggcagaatgagaacaaaaggcttccttagaacaaggaattgaacttacagacagacccttgaagaagctgttattctgtgacagtgagacagttattcaagagagtggtgttttgattgggaattgagaatgattccaggcctagctctactattcagtacctttaccttgggcaagtcgtttaactttatcatgtcttaatttccttatctatgaaataagtgtgttaaactagatggtcttgaaagatcactttaacatgaacccgagttaaatatgaaacaaaatgctattctggctttaaccccaactaactaaaattcagtgtgccaaagcactatttctatttttccggtaccttctatttagttgaagttttattctatctggagaatttcatccatttgtcctttgaagtaagagcagaagagattttagtgtcctccagaaagtgattgataagtaccttgtttttactaacttgactgatgtaacatgaattgagggctgctagtatagttgtcacctgaatcttttgagtgtcagtcttctgtgataacttctaggcaagagtattccttgatgttttactttttgtcttccatatttgtagctaaatcttacctaagaaagtgtttcctattgttttttatgaaggcataattattcccttacaattttttcagccttgtatctgagatgaatcattccgtattagagaggaaaaaagttgagtgcatattaccttccatcttattaactaggtaaagggatgttttaaaacttagattaaaataagcatttacataaataaggagatacatcttttccgcttgactataacagttagtattactattggctttgagtaaagattccctaacaagttttcatatgtgcttaatttagcaatttcctaatatattaaataggactcttggtctatatttgcagaatagatgtcttggttattaagttaggactggacaaaatcaactcgagtttgttaaatgatactatgtcatttttctaggtcaataaaaagtagtcactggagcaacgatttccagtctctcttgatagtacatctcaccacatcagacctttaaaattttttgttgtcaaccatttactgttccttcttctccttccgtactggtaggtggtaaagtccagcaatgattcatattcgtctccctaaagcacttgagtgttggtacattaaaaacctaggaatctcagaaatctggaagctaaaaagcagccttttcttgtaggaagtcttaatagcgggctttaaaatagagtgaccgtacggtttatcttctaatccgataatacttttgagagagaaagaggtgttactaataattatcccaggacaacatataaaccataactatcctgggtaaaccaggatgtatactttaaagtatttttaaagtgtattaattgagttaatttttagagttttctctccggattgaggttttcattttgacttaaaagtggattatttttataatttatgaagtaaaatgtattcctccatcattgctattccattttatttaaaaatgatatatttaagcacatttttctgtcagtcatcgtagaacataaaaatcaaccatttaaagtcttaaaagtgttcctcagaatttgcactgacttaaagcccagtgtctgggagagattgaacctttccttgctgctcctctgtaagtacctactaatatgcagacgttgtgtcactttgacttatttttaagtaagataaatagatagtcctgccatagctttggtactgtaatcctctgtctagttatttgaagacttgaatacctgaaatttgataataatagactttaccctttattagaatattcttattggtctggtttcctctttggtaagtttaacatttaattaaaaagtacattaaggaagtttgatgactttttctctgttttggattgtataatcttttcctgtgatatttactctgtgatcaaagtaaaattagaaaacgtatacaaacacttttcagctcaagctatgaagtattaataaggcttcgttttcttttctctttagatgtatggtctgctggctgtgtgttggctgagctgttactaggacaaccaatatttccaggggatagtggtgtggatcagttggtagaaataatcaaggtgagaaggttgaatcagaatatttgaactggaaggaatttttgtgctactttaataatatatatcacacatgtacacatacgagaaacttaaaaaatggaggaagcaatccaaggtcacgtagcaagttcagctataaagtgggtataagaacgcccccccagccttttggtttttatttgagttactcttctcttgtatgatcctagaaaagctattatgtatatctgatgcaagaaaggtatatatctgttatttaaagtacttaaaagcaaaattagactataagctcactaaaatgtatgtaagctgaataaaagcagaagctgtgtctgcttttgctcactgtatactccctgctcctaatactctgtggagcacataggagatacttgtatttggtcactatttatatttattcagtatttataaaatgaaagaatgaatgcctacctgtctggctagatcattgatagaaacaaaattttgacttatggatttaatataattttatttattgggtctatatgaatttaatataattttatttattttaaattttatttaaagctgaataaagatatgtctggttttgctcagacacagcttctgctttcattcagctttgcatacatacacctttattaacaaatatttaaatattatcttagaatatatgttttcttttaagaacagtagagctgagattttaattgagtttttaaatgtaatttttttaagttttaaatatttgagatcttaaaggaagtccctttacttaaaaaaaaaaaaaaaacctagtgtctagatttatatcactagattgtttcattctaaggtttcagtttggacaaagcaacaacacctactgtctctggggctttgtttgtgatatcccaaattttgcatagaattgtaaaattgaaaaatctccttagaggtcatgttgtccaatgtacaataaaataaccttttagttacattcctaacaaatggttttctagtattttcttgaacacttccagcatcagattactacttagggatgcagcttattctgtattggacagcttgaatttagtttttattgtactgagctgtaactcctcagtagcttctaccagttgaacataggactgtgatcaagttcttttcaaatccttgtacttcattctataaaaataggaattttaaaaatcaaaggtggttttgctagatgtcagtgtgtgcaagtatggttgaaaagactagtgtgtaataagcgatcttctttattatccatataatttcttacaatttgcagcttagaccactgtggtatttgcatcttctttttggtaccatttttgagtttcctatctaatcttttacacttgttaacagaactattttctttttcatttctgtgctacacttagatgtaaatttgaatagtttgctttctttattttctaaaagcctaccccatcatgtaaattaatgcttgaatacggagcttggccaactttttctgtaaaggactagataaaatattttaggctttgtgggccaaaaggcaaaatcgaggatatcatataggtacttcagtaacaaaagagaaaacacattttcacaaatttttttattgatgaagttagaggtataataattgagtcttttttttatttttatttttaaatacagctctactaaccagaagaattgagttctttttggggaaatataacctttctcttaattgatgttcaaagttagtactttctaatcatcaaattgatggcaaatctgttaattcttagcttatgggcttcacacagacaatgggctgtagtttcctggtctctgctcttacacaaccattcagcatttctcctgtccctaggcttttatttgacgtacttgagctcaccatcaccatgctacaattctgtgggcatattagctattctatttgtcatatatgtttagctcattacaattgtatcatttctctactgattatattttagaattttgtggcgatttgtcattaattttaattcatacataatgcaagtagaatttctgaggaagctaagtgagcatctgtgataattccctatctcatgcttgccgtaataccttataaatctttaagtctagagcttgtttgtacaactttttgttagtccttgtaattcattttctgctaatactattttatgctacctgctattaatatattttgttactgataatttcagacttcattttcattaagtgcttattttttccaaaatggtccgtgatcaatatgtagtagcaggccaactctgaaataaaacactcatcatttatgtgctatgctgtcatcttgcatttatacttaagaaatgtacttatcaccttactatagtaagtaagttccaaaatcactgcttatagaaagttttgataatgatataagataagagaactgtaagtatttccaagcatccagggcattttgggatatctggtttctaggtgccttctatgttcttgaccgtggctgcccaggatagatggaattaccagtagtagtttaccatccatttattgactggaagttaccacacattgcacggaggctctaggctttgtattttgtgcaagtgtgtatatgtttagtatagttctaagtcttattgcatgaaggttaataaacttcataagagtgttgtgtgtgtgtgtttaatgtagccccaaatcttactggtccggccaggggccgtagttcacgcctgtaaccccagcactttgggaggccgaggtgggtggattgcttgagcccaggagtttgagactagcctagggaacatggcgaaacccagtctctacaaaaaatagaaaaattagccaggcatagtggcacatgcctgtagccccagctacttgggagcctgaagtgggagggtagcttcagcccgggagacaaatgttgcagtaagccaagattatgcccctgtactccacctgtgtgacagagcgagactgtgtcaacgagtccatcaataaaataataaataataaatatcacagccaagcgtggtggctaacacctgtaatcccaacactttgggaggccgaggtgcgcagatcacttgaggtcaagagttcaagaccagcctgaccaacatggtgaaaccctgtctctactaaaaatataaaaactagccaggtgccatggcgcacacctgtagtcctgggtactccagaggctgaggcaggagaattgcttgaacctgggaagcagatgttgcagtaagccaagtaccactgcactctagcctgggcggcagagtcagactctgtcacaaataaataaataaataataaatatcaaatctttttctttctccttccttcctgcttgcctccctccctccctccttcctattattgctaacatctgttcttcaaatgttaaatatatatgaggtttcatttgaataaaaattcctaagaaatgctgatctagctattctagttttcctgatttaagttggaaaagagaagttatatgtgtttgttttcaaagttaaatgttattaagacgttctgtattttaaacaaatactttcatgtactgataaacatgactttaagaatagatttgaaattttcttaaaggtggtctcataaatgcccagttcagttctcacgttttttaaggtaatagtcatgcacccttagtgcttatgataagtagggaactcgtattaaactgttcttttctgctttcttttcattttctttctagaaaacctcataagagagtatctgggggaaactaaccctggaaaaatggataactcttctctgaatttaagtttaagcaaaaaaaagaaagtggataatcatataataagctttatgaagtttttttatgtggtagacttgcctcattattttataaacttttgcctcattattttataaactttctgagcagggacttttttttattcttatgttttaatatctgagggcaccaaaataattttcaattaaggtttgtattgaattaacttgttaaaccattgtccattcatgaatttgaaataagctgggaattaataagaaaagttaatgtggttgttgtgtatgttgtttattgactcagatgtccatgccagtaatttacagcatgagtaaaacagtttaattaaacgaactgtatctcattctagctaacatttttaattttcctcactgccctctaataaggtttttgtaaaatatttagaataaaatccaaatacattatctttagaataagttaaaagtataagaacagatttgaaagtcatggaaaatcatttctaagcatgaaaaacacaagtatttaactttctgaagttaatttcatgcatagaattgtcagatcagcatttcattatgccatttgacctctacatatcaaaaatgcttggattttcacggcatttgtaaaaagacaaattccattaggggcttctcagacaatgtgctttttttcctttaaattcttccttaatataaaaacctcaagaaatatatgatatagttccagatacatgggaaaaaataaaatatttttggaaaatctctaccaggaattttgccaaatgtaatgttcactgacatacaacattgtcctttggcacctctgcagttggccatagcagtggagctttgtagaagagagtcctgtgcaggcttgtataatgctggtgaaggtgataatttttaatgacagttactttttttataactacatagtcttagattttatatgctcaatgcagaagactgagaaagcacagacaaatagacaaaaatcacttactgttctagcacatcatgaaaaatacttctataaatattttcatatactatattttctttttatttaaataaaaataccatcttactgctttgtagacttttaaaaactgaacaattaagtatattttttaatgactacatagattccactgcatgattgtattttgaattacataattgatgctgaaaataacagcattaaggtaaagtgatacagcattgtgtttcaaaaagtttacatgatgtattggttttcgtaagtttgattattaactttcttacataaatgacaggtgtcccccagaaattccatataagcaatctaggaagttttggacctgggaaagctgtaagttttactaataatttctcccctctactcttcaaactttaaaacataaatttcaaataaggtaagatgaatttcagtccagagtttaatttctggattaaactatcacaaaactaacaatttaaaaataaaccaaccatttgtagaattttttaattttaaatattaaaattcctacagagatagtttcatttatatcataaacatttacttatgctatagtaataatttgactattgcttttgaatagaagagttttaggacagaattagtcaatgctacacctcttgataatagaatttcagtatagctttcatagtttttgtaactggcctttagaatttgggtatatcttctctaaaaagcttgacattaaatatattgatacttaaattatttgcaataataaaattattttataaggcaattaaattttcttaatttcttttgtaatacagctttactaaactactgtttagttttctcttaacattaattaaccaacctagatatctatgtagatagcttttacacagggaagagctagtaatttcagttatctgtaactgaaattttatgtaagaaatttgaaagtacaacacagatgcaaataaacttttgattgtgctatgggagccaagtagatgggcgggcttcatttcttatgatgcctatccgtagaaaaagatttagaaggacctacaaattctacaatggagaatagaattaagggaaggggaaaaggagagaatttttattttttgctttttactactatgtgatgttgattttttctaagaaatcatatctttttacagttgcaggacttatgaaaaagtagttgctttcaattttttgtttttgttagtaaacaaaaacagttctacagaagttgagagttacatttttgttacaaatgtttgtctaaataattgttttcctaggctttcaatttgatgcttgtttgtggtataatttagacaaaaagatcagtggatccctggctcctcagagttctacgttttccaatattgggaaatgactgctcatcataaaagctgccactcagtgagaccttactttgtgccaggcactgggttaagcactttacattcattaccacacttaattattatattaaccctaagaagtaagcatcactactactactattactgttactacagtagtctcctgatatccacaggtatcactttctactgttttagttacccagagccaaccacagttcagaaacattaagtggaaaattttagtaataaacaatttatgagttttaaattgcatgctgttctgagtagtatgctgaaatctcatgctgtcctgcttcattcctcctgggacgtgaatcctccctttgtccagtatatccatgctgtatacactacctgcccgttagtcatcgacatcatttgctcctgacatccagccatggacattattatggctcactgatccaggatcattccaagcagatgatcctcctcctggtgtacagtcagaagatcattagtagactaaggctatgtcacaaggcctgtgtaattcacctcacttcatcttaccacataggcattttatcatctcacatcatcacaacaagaaggataagtgtagtacaagaaaatattttgagagagagactacattcacagaactttacagtatatcattttaattgttgttgcttttaatctcttactgtgcctaatttataaattaaactttatcataggtatgtatgtataggaaaaacatagtatatagtttggtactacgtatgttttcaggcatccactgggagtcttggaacatatcctccacagttaaggggaaactactgtattaccgtatatacaggaagagtagggtggtggaaaaaccatctgattcatctgtagctcaatttatagcctctattcatggaattttgtaccttcgggaaaaactatatgatccttaatgtcaacttgatacccagaacccattgagttgtgtttattacttattatatgtatattgtaaaaatttgtgtctaccagcaaattagtgatgcaacccctaaatcttacagtagtgaaatcaatttgaggtatctttaggtttaattctgtttcaaccattgtaatggtcagttcctttgctttgctttcaattacacatggtccccacaattcctttgctttgctttcagttacagacagtccccgacttaggatggcttaatgattttttgactttccaatactgggaaagcagcacagattcagtggaaactgtattttgagtacctgtacaaccattctgtttttcatttttaagacaatatttaataggttacatgagatgtttgacactttatcatgaaatagtctttgtgtttgattattttgcccaactgtaggctaatgtaattgtgctgagcacatttaagggtagtctaggctgagctgtgatttcagtagattggacatattgtttgcattttcagcttaggatattttcagcttactatgtatgggtttgccatgatatgaccccattttaaatcaaggagcttctgtacagttcaaccttattttcaccatcttcatttagcaaatattttggattttaattatccctgtcctgtcctatagtttgttcagtgcccatgtgtacctacactcttgtttcctgacttaacattttcattgtcagacaatctttctttttctttttcttttttttttttttttttgagacagagtcttgctctgtcacccaggctggaagttcagtggcgctgtcttgactgactgattgtgttagttggtacttaatttgttcttgtattatcagttaaattttttcatttattgctgatcagagcagcagctcttaatagtttttaaatcatggactactttgaaaatctattagtaatagtggacacttccccccagaaaaatgaataaataccaaacagtttgtatgcaactttaggtgattcttggccttcccgtagctctcccaagaactccagggtaaacatctttcccctattatagtacaaggaaataagactacagttaataataaccacctgtagaaagtgggtattacacatacgatatgatgccaacatttttaaaataagaaaaacggaggcattaaaagaattattctaatgcaataactgctcaacattagtataatgttttccatagaccaaaagaaacattaattttaactaatattatgaaattgtatttggtctacagtaacttacccatacttagttaaatccctgtatacttaaaagtatgtgtgcctaggcatggaaaaataactcctggcgatgtagctcagtttgtcttttgacttgcagataatggcatccatttgaaatcgttacttttcccagaatatgcagtgtagttatttactaatttgccagtgatgttttatttatacaaagaaacttttttccagacaaactgaagtttcatgacattgtcacttcaaaactaatttatttatagttgtattttaggacctgagtcaaaggaccatccatatgattacttttcctgggatataataccctgtagtcatcaactgcagattcttgtgcagggttgccttttgagttattttgtgtgttgacctaatttcagggcctcaatttaatacattttctttggagaaatctataaagtatatgaaggggcaggtgagagatgggacagggacagaaatctgtcaggttgatagcatgcatatgctagacccctgacttcaggatctatgttttactaaggaattcttgcttccttttttgcaataaaaggttaaaatgtttgacaccaatgattcttaatagttggctagtaggaaaagtataagaaactagcaatcttgtagaaaggggcatttctagtttttaaaagtatactgaatgctatttcatatttataaagcaatattttattgtttatttgatgtataagatattaagacagtatgaaggaaatcttggcttgtgatgctacaaagaactatagtgttagtgattctcgaaggaaagtaaaactggttgatgtgtatacagatttttacatgtatttttttttaatccaagagattcatatacccccctttctattttatactgcttccttgagaataccactttagctgaaattcacctagaaaaaaaggtcttttgactcctatgaatatgtatcaaccaaaaatgaatttttcactttatgttcaatagtttacgtaatcaaaactttttggcggggaggaagatgatttgtttccaagtataaatttttatttagtaagaaaagaatgaagtccaaaacagtcttgaaaaagaataaagttggaggagccacttttttttttttttttttgagacagagtctcgctctgtcacccaggctggagtgcagtggtacaatcatggcttactgtagcctgcaccttcttggctcaagctgtcctcccaccttagcctcctgagtatctgggactacaggcacacaccaccatgcctggcaaatttttgtattctttgttatagataggttttcaccatgttgcccaggctgatctcaaactcctgggctcaagaagtgatccacctaccttggcctcccgaagtgctgggattacaggcatgatccaccacgcccagccacacttcttgttttcaaaacttactacacaactgcagtaatcaagacaatgtattactggcgtgaatgtagccatacagatcaattaagtagaatggagagtctagaaataaacctttatatttattgtcaattgatttttgacaaggattccaagataattcaatggggaaacaatagtctttttgacaaatggtgcttggacaactggatccatatgcaaaagaatgaagttggacccccacctcacaccatatacaaaaattatatagcttgtggagaaaagggaatccttgtacactgttgttaggaatgtaaattagtacaaccattatggaaaactatatggaggttcctcaaaaactgaaaataaaattacagtatgatccagcagttccacttctaggtaatctatcctgaagacttgaagtcagtatgtcaaagagatatctgtactctcaagttctgttcataaaagccaagatatggaaacaacgtaagtacctatcagtggatgaacggaaggaaaaatatggtatatatatatccactacagtggaatactacttagccttaagaaagaaggaaattctgtcgtttgtgacaacatgggtgaacctggaggacattatgctaagtagagtaagtcaggcatagaaagagaaatattgcatggtcttactaatatgtggattctgaaacgattgaattccagaaggagagagaagaatagtgtttaccagaaggtggagaggatgggaaatgttggtcaaagggtacaaaattatagttagacaggaggaacaaatgataagtatttaatgtaatcggtatgttaattagtctgattctgttattccacattgtatacccataatatcactgtttacctcaaaactatatacacttataattcgtcaattaattaaaaacaaatgaattcagaatgaatcaaagacctaaatgttagagccaaaacgataaaactcttagaaggaaacagatataaatctttgtgaacttcaattagtcaatattttcatacatgtaacacaaaaagcacacattataaaagaaaatagaagtaaattggactttgtcaaatttaaaaacttgtgctttagaagacagcatcaagaaacttaaaagacaattggagaaaatttttacaaattatgtctggtaagggatttgtatctacagtatgtaaggaattcttacaactcaataataaaaagaaagcccacttgaaaaatggacaaagaatcggcatagacacttctctaaagaagatacacaaatggccagtaagcatacaaaaagatgctcaacatcattattcatcaaggaaatgcaagtcagtaccataatgaatactattatcacactctccataatgactaaaataaaaagacagtaactaatgttggtgaggatatggggaaactggaaccttcatgcattgctggtgtggttgtaaaatgatgcaaaccttgaacaagtttggcacttcctcaaactattaaacattgagttatatgacccagcaattcagcttgtaggtatatatcctaataactgaaaacttacatccacacaaaacatgtacgcaaagtttatagcaatgttatatagccaaaaaaggaacaaataaaatttctagcaactgatgaatagataagaaaatgtcacacacgaaaggccacatattatataattccattctatttatattaaatgtccagaagagacagattgtggagacaaaaagattagtggttctggggagctgggggaaatgagaatgatggctcatggatacaggctttcattttgaggtaatgaacatgttctaaaattagacagcagtgatgattgcacacctctgtaaatgtagtaaaaatcactcagttgtataaggggtgggtttcatgatacatgaagtacagtaaagcttttttttttttttaaggcaaaagtgagcacatctatctgtaaaagaatgtctttgtcatttccactgcatcttaaggaaggcaataaaccctgactatagttttgcaaaggactctaaagggtttctcagagttgtaccatccaccccgattgccccctaaaagggaaggagagctgatcaacttgagttaaatgacaggcaagaaagagtgaatgaaatagagtataggcacacggggggaccctcctttagtagttcattctgtctagtttaaataaatactttacccaaattgtttacttgtgctcaaataatgtgacagcttgctgctctgaaaaatgatgaaaatcactgaagttcttaatggggctttaagactcatattgctgcagctatacctttttgctctttgccttttggaaattagaaggaaaccctgagagagaaatattgcaccccaagaaaatggttgcttataccaacttgtaatcatatagtataggggagaggtataaactgggtaaaatctgcattattgagcttattactttagctacctacaactagtagatttaaaaagtaaaacctgctgggtgcggtggttcatgcagtaaatcctagcactttgggaggctgagatgggtggatcacttgaggccaggagctcaagaccaacaggaggaagcttcatctctattaaaaatataaaaattagctgggtgtggtggagcacacctgtaatcccagctactcaggaggctgaggcacaagaatcacttgaacctgggaggtagaggttgcagtgagccaagattgcaccaccgcacaccaaccaggctgacagagcaagactctgtctcaaaaaataaatagtaaaatctgtaaattgtaggaaaatgccagaggagatgacatttcatatcagtcctctataagtgaaaatgatagtgattctattgtcattagactgatattttgaaacagaatacatgttttctatttagaaaaatttgtccttttgaggatatagcagccaatctaacacaacccaacactccaaattccatatgcagaaaataaatgaagatgtttaggtattcaaccatttatacaatgattatgttgtgttatataatttcatttttaaaacctttattgaattcattatctataagcctagccatacatccctttagtctttcaaggtgcataccacattaactacatgcgttaatttagtgaacagtttttactaggtgctgggcaagaacttagaggtttcttgccttcaggggcttcagagactgatggatgggaggagaggaactgagggcaatagggcaagcccagtattattccagatgtctgagcagaatacaaaggcagaagcatttcactcagagtagtgctgtctggaaagccttcttaggagagctaaccattgagctgaatattgaaacatgaaaaagtatttaccagatagggagcggaagggagtggataaatatttagtaaaactaggtttggtgtgtttctttatggttgtcgcttaaggttctgcctggtggggttgatagaatatgagtttaccaggaaggagaagactggagctgttgacagtggccagatcacattagcttgtaagcagttggaagcaattgcaggattttaagcggagtagcgacatttctaataggttttcttttacttcgctcttaaaggctacacatgcacctctattgctgaagtttggcgacctctggtgttgaactataaaataaacaacgttgtggaagaaatactagcgtttcagagcttgtcagttgctttttgtgaggtgcttcttgggccgtagtacttctttgaagacagtaattatcaactcataattcctgtgttgatatttttcagtgtttggataaatttctaactaggtagggccaggaacagataaattgattgatttaatagtaaggggatgaacaacatccccaaaggggtggggcaggctgatcttgatgctttttgcaacaaatgaaaagatcatgagttctagtaatagataactgagaatatgttcccttcccattggtgtgataatttttaaaattcccccctcttttgtatgaaatacacttacagaagagtgcataaagcatttccatctagtttaatttcctttaagtcaaaaaaggactcagttttcaggaaatccaggaataagtattaattactcaaaatatatgtctttagtctaaacgttttggaggaaaaaaaatttccatctcttaaaattcaagtgaaccctaagacttgttttgtgtatgaaatgataatttgtcaaacatcttttttatctataaaattattcaattattctgtttcaaaagttccaaatgtagaattcagctggagtaaatcaggtagaaatatactctgaacctaataaaaacttaatataaaacctaaagatatactcagaggttgaaataggatagttgttaaaatgtcttatgtttgtggtgttaaagatagtcatgcgttctgaatctttgctcttcatatctatcacttgcttaccttttagcctttaattatatcatccatctataatccagtccttattctcttttagaatgctgtttttcacctacacatctagtgtgcatagaatgtactttttactcatttgcctgctacattccttaagtaaaatcagctcttgactccccttccagaacctttcctgaatcccttcctctttccagtgcagtgtagcactttcacctcacacattacctatacttactttgctatactattgtcacctgattgttgttgatttacgtacaagtctctctgccctagagcttcttgaggaataggctctaagtaaacgttttgaaatgtgaataacttcagaggctgaaaagttttttaaatgcctcttgattatgtctcatctttgctttcttaaaaaatatgcatattgtatataaaatagttctgattttagggtttgacagattctgatacttttaatgctagccttataaatagcaatttaagaaggttaaacttctgtttgcatttagtaaaggcaacacaaattacataatggtcccttttttttttttttttactttttaaatcaagttaatagaaatataatttatatgtaataaaatataggcattttaaatgacagtttagtaaattccaacaaacatgcccatataaccactaccctggtcaagatttagaacatttctatcaccctaaaaatgtttctttttatagcctggaatgaccaaatgtctcagtttgcctggactgaggggtttgccaagatggtcccaggcaaactgggatggttggtcaccctgttcattctcctttgtggtcagtttcccatcttctgtctcaggtgatcattgatctattttctgtcactacagatgcctattctagaatttcataaacgaaatcatatatttcttttgtatctggctttttttgctcagtaccatgtttttgagattcgtcagtgttgctgcctgtatcacctgtttattcctttttatgctaagttgtattctgttttatgagtataccacaatttctttatccattcacctatgaatggacatttgacttgttttggggttttggttattatgagtaaagctcctatgaatgtttgtttacaggtgtttttgcagacatatgtttatatttctcttgaataaaaaagtagggctgaaattgctgatcatatggtaagtgtatggttaccaaactattttccaaagcagttttactattttacactcccaccaacaacacatcagaattccagtgcttcacattctcatcaacacatggtattctcaataattttagatttagtcttaaaatgacttttatcattaggaaatgcccctctttatttttgtttaattttcttatcctgaagtcttcattttctaatattaatataggcactctatctttcttatgattttgcatggtataacttcttgcattcttttacttttactctatgtgttttaatttaaagtgcatttcttacagacatatagttaggtgttttttaattgagtttgacaatttctgcctttgtaattgaagtacttagactatttacattcagtgtaattatcactgtggttgagtttaagttttgcaccttcgtatttgttttcctttcatcccatctttcctttgctctgtttttccccctcttttactgccacctatggattaaatcagtggtttttattttttctattggcttttaagctatacctccttgttgcatttttagaggttggtctaggatttaaaatatgcatcaatatattacagtcagtcttcaagtagtaatgtaccatgtcatatagaaaacaagaaccgtgtgacagtatttccattccccttcctgttctttgtgctatagttttcatactttttaattctacatgttataatccttacaatatttgttgtttatagggggaacccgcccctaatatttcaacataggttttttctattttccatgagtgtcggctggctgagaaataaagagaaagagtacaaagagaggaattttacagctcggcctccgggggtgacatcacatatcagtagaaccgtgatgcccacctgagctgcaaaaccagcaagttttattaaggatttcaaaaggggaggggatgcaagaacagggagtaggtcccaagatcacatgtttcatagggcaaaaggcagaacaaagatcacatgcttgtgaggaaacaggacaaaggacaaaaggcagaacttttgataagggtctatgttctgcagtgcacgtattgtcttgataaacatcttaacagaaagcagggtttgagagcagagaactggtctgacctcaaatttaccagggcgggatttttccccaccctgctaagcctgagggtactgcaggagaccagggcggatttcagtccttatctctatggcataagacagacactcccagagcagccgtttatagacctcccccaaggaatgcatttctttcccagagtattaatccttgctaggaaaagaatttagtgatatcttccctatttgcacatccatttataggctctctgcaagaagaaaaatatggctctattttgcccaaccctgcaggcagtcagaccttatggttgtcttccctagttccctgaaaattgctgttattctgttctttttcaaggtgcaccgatttcatattgttcaaacacacgtgctttacagtcaatttgtacagttaacacaatagtgggcctgaggtgacgtacatcctcagcttatgaagataacaggattaagagattaaagtaaaaaaaagacaggcataagaaattacagaactgttaatttggggaactgataaatgtccatattaaaatgaaatcttcacaatttatgttcagagattgaagtaaagacaggcttaagaaattataagagtattatttgggaactgataaatgtccatattaaaatgaaatcttcacaatttatgttcctctgccgtggctccagctggtccctgtgttcagggtccctgacttcccgcaacagttgttactttgctttaaacaatttcttcaagaggaattttttaatgagggggaaatatcatacatttaaccacatatttaccatttttggtgctctctattcctttgtgtagatcagaatgcccatttcagaagtctttctttaatcctgaactacttttttgtggtgaagatctgctggtgacaaacttggttagcttttgtttgcctaaaaatgtattttgaaggatattttcacttgatgcagaaattggtttcccttggtttccagggtttatttttccttcagtttttggagagatctctattttcttctggctttcattgtttctgttaagaagtcaactgttgataccctgtatataccctgtctataatgtgtttctggcattcttgaagattttcactttgtgagtggcttttatcagttgattatgagtttttatagtattctttgtatttgtcctcattaaggttcattgaacttcttgtgtttgtgggataatcatggtttttataaaatttgggtaagttttggtaatgtttcttcaaatagtttttctgcatctgccctacttcccaccccatacttcccaccatcagactcaagtcacatgtgtacatgttttttccctccatgcttcagctttgatagtttctattgctatttttttaattcagtgattgagtttcttccctgtgtctaacctgttaatcctctcctgtgaagttttaatttcacatactgtattttttaactgtacaatttccatttggttcttttttatatcttctatttctctccttgttataatagctattttaaagtccttatgtgccaattctgtcttctctgtcatttctggatctgtgtctgttgactgacttttgattttttctcttcttatattttcctacttctttaaaaatgttgaattttgtttgtcagccagtttcttagttgtttacacctggagggcagtttcagtgttcgttatttcatcacagctagatgcagaagtctataatggccttttaaaaaaataataataccatgcaagcctttgcatggcattctattaagtatatttcacaaagcattttaaaataagttttagattttttatactcaattttatcttgtttagccagttagatgactgtttcttcatagtattcttctgttgctaaagacctctgttattaacagtttaaaatatttttttccaacttttattttagattcaggggtacatgtgcaagtttgttacctgggtgtattgggtgatgctgaggtttgaggtatgaatgatcttttcacccaggtactgagcatagtacccagtggttagttttgcagccttctttcctgctctggttgtcctcagtttgtattattgccgtctttatgtccatgattacccagtgtttagctcccacttataagtgagaacatgcagtatttgcttttctattcttgcagtaatttgcttaagataatggcttccagcagcatctatgttgttgcaaaggacgtaattttttttatggctgcatggtattctatggtgtatatgtaccacattttctttatctagtccactgttgatgggcatctaggttgattccatgtctttgttattgtgaagaatgctgtggtgaacatgcaaatggttgtatctttttggtagaacaatttttcttttggatatatacccagtaatggcattgctgggtcaagcaagttatttgagaaatctctaaactgctttctacagtggctggactaatttacattcccaccaatagcatataagtgttcccttttctctgcagcctcaccagcatctgttgtttttttactttttaattagccattgtgactgatgtgagatgatatctcattgtggttttgacttgcatttctctgatgattagtgatgtgaagcatttttttcatgtttgttggccacttacatctcttctttagagaagtttctgttcacgtgttctgcccattttttaatggggttatttgttttttgcttgttgatttgttttaaatttcttatagattctggatatgagacctctgtcagttgtgtagtttgtgtctgttttctccaattctgtaagttgtctgtttattctgctgatagttgtttttgctatgcagaaactctttaattagttctcaccaatttttatttttattgcagttgcttttgaggacttagtcatagattatttctcaaggccaatggccagaatagtgtttcctaggttttcttgtagaatttttatagtttgaggtcttatatttaaatctttagtccatcttgagtcactttttgtatatggtgaaagataggggtccagtttcattctttagcatgtggttagccagttatcccagcaccatttattgaatagggaatcctttccccattgcttatttttgtcgactgtgacgaagatcagatggctgtgtgtggctttatttctgggttctctgttctgttccattggtctatgaatctgtttttgtgccaatatgatgctgttttgattaatgtagccttatagtacagtttgacgtcaggtaatgtgatgcctctggctttgtcctttttgcttaggattgctttggctatatgggctcttttttggttccatatgaattttagagtagtttcttctagttctgtggaaaatgacattggtagttcgataggaatagcatgaaaaatgacattggtagtttgataggaatagcattgaatctgtagaatgctttgggcagtatggccgttttaacagtactgattcttccaatccatgagcatggaatgcttttccatttgtgtcatctgtgatttctcttagcagtgttttgtagttctccctgtagagatctttcaccttctcagttagaggtattcctaggtattttattttattttattttattttattttattttattttatttgtggctattgtaaataggattgtgttgttgattgggctctcagcttgaacattattggtgtatagaaatgctacttattcatttgttgattttttatcctgaaactactgaaggcatttatcagttccaggaaccttttggcaggaacctttgtctttagggctttctaggtatagaatcatatcatctgcaaagaaaatagtttaactacttatttttctatttggatgccatttatttttttctcctgcctgattgctctggctagcacttctggtactatgttgaataggagtggtgagtgggcatccttgtcttgttccacttctcaaggagaatgcttccagtttttgcccattcggtataatgttgatgttcattttattaagttgaattaccatgataggggtagattttatttaaatcagggttttttttaagttgccaaaaagtctatttaacttttgaaacttgtttataacctcgtgatcttacaaaacctttatatattttatacaactcaaaaggattcatgtttcttttttatggtataatcactgtagtttaagaaaatgactttttaaatgttattttgtaaaaactcttccttgttttctgcccttaacattggatagaccattttagggagtcagtgacacatgcatttgtatccctatctaccactgacaaattgtaaccttgagacttctgtcagccttaggtttttcacatgtaaaagaggataatattgtatctgtaaaattcaaatgagaaaagaaaattgatgtgtctagcacactgctcttacatgctatgtgcttaataaatatttgttctttgttctctcctctctctctcactggcagtactttgaaaggaaaacttaattccactaaactcatgtcaggtgattatggtcggcttgctcttcccgtgacctcagcaagatcagaaaggaaaccttagtggccacctgcagtgaacaccttttgggctgtattttttgcctgggttcattgtgtcataagatacttctctgtcgtttaagtgttcgccttcctttgcccccatggtgtcactgtcttatgggacctcacccaactccttgtacttctaactcagccttctgtttcaatttctcttttttctgtatatttgtatgcccatctttagcactcttctttgtctgctttgcctgttatacactgtcacagccacctagcacttataccgcaggtactaccaaagcagttcttttagcctcaagctctcttctaagtaatatatctaatgttcaccacttggtggagatctccttctggttcttcctgaatgtctggtctcaaatattattttctcttctgatctactccttcatgcctaataccatcaagatttttcacaactactgaaattgtttaccttccagccacattcaataaccagattctttgatgccacctcctaaaatttgtttaccactgataagatttaaattactttctagctctgtttctagttatgacctctgaaaaggtactccttctgggtctgtttttctcatgtttaaaaatgtttgtactagatcaagggtcaacacagtagggtccaggggccaaacacagtcttctacccatttttgtaaataaacttttgtttgtggttgttttcatgttgcagagttgagtagtgacagagactttgtgtcccacaagccttaaatatttactgtctggccctttccagaaaacatttgctgacccctgaactagaaaatctctcatctagtttcctcttttttttaataatttttttggggaccattcatgatcacgtctaaggatagtaggcatttttaaattgctccctaatgttttgttttaattggtttatcattagaagtcaccaacagaaataagtgctttttttcctatattgtgaggaccttgtcataataaaatattcttcagtgtttcaaatttgagagctttttgaatttttagtattttttaatatgttgtaaatcccactgtcagccaccctaaggagaacctggtgttaagatttaaaatacagtaagaaaaagactagaaaaatgatagtagttgaattaaacgtggaacatactgtaatgttaggatctctgtttcccatgtaatagccttcgtgattcagtcatgtgatggtaaatggatgaataggattttctttgccattttgtgctgacagacctttttttagttaattgctttttaaaataatttatagtgaatacaccaacaattctatttacaggttggtagttaaagcctcttaacagtcactgataactatattttgaaggtaaagaatatagctagttaaaactctgaccactgattgtaaatataataaagaaacctttttttaaccagtggggaacccaggtgaaataaatgcaaatcaacagcaaaactgatggtaaaagtgtattaatctttataaaggaatgagttgcagaagaaaaaaaatactttttaaatatcatcactattcatcaaagcagattatttcccattgtggtaaagattttctacgtgcattttctggtatatattaaaggaggattcaaggtatgtaaaaaacatacttacataaaaagttgtaaatatcaaaccttaattatatgcatgctgatgtgtactgatacctgcaacttattttgaaatgcatcaaaaaataggtggattgatgtgtggatagagggttgtgtatgtgatgaaccaaatatagtaaaatgttagttggagaatatggatagtggatatagtgggttggtgttcactgtaaaactttcacaacttttttgtttgaaattttccttaacataatattaggtaaaatgtttagatctaagtggctgtgagacagtgaatttagggagaaggatgtgagagtgagtacacacacatggtataccttaaataaattattaaaagtttatatgttaagcataccgtgtatataagcatatgtttctcaactgttttattgtgtatttcacagatttgcttctattaggaatattcttaggttataagtaaaaaaaaaaaacaagaattgtaaaactttcatttcagggaacatcctgacttttacattatacattgtaactgtattagtccgttttcacactgctgataaagatataccccagactgcataatttaataaggaaagaggtttaattgacttacagttccacatgcctgggaaggcctcacaatcatggcagaaggcaaggaggagcaaagtcatatattacatggatggcggcagtcaagagacgagagcttgtcttgtgcagggaaactcccctttataaaaccatcagatcttgtgagacttattcactataatgagaacagcatgggacaaactactgccgtgattcagttatctcccactgggtccctcccatgacatgagggaaataggggagctacaattcaaaatgagatttgggtgggggcatagccaaaccatatcagtaactcttgaattatcccccataacctctatgaaagttagccgaatgcttaagaagagaagtccatgtgatttctgttcacacagtaaatggatacatttactaatggacatcacaagagttagtactgtgtcttatgttagcaaagtactgaatatgtagaggatggctgtacaacaaatactgattcaagagtgggcatttctgaagtgtcattttggttatttcgaaatatatcaaaatgaaaaagtatatgcaaatatattgttttaagtacaatagtaaatattcatgtaactacaactcatattaagaattagagctagaagaattacttttgccccatcccagtcacagcccccttttccacatcctctttgttaaactggataatctgtacttgattttaatgatgaatatacttatgttttttatttatttttacacagttttactatgtaaactatagtgttgtctgattttgaactttaaggcagtcatgttgtgtgcattcattgttgagtaaaagctcaccactgtgtgagattcatccataaaggtatgtgtagctctggtttagtcatcttcattgctagttagtatgctattgtatgactatgccacaatttatttgttaattctactgttggatttttgcttttatcggcagtattttattattatgaacaatgttgctttagaggtataagaatttctctgttagggtatatagcttgaaatagaaatcagtaagttgtagggtcactgaaggtatacacttataaacactcatttattcatgtcattgccgctgttgtttgttttcagctctcaaaagaatccaatagagttgcatgtgacggaattgttgataagcttattagtactactccctcattcttcactcctcaccctctcaccaggtacatgtatacacatacacatgcacatatccccagaacaatgttagaaataggggttagaataagtgttaataatttagattatttttacagtcacctttttattttctttccaataacattaggagtttgctcagaaattaccttatacacagttaaaggtcggtttatttttaactagattcctttttgatttatatcagtgaataattctgtagctcagaatatccttccatctatcttacccaaactttatgtattctaatagttaaataatttctgaaagtttttattacaggggtaattggcatacaagaaactacacatagggaccacatcaagataaaaagcttctgcacagcaaaggaaacagttaacagactgaagagacaattcacaaaatgggagaaaatatttgacaagagcttaataaccagaatatataagaagctccaacaactttataggaaaaatatctaataatctgattttgaaatggacaaaagatctgaatagacattattcaaaaggatacatacagatggcaaacaggtatatgagaggtgctcaacatcattgatcatcagagaaatgcaaatcaaaactacaatgagatatcatctcaccccagataaaatggcttttatctaaatgacaggcagtatcaaatgctgacaaggatgtggagaaaacagaatccttgttatgttgctggtggcgatgtaagttagtactgcttccatggagaacagtttggaggctcctaaaaaaactaaaaatagagctgcctaccatctagcaatcacactgctaggtatatacccaaaagaaaggaaatcagtatattgaacaggtatctgcactcctatgtttattgcagcactcttcacaatagccaagatttggaagcaacctaagtgttcatcagcatggacacttcatgaatggataaagaaaatgtgatatatatactcataaagaatgagatcctgttatatgcaacaacatagatggcaccggaagtcgttgtgttaagccagacacagacaaacttcgcatattctcagttatttgtgagagctaaaaattaaaacaattgaactcatggagatagggagtagaatgatggttagcagaggctgggaagggcagttgctggggaagtagggatggttaatgggtacaaaacaatagaaagaatgaataatatctagtatttcatagcacaacagggtgactatactcaataataatttaattaaatatcgaaaaatacctaaaagagtataattggattgtttataaaacaaaagataggctgtgcgcaatggctcacacctgtaatcccaacactttgggaggctgaagtgggtaaatcacgaggtcaggagatcaagaccatcctggctaccactgtgaaaccccatctctactaaaaaatagaaaaaattagccaggcgtggtggtgggcacctgtagtcccagctactcgggaggctgaggcaggagaatggtgtgagcccaggagccggagcttgcagtgagctgagatcacgccactgcactccagcctgggcaacagtgtgagactccgtctctaaataaataaatataaataaacaaaggacaaatgcttgaggtgatggataccacatttaccctgatgtgattattacacattgtatgcctatatcagagtgtctcatatattccacagatacatacacctactatgtagccacaaaaataaatattttttaaaactgcacatatttaaagtattcaacttgataagtttttccatacgcttacacccgtgaaaccaagataatgaacattcataatcccagtagtttcctagtgtccctttgtaatcttgcttctcaaaactatagatcttctgtcactatagatagaaaatttactagaattccatatgaatgtgattatactttaggtactctttttggtctggcttctctcactcagcttaattatttttagattcatccatgttgttctttttaaattactgagtaatgttttactgtatggctatgtaccatttatttacttgatgatgccattttggtttgtttctactgtttagcagttaaaaaaaatatgctatgcatatttgtatacaagtctttgtatgaacatgtgcattcatttctcttgggtatgtacctaggagtgagaatgatgggttgtatgatatgtatatgtttaacttttaagaaacttccaaactgttttccaaggtgctatataccatttcacatttccaccatcaatggtgagagcttcagttgctgtacatccttgccaagatttgttatacatagtctttaattttagccttcttattgggtatatggtggtatcttgttttaattagcatttccctaatgattaatgatgccaagcatcttttcttgtgcttatttgccatctctctgtcatccttggtgaagtgtctgtacatatcttttactgatttgtattggtttgtcttatttttgaattataagagttctttatatattgtaaatacaagccctttgcagaatatattttgcaaatatattctgcaaagttcgtgactagccttttcattttcttaagagtgtcttttaaagaacaaactttttaaaatttgaatgaagttctgaaagttttagcagtgtctagagaattagaaccaacaacaaaaacataacaaattctcactttgccctgaatggttacagaattatgcctgattattcactggttgccaccacaaatctagtacttaccattacattcttctacttctgtgcagttattcttgaactgtaactggtttaatattttatttcaataaatttgatttatacacggtcttataaagatgagtaatatgtggtatttctcttcaaagtcctcaatttttagcatttaatgtttcttcctgtcatccatcttgtacagccaggtccatgtactttcttttgttcagtacctagtatagctctagagacaatgaagtgctcaatacatttttgttgtgtactgtaattatacttttttgacatacaaatatgtaagtttatggtagcaggaaatctcttgttctttgtatcctccaaaggtcctaacatagttccttgtatacaagggacactattcaacgactgaatgaattgttgacggttttaaaataaactttgcctgatatatcgataatacaattggccctccatatctacaggttccgcatctgcagaatcaaccgactgcagatcaaaaatattcagaaaataacaatttaaaaatacattaatgaagaataatatacaacttaaaaacaatacaactatttatatagcatttatgttaagtattataataacctaaagatgatttaaagtataggaggatgtaaggttatatgcaactattatgccattttatataagggactcgaacatctcttgattttggtatcctcgggggctcctgaaacaatctgaataccaagggacagctgtataatctttggctatactgaactaagtgaagaaaaaatactctcagatgtgattattgcaatagatagtttttctagataaagatatctgatttttacttgaatatttttgagtatggtaatcttttttatgacccaaatttaacattattgttatcatagaagtctgtctatggtaatacagcatggaatctgtagtcagctacagataaaaggctttcacaaagtttgtttgcctttgtctgttttgtgctactataacagaataccaaagactggataatatataaacaatagaaatttacttggttcactgttctagagactggaaagtccaatgtcaaagggcccacatctggtgatggccttcgtgctgtatcatctcatattggaaggcagaaagtcaagacaaggcaagagtgagagggagccaaacttacatttctaaccagcctgctcttgaaataacaatcctgcttccactttaatgacatgagtccattcacaagggcagagccattacctaattaccatttaaacatcccacctctcaacactgttgaattgggtattaagtttccaacacatgagctttgggggacatattcaaatcatggcactgctctatttaatgagttacgtattttatttaacttgggaattccttttttcagtgaatccaatgcctgaaaacatggagactaaatttgtaaatatataggaaaagatggcaataacttgctttctttctaggtcctgggaactccaacaagggagcaaatcagagaaatgaacccaaactacacagaatttaaattccctcaaattaaggcacatccttggactaaggtgagttagatttttttttaagtatattaatcagtagttaactaaaacaggtttctcattgttcttaaactattttgagatcagatagttgcatgaatacaagcagttctgcatgtaaagatagttgtatagatgtacttgagtgtttcattgactttatatagtttcatcctcattgatttcctgtagatagtttctagcctggttttttatttgcaaggtaagggcagtgtacttatatcattaaagtacagaatttgatttaggagtaggatcttatataaaattttgacaacctaaaattcagaagtttatttacatagaggtgtatagtttaatatcatatagcatatcatacatttcaaacattttctttattcttttaaactgttaaaacatatatcttattatagatggcattttgtgtttttcctctcttgtttactttcctactggcattttggaaacaagattagaaagcaaatgtaatcttgctgggagtatacattaaaaacaattgatatgttaactggatgtcactagcctttggaaaaacccaaccttaacattagatagaaccagttctaaacagcatagttgctaatgtaccagggaacttttttttttcctttggtatttaattggaatgattttaaaagagattttattaaaaagtgttttcaccttagtttgatttgtgagattaagtatgaacagaaaaaaaatagaatttctcagtagggtttataagtagagtgaatctctagcttcttcattttacaagttcagaaactctgaccaaagattcaaagtgacttacccaagattacatggatggcttgtgacaaagccgggattaaacttctgtgtgtgtgtgtgtgtgtgtgtgtgtgtgtgtgtgtgtgtgtgtgtgtcacgggttaccaatgatcgactttataatacgtattttaaggagagacttactgtattttaaaatgtacatataacaattcattttatatacaattaaattcatttagttatagatcttttataattcgaggtgaagtcatttagggctataggtgaaataatttaccctctgtagaatcaaagtatgtttctttctctctctctctcttaatcagatatattatgattccagttactttttcttcaagttcttataatctcattatctatcttcagaaaattctcgttactaattatttatttcctatttagaatcaaagacctcaatagtattttagtgccagaattataggttgtaacttttactacaacctggccaacagaccacgatcttgcctcccttttttaaaagtttatggtaagcactaagtagtctgagattcaaaagtgttctttgatctttgtattcaaggcttattttaaatctgggtcagaagatatttagggactctcacctggctctaaacccagaagatctcaggtggcctacttggatttagaggctgcattacagtttgagtccattatagttattgcaagttaaattgtttatagtaagtgatttgtagtatactatgacttcagccatgttaagagaaaaacaacaaagagaattctgtgtattaaataaatacagggaaatattccaaaatgccaacagtagtttgggtaagaagaaagtgcatgattttattttctttgttctaccttttcaaactttcatattttctttaataataatgttatttttatgaggaaagggaaaaattctttttttggtgtaatctaagtattcatcacatattatcctccttatagagtttctgacaaatataaagctgaaattgagtccatgagatcctatttaacagtccccatcctccattattattattattattttatcaagactgccactagtcctcctttatctaaggtttcagttacatttggtcagcctctgtctgaaatcggtgatgtagtacaataagatattttaagagagatactacattcacattattttattatagatattattctaattattctattttattattagttattgttgttaacctcttactgtgcctaatttatgaagtaagcttttatcactgtatgtatagagaaaaacagtatatgtagggtttagtactacttgcagtctcagcatccacatgagggtcttggggacatatcccctgtagataaaagggaggggacactgtatttagtgtgtgtcaaattctgcattttccccctgaattatatctctctaacgcaagtattagctagagtagaatgtgaggaatggttgattataattaaaatgcagctaatgaaagagctgtactaaatatgtatgctgtaaatcttcctttgccctcttcaattcttgatgtttttatatcttatactaatgcttttcaaacttgaatcgtctatgtaaagcacatttatttaggtttccattcctgtaccatgtgtagaataatgactcagttctcccatcttttttgtctattaaaaggactgtgaacctttcattcgtctggtgttttttgacataatttggttttcacttcatacacatgcattatgtaataagtgcacttatgttggttcctcatttgtgcatactagagtgggtgttactgggattgtaaacaaaaaagtaaattcagatgcagaaattttattgtttttctctgtcatctagatgattgagaatatgcttgtgttggtttttttaaataggttattataagaattaaaatgcaaaacttaaaattcctagagggaactcagactcctttccctaccactgtgatagtggacagcaacttgagaaatgctcttagggtaagcagccaattgagtcatttaaaaaccatatagaatgtcctccaaagagaagaccattttgcccttggagggcaggtaaacaggagtctggaaacaggaaccactttacctttgttgctgtcttctgttagtcctatctcctgaagctgtggacatgtgaaccctggtttcctgtgctgcagtgttgtgcagctatatccccaagattgggacctggtgttctaaaaaaaaaaaaagtagacagaaataaagaccaaaagagaaaatgtcttttgtggaaacattgaggtaaggggatacaggtgtgtaaaagaaatagcagaaaaataacactgagatgatcccagtgtggtgtatggcagaaatcttgagaattaaaaactccaataagttgccctgggctattacataaatagcaatagccagtgcaacataattcattaataaagcacttgtggctttagtaggaaaggaaataggtgctaatattctgaaacaaggattattattggaggtgaggggatgaagacctatgccttggtgtacgctgtacactgcaaagaagaaaaacacatggaaagaaaaggaagtgatgcaatagtagattgagaattaagcagagacagagattggttggaagaagagaagaaatttaaattctgcatttagtatgtgcaggtgtggttaaatgatgggctattatatacagagtgagaatctagtttaaacactgctcctaatgtttatgcctcagcttttttcttttgtaagatagcgattatctgctgtgtcttttctcaggattattttgaaaatacagccataatatacatataaaggattactttagttttgaataaatgtgaaggatgtagcactccaatggagttttctgatttgtcttctctgtctccactgccctgaccttagtttaggccaccatcatatcatagaattattaaaaacaacctcctacctcctgatttccatcctttggtttcacatcattttaatccatcctcctcatttctgtcaaagttggcttttttaaaaagcacatgtgatgctgtcactcctatttaaaattcttctctgattcctcatagcctttaggaaaatttagcgttaaatacatataatgacaagggtgatcttatgtgccagtttttgcttgtccagtgtaattattaattgcttgcctcttcactctcaggaaggtcccaatttgggtggcagattatttagtcactctgttcttgatcttttctgcctttcttttccagaaaaatagagtatttagtatttgtcctattcccaaccttcttgaggtatctagttcttgaacccaggctctttcccaagttttacagcttcctgtatccttaaatgcatgttgttaggcaatgaatttgaaagtttagtgtctagttagggttgattagggatttttaataaggttatatcatagtaggtgtatgaggtgccttcccgcccactttcgagtgcctgatcctgctaccttgtttaaatttctttatgacaagcatttctcctctatcctagtaataaacaatcctgtgaggtcagttaggtaacacagatgtctgttttattctttcagagtaacaaaagaataaagtaataactggttagtagaaggaagtagattacaatcctaaggcctgctgatctcttaacagttacattagtgctatttaatatgttggctacgtattggagtgttgtccagtttgtagctcactttgaagggaccatgcagccatgctggattttctggtttagtcagattgtctcctagttgttccccagagaagccacattcatttctgcctcttgctagtgtctcccttccctctctctactcccccttccactgtcttagaatgttctttttttcttctctgcttaccaactcttaacctttcttaagttctcaaatctcacttctcccatgaaatattttctcattgtctaagttctttatgattatcctaccacataactcctatagtacctaccaaattataccatatatattatagcaattcctcattgcttttttataataatagtattaatatgaatgccaattacttcaaaatatttgttgccaagttgttggcatatatggtagtttaatacttaatagtaaagtgtctgtagcttttgtatgaccataggtcatttggtaagtctcccagaaagcattattcacaaaaattaatccttgcatacttgatttcctaaaacactgtcaatatgcttcatagtgaatcaatactagtgtttctaactggctaacagagtaagcagaaaattgaattctcaggtcatttcagctttcaaaggaattatttttgactgaaaaagttacttattctctatgcttacagctgaaccaaaaagccccactgtacttatttctttttaagactttgtccataatgctacagtttttcccctgagccattcaatagaattttcagaacttggtaaacctacctgttttaaccgttgttcaatgtattattaaaatgccttcagagaggcctagagggcacaaaacaaaaattgctgccaaagttttctttcacatccattctatctaccttttctggtctctctgcattactcatttcattcacatgtattccagaaagcataaaaccatgttggggatttctattggctcctaagcttgccacttgtctgttttgttctattgcttatggtaaatcaatttttatattattattcaacccccctctgccctgaacattctacctagaccattctcacttgcctcacaaatacatatagtatgttctctactgaaagttctacccacatccttattattgctataataatcacttcctctccctttctaaagccactttcctatgttagtagacctcacttttttatttctactggctatatatttttatttgtttgctggaaacccacattaaaatccagcttttagggtggctaatccaaatgtgaaaggaataatgtgaaaaaggaagaatactaagtaagacataattttcagattgcttgtgggaggcaaaggatgaggttggggtggatttgaaaagaagacaaaattctatttttaatttatcatttattctttaaatcttgtatcttctatgtaaccagtgctgtgctgggtaccataaagcatgcaaaagaagtaaaatatatagtctttgtccttgaaaagctgacagttgaaaagcttagttgaagcaagtattgaataatgtgagatactatgtaaccgtatacttaattctgtgctgtaagtgttcagaggtgttccacatggctgtaggtgttacaaggcagacttccagtgaggatggtggagggaaaaaggaacccccacttagggataatacatgaggtgggatgttgaaggggaggtttaagatttgcctaggtgtagaaaaggttaggaattgttatagcctgggtgaatatctctcaaggtggagagagctgaatgaaaagaaaatatttctatattcataggagagccaacctgactgtaataattgttttgtgaacaatgaaagattaaattggataagcggacatttggcaaatcccactcctgtgtaggcatgtagtactatttcatcagcacatgatgtgagggagagaaattatgttacatagttaaatgctgatatctacatatatacagccagtcatttaacatcgttagtaggtgcttagaaactgcaactttaagtgaagttatgcacaataggtcctcaaataatgttggttcattcagcgttgatgagggaaaacatttggttttattatacgtttcactaaaagtcacagttttcaagaacctatcaatgacattaaatgaagatttactgtataacttttcttacctatagtagaacttgggaaaactcgataaggcttttaattagtacagctgaaattcactggatgttagatttgtcatggctaaggaaaggattctttgtgtttcttatgctcaaccttctttacctgaactggtatcttaacgaaaaggcttagaaaattatgcagtagacttgagctgaattcagtttgtgtatcctaccctgttagatgccaacttcatagatagcaattttacttgggaggaggactctgaaagttgttttcagtagcctctggtaatcatttttgatagtacttaaagtgaattggattaccttgtatgtcagtgcatcattagatcaatgtagcaaagcatgtgaaagactgccaaagaatatatgtttttttttttttttttttttttgagatggaatcttgatctgttgcccaggctggagtgcagtggcacgacctcggctcactgcagcctccacctcccgggttcaagcaattctcctgcctcagcctcccgagtagctgggattacaggcacccaccactacacctggctaatttttgtatttttagtaaagatagggagaatattttaaaaaatagtatagactaatttgttaagtaattattgaattatgtgctacatatgagcacttaagtagacttgatttctttcttagttaacataattacctattcaacaatccatggcattgtctcacttttaatcctatgtttaggtttacaaattgcatataatttcagatgattaatagattacttgagagtcaatattggagttgctctttaggagtattcatggtttattagtttttaaaactggatgcttatattttgtatatgaccaggatataggatgagtgttgtacatttatagtaataaaaactaatgaggaatagctatttggaacagacatggctcttgttctgtgttctgttagattaattatcactgccaacagccatttaaaatggacatttttacattgctctgattggctacataatacaatagagttgctaagagctgatgctgtgagatccatgttaaccataggtcttagtatggtgtatgtgattgtttaaaaatggcctttttaaagtgcttgagattcagtcacaacacttagctccaggttgaaatttgaaggacaaagtctattctaggacatgtagggcagaaattgcctctgtcattttttagttacccaaatgcaaatttattttgaataattcttcatttaaggacttctttgcatttgttcaatactgcagaaactttctttttgaaaaaatgatttttttctgttagtttcttctatgggctagtatttgtgatgggagttgatatttgtttcaatttaagaaaatgagttgctgcataggtgaggtagtatttgaatcactttaaaagttcatgtcagtgttttggttcttttcagtatttcctttctcttttctttcttttttccttcctctttttcttttctttcttcttcttcttattttttgctttgttttattttgttctgttttatttatttacttttggttttgtttttaaaggggtgttttgggatctctgtggccttgctgcatacttcgctggccatatttagtctgcgtactaattgttagttggcccagttgagtcttattttgtgctctcagttctttcagtctggttttctatttttgtccacctggttgtgttttagttcatgagagaatttattctgatgggccagattttaatctgtgtactaaattttagtctgcagactaaaagtaatccgcggattatttatttttgaaaaatatttaattcccccactggaaactatcctaaaagaatgttaaattctcttagaatagccaggggagttcacagatactggtgagttctgccattttttgttctcaatacctagacataatagaccctgctacctcccttgcatatttggctccgttgtgcctaggtgttggtatacattactatgcctgtcacctgtaactgtgactgtcccttagttgtattgttaattttatatattttgtttagcttttggtgggtgaattgggcatatttgcagatggaactgtgtttaatttgtagtgctggaagatgtaggatttattttttcagagagtttttccaaagaggaaaaaatgttctctatttatttttcctaagtaccgggtgcaagggtatgagcgggcaaagcccacaatagaaaaagtaagttaatacccgcactcctgaggtgatatgtcaatttcctgatgagtcctaagataatgagaatgtcagcaaagaaacatcttttagcagagaagtcaatccaaaatggagaataatccgcggtggcctgatccttcttagctcacacaaattgaaagtatctggcttgaaatctatagtaatataacttaaatatatttattactttcctctcatcactaataacttagcaaaaaaatgtttatactgtgtaactggaacacattaatagagaaaaaagtagcatattgggaacttgaaactttaaaagcaggctaatatgccccctcccagccatgtctgtggatgatgaatccaggtcatagaagaccattttactttgggaactttacttctagatttcattttgattttaactttttttggttttgtatttttttaataaacgcactggcattggaacataataaactaaaattaaaatgtttctttactgtgcctcaccattgcacaatcaaaaaaaatttttttgcatacactgtggtgaaataatttttaaaacttggacttgctactgtgaagactgatgtttaaagtttataactgtataaacattttctatgtccccccccacaccatggatttcttataacattggatatatttttccttgctggactattctcttttggattgacttcccctgtttggattatttcactgcattatcatctttaggattcgtcaggaacaggacatttcacctcaggagtgcgggtatgtacttaatttttttttcccaaaatttcagggcaggtctataaaacttgggggaaatcttctttgagaggctaaataattactcattcttgaaatgtaatagtaaacaatgtaagtagctgtgatttctgtttttattcactgtatggcataggtttcacttatagtatatttagattgtcagcctcttgggtgattcattgacatctaggctgaagtttaggaattgtggggttttaaaattattatttctattacaagaataaatattgatgaagatccatgaaaccaaaggtctgagtgttaagcaatttcagtgagtaaggaaagaagtgacagcggaagcttctttaattaaaagacaaatttcagttcttggtggtttccttttctttagaagcaaaaataattcagatgagctagtttctagtttgccctataatttttagaagttacatgctaactcaccctgtattatggtcagaaatctgaactgtgggagatactgggttttgaccccacgtaattttccacttaacctttattcacagagtactgaacctaggcttttctcatcaagaatctctcaagggtttaaaatgacagtgtatagtttttgtaaaggcaggttaaatcttgattttaatgtaggcttttgacatgtattattttcttcattgtttttaactcttgaactttatgagttaggattccctgacaaatatacgctaataaatgtcttagtaccgatatggatgatcacttgtaggcagtaaaacaccaaaagatatgggtgacttaaaaaattactggaaataggccgggcacggtggctcacgcctgtaatcccagcactttgggaggccgaggcgggcggatcatgaggtcaggagatggagaccatcctggctaacacggtgaaaccccatctctactaaaaatacaaaaaattagctgggtgtggtggcgggcgcctgtagtcccagctactcgggaggctgaggcaggagaatggcatgaacccgggaggcagagcttgcagtgagccaagatcgggccactgcattccagcctgggtgacaggaagactccatctcaaaaaaaaaaaaaaaaaaaaaaatatatatatatatatatatatatatatatatcggaaataagcttataatactggtttattaatagctttaacacaaaatctttgaaaataagaaatttttttatttaaaataactcagcatccagccatcctacatttgaatgttagtgggagatatttatttctacttttggtgctacttttaattccacttatgtgctgccatattggtgcaaaaacattttggattagctagcaaaataaatccatatactatcaaacagaagcaaacttgccttcagggataaaatatgccaatactgtttaatatttttacgaaaattttctgagttttcccactgattattttctttttacattctgtatatgattatggaccttgggagcaagagaggggaccagtaacagtggaagagctaaacatttcaaattcgacagtaactctaaggtttccttgactgaggcgagtcagagtattcaaaagaataaagataaggcaggttttgaccttacaaaaaggtttctttgtaacataaccaagaaattgactaaggcctttctttatgttccgaaataatttcctaagagcctactttacctaatttctcaattttcatggaaaatgtctttttcaataataaattaaaaacaaatacaatcctcttttcactgtagacagcactcataaataatctctggaaagtctaatgagctctgtagttgtaactagttctactaactgtcagcataagtgaacaaccaaatgttacatgagccattcttacgaggaaaccttagccttccaggctgctccatgccctgtcacaatctgcgtgggcttttctcacttgtgggtccgtttccctccatttataagtgtctacttctctgcccacctccatcgtcacaccttttatttcgttgtatgttcttcctgtcgagggatatatgtttcatttttctttaatttcaaaggcttctcgtctgtttatttctatccttacctttttatttttattctgtaggtggccataatttagaatgaagaatatttttatagtttttgtatttattagctttatttaagacacaaattagtagaaagctatctattcctttaagtagattaatttaattgaatttttcttcaatatagccttcagttgactttttctttgtcttaatcattcttaatgaataatacatttttttctccttgattaaaaccctggaatattcattcaagtgggggaaatctggccacattcatggtaacacctacttgagaaaaaaaatgcaggacattttatcaaattctaggtattcaagaatttcttcaaataatggtaggctctgtttattggaaataataccaaatcagtagcatttatagacctagagtaaatagctcttctttctcttctgtttgattgccaagttttcgaacagtgctgctcatcattaacaattgttcggctaaaaaaacaatgcaaaagaggcaatcttttatttactgccatagcagtgaaaactttaaaatttaaaaaatatttttctagttgtgcttgccaccagaactcagcctttcccatgtgcctgtcatattttgtatgcatgtctgattgttattactttatgattagcatatatagatttttctctgagcatttctcatataatctttaattagcaattcacccctgtgaatcatatgcatttaactctttccattctagaaggtcctttcccaaatgcaaccatgtttctgtacatggtgggaattaagtcattattcacagggtatttgcttagattcttaaaataaaacattaattgttctttccctttttttgtactgcaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaaggtaactccaaacacttagcctcttcaccagttctaggaaagctacaccctgggtgtgaaatgacattctattaattaaaatctgtggaggacaaaaacggacatattaagtacttaggtaggtgaaaagagcatcctttctaacttactggccactacttacatggttttgaacagaagagtagcacgtaaaagttttccaatagacatagcttctttaatttttagcagaatataatgttggcacatgagaagttacagagagttcagtgagtgaagtttatcactaattggacaaatgtacaatttaacttttaggtttggatctgaaacaaaatagtagcatttcttgcttccaaagagtttgtatcaagtttttttaaacctggaatgaattggctatatttgtgtaaattcagttgtcttccaaacaggaggaaaagtagagccctataaaccctttctaagaacttaaacaatagacacaaggaaatttccatcccctctgagactggcaccattgctgctgctgagataatactgaaagggaaacgaactttgtgcattgtggtttctgggagaccttgggatagtcactatttcttcactatttctgtgttcagttttcttttctccttgattaagacctaagtgattcattcattcagtcacaccgcctctgctgatgtgactttcttattacactgagcataaaaagtgtacaattcattacaaaaaaaaaaaaaaaaaaacaagaaatagttgatatattgattagtaagcatactcaggctagaaaacaaaaaagggagatgtagagttttatgtgaccaagaaagaaatatatttcctttattcatatgtatcatatctttctttttttttttttttttttattatactctaagttttagggtacatgtgcacattgtgcaggttagttacatatgtatacatgtgccatactggtgtgctgcacccactaatgtgtcatctagcattaggtatatctcccaatgctatccctcccccctcccccgaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggatatgaactcatcattttttatggctgcatagtattccatggtgtatatgtgccacattttcttaatccagtctatcattgttggacatttgggttggttccaagtttcttttaaaaaaaaaaaaagaagacttttattttggtattttaaaatcttatgtttgcccagcacattagatataaatatgtgtgtcttgaacatttatgtttttgagggtttggagaatgcatttgtatttggatgtaatgtgtatagatacagtgtgtggtctctgtggttatcagagtgcttatgtgattctgtgagtatcagtgaaaaagtggaccctgtaaacacagtcatgtattgcttaatgatgcagatatgttctgagaaatgtattattaggcaattttcatccttgtgcagacgttacagagtgtgcttacgcatactaagtatattattgaaagtgctatcattttgaggtatggccccttgttaaaaagtgttcaaatgctctgtcttagcacccattctgaaactgggctgtgatcacaaagtagtactttctcacggtctttgtaaatcgctattgttactgtttttgaacagtgtttctatgcaataaagacacagatgtccagggaagaggtggggtttttttgttgttgttttttggtttttttgtttgtttgtttgtttgttttgttttttttttttttgagacggagtctcactctgtcactcaggctggagtgcagtggcatgataccggctcgctgcaacctccacctcccagattcaaacaattcttgtgcctcagcctccttagtagctgggattacaggctcctgccaccatacctggctaatttttgtattttcagtagagacggggtttcgccatgttggccaggctggttttaaactcctgacctcaggtgatctgcctgccttggcctcccaaagtgctgggattacaggcatgagccaccatgcctggccttgatgatgccattctatttgatatgatactctgtattctaattttggagcagtgtttccaaatggtgatcttgttttctaaggaagctgtaactgtgtacagatgcagctttaaagcacatactagttagtatgtggatgactagttagttaatggatgaccagaataaaactgggtaatccaaggggttgaagaaggagaatcaattttccacttggttgtatagctaaaggaaatctagagatagaaacattgtgggtctggtgaagggtgaggggtattagtgaggttcttctaaaggcagcataaagccctcctccatcctcataacaaattgtcagtgacatctgcagcactacttcccccaaataaaataaagtacatatagtaggtgctcagtgaatgtttgagtgccgtgtaccactttaagtaaacataatattcattaattcatacaaccagtatttattgggcatttctaatgttccctctggttgttattactattgttgttttaattagtaaagtaaacctgctatatgtacagaactataaagattaaacacttgagtaaaatatagtgttttattaaggtaaagcttacaataaagctaactgcattttgatgtttgttactgtaccatgaggtaattagccataaacagtaattagtattactttcatgtaatcacagtatttccatattcagatagaggaaggagagagatgaacagatatgagatggacaactttaagatctttctgattgcctaaatccataactcaggagaatatcagacctctcatttttccagcactgaagcttcagtgttccaggaccccatgactgacaatattatctgcattcagatgttggaaattggaataaacctttggcacacccccttctttctatttcttcctgtcattagaatttcaaattcagccatagctgaatcagaccttaggaaacaggtagaatcaggaaagcccaataaggatcagtattttcttatgaactaatttgttgcctattccaagactggaatatattagggaatcaattgttttgtatgtttaaaggtgagtctccccctaactctatcacacacatccccaatctcagttttttacctaatttgtatgaaattttaccaattgaagttatttagtaaacattgtcctcctgagagtgtagggtaggatgggggtggtaaagatattatccatttactttagtatgagctaatgtgaacattctattcctctaccacttggctagattatgtgagaatggtagctttgtagggagataagtaagagtggcagcatgttggctaggccagaagaagcagtgatttagtaactctggggaactgtttaattgagagttgaaacaggatatttggggcagccagtgagcaatatgcagactcagacagactgaatattttggggcggggggtaagagtatctggacacgagaaaagcaggtgatgagatagaaaaggtgttccagaaagcaagtaaagaaaagaacagtaaaaggaagattgagatataaaaaacaaaggaaaataaggaaagggttagaaaatattttttgaaagagtataagaatgtgttacagaataggaattgatgaaaacatggaattgaaaatgagtaggagatgggcaacaaaagcaagtgttctagaatccaagctcatcaaaagtttgtatttctttcatttgaaagcagtaggctacttatttataaaatctattgtaattaattatgataactagcagattccatatttgtaacaatgatttttaaacttattttcctgttataaaataacaatagacataaataaggtcagtgcatctatttatctgcccatgtgctaggcattttcttctaagttgccttatcctcagacacatctgaagtgaatatagtattacaactacctgagattaattaaagtatggacatacattgttttattgagcttcactttattgcatttcacaggtaatgcttttttttttttttatatacacaaataaaacgttcgtgtcaactctgtgttgactaagcctgtttttccaacaggctatgcttactttggtgccatttttccaacagcttatgtttattttgtgtctctgtgccacatttggtaattcttgcaatatttagagctttttcattgttattacatccattatggtgatctgtgatcagtaatttttttgatgttactattgtgattgttttggaagtttgtattgaactaattgatcaatgtgtgtgtcccaactgctccaccaatcagccaatccccatctctctccctcttcttggatcttcctattccctgagacacaacaatattgaaattaggccatttaataaccctacaagagcctctaagtgttcaagtgaaaggcagagtcacacatactctccctttaaatcaaaagttagaatgattgagcttagtgaggaaagtgtgtcaacagctgagataggccaaaagctaggcccattgtgctaaacagtttgccaagttgtgaatgcaaagaaaaagttgtcaaaggaaatttaaagtgctactccagtgaacacacaaagaagtaaatagagatcaaaacagccacagtattttcttaaggcaaagcctaatccagagcaaggccctaatgtcactcagttctgtaagggctgagagaggtgaggaagctacaaggaaaagtttgtagctagcagaggttggttcatgaggcttaaggaaggaagccatttccataccattcaagtgcaaggtgaagcagcaagtgctgatatagaagctgcagcaaattatccagaagacctctagctaagatcattggtgaacatagctacactaaccaacagattttcagtggagacagaacagccttctattggaagaagatgccatctagaatcttcttagctagagaggagaagtcgatggcctggcttcaaagcttcaaaggataggttgactcatttgttatgggctaagacagctggtgattttaagtggatgccagtgctgctttaccgttccaaaaatcctagggcccttaagaattatgctaagtctactcttcttgtgctctataaatggaacaaaaaagtctgcatgacagcacatctgtttacagcgtggtttactgaatatttgaagcccactgttgagacctactgctcaggaaaaaaagattctttcaaaatgttactgcttaatgacaatgtatctagtcatccaagagctctgatggagaggtacaaggagatgaatgttgttttcatgcctactgacacagcatccatttctgcagcccatggatcaaggaatgatttctacttttatgtcctgttatttaagaaatgcatttcataaggctgtaactgccattacatagtgattcctttgatggatctgggaaaagtaaactgaaaaccttctggaaaggattcaccattctagatgtcattcggaacatttgtgatttgtgaaagaggtccaaatatcaacattaacaagattttggaagaagttaatcccaagcctcatggataactctgaggggctcaagacttcagtggaggaagtcactgcagatgtggtgaaaatagtaagagaactagaattaaaagtgaaacctgaaaagtgactgatttgctgcagtctttatgataaaacatgaatgaatgaggagttacttcttatggatgagcaaaaaaagtggtttcttgagaaggaatctgcttctggtgaagctgctatgttgagatgacaacagaggatttagaatattccataatcttagttgaaagcggtgacagggtttgagagggttgactccaattttgaaagaaattctaccagggtaaaatgctatcaaacagccttaaatgctacagagaaatcttttgtgaaaggaagagtcaggcattgtgacaaacttcatggttgttttgttttaagaaattgccatagccaccctaaccttcagcaaccaatatcctggtcaatcagcagccatcgacataaaggtataagaccctccacccacaaaagatgactcgcagaaggctcaggtgatctttaggattttttagtgataaagtatttttaaattaaggtatatatgtggttttttttagatgtaatgcttattgcccacttaatagacgatagtgtagggtaaactcatcacttacatgcagtggaaaaccaaaacatttgcgtgactcactttattgtagtggtctaaaactgaatctgtagtatctccgaggtattcctgtatcttctaaggttattctcaaattactatgaactctgaggtgagtcataatggtttttagacttattatttacaatagccatgataaatttacataggataggaactcttgatgtgtgtaggacacatcaatagaattgatgtcagaattattcttcagtactttctaagtaatctagaagagtaaaatttctatgtaatcaggtatactcttctgggcaatgaaatgctaggtaggccagtggagacacaccacagggagaacttgagaaaatttatgagtggactaaaagggggcaggtaagatttagttcaggtaactacagcagtaatgacaaataatgagtgactgtattcaagattttaggctctaaattatctattttatataagaatttttattgttttgcctgagattatttggtccaccctgctactttggatgtactctactggggaaaatgtagagtggtgtaggaacaaacaatcaaacactagtatcttgaatgaaagttctgtgtgtagtttggacagatatacttacagaagaccagaacactggtggtatatgatatataaccaagacagtaaagaaatgcaacattgcaggagcaaagagtaagtcagacctaaagcctagttccctttaactacacaggtgtagaccttggtcaacacactatccctacctgagtttccccatataaataaatgagattgttggattaaaggatattcaaggacatatgcatcttcaaaattttaattataaaaatgaattgactccagaaagataaagaaaaaatgacaaagctaaaaatgtagaccacaataggattgagatacaaaatcatgatataaataaacatagaaaaatgtagactggttctcccagaatcaaaatattagcagtattattatactttaagttctagggtacatgtgcacaacgtgcaggtttgttacatgtgtatacatgtgccatgttggtgtgttgcacccattaactcgtcatttacattaggtatttctcctactgctacccctccccctccccccaccgcacgacaggccccggtgtgtgatgttccccaccctgtgtccaagtgttctcattggtcaattcccatctgtgagtgagaacatgcagtgtttggttttctgtccttgcgatagtttgctcagaatgatagtttccagcttcatccatgtccctacagaggacatgaactcatccttttttatggctgcatagtattccatcgtgtatatgtgccacattttcttaatccagtctatcattgatggacatttgggttggttccaagtcttcgctattgtgaatagtgccgcaataaacttacatgtgcatgtgtctttatagcagcatgatttgtaatcctttgggtatacacccagtaatgggatggctgggtcaaatggtatttctagttctagatccttgaggaatcgccacactgtcttccacaatggttgaactagtttacagtcccaccaacagtgtaaaagtgttcctatttttccacatcctctccagcacctgttgtttcctgactttttaatgatcaccattctaactggtgtgagatggtatctccttgtggttttgatttgcatttctctggtggccagtgatgatgagcattttttcatgtgtctgttggctgcataaatgtcttccttatgactactggatacataacaaaatgaaggcagaaataaagatgttctttgaaaccaatgagaacaaagacacaacataccagaatctctgggacacatttaaagcagcatatagaggaaaatttatagcactaaatgcccacaagagaaagaaggaaagatctaaaattgacaccctaacatcacaattaaaagaactagagaagcaagagcaaacacgttcaaaagatagcagaaggcaagaaataactaaggtcagagcagaactgaaggagatagagacgcaaaaaacccttcaaaaagtcagtgaatccaggagctggttttttgaaaagatcaacaaaattgatagaccgccagcaaggctaataaagaagaaaagagagaagaatcgaatagacgcaataaaaaatgataaagggggtatcaccaccgatcccacagaaatacaaactgccatcagagaatactatgaacacttctacgcaaataaactagaaaatctagaagaaatggataaattcctggaaacatacaccctcccaagactaaaccaggaagaagttgaatccctgaatagaccaataacaggctctgaagttgaggcaataattaatagcctcccaaccaaaaaaagtccaggaccagacagattcacagccaaaatctaccagaggtacacagaggagctggtaccattccttctgaaactattccagtcaatagaaaaagagggaattctccctaactcattttatgaggccagcatcatcctgataccaaagcctggcagagacacaacaaaaaaagagaattttagaccaatatccctgatgaatatggatgcaaaaatcttcaataaaatactggcaaactgaatccagtagcacatcaaaaagcttatccaccacaatcaggtgggcttcatccctgggatgcaaagctggttcatcatacgcaaatcaataaacataatccatcatataaacaaaaccaaagacaaaaaccacgtgattatctcaatagatgcagaaaaagcctttgacaaaattcaacagaccttcatgctaaaaactctcaataaactaggtattgatgggacgtatctgaaattaataacatgtttatgacaaacccacagccaatatcatactgaatgggcaaaaactggaagcattcctctttgaaaactggcacaagacaggggttccctttctcaccagtcctattcaacatagcgttggaagttctggccagggtaatcaggcaggagaaagaaataaaggaaaagaattaggaaaacagggggtcaaaatgtccctgtttgcagatgagatgattgtatatttagtaaaccccatcgtctcagcccaaaatctccttaagctgataagcaatttcagcaaagtctcaggatacaaaatcaatgtcaaaaatcacaagcattcttatacaccaataacagacaaacagagagccaaatgatgagtgaactcccattcacaattgcttcaaagagaataaaatacctgggaatccaacttacaagggacgtgaaggacctcgtcaaggagaactacaaaccactgctcaacgaaataaaagaggacacaaacaaatgaaagaacattccatgctcatggataggaggaatcaatgtcgtgaaaatggccctactgcccaaggtaatttatagattcagtgccatcctcatcaagctaccaatgactttcttcacagaaatggaaagaactactttaaagttcatatggaaccaaaaaagagcccgtatagccaagacaatcttaagcaaaaagaacaaagctggaggcagcacgacttgacttcgaactatactacaaggctacagtaaccaaaacagcatggtactggtaccaaaacagagatatagaccaatggaacagaacagagccctcagaaataatatcacaatctacaaccatctgatctttgacaaacctgacaaaaacaagaaatggggaaaggattccctatgtgataaatggtgccgggaaaactggctagccatatgtagaaagctgaagctggatcccttccttacacctgatacaaaaattaattcaagatggattaaagacttaaatgttagaccgaaaaccatgaaaaccctagaaggaaacctaggcaataccattcaggacataggcatgggcaaggacttcatgactaaaacaccaaaagcaatggcaacaaaagccaaaatagacaaatgggatctaattaagctaaagagcttctgcacaggaaaagaagctaccatcagagtgaacaggcaacctacagaatgggagaaaatttttgcaatctacccatctgacaaagggctaatatccagaatctacaaagaacttaaacaaatttacaagaaaaaatcaaccccatcaaaaagtgggtgaagagcagtattatttttaaatgcccaataagtttattttttaaattggatttgaaaaacttggtaatgcctcatttattgcatttcattaagaatagagttctgggctgggtgcagtggcttacacttctaatcccagaactttgggaggcgggggtgagcagatcacatgaggtaaagagttcgagaccagcctggccaacatggcgaaaacccgtctctactaaaaatacaaaaattagccaggcgtggtggtgggtgcctgtaatcccactactcaggaggctgaggcaggagaatcgcctgaacccaggaggcagaggtcacggtgagctgagatcacaccactgcactccagcctaggcagcagagcgagactctgtctttaaaaaaaaaaaaatagagttctgaatataagattttcaagtacatcactagaactttaaaaatattaacactttgctagataacttctaaaaacacctgtactataatgtagttcaataaaagcattcagacattttttggtgagtggtataaattggaaggatcccaaagtcatcatggctttcagcaaatatcatattatatagtcctcacatgaaacatcaccagtattaccaaaacagtatcagtaagccttgtaatttgcttactatttgtctgattttctaattagtttagcattaggaacatggtcaggaatttcctgggcactttggaattgttaccaaggagagaagcagttatgatgtggtttattcattcatctacaaggaaatatatacaagtaagtaagcaccattagaattcagaaatgtgtgagagcaagggacatcatcttagcatttgaattattgcctaaaaaaatacacaaatgtaatgggggcactccattattaagtcatttaaacctttttgtgaatagagagaaatagcacttttaactgtatagacaaccactgacaattattatatgtaataagaggcctggtacacacatgaggaggtcagacttcttgtagaagaaaaagaatgtctgcttattttagtaactcctttttggagatctgcagggtgagctccttcaaagctagtagaggaggaagattcccttttatataatgactgctaagtattttattacatcactgtaagaggagatctagtcagtagaggggttgattttcttccctctgattcacacattgattgctgctctcaaatttgtcctcatctttcacacaacagtttgtacaaacacgttaccactagcacctacctgccccctcagaaaactaggaaagttcttacctgtcccattttatcaatgaatttggggatcttgtacttggaagaatagaaacttttaaggcaaatttcagattttaccaactagatgtaggtttcatttttccattggaaaagactgctttatcaggcatatttgagtttattgagtttgtcttttcatgggaatttttcttctgtgttttgcagtataaaacaatgcagcaaaatttagttaaatggattttgttaactttttggttgattcatatctatatttaaactcattttgcttttgtttaaattgtgcattaaaatgtttaattatggaacacataattattgatgcaccatcaaaacatttgtagttttccctaataaactacgtggctttttaatgaatagttttcatgacttcttcagttggctgaagaaatttttatcacattaatttttctaaagttttctggaatttcttgccgttttgttttaaaccatgtctctaggtaacacagtctatcttttatccttcctatttttcattttcctctcacttctccagagctgtcccacttcattatggacctttttgttcttatccccctactctcaagcttagcacacatatctagtaaataaatattaccttacatttcaatttcagtctcttcaacactcctctgccttcaagaatacatacactgtgtctgatgctaacaggtgctgttactttgctgtttctatcctccaaaataccaccattttttctattctttcactgcagtttatattcaccctcactgactccatttccttatcactcattgccatgggtagacaaagtgattagaagtaggtagtaatggttgggattaaaaaaaaacattttgaactggttgacatagccctatagaaatgcccatagtctgtccccataaaaagtgtggacttacagtggcaagaataggactatagcaagagtgcaaggaggcttcctcacagggaaaagttgtaagtaacagttaacaacctctttgaagtagagaagactgaacagatgaaagcaatggattttaaggattgcctgtaattaaggaagagaagaagaacaatggggtgtagaagttggaaggatgtagaagtagtagtgattgttcattttagaattcttaacttttaggttcttgctagaaaaaggttcacaaagtaccccccttcacatcttggaccccaccctcacccgcactccgtgtctttgggtgtggcaaagcctccgagatcccaaatcttttctatttcttgtcttcttaatttcttcactttttttccccttacccttcattagttacccctctctccttatctaaggccaaaatgacagtctttttacatacatacatacatacatacatctatagatttacattacatacatctatagatttacatctataaaactatttacatagtatttacattgtattcggtattataagtaatctagagatgatttaaagtatatgagaggctcatgcacgagaatcgcgtgaacccaggaggcagaggttgcagtgagccaagatcacatcattgtactccagcctgggcaacagagtgagactgtctcaaaaaataaaataaaaaataaaaaaataaagtatatgagaggctctgtgtaggttacctgtaaatactatgccattttatgcaagggacttaagcatctttggatttgggtatctgaggagggtcttggaacaaatcccccaggataccaagggatagctgactatacattacatattcttgtccagactaagacacaaaactgctgtggccttcaactggcattcagatacacagaaggataggtgatgtggtaaaagagagtaaatatatgggagaaaatggacagcatgggccataaggagagggaccacagaccactgatactcagtttattttaaagattatttttaacagctttgtcaaaatgtcttattaaaaactccggtcctcttttcatttttaggtttattttaggtatcgagcacatggttattgagatgttttcatttgacagtgcccctgtcctaagtggttgcctttcttgccagattgttttttcagttgttcttgtcaatttctgttgatcagacaaaggacagttgtctttttatctccctatcctgggaccttacacatttcatgttcaatattgttggatggatatgtgggtgggttgatggatggttggattttaaagaagcctttctattttagatgcttttaattgagaatggtaaatctgaatttctttagaataaaaaccacatattaatgcttttttttgtacctggagccataaagttttcagccatcacaattgctagcaattattttgctttcctgcatataagattatgggttaaaattgaactgacttgaagtaaaatggcataggagctttctgttccttagtgagaacccagatagctaccaagtctgattgtgagaaaacagtatctcctagaacctgataattagggcagtatcttcaactgtcttcccaggcaagactatttgatgagctggactaaattttctcatttaggggaaaggaaaatgaatagttttctcagttttaagcttttcctttcattcatttttaataaatcttagcatcaatttattgcttttctttaccacaatctgatatcatttagtatgtaatttagtacaaaatttagatgtttaggtactgatttgaataaatattattggcaggggctttgcaccagaaaacattttaaaactactacattaaaagtacaatttcccttgctagggtcttaggggaagggattaatgtagagtgactgcttgcgagtttggtgtttccctttggggtgatgaaaatgttttggaactagaggagatggttataaaccttgcgaatgtactagatgctgaattgtgctgaattgtacacttttaaatggtttatgttaacataaattataccttaatttaaaaaaaaaagcttttcttgaactttttaaaattcatgctgtatctgtgaattgccaaagtgtgtgctgtagctcagaggataatgtcctcctgtagcttgttatggttatgattgttccttcaaactgtgacacatattagagtgaaaattagtacttacaggctaaatacttaagtaacaagcataaaccagggttatcccaggtaactgggagaacagtcacattacctaaaactaacaaaaaatgcttttgtgtacaagctcgctaccactacttgctacagaacttgattcttcaaagaagtaagtacagttgacagagacagcttcccccatgcagataaaaagcctatttcaaacttgtaaaaagtaacaaaaataatcttactgagtaaagaattggataaagacattttcagttttaataaaagcagtgacactttaaacagatgtttctcaaatcctttttccactagtgtctagataagtcttatctgccattggctcataaacagttggataggttggtcagtacatccagttgtacatacatacacatacagacacacattctcattcatattctctttgctatgacaccaccaccttttaaaaacataaagaatatgtttgtacagtatttcctgtagtcaaatattgacatagtaggacaggaaaacctaatatatttaaatcaatgaaagatgcagatatgtcttattaatatacctactccaaaactttggccacatctttccccgagatatttttagttctcagacataattttagtagataatgcctttgttatatatcacagtacttctgtgggctttttctgtaagttgattactaactcagttagctagtttgagatataggtttgtcatgtgggtttttttttcccctgttaaactggtagggtttgttttgttttgttttgtttttgagacggagtttcgctcttgtcgcccaggctgaagtgcaatggcacggtcttggctcactgcaacctctgccttgcgggttcaagcaattctcctgcctcagcctcccaagtacctgggatcacaggtgtgtgccaccacgccaggctaatttttgtatttttttttaaacttattctttttttatgattattattatactttaagttctgggatacatgtggagaaggtgcaggtttgttacataggtatacatgtgccatagtggtttgctgcacccatcaatccatcatctacattaggtatttctcctaatgctatccctcccccttcccccctaccccgcaacaggccctggtatgtgatgttcccctccctgtgcccatatgttctcattgttcaactcccacttacgagtgagaacatgccatgtttggttttctgttcctgtgttagtttgctgagaatgatggtttccagcttcatccatgtccctgcaaaagacatgaactcattctttttttatggctgcataatattctgtgatgtatatgggccacattttctttatccagtctatcattggtgggcatttgggttggttccaagtctttgctattgtgaatagtactgcaataaatatatgtatgcatgtgtctttatagtagaatgatttataatcctttgggtatatacccagtaatgggatggctgggtcaaatggtatttcaaattctagatccttgaggaattgccacactatcttccacaattgaactaatttacactcccaccaacagtgtaaaagcattcttgtttctccacatcctctccagcatctgttgtttcttgactttttaatgatcaccattctaactagtgtgagatggtatctcattgtggttttgatttgcatttctctaataatcagcgatgatgagctttttttcatatgtttgttggccacataaatatcttcttttgaaaaatgtctgttcatatctttcgcccactttttgatgggattgtttgtttttttcttgtaaatttgtttaagttccttgtagattctggatattagccctttgtcagatggatggcaaaaatttttcttattctgtaggttgcctgttctgatgatagtttcttttgctgtgcagaagctctttagtttaatttgaccatttgtcaattttggcttttgttgccattgcttttggtgttttagtcatgaattctttgcccatgcctatatcctgaatgatattgcctaggtttccttctagggtttttatggttttaggtcttacgtttaagtctctaatccatcttgagttaatttttgtataaggtataaggaaggggtccagtttcagtcttctgcatatgcctagctagttttcccaacaccatttattaaatagggaatcctttccccattgcttgtttttgataggtttgtcaaagatcagatggttgtagatgtgtggtggtatttctgagggctctgttctgttccattggtctgtatatcttttttggtaccagtaccatgctgttttggtgactgtagccttgtagcctttgctgatgccgccagctttgttctttttgcttaggattgtcttggctatacgggctcttttttggttccatatgaaatttaaagtagttttttctaattctttgaagaaagtcagtggtagttcgatgggaatagcattgaataatctataaattactttgggcagtatggccattttcaccatattaattcttcctatccattagcatgtaatgtttttccatttgtttgtgtcctctcttatttccttgagcagtggtttgtagtcctccttgaagagatccttcacatcccttgtaacttgtattcccaggtattttattctttttgtagcaattgtgaatgggagtttgctcatgatttggctctctgtctgttattggtgtataagaatgtttgtgatttttgagattgattttgtatcctgcgactttgctgaagttgcttatcagcttaaggagattttgggctgaggcgatggggttttctaaatatacagtcatgtcatctgcaaacagataatttgacttcctcttttcctatttgaataccctttatttctttctcttgcctgattgccctggccagaacttccaatactatgttgaataggagtggtgagagagggcacccttgttttgtgctggttttcaaagcgaatgcttccagcttttgcctgaaacgttcagtaccgtattggctgtgggtttgtcataaatagctcttactattttgagatatgttccatcaatacctagtttattgagtgtttttagcatgaagggctgttgaattttattaaaggccttcgccacatctattgagataatcatgtggtttttgtcattagttctgtttatgtgatgaattacgtttattgatttgcatatgttgaaccagcctttcatcccagggatgaagccgacttgatcatggtggatacgctttttaaggtgctgctagattcggtttgccagtattttattgaggatttttgcatctatgttcatcagatatattggcctgaaattttctttctttcttgtgtctctgccaggttttgctatcaggatgatgctggctgcataaaatgagttagggaggattccctctttttctgttgattggaatagtttcagaaggaatggtaccagctcctctttgtacctctggtagaattcaattgtgaatccatctggtcttgggctttttttgttggtaggctattaattactgccattggtctattcagagattcaacttcttcttggtttagtcttgggagggtgtatgtgtccaggaatttatccatttcttctagattttctagtttatttgcgtagagctgtttatagtattctctgatggtagtttgtatttctgtgggatcagtggtgatctcccctttatcattttttattgtgtctatttgattcttctctctttcctttattaatctggctagcagtctatctattttgttaatcttttcaaaaaaccagctcctggattcattgattttttttttttaagagttttttgtgtctctgtctccttctgttctgctctgatcttagttatttcttgtcttgtgctagcttttgaatgtgtttgctcttgcttctctggttcttttaattgtgatgttagggtgtcaattttacatctttcctgctttctcctgtgggcatttatttagtgctataaatttccctttaaacactgctttagctgtgtcccagagattctggtatgttgtgtctttgttctcattggtttcgaagaacttatttatttctgccttaatttcgttatttacccagtagtcattcaggagcaggttgtttagtttccatgtagttgtgcggttttgagtgagtttcttaatcctgagttgtaatttgattcactgtagtctgagagactgtttgttatgatttctgttctttggcatttgctgaggactgttttacttccaattatgtggtcgattttagagtaagtgcagtgtggtgctgagaagaatgtatgttctgttgatttggggtggagagttctgtagatgtctatctacagaacttggtccagagctgagttcaagtcccaaatatccttgttacttttctgtttcgttgatctgtgtaatactgacaatgtggtgttaaagtctcccactgttattgtgtgggagtccaagtctctttgtaggtctctaagaacttcctttatgaatccggatgctcctgtattgggtgcatatatatttatgatagttagctcttcttgttgcattgatcccctaatttttgtatttttagtagagacggggtttcaccatgttggccaggctgatctcgaactcctgacctcaggtgattcacccaccttggcctcccaaagtgctaggattacaggcgtgagccactgcgcccggcataaactggtagtttaatgcagaatcttattttcactcttaccaataaaatagttttgcatacaaagttaagagtggactctaacatttgtgtcttaaccttctatagaactgtcaagtaatccacctctggctaccatccttattcctcctcatgctcggattcaagcagctgcttcaacccccacaaatgccacagcagcgtcaggtaagaacgttctaagtctcttctataaaaacatattctgggctgggcatgatggctcacacctgtaatcccagcactttgggaggccgaggtgggtggatcacctgaggtcaggagtttgagaccagcctgaccaacaaggtgaaaccttgtctctactaaaaataaaaacttagccaggtgtggtggcaggtgcctgtaatcccagcttctggggaggctgagacaggagaattgcttgaaaccgggaggcggaggttgcattgagccgagattgcgccactgcactccagcctgggcaacagagcaagactccatctcaaaaaaaaaaaattctgtttttcaaaaagatatgattacctttcatttcatttgatgagtatgaacatttaatatcttttatatatcaaaaacagcacagccaaatgagtaaacctgaataaaattttttgctttatttccaaaaagcataaaacaaaagcacccacccaggtaccttatatataagatataaatatcttctgataatgttaaattctgtcatcttagtgagaggatcactcgaggccaggagttcaagatcatcctggacaacatagtgagaccccatgtaaaaaataaaaataaacttaaaaaatctttgaagttacggtttttacataatttcaataggaaatcaaatcaaatgagatgtttatagctgaaaacatataaatatgttgttactgaaaataatacacttttgtttttttcccctcgaatgtaaattgcagtagtatactagagaggcaaatattttcaagaagtataaattataagtttatacttatatttaatcatttttttaatcatttgaaagggctaaaactgcaatgcaaatatgtttaactgagatttagatcagaatgtgacaagactcttatataccaaaaaaaaattattcacgggttagagctagggtgtgctgcagggcctgaagccagctaatggtggggcctgatttagtgagaaccggtgtccttgcaaatattctgaaaatatccaacccttaagagtgaagtgaaattcttccaaataatgactggatgcttttaaaacttaaaagttttaagttagaaacataataggtagcacagttaagggctcagaatttggcaccttttttttctctctctctccaaaaagctaatggcaggagctgacttcattttctttaaatagtcttgaactgagttggatcagatccatttttgcctaagggtatttatgctgcaggagataaaatctttcattactagttcttatcattaaacagtaaggtcaaaatccatgtcttgttgtttgatggtgttgtttctgtctaatcttaaaacttttaaaatgttgctaataaagatttaaaacatttcattctagttcatgttcaggaaaagtctggaaagaaagatctcttttcttgttccaagtttatcagcacatgctaccttgagaaagtaatttgaactttattagtttaattagtgtcttattcattttcccagtctgtaaaatggagtggcaattactttaggacaacatttgtctttacttgtctttatttgtctgtactttaggacagcattgccactccattttacagattgagaaagtgtgctgaaatctgctttaaaatttttaaggtaaaaagattttttaaaagtatgacctgtttagaatcaagacagaattgctataagccattaacaaatttaatactggcccaggttacgttaattgttcacaacatgaaaaaggccctaactctgaactaacacatttacttactgagctcatttctacagttttactagaatggctttacactgtgtataaaaatgagtaggttttgagcagtttaaagtaactttcttaatgcaagatagcgaactaattaatgattgtgacttatgtttttgagcaaattgtctgcttagtaggagcctagtataacctgattttcatgttgttttgatacaggttacagcaaactgaaaaataataataatttacatgtgtatatttttacagctatagaaatcagatttcaacataaaaatagaaactgacattgaactttgactctgagagtttactccaaagttgaattggggtatagttaatagaggaatagtaagctgaggtcagagctaaaattagtttagagaaatgcatgccttgagtaccttaatagttaccgaagaggatatttcagatattggtctgagctaaagtaaataaagaagtgttataattctgttaaaatttcccattgttcattagattgaaagcacactggtggcttagaaaaaacggtgtttctggccctgctgtttaagaaatatttctcctttagggcctcagatttggtactgaaagagatacagtaaatgaacttaacatcatcattctaacaaaaaatgtgagtttcaaatagatacttttttttaaagttccccttattgtaacccaaaaacagtttaaccacagtatagttaaacagttttctatggcgctgcagtagtgaaggcccaagtatgtcaccaaacatacttcaggaaatgctggtctagaccaagtgattgattgcttggccttccggctgcctgtagtttaagattcatgtatgttttaaataagaatttgtcattgtgagttacattcttttgtaagaccttaaaatatatatgtcctcggccaggcgcggtggctcacacctgtaatcccagcactttgggaggctgagacaggcagatcacctgaggttgggagttcaagacgagcctgaccaacgtggagataccctgtctctactaaaaatacaaaattagccggacgtggtgatgcatgcctgtaatcccagctactcaggaggttaaggcaagagaattgcttgaacctgggaggcggaggttgcaatgagccgagattgtgccattgcactccagcctgggcaacaagagcaaaactttgtctcaaaaaaaaaaaaaaaaattctcggcatcagacatgttttttttaaaaagccatgtggagggcaggattgacatccatcctcttatgacaaaagaacctatgacttttctttcctggataaatggacatgcatctctccacacagacagagtgagggactgctaagctacagacatagccgaagctttcctcacaaagcatttcaggatctgctccagagtacaaaataaataaataaactattaaactataaagctctagaccttgaatatctgaacctggaaatatttcaaatttaaattttcaaagaatttctaatcacccatctaagctacatcaccacagtaatatacccccacataccctggggcgaattaattataatatgaacccctaagtaactaaactagttgtcatacttgatttaaatcagtgtaatggagcacataacatcaaaatgattcgtatcttagaggtggagccacttttagtttatgacagtcatatcaacaagaagcaaaataatacttaaataatacttaacctgttttcagtagcctgagaatgtgcctccaaatcatctaaaaatgaccttacatcactaaaatacaaattcaaaagactaccataactgaattatcatctgatctgttaaaataccattcctgttgttgctcagcatcattgcaattcaggtactgtcaagtatatttgtgtctagagcaagatcatcagaataccgaggctcctagataaactgcttgggacactttttatgtttatagctggcttaaacaagaatggagttttctgacatttgtgtagacctgggaacaaagctcaatcctaaagtatgtggaataaaagcagaaaaacaaagcatattgacaaagatgccatcaaccattatcatactgactatattatggtgaaataagctttggattttagtcagttactgttgttagccaaagctgttaaatggctttcagatatttaagtcttattaaattttaaatacattataggcagaaatctaattctctatattgctgcttctgaaaaagcaaaagaaaatagacattttcatacccagattgtggcctaaaatcatactgtgctaattctgggaatatgtaattgttctctgtcatcaccaacaaactttgactagtacaacaaaatatcacaattctttctcacctttgaaaatggtaaagatggtgcaaatattaagtttgaggacattaacactgagggaactgcttttgcagatgtttcaactttccttttgttgtgttctgcaactgtatgtgagctgataaaaacttgtgggaaagcattatagtataaaacaagagctgtcagaattcagaatttttagttcagtaattccactttagggattttaacttcaggataaatctattggaaagaaaaaggctatcatatacagagaggggaatcacataatatatataatcataaatcatataataaaacagtatgtattaatatataacaaaaaggagagccaatgtaacctgaatggtcaacagtaggaaattgttttaatacattgtggaatctccatttttatgtagccataaaaaacaattttcatgaagcttgtggacattcatggaaatactcatcctaatttatgtagagaaaattttaaaatctcatgaaccttgttggttactattgtcaaaatgtgtattttcatgtggatagggactagacagaaatatacaaaactgtaaactgttgtgctaaattagtgggtatggatgttgatttttttcctaaaaaacattctttataagttgtttttaaaatgctttagtatctagagacattattgttaatcatggctctgattgacatgagaatctcttgaaaattacccatactactatcaattcctaaagtataatttctttgtttctttttttgagacagagtcttactgttgcccagactggagtacagtgtgtgatctcagctcatgtaacctccaactcctgggttcaagtgattctcatgcctcagccttccaagtagctcggattacaggcgcgcactaccataccctgctaattttttgcaattttagtacagacggggtttcactttgttggccaggctggtctcgaactcctggcctcaagtgatctgcctgccttggcctcccaaagtgctgggattacaggtgtgagccactgtggccagccaaaattcatagtataatttcttatgtcgactgaaatatatatatatatatatatatatatatgtgaatggtcaacgtaggaaatgttcaacatttatatatataaaatatatatttatatataaaatataaatatatattatatatgtcttattttctttgtacacccagtcatctcattcactccatttaggagaccattcatctggaacatctaaagtaataaatctcaagacattatcattttcctaatcaaatacaaaccattttagagagcatctgctcagatgagttgaatgtgagatgttgtagggaatttcaactagagcagttttctggttttatctgtttggtatattggtcatcagcataaagtttctttcaatgcaatggttctgctgttaaaaaccatggatttcagttcacctttcattatctaccgagaagtggtcatctatcccatggttatgtatattcagtaatagacattattgcaagaatgagcacatttttatctttgcatcactctattagaaaagtttggccaggtgtggtggctcacgcctgtagtcccagcactttgggaggccgaggcgggtggatcacgaggtcaagagatcgagaccatcctggccaacatggtgaaaccccgtctctactaaatatacaaaaaaaaaaaaaaaaattagccgggcgtagtagcgggtgcctatagtcccagctattcgggaggctgaggcaggagaatggcgtgaacccgggaggcggagcttgcagtaagccgagatcacgccactgcactccagcatgggcaacagagcaagactgctctcaaaaaaagaaagaaaagtttatccttttttcaccctgaaatttatttctgtatgccttcttccagtcagtgattctactatgaaattataacagaaccatccagatccttccatgtaaaagtccttcaaatgttaatgatagctaacttgccctccatactaatgacagctttaaatcctaatcctggatttgttctaaagtattagggtattggattttctacccaattgtatgtcatctgtacatttcacaaacatgtcagttttttacctccatccaagcaactaataaaacatatcaaagaggacaaggcagagcctactagaaatgttttgcctttattcaagttactgataaattcattggaactgggctgggtttcctagagattactcttctggtggtctcttagtgattggtcattcctcttcgtgactggtcattcctcttgtttgcttaactacagaccaacctttaaaatgcccatttataggaggaggttataaaaagcactttgtaaaatgcttcattgttttaagaaaactttcagcctttttccattaaaaaaaaagtaaccatatcaaagatggaaatcaaaccgttgtttttaagacggggaaatttctagtgcctaaatatctgtaagctgtgagtttctgtaatccgttttagatttgtttcttggactatagattgtgagtttaaagacatttaaaatttttgattgccctcaccttccttggggcacattttatttattaggatttttattatatcttttcaagttgaaaacccttctaacagaagggggagtcatgataacatttggatgaggtctgtcattttacgtggtctgcaggcttaacttgtcttcatcttggtttgaaaagctcttactgttactttatttgcattgagattatttgcaattacttgatcaaaattttgtttatgcttttctatttcttgttagttattagcctcctttgaagactttagccacaaaatcatacctatttgttcttcaaacttctaaaaattcatttggctatagtctcgttcagattttgctatggctagtggcccctttgtcttacaattacaattattttcaccccagaattcctgttacctacataccagccaattctttttagttgggcagaattaaacacagaataatttctctcattgtttgtttctactgtttttgagaggtgaaatgttaagcaaattgtcagtttattagatgcttgcttttctaaaatgacagttaccctccctcatcctttccccaggtactactaatattattcctggtctttgggccagttttacagtgtacattgggaaagctgtgttcatattctgtctagtgtagaagctgcagtgcatgccctgttgttcctctttgtaaacttgttcttcctgttttctctttatcttaacccaaatagattttaaatttcataaaagtctgcaactttttaaaaaaaaacaaaaaagcaggcatttttgtctatcaagaacatatagtgtatagttattttaggaagtaaaaattgcctctattctactttcaaagataatcacacataacattatgttttagtctatatatgaatgggcagagagacatgttttttaaaaaatgggcttaggctatttagagggtgaaggtatagactgtagtattcaaccaatatcctaaggctataccatagttaatttcttattcttggacttttggtttgtgtcgaagatggggttttttgtttttgttgttctgaaaaatgctttgaagatatctttgcataaagctgtacttattcttctaaatttttagaagtagagtcaaaaagtataaagaattttaaagtttaatgtcaaattgctttctgaaagtttgtcccgctgtcagttcatactccccactgtcagtacaagtacttttctatttccccattgccccatgtcctcatagagtgggagtaggggaagtacagtgtgcatgtgtgcacatacacatttttaggtgttaccaacttggtagccaattaatatgtgcatcttttttaggtacagtgttgcatccatttcctcctgttggctctgcatttgaaatacagacttccattttgaactataccattttgacaaattcactgacaccaatgagattgtatctaccccatgttagggtttcaggttcactttgtgagtttgtatatagatacctaaaatcaaaccagcttagtcattattctcaccagagcagtcctagacatcacttctagaagttcttgctttctgtgcaaaacatgttctctcctatcaagtcaaaaattttatctcggtttttcccctcctctaaaagtaatttaaaatctggattaagttggaattccctatcagacatttttccgtgtgtccctgaagtgttcctcagttccttgcctgaagtcacctacttttatttatatgtccttttttttctttattcctaaattaagcattttaacttaaaggaacagtgaaaatgttacctgtgtgtccccatgaccttcagttttctaccctgaacagccaaacttcttaaatacaatgtgccctttccctgagctcacagggaactgagacctctcagctgccagcagatcaaatataaacagtcttattgacaggtcttccaggtatcctggtggatggggttggctcacaggcatccgaattttactgctatttttataatcactgaaggctaccttagtgttctgtgccacatcttttccttgcaggtgtactttgatttcatgagtgtaaattataatttcaaattaaatataagtttagggtatactttgattctctgtgagtaattatcttgtttgttaatgtgccagttaataacattaatatctaagacatagttttacagtagaagcatttccacttggaacagcttgagtaggaacatcctgagttaggtacacagtataaataatatctcccaggctgttaattttatcttctagagagattgacctgtcataagacatttctaactattatagaaagaggatacctgataagtagaaacacgtaaaatgtgcttggaagagattgttattgggcaagagcgtagtaaaggaaatacgggaataaaaatatacctggcggggtgcagtgactcacacctacaatcccagcactttgggaggtggaggcggtcagattacttgaagccaggagttcgagaccagcctggccaacatggcgaaaccccatctctactaaaaatacaaaaaaaaatttagccaggcatggtggtgcgtgcctgtttaatcccagctactcgggaagccaggagaatcccttgaacccaggaagcagaggttgcagtgagctgagattgcgccactgcactccagcctaggtgacagagtgagaccctgtctaaaaaaaaaaaaaaaaaaaaaaaacaacctaatgtttccaagtgtacttataaagtgcatgtaaaacacaaaaaggctcatttgatttgtctaaaatatgttattttactaatatgccaacaatataaatagtaaaatgtttaataattttgtagctgagaacttgaaatgtgttgagagcaaataaggaactgaattttaatctttaacgtcaattactttaaataaatagccttatgtagttagcagctactatattagagaatataaatagaacatttctgtggttgctgctgggccagatagtgagcatttcaataaatcagaagaatttaattttttatagacacacagatctagaatactaattcatggtggcagggcctttggaaccctgtctcctatgtttattttctgtcttctcctcccaacccatttccttgttttctactctctttttatttaaatcctttggcagtaggtgtaaatggagaccttccattcactctccttcctgttcctcatttgcacctttgatcaaccacatttgggacagggagtatggacaggaccatttgtgggcggggggtggggggatgcagacagcaaggaatcaaggcaagaaattctgcgtttatgacttgaacatttgggtggggcagtttcctcagtattgagacagttgaccattaatccatgactccattatctagccaactcttacagatctcttgagaatctgaaaaaaacaaacaaacaaaacaaaaaatgctgtagatcctgtcctataaaaagaaattatgataccagatatagaattttacatatgaaaatttactccaggacaaaaatcacagcctcatggactcttttgggaacctgatacttaagggttctacccttggtttcactgtgggactgcctaagttgtgttttacctcttatgtcacacagaataaaattggttttaatacatgtgccaatttgctttgcagcatctggtacaaagccatagagccatagacttttcagaaaacaattgattcctgagaaaacaattctggaaaaaagaattaaatctaatataatccttttttgtcttgtacagtttcttagaagcacacctaattgtgtagggagattttgtcagcgattccctcattggcttgtgagtgcaacccctaaatggcatttaagttcttcgacatatacatgtgtgttatctatttctgcatattagtttacccccccaacatttagctatttaaaacaagagtaaacattatctcacatagtttctatgggtcaggaatttagaagcagcttagctaggtggttctgggtcagagtctcatgacatagtcaaaacattagccagggatgcagtcgtctgatgacttgcctggaggcagaagctctgcttccacagtggctctcattcatgtggttatcaaatatgtgctgcctgtatgcaggaagcctcagtcactcaataagtggccctctccatagtgctacttaagtatctcacatcattgcagctggcttcccctagactaagtaattcaagaaagaaggcaatgcagaagccacagtatcttttatgtcctagccttggaagtcacacaccattatttctggaatatcctattagttacacaggtcagtcctattcagtgtgggaatggactgcattaggcatggataccaagaggcatgaatcattggggtcatcttggaaagtagctaccacagtgtgtagggatgattttgtaccttcctttccttccccacaaagctgattgttctatttcctcttaagtccatccttggtttgggggcatttttttgtttacttgtttttccatagtagcattagacaggtgataatgggcattggtttataggaagcttcaaagtcagtgaaatgtcagttctgtagttgtttgcagagcacttatatctctgtacaccccatactgaaaataaagtaactgtgctcttaatcttaatttttcttatcataaactcactcttctcttatttttttttttttttgagacggagtctcgctctgttgcccaggctggagtgcagtggcatgatttcggctcattgcaacctctgcctcccaggttcacgccattctcctgcctcagcctcccgaatagctgggactacaggcgcccgccactatgcccagctaatttttggtatttttagtagagtcagggtttcaccgtgttagccaggatggtctctatctcctgactttgtgatccgcccacctcggcctcccaaagtgctgggattacagtcatgagccaccgcgcccggcccactcttctcttttaatgtaaagtctcttacagataatagaaatgtgcctgtagaaatcacagtgattttattgaggttagttctaaatgaaagtcatcttaatctcttttcttcttttgctaggaaatcttttctgctgccacctactgcccataagatggattattctcctcccttgtgctctgtgttccactcccctacttgttagctatatggtcttgggcacattccttaatctatttgtgcttcctttctcctatttgcaaaacagaggtaacagttgtacctatttctcagatttgctgtgtgagctaaatgacatgactgcaaaatacttagtgtggtgtctggcatattggtaagcagtatgctgtcactagtatttactttggtgatgatgttggttattgcagtcacactagattataaatatatgtatttttagatgtctcttttaccttcccatctattgaacaattgctgtatgccagatactgtgctaagcacttcataggcctcatttcataggtgggtggtattactaccattttaccaatgaggaaatgagagatagattaaataacgtgttccaaaatcacacagctattaagttgtacaagtaggcttcatactcaataaatgtttatagagttggtttagttttaagaaagatgtatcagtgtgaagttcagaagagcatctctggttgaaaacaagagactagagtgggttaatgatccatagtcagatcatttataatgaaaatgatcttagcctctcttttaaaccataatgccatttagcgtcactttaccagtgcatatgtaacgggaagggaatgtaaggagaagtaaaatgaactttgctgtttttatgttgtggagtcactatttaaatgtttgagaagaaaagttacagaaattacactaaactaaatccaatgacaatttatcggaattttaaattaataaacattttaatagaaagctcattaattcagattccattgtgtaaaataaatttacagagtgatggtgggaacaaaataagcatgtgccaagatttgtttcttgagttatttctaaaaaggtggtaatctgtgttgtcttgtttcctgatcttgaactagtttcttcccagccaccaagaggtcagctcctcctactttacctggcccaggggccagtccatctttatgatgggattgtagagattatgaaatgtaactttactcagtcagtaagctgggtggtcttttaaacctactattgagtattgaatttatagaactttgcctttttttgttttactttttccctctctgtactttgtcttcctctgaaaaagagaactctgttcttctcagcaaggacatgtgtgatttgattttaagtggagctcattgcatcaggcaaaagtttttttgatgaatgtctgtaggactgtgactttcgatatcaaagatatttataatcaaaataattagcgttaaaattatgttcattaagtgatttgaaaataattcaagagaaaaatatgagatcattacaggtctgaagagcatgggtgtctatcaaattttattgttcagaaagtgaatttgtttctggttttatgctcacttaaaaggcttgtgctttttactgtgatcatgcaagcagtcgttgaatttatccaagaaagaggagactggttttggcaatgtcacaagctcacaggctgtagtcagtgaaaggacatctgcacattaatgccaaataggattttgctaaagagcaaattaacataaatgggctggtggccatgtagatgaggtaaaactgatgtcagccagtgtagttaatggctgccgtggccagagctgctctctccatgttggcatgggtgctgatgtagtggtcaaacagatagataaatttctgatgatcagtgcctcccataagtcctggggaagaccagaatgacagttctacctgttatcttcccatactgaaaatgagaaggaaatgctgttggatgatggtcatgcataggttctgagtgtcatcagagcatgtgacatgctgcaataagcatgggtttgtggtgttccaggttttctctggattttttttttaatgcagcctgtgcatgaatcatgaaaatgaacattttatctgagaaaggcttggaggaatctgtggtctagacaggaattgatgttaggttattgatgcacagaatgtggatgatcattaagggtaaaacttctttaaacagggcatatctaaggaaggtcaggagtcacgaatgaagtgttagcatagttcctcagtgattgttagaaaaggtggtcagcctggctttggagagtaggttctgtgtcatataccctttcctgccacctgttcatttgccatactgatgaattaatatgaccccattacatgggctatatgaacctaatgtgtggattctgatgccataatatcttcacttgtgtacatgacaacaagattagaagcagctttagctattttgaaggaaattaaatgaattaaacctaatcattcacattaagagatattcacattccttccccaaaggctgtgctaaatgtatataagggcagcttatcgggaaattaggaagtataatttttatagattggctttagaaatgataaactgaacagctaatccatcatagagttccctatgtgtccttggatattctgtgtctccctctaattcagaaattcaaagtagaaagatccaggaccaaacgttgcattcaaccatgacattggttattctactcaccatagcacatggtagatctacaaatcccttcagtcctagagagaaaaaaacttctgagtctgcctttcggtgtcaccaaccttgatggcattgtggcttctagagagatggcttttcaaagttgcccatcccagtcaagctacctctcttgctcagggcttagtgtgaaatctttgggagaaaatttggccagcagtaccttctctgtcattaagagagatgacaaggatgcaacttgatagccactaaaacagcagtctactaaaccaatttggacacagttagatgcccacttaaatgttaatggatgctgtgaaataataatagaccttaaaaggggggaggagataatggttagttgtagagttgtacgtatttctaaaagattagggcaaagtaagctctaagtaagttctacgtcaagatttgactacaagatgctttccttttcttaatttttaatttttgtggggacatagtaggtgtatatatttatgggttacctgagatattttgatatagacatggaatgtgtaataatcatatcagggtaaatgggatattcatcacctcaagcatttatcctttatgttacaaacaatccagtcatactttgttattttaaaatgtacaattaaattatttttgtactgtagtcaccctgttttgctagcaaatactaggtcttatacatttttttttttgtacccattaaccatccccacttcccccacccccgctacccttcccaacctctggtaacaatccttctctctaactccgtgagttcagttgttttaaattttagctcctacaaataagtgagaacatgcaaagtttgtctttctctgtctggcttatttcacttcacataatgacttcagttccatccatgttgttgcaaatggcaggatctcattctttttctttttcttttctcttttctttcttctctctctctctcttttttttttttttttttttttttaaagacggtatcttactgtgtctcccaggtggtcttggctcacttgcagcctcaacctctctggctcaagcagtcctcccacttagcctcccatctttctctcttatggctacatattaccccattgtgtatatataccacattttcttttatccattcatctgttgatacaagatgctttattaaattgactaagtgaattggtactgcagttgctgtttcttttttgctggttgtctgtcttccatctctatgaataatgaagagggtctgttctgcccatcaacactgacccacgttactttcattgcctccacctatttacttacttaagcagatggcatattgtaacatggttcaaatttttagcaagttttcccagctgtgcactgttcatttattttcaagtgaactagactccagctgagtgttcatatacagctgatcccagctgtagcaagagctttgactgaaatggtctctggctccctgagtctgcaacttattcaaagacactgtcttcccggtaccaggaccatgctgaggcatgaacttaaatccaagaatagagaaattcagatttcatagtgttggccttatttaaaacaacgttccaaataacagcttggaaggccatacagttcacagcctgcagttgaaacagtgtacttgttctcttttgttgttatagcattgctcatgctctctcttgggactttttgacttgcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatatt

SEQ ID NO: 2 - DNA, 1639 bp, Homo sapiens
  CDS (233)..(1534)
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 3 - protein, 433 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 4 - DNA, 1600 bp, Homo sapiens
  CDS (233)..(1495)
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 5 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 6 - DNA, 1540 bp, Homo sapiens
  CDS (233)..(1435)
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 7 - protein, 400 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQDANTGDRGQTNNAASASASNST

SEQ ID NO: 8 - DNA, 1501 bp, Homo sapiens)
  CDS (233)..(1396)
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 9 - protein, 387 aa, Homo sapiens)
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQDANTGDRGQTNNAASASASNST

SEQ ID NO: 10 - DNA, 24 bp, artificial sequence
  Synthetic oligonucleotide designated GSKEx3F
tataaaagctctaaacactctcaa

SEQ ID NO: 11 - DNA, 24 bp, artificial sequence
  Synthetic oligonucleotide designated GSKEx3R
aatgctttcctgatataactaatg

SEQ ID NO: 12 - DNA, 24 bp, artificial sequence
  Synthetic oligonucleotide designated GSKEx6F
gcttttggtgccttcttaggtgac

SEQ ID NO: 13 - DNA, 21 bp, artificial sequence
  Synthetic oligonucleotide designated GSKEx6R
cgaaacattgggttctcctcg

SEQ ID NO: 14 - DNA, 23 bp, artificial sequence
  Synthetic oligonucleotide designated GSKEx9F
gccatgtctgtggatgatgaatc

SEQ ID NO: 15 - DNA, 22 bp, artificial sequence
  Synthetic oligonucleotide designated GSKEx9R
caatgaatcacccaagaggctg

SEQ ID NO: 16 - DNA, 22 bp, artificial sequence
  Synthetic oligonucleotide designated GABABL.SNP1F
tctgatggcttctctggttccc

SEQ ID NO: 17 - DNA, 23 bp, artificial sequence
  Synthetic oligonucleotide designated GABABL.SNP1R
tcttgtgctgtgtgtgtgctgtc

SEQ ID NO: 18 - DNA, 23 bp, artificial sequence
  Synthetic oligonucleotide designated GABABL.SNP2F
ttgagggagtgtttctgggatag

SEQ ID NO: 19 - DNA, 22 bp, artificial sequence
  Synthetic oligonucleotide designated GABABL.SNP2R
tgtgtgtgtgctgtccaagtgg

SEQ ID NO: 20 - DNA, 24 bp, artificial sequence
  FSTL1.SNP1F
gcgtctcaaggacagtgaaacatc

SEQ ID NO: 21 - DNA, 23 bp, artificial sequence
  Synthetic oligonucleotide designated FSTL1.SNP1R
acaggggctgaaatccaaactac

SEQ ID NO: 22 - DNA, 23 bp, artificial sequence
  Synthetic oligonucleotide designated FSTL1.SNP2F
gggtccacgcaatagtttcagac

SEQ ID NO: 23 - DNA, 24 bp, artificial sequence
  Synthetic oligonucleotide designated FSTL1.SNP2R
aagccaaccgctcaacaacactgg

SEQ ID NO: 24 - DNA, 24 bp, artificial sequence
  Synthetic oligonucleotide designated NR1I2.SNP1F
cctttacttcagtgggaatctcgg

SEQ ID NO: 25 - DNA, 23 bp, artificial sequence
  Synthetic oligonucleotide designated NR1I2.SNP1R
tcacacacatcttttggctggag

SEQ ID NO: 26 - DNA, 24 bp, artificial sequence
  Synthetic oligonucleotide designated NR1I2.SNP2F
tgtttgaggtcagcatcatagtgg

SEQ ID NO: 27 - DNA, 22 bp, artificial sequence
  Synthetic oligonucleotide designated NR1I2.SNP2R
ccagagagcatcagtaatgggg

SEQ ID NO: 28 - DNA, 22 bp, artificial sequence
  Synthetic oligonucleotide designated COX17.SNP1F
caggacatattcaagcagagtt

SEQ ID NO: 29 - DNA, 22 bp, artificial sequence
  Synthetic oligonucleotide designated COX17.SNP1R
tttagcactgttattagcattc

SEQ ID NO: 30 - DNA, 22 bp, artificial sequence
  Synthetic oligonucleotide designated COX17.SNP2F
ctgtgtcctactgtctctaaat

SEQ ID NO: 31 - DNA, 21 bp, artificial sequence
  Synthetic oligonucleotide designated COX17.SNP2R
gcttcaaccactcattccttc

SEQ ID NO: 32 - DNA, 22 bp, artificial sequence
  Synthetic oligonucleotide designated GSKRT-2F
tgttggagttcccaggaccttg

SEQ ID NO: 33 - DNA, 24 bp, artificial sequence
  Synthetic oligonucleotide designated GSKRT-R
agtaactggtggtttttcctgtgc

SEQ ID NO: 34 - DNA, 24 bp, artificial sequence
  Synthetic oligonucleotide designated GSKRT-1F
cgagggacactaaatacagttcaa

SEQ ID NO: 35 - DNA, 20 bp, artificial sequence
  Synthetic oligonucleotide for fluorescent single nucleotideprimer extension analysis
ttctgcccttgcttctttgt

SEQ ID NO: 36 - DNA, 20 bp, artificial sequence
  Synthetic oligonucleotide for fluorescent single nucleotideprimer extension analysis
tgttagtgcaaagcagctgg

SEQ ID NO: 37 - DNA, 38 bp, artificial sequence
  Cy5 labelled synthetic oligonucleotide for fluorescent single nucleotide primer extension analysis
catatatgaaaaagaaaatgtaaactgtaactatctct

SEQ ID NO: 38 - DNA, 20 bp, artificial sequence
  Synthetic oligonucleotide for MALDI-TOF analysis of SNP in GSK-3 beta
ttctgcccttgcttctttgt

SEQ ID NO: 39 - DNA, 20 bp, artificial sequence
  Synthetic oligonucleotide for MALDI-TOF analysis of SNP in GSK-3 beta
tgttagtgcaaagcagctgg

SEQ ID NO: 40 - DNA, 38 bp, artificial sequence; identical to SEQ ID NO: 37
  Synthetic oligonucleotide for MALDI-TOF analysis of SNP in GSK-3 beta

SEQ ID NO: 41 - DNA, 20 bp, artificial sequence
  Sequence specific synthetic oligonucleotide for fluorescence adapted SSCP analysis of SNP in GSK-3 beta
ttctgcccttgcttctttgt

SEQ ID NO: 42 - DNA, 15 bp, artificial sequence
  Tail synthetic oligonucleotide for fluorescence adapted SSCPanalysis of SNP in GSK-3 beta
ccggcagcaaaattg

SEQ ID NO: 43 - DNA, 38 bp, artificial sequence
  Synthetic oligonucleotide for fluorescence adapted SSCP analysisof SNP in GSK-3 beta
tgaccggcagcaaaattgttctgcccttgcttctttgt

SEQ ID NO: 44 - DNA, 20 bp, artificial sequence
  Sequence specific synthetic oligonucleotide for fluorescence adapted SSCP analysis of SNP in GSK-3 beta
tgttagtgcaaagcagctgg

SEQ ID NO: 45 - DNA, 18 bp, artificial sequence
  Tail synthetic oligonucleotide for fluorescence adapted SSCPanalysis of SNP in GSK-3 beta
tgtaaaacgacggccagt

SEQ ID NO: 46 - DNA, 38 bp, artificial sequence
  Synthetic oligonucleotide for fluorescence adapted SSCP analysisof SNP in GSK-3 beta
tgttagtgcaaagcagctggtgtaaaacgacggccagt

SEQ ID NO: 47 - DNA, 18 bp, artificial sequence
  Cy-5 labelled synthetic oligonucleotide for fluorescence adapted SSCP analysis of SNP in GSK-3 beta
tgaccggcagcaaaattg

SEQ ID NO: 48 - DNA, 18 bp, artificial sequence
  Cy-5 labelled synthetic oligonucleotide for fluorescence adapted SSCP analysis of SNP in GSK-3 beta
tgtaaaacgacggccagt

SEQ ID NO: 49 - DNA, 4356 bp, Homo sapiens
catttatatttaaatatgttattgacgagttattctaatttgtcttcctttccaatcttgatccaaatgtacttatattttacagattggaaatatggtagatatacaacctgaagttctatatttttcaattagtattgtttcctaaacatatttctgagttacttttggtggttgtatttatttttactggctgcctaatatggagttaatacagcatagtttactttactcctctattatcctgtgatattttcctttattttctcttggcccacatcttagacattacataatttcaaactatctcaaggtctccaaataccttgttaaacatttcagccattgcataaccttttctctttgcctggaatgttctttccttccttatccatctgtacaactgtacaactattctttgtctaaactagtggttcataaacttttttttttttttttttgagacggagttttgctcttgttgccttggctggagtgcaatggtgcgatctcagctcactgcaacgtccacctcctgggttcaagcaattatcctgcctcagcctcccaagtaactggtattatagacgcccgccaccacgcccagctaagttttaaagttttagtagagacggggtttcaccatgatgaccaggctagtcttgaactcctaacctcaggcgatccacctgcctcggcctcccaaattgctgggattacagtcataagccaccactcctggcttgtccataaactcttgatcttgagctaacatgatatacacattataagatacacacagaaataaaaaaaggattaaaggtgcactattttaaagataaatttcaaccggcccggtgtggtggcttacgcctgtaatcctgacactctgtgaggctgaggcaggcagatcacctgaggtcgggagttcgagaccagcctgaccaacatgaagaaaccccgtctctactaaaaatacaaaagtagctggcatggtggcacatgcctgtgatgggaactacttgggaggctgaggcaggagaatcacttcaatcggggaggcggaggttgcggtgagccgagatcgcgccactgcactccagcctgggcaacaagagtgaaactccgtctcaaaaataaataaataaataaagatacatttcaattatttaaggtcccaaaccattttgttttcaccctcctactgctatcggggcccctaacaattattaacactttttccaatgcctcattggccaagggatgtttttaggttggcctgcagggtttgaaaatttggttaccaacatttaaaaatgcagagatttcacacaaaaatccaattttggggtttcttttgggaggaaaaaaattgaacatctgagaacactggacccacattcctgagggcaacaatctgctagagttgagcagctgttccttttagttatggcatggatattccaatttgccattttccctatcattccccatttgtcttacacaaggccagtctcactcatttgctttacttgtctggcctctgtagccatttgaatctgcaaacactatctagacctttcctgtcttcccatcaaagcttactttgttctgtcccaagtccttcatcagtgtttcaaagcaagagccaggtaatctgatcaaatataggtcctttwacatgtgctttctggagacagcatcttaacagaaagagatgactctgcaggtagaaattacaggcaccactggattacaatcaaggatggcaactgtctgttctactttctctgttatcttgacgtagccatacctcactgttctaaagcagaccaaaggaactggttctatggtagaacaatggacgctggtcttatgtatgaaatcttctcaagctgcacttttatagatcaccctagttccaaaagatccaaagctaccacgggctcctctgacccccccatttcctcggaggcttcaggatatccggcctgacggcatttcccctcacctactttcggggcccgttttgcgctgggcagttgcgcccaaggagcgcgcgcatccagcatgagctcatttctcatgggcgtttccagaggcccggccgggcgcgcacaccaagcgcagcaagcctctgtatcaatggcccccgcggctgggcggggcgcggccgcccaaaggcgctgcctttctggaagctttgttcccattttagcgtctgagagcttgcagccggctgggaaggcccccttggtccgtctggccctttcggggaagaggccaacactcggcacacgcgatccacggcagaggggagccttgggcgcgcagaattggctgcgccccgccgagagcctcctgtgggtggggagagcccctccacccctctgctcgcttgagcgctcagagcccagggccgccgaccgcagcactttccgatttgctgcaccgagggcccgcggtccctgtgtgcggtttccaccgttgttggaggcggccgcaggcgaaccctcgggtcgtcagccacgacccgagtcaggcatctccccgctcctgggaccggggccgaaggccaatcacactgcagctaggtctttgcggattggacggcagtgagagccgattggccgccgccgcgagtttcgggctccctccctctcccttttgccccaactccagcgcctaagctttaggccaatgagacagcgctttatagacgccctcccttcgctttcttctccccaccttggagagggaggggaagtcctgactggccagactgtcctcggaagcccccttctcttcaccaatcaccgaaggaggtacgctcccagcggagttttccagccaatcacaaagcggcggcggcgcccagccgtgcagtttcaccagcgtctctgggtttcaccgtcctcaactcttcaagcctcttcgtaagggccggcgactctgattggccactcttgccattgtcaaatgtctcctccagccagccaccgaacaaggcgaattcaccctttccgttcggctaccttcggcattttccgctctttgggcgtggcttgccagcgtcactttctaattggtttctcaggctgatcggctttttccgggaggagccgcaaacaaacgacgtccgtgattggctccgttcgggcttcggctcccagccgaagcgggcgagcgtggggctcggccggcgattcccagacgcctgttacgcgggcggcggggcgctgggcggtgtaaggctgggtgggggaggaaggaggaggaggacgagtaggaggggggaggaggagtggggaagtgcaaggcggctgcgcagacagcgctcctcacacagagcagcycctgacccgggcgaatgcgggcttgtgccgccgccgccgccgccgccgcccgggccaagtgacaaaggaaggaaggaagcgaggaggagccggccccgcagccgctgacagggctctgggctggggcaaagcgcggacacttcctgagcgggcaccgagcagagccgaggggcgggagggcggccgagctgttgccgcggacgggggagggggccccgagggacggaagcggttgccgggttcccatgtccccggcgaatggggaacagtcgaggagccgctgcctggggtctgaagggagctgcctccgccaccgccatggccgctggatccagccgccgcctgcagctgctcctggcgcaatgaggagaggagccgccgccaccgccaccgcccgcctctgactgactcgcgactccgccgccctctagttcgccgggcccctgccgtcagcccgccggatcccgcggcttgccggagctgcagcgtttcccgtcgcatctccgagccaccccctccctccctctccctccctcctacccatccccctttctcttcaagcgtgagactcgtgatccttccgccgcttcccttcttcattgactcggaaaaaaaatccccgaggaaaatataatattcgaagtactcattttcaatcaagtatttgcccccgtttcacgtgatacatatttttttaggatttgccctctcttttctctcctcccaggaaagggaggggaaagaattgtattttttcccaagtcctaaatcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatc

SEQ ID NO: 50 - DNA, 23 bp, artificial sequence
  Synthetic oligonucleotide for amplifying GSK3 beta SNP rs334558
aaaagatccaaagctaccacggc

SEQ ID NO: 51 - DNA, 24 bp, artificial sequence
  Synthetic oligonucleotide for amplifying GSK3 beta SNP rs334558
tccttccttcctttgtcacttggc

SEQ ID NO: 52 - DNA, 24 bp, artificial sequence
  Synthetic oligonucleotide for amplifying GSK3 beta
ccagtggtgagaagaaagatgagg

SEQ ID NO: 53 - DNA, 21 bp, artificial sequence
  Synthetic oligonucleotide for amplifying GSK3 beta
tgttggagttcccaggacctg

SEQ ID NO: 54 - DNA, 24 bp, artificial sequence
  Synthetic oligonucleotide for amplifying GSK3 beta SNP rs3755557
gccatcctgattgtaatccagtgg

SEQ ID NO: 55 - DNA, 25 bp, artificial sequence
  Synthetic oligonucleotide for amplifying GSK3 beta SNP rs3755557
gcttactttgttctgtcccaggtcc

(19) United States
(12) Patent Application Publication
(10) Pub. No.: US 2009/0041862 A1
(43) Pub. Date: Feb. 12, 2009

(54) Detecting disease association with aberrant glycogen synthase kinase 3beta expression

(76) Inventors: Peter Robert Schofield, Marsfield (AU); John Bing Jee Kwok, Potts Point (AU)

Correspondence Address: PEPPER HAMILTON LLP, 400 Berwyn Park, 899 Cassatt Road, Berwyn, PA, 19312-1183, US

(21) Appl. No.: 11/579,796

(22) PCT Filed: May 6, 2005
(86) PCT No.: PCT/AU05/00648

§ 371 Date: Jul. 21, 2008

Related U.S. Application Data
(60) Provisional application No. 60/569,098, filed on May 7, 2004.
(60) Provisional application No. 60/626,455, filed on Nov. 9, 2004.

(51) Int. Cl. A61K 33/00; C12Q 1/68; C12Q 1/48; G01N 33/573; C07H 21/04

(52) U.S. Cl. 424/722; 435/6; 435/15; 435/7.4; 536/24.31

(57) ABSTRACT

The present invention provides a method for diagnosing a disease or disorder associated with aberrant GSK-3β expression and/or activity or for determining the predisposition of a subject to the disease or disorder. In particular, the methods of the present invention comprise detecting a marker that comprises one or more polymorphisms and/or one or more allelic variants of a glycogen synthase kinase 3β gene. The present invention also relates to a method for identifying new markers that are diagnostic of a disease or disorder associated with aberrant GSK-3β expression and/or activity. Furthermore, the present invention relates to methods of identifying and producing candidate compounds for the treatment of a disease or disorder associated with aberrant GSK-3β expression and/or activity.

### FIELD OF THE INVENTION

The present invention relates to a method for diagnosing a disease or disorder and/or for determining the predisposition of a subject to a disease or disorder. In particular, the methods of the present invention comprise detecting a marker that comprises one or more polymorphisms and/or one or more allelic variants of a glycogen synthase kinase 3β gene.

### BACKGROUND OF THE INVENTION

1. General

This specification contains nucleotide and amino acid sequence information prepared using Patent In Version 3.3, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Unless specifically stated otherwise, each feature described herein with regard to a specific embodiment of the invention, shall be taken to apply mutatis mutandis to each, and every other embodiment of the invention.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purposes of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, histology and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Immobilized Cells and Enzymes: A Practical Approach (1986) IRL Press, Oxford, whole of text;
6. Perbal, B., A Practical Guide to Molecular Cloning (1984);
7. J. F. Ramalho Ortigão, “The Chemistry of Peptide Synthesis” In: Knowledge database of Access to Virtual Laboratory website (Interactiva, Germany);
8. Sakakibara, D., Teichman, J., Lien, E. Land Fenichel, R. L. (1976). Biochem. Biophys. Res. Commun. 73 336-342
9. Bodanszky, M. (1984) Principles of Peptide Synthesis, Springer-Verlag, Heidelberg.

2. Description of the Related Art

Neurodegenerative diseases are a group of disorders characterized by changes in normal neuronal function, leading in the majority of cases to neuronal dysfunction and even cell death. Currently, it is estimated that there are in excess of one hundred neurodegenerative diseases. However, we still have little understanding of the etiological cause of these diseases. The most consistent risk factor for the development of a neurodegenerative disease, such as, for example, Alzheimer's disease or Parkinson's disease, is age (Tanner, Neurol. Clin. 10: 317-329, 1992). For example, such diseases are more prevalent in aged or aging persons, with a doubling of risk every five years after the age of 65.

Over the past century, the growth rate of the population aged 65 and beyond in industrialized countries has far exceeded that of the population as a whole. Accordingly, it is anticipated that, over the next generations, the proportion of elderly citizens will double, and, with this, the proportion of persons suffering from a neurodegenerative disease.

Two of the most common and most studied forms of age-related neurodegenerative disease are Alzheimer's disease and Parkinson's disease. Currently, it is estimated that there are 4.5 million cases of Alzheimer's disease and 1.2 million cases of Parkinson's disease in the US alone. It is estimated that in the period from 2001 to 2010 an additional 1.5 million Alzheimer's disease cases will be diagnosed in the US, while currently there are approximately 480 new cases of Parkinson's disease per million people per year diagnosed. Alzheimer's disease alone is the third most expensive disease in the United States, costing approximately US$100 billion each year for therapy and/or care of sufferers.

### Alzheimer's Disease

Alzheimer's disease is a complex multigenic neurodegenerative disease characterized by progressive impairments in memory, behavior, language, and visuo-spatial skills, ending ultimately in death. Hallmark pathologies of Alzheimer's disease include granulovascular neuronal degeneration, extracellular neuritic plaques with β-amyloid deposits, intracellular neurofibrillary tangles and neurofibrillary degeneration, synaptic loss, and extensive neuronal cell death. It is now known that these histopathologic lesions of Alzheimer's disease correlate with the dementia observed in many elderly people.

Alzheimer's disease is commonly diagnosed using clinical evaluation including, physical and psychological assessment, an electroencephalography (EEG) scan, a computerized tomography (CT) scan and/or an electrocardiogram. These forms of testing are performed to eliminate some possible causes of dementia other than Alzheimer's disease, such as, for example, a stroke. Following elimination of other possible causes of dementia, Alzheimer's disease is diagnosed. Accordingly, current diagnostic approaches for Alzheimer's disease are not only unreliable and subjective, they do not predict the onset of the disease. Rather, these methods merely diagnose the onset of dementia of unknown cause, following onset.

Furthermore, not all causes of dementia are easily detectable by methods currently used for the diagnosis of Alzheimer's disease. Accordingly, a subject that has suffered an ischemic, metabolic, toxic, infectious or traumatic insult to the brain may also present with dementia, and, as a consequence, be incorrectly diagnosed with Alzheimer's disease. In fact, the NIH estimates that up to 45% of subjects diagnosed with Alzheimer's disease actually suffer from another form of dementia, not necessarily caused by a neurodegenerative disease.

Genetic studies of subjects with a family history of Alzheimer's disease indicate that mutations in genes, such as, for example, amyloid precursor protein gene, presenillin-1 and/or presenillin-2 cause some forms of this disease. However, these forms of Alzheimer's disease represent less than 5% of total cases of the disease.

Studies to identify polymoiphisms and alleles that confer susceptibility to Alzheimer's disease have identified a large number of polymorphisms and mutations (reviewed in Rocchi et al., Brain Res. Bull., 61: 1-24, 2003). The most widely studied of these is the ε4 isoform of the apolipoprotein E gene. A number studies have shown an association between apolipoprotein E ε4 (ApoE-ε4) and late-onset familial and sporadic forms of Alzheimer's disease (for example, Corder et al., Science 261: 261-263, 1993). However, less than 50% of non-familial Alzheimer's disease sufferers are carriers of the ApoE-ε4 isoform (Corder et al., Science 261: 261-263, 1993).

### Parkinson's Disease

Parkinson's disease is a progressive disease with a mean age at onset of 55, and the incidence increases markedly with age, from 20/100,000 overall to 120/100,000 at age 70. This disease is characterized by resting tremor, rigidity, slowness or absence of voluntary movement (i.e. bradykinesia, hypokinesia and/or akinesia), postural instability, and freezing. Over time, symptoms worsen, and prior to the introduction of levodopa, the mortality rate among Parkinson's disease patients was three times that of the normal age-matched subjects. The majority of Parkinson's disease patients suffer considerable motor disability after 5-10 years of disease onset, even when expertly treated with available symptomatic medications.

Parkinson's disease is diagnosed by clinical evaluation of a subject. Subjects that have two or more of the principal symptoms, one of which is resting tremor or bradykinesia, are diagnosed as suffering from Parkinson's disease. Positron-emission tomography (PET-scan) using radio-labeled dopa is helpful in confirming a diagnosis in difficult cases. However, this test is not widely available. A SPECT-scan is a simpler test using a variety of different isotopes and is widely available but is less reliable in confirming PD. Magnetic resonance imaging (MRI) is useful in excluding other conditions such as tumors, strokes, and hydrocephalus. However, MRI cannot confirm PD.

As with Alzheimer's disease, diagnosis of Parkinson's disease is only accurate, or even possible, following onset of the disease. Accordingly, methods currently in use for diagnosis of these diseases are of no use in determining a predisposition to a neurodegenerative disease, or in a prophylactic method of treatment for a neurodegenerative disease.

The observation that a family history of Parkinson's disease is associated with an increased risk of developing the disease has provoked a considerable research effort to determine genes, alleles of genes or mutations that cause neurodegenerative disease or are associated with a susceptibility to a neurodegenerative disease.

Several mutations that cause Parkinson's disease have been identified, e.g., Ala⁵³→Thr (A53T) and Ala³⁰→Pro (A30P) in α-synuclein are associated with dominantly inherited PD (Polymeropoulos et al., Science 276, 2045-2047, 1997 and Kruger et al., Nat. Genet. 18, 107-108, 1998), loss-of-function mutations in the gene encoding parkin cause recessively inherited parkinsonism (Kitada et al., Nature 392, 605-608, 1998) and a dominant mutation (193M) in UCH-L1 was identified in one family with inherited PD (Leroy et al., Nature 395, 451-452, 1998). Notwithstanding the discovery of genes responsible for several forms of Parkinson's disease, these mutations are relatively rare and the number of subjects suffering from these forms of the disease represent a small proportion of Parkinson's disease patients.

Researchers have also attempted to use linkage and association studies to identify genes and/or alleles of genes that are associated with a susceptibility to Parkinson's disease. However, these studies have met with limited success. For example, Persad et al., Neuroepidemiology, 22: 357-361, 2003 showed that there was no significant association between polymorphisms in the CYP2D6 gene and Parkinson's disease in Asian populations, while Oliveira et al, Arch. Neurol., 60: 975-980, 2003, showed that there is no association between polymorphisms in the Parkin gene and Parkinson's disease in Caucasian subjects.

Based on the discussion herein it is clear that there is a need to develop improved diagnostic methods for determining a predisposition to development of a neurodegenerative disease in a subject, and for the early diagnosis of a neurodegenerative disease. Diagnostic assays that rapidly and reliably diagnose a neurodegenerative disease prior to onset of the disease are particularly desirable as are indicators of whether or not a subject will respond to a particular treatment.

### SUMMARY OF INVENTION

In work leading up to the present invention the inventors sought to identify polymorphisms that are significantly associated with development of a neurodegenerative disease for use in the development of a new diagnostic and/or prognostic method. The inventors studied polymorphisms associated with Parkinson's disease and Alzheimer's disease as a model of neurodegenerative disorders generally.

As exemplified herein, the present inventors have identified polymorphisms in the glycogen synthase kinase 3β (GSK-3β) gene that are associated with a Parkinson's disease or an Alzheimer's disease. In particular, the inventors found that a polymorphism in GSK-3β associated with altered splicing of encoded mRNA is also associated with a neurodegenerative disease. Individuals homozygous for this polymorphism have a significantly greater risk of developing a neurodegenerative disease than subjects that are heterozygous for the polymorphism.

Furthermore, the present inventors have determined that two polymorphisms located within the promoter of the GSK-3β gene are associated with increased transcription of the gene. Detection of either of these polymorphisms is indicative of a neurodegenerative disease.

Furthermore, when a polymorphism in the promoter of GSK-3β is detected in combination with the polymorphism affecting GSK-3β splicing there is a further increase in risk of developing a neurodegenerative disease.

Each of the polymorphisms have also been shown to be correlated with the development of a psychiatric disorder, for example, a bipolar affective disorder.

Accordingly, polymorphisms within the GSK-3β gene are statistically correlated with the development of one or more neurodegenerative diseases or psychiatric disorders, and represent attractive targets for diagnostic assays for determining the predisposition of a subject to such disease(s).

The polymorphisms identified by the inventors are also useful for the diagnosis of another disease or disorder associated with aberrant GSK-3β expression and/or activity, such as, for example, a disorder associated with aberrant glucose metabolism (e.g., diabetes), stroke induced ischemia or an inflammatory disease.

Accordingly, the present invention provides method for determining a disease or disorder associated with aberrant glycogen synthase kinase-3β (GSK-3β) expression or activity or a predisposition to the disease or disorder, said method comprising detecting a marker within a GSK-3β gene or an expression product thereof that is associated with the disease or disorder in a sample derived from a subject, wherein the detection (or detected marker) is indicative of the disease or disorder or a predisposition to the disease or disorder in the subject.

For the purposes of nomenclature, the nucleotide sequence of a human GSK-3β gene is exemplified herein as SEQ ID NO: 1. Preferably, a GSK-3β gene comprises a nucleotide sequence at least about 80% identical to the sequence set forth in SEQ ID NO: 1.

As used herein, the term “marker” shall be taken to mean a nucleic acid that comprises a nucleotide sequence associated with a disease or disorder associated with (or characterized by) aberrant GSK-3β expression and/or activity. Alternatively, or in addition, the marker is linked to a polymorphism in a genome wherein said polymorphism is associated with a disease or disorder associated with aberrant GSK-3β expression and/or activity. Preferably, the nucleic acid is associated with a neurodegenerative disease and/or a predisposition to a neurodegenerative disease.

In those embodiments related to detection of a marker that is in a region of the genome that is transcribed or that controls transcription, the term “marker” shall also be taken to mean an expression product of a gene or an allele of GSK-3β that is associated with a disease or disorder. For example, the marker may comprise or be within a pre-mRNA molecule, a 5′capped mRNA, a polyadenylated mRNA and/or a mature or processed mRNA.

In those embodiments related to antigen-based or antibody-based assays, those skilled in the art will appreciate that the term “marker” also means a peptide, polypeptide or protein that comprises an amino acid sequence encoded by an allele of a gene that is associated with a disease or disorder that is associated with aberrant GSK-3β expression and/or activity. Alternatively, or in addition, the proteinaceous marker is associated with a polymorphism in a genome wherein said polymorphism is associated with a disease or disorder that is associated with aberrant GSK-3β expression and/or activity.

As used herein, the term “associated with a disease or disorder” shall be taken to mean that the detection of a marker is significantly correlated with the development of a disease or disorder in a subject or that the absence of a marker is significantly correlated with the development of a disease. For example, a marker that is positively associated with a disease is a polymorphism, the detection of which is associated with the development of the disease. A marker that is negatively associated with a disease is, for example, an exon of a GSK-3β gene that is spliced out of a GSK-3β mRNA. Those forms of GSK-3β lacking the exon are associated with the development of a disease.

In a preferred embodiment, a marker is significantly correlated with the development of a disease or disorder in a plurality of subjects. Even more preferably, the marker is significantly correlated with the development of a disease or disorder in a plurality of unrelated subjects.

By “detection is indicative of the disease or disorder” is meant that the detected marker is indicative of the disease or disorder associated with aberrant GSK-3β expression and/or activity by virtue of the association between the marker and the disease or disorder.

As used herein, the term “predisposition to a disease or disorder” shall be taken to mean that a subject is susceptible to a form of a disease or disorder or is more likely to develop the disease or disorder than a normal individual or a normal population of individuals. In this regard, a marker that is indicative of a predisposition to a disease or disorder may itself cause the disease or disorder or, alternatively, be correlated with the development of the disease or disorder.

By “disease or disorder associated with aberrant GSK-3β expression and/or activity” is meant any disease or disorder that is caused by increased or decreased GSK-3β expression and/or activity or in which the level of GSK-3β expression and/or activity is altered compared to a normal and/or healthy individual. Accordingly, the present invention is useful for diagnosing, for example, a disease or disorder selected from the group consisting of a neurodegenerative disease, a psychiatric disease, a disease or disorder associated with aberrant glucose metabolism, an inflammatory disease, a cancer, muscle hypertrophy, epilepsy and stroke induced ischemia. Preferably, the disease or disorder is a neurodegenerative disease.

While the present invention contemplates any marker in a GSK-3β nucleic acid or polypeptide, it is preferred that the marker comprises or consists of a polymorphism within a GSK-3β gene. By “polymorphism” is meant a difference in the nucleotide sequence of a specific site or region of the genome of a subject that occurs in a population of individuals. Accordingly, a “polymorphism that is associated with a disease or disorder associated with aberrant GSK-3β expression and/or activity” means that a particular form of the polymorphism is correlated with the development of the disease or disorder in a subject, i.e., in a population of subjects, those that suffer from the disease or disorder are more likely to comprise the specific polymorphism that those subjects that do not suffer from the disease or disorder. Preferably, the polymorphism is associated with a neurodegenerative disease.

Preferably, the marker that is associated with the disease or disorder comprises a polymorphism that is in its homozygous form. By “homozygous form” is meant that the same form of the marker associated with a disease or disorder occurs at the same locus on homologous chromosomes in a subject or sample derived therefrom. For example, the same nucleotide is found at the site of a single nucleotide polymorphism (SNP) located within a GSK-3β gene on both copies of chromosome 3 in a subject.

In a preferred embodiment, a marker that is associated with a disease or disorder comprises a SNP. Methods for determining a SNP that is associated with a specific disorder are known in the art and/or described herein. Preferred SNPs for use in the method of the present invention include, for example, a thymidine or a cytosine at a position corresponding to nucleotide position 181,700 of SEQ ID NO: 1 (also designated rs6438552 in the NCBI SNP database at May 6, 2005) and/or a cytosine; a thymidine at a position corresponding to nucleotide position 231 of SEQ ID NO: 1 (also designated rs334558 in the NCBI SNP database at May 6, 2005); or an adenosine or a thymidine at a position corresponding to nucleotide position 1679 of SEQ ID NO: 49 (also designated rs3755557 in the NCBI SNP database at May 6, 2005) or mixtures thereof.

In the case of a nucleic acid marker associated with a disease or disorder, the marker is preferably detected by hybridizing a nucleic acid probe comprising the sequence of the marker to a marker linked to nucleic acid in a biological sample derived from a subject under moderate to high stringency hybridization conditions and detecting the hybridization using a detection means, wherein hybridization of the probe to the sample nucleic acid indicates that the subject being tested is predisposed to or suffers from a disease or disorder associated with aberrant GSK-3β activity and/or expression. Preferably, the detection means is a nucleic acid hybridization or amplification reaction, such as, for example, a polymerase chain reaction (PCR).

Not only is such a method useful for, for example, detecting a specific polymorphism in a sample from a subject, but also for detecting a marker in an expression product of a GSK-3β gene, for example, an alternate splice form of a GSK-3β transcript. The present inventors have clearly shown an association between the expression of an alternate splice form of a GSK-3β gene and a disease or disorder associate with aberrant GSK-3β expression and/or activity.

In an alternative embodiment, the marker is within a GSK-3β polypeptide (for example, the marker is encoded by an alternative splice form of a GSK-3β transcript) and is determined by detecting a polypeptide encoded by the transcript. Such a marker is detected, for example, by contacting a biological sample derived from a subject with an antibody or ligand capable of specifically binding to said marker for a time and under conditions sufficient for an antibody/ligand complex to form and then detecting the complex wherein detection of the complex indicates that the subject being tested is predisposed to or suffers from a disease or disorder associated with aberrant GSK-3β activity and/or expression. A suitable method for detecting the antibody-antigen complex will be apparent to the skilled person and/or described herein.

The polymorphisms identified by the present inventors are also associated with increased expression of a GSK-3β gene or an isoform encoded by the GSK-3β gene. Accordingly, a subject at risk of developing or that suffers from a disease or disorder associated with aberrant GSK-3β expression and/or activity may equally be determined by determining an enhanced level of a GSK-3β expression product in a sample from the subject. Suitable methods for determining the level of a GSK-3β expression product will be apparent to the skilled person and/or described herein.

The markers of the present invention may also be used in a multiplex reaction to determine the predisposition of a subject to a disease or disorder or to diagnose a disease or disorder. In this regard, such a multiplexed assay may detect two or more nucleic acid markers that are associated with a disease or disorder, for example, two or more markers described herein. Alternatively, or in addition, a multiplexed assay may detect two or more peptide, polypeptide or protein markers that are, associated with a disease or disorder. Clearly, the combination of nucleic acid-based and antigen-based detection methods is contemplated by the invention.

In one embodiment, the method of the invention further comprises determining an association between the marker and the disease or disorder. Suitable methods for determining an association between a marker and a disease or disorder are known in the art and/or described herein.

Clearly, the methods of the present invention are also useful for determining a subject that is a carrier of a marker that is associated with a disease or disorder associated with aberrant GSK-3β expression and/or activity. Such an assay is useful, for example, for determining the likelihood, or susceptibility of a child of the subject/s being tested to develop a disease or disorder disease.

The diagnostic method of the present invention is also useful in a method of treatment. For example, a method of treatment comprising:
(i) performing a method described herein for determining a disease or disorder associated with aberrant GSK-3β expression and/or activity or a predisposition thereto; and
(ii) administering or recommending a therapeutic for the treatment of the disease or disorder.

In one embodiment, the administration or recommendation of a therapeutic for the treatment of the disease or disorder is based upon the diagnosis of the disease disorder or the diagnosis of a predisposition to the disease or disorder.

Preferably, the therapeutic compound is determined using a method described herein, such as, for example, an antibody.

The present inventors have shown that subjects suffering from a disease or disorder have dysregulated splicing of GSK-3β. Accordingly, in one embodiment, the method of treatment comprises administering or recommending administration of an effective amount of an alternative splice form of a GSK-3β or nucleic acid encoding same or a compound that modulates the alternative splicing of a GSK-3β transcript.

As will be apparent to the skilled artisan, the present invention also encompasses methods for the prophylaxis or prophylactic therapy of a disease or disorder associated with aberrant GSK-3β activity and/or expression. Accordingly, a preferred embodiment of the invention provides a method for the prophylaxis of a disease or disorder associated with aberrant GSK-3β expression and/or activity comprising determining a subject at risk of developing the disease or disorder using a method described herein; and administering or recommending a compound that delays or prevents onset of the disease or disorder. Preferably, the administration or recommendation of the compound is based upon the determination of a subject at risk of developing (i.e., predisposed to developing) the disease or disorder.

The present inventors have also demonstrated that the method of the invention is also useful for determining a subject that will respond to a specific therapy (or therapeutic compound) for treatment of a disease or disorder associated with aberrant GSK-3β expression and/or activity. Preferably, the method of the invention comprises detecting one or more markers described herein to determine whether or not a subject will respond to a treatment with a therapeutic compound. This method has been clearly, demonstrated to be effective in determining a subject suffering from a bipolar affective disorder that responds to treatment with lithium. Such a method is clearly useful for pharacogenomic analysis to determine an effective treatment for a subject.

Any probes and/or primers useful in determining a disease or disorder associated with aberrant GSK-3β expression and/or activity are also contemplated by the present invention.

Given the tight association of the human GSK-3β gene to a disease or disorder associated with aberrant GSK-3β expression and/or activity, and the provision of a plurality of markers, the present invention further provides methods for identifying new markers for a disease or disorder associated with aberrant GSK-3β expression and/or activity.

Furthermore, the observation of altered splicing of a GSK-3β transcript in a subject suffering from a disease or disorder associated with aberrant GSK-3β expression and/or activity provides the basis for a screening method for identifying a compound for the treatment of such a disease. Using such a screen, the present inventors have shown that the compounds lithium and TDZ8 are useful for modulating the expression of a splice form of a GSK-3β transcript.

### BRIEF DESCRIPTION OF THE DRAWINGS

FIG. 1A is a diagrammatic representation of the genomic structure of the GSK-3β gene. The relative position of several polymorphisms in GSK-3β is indicated (vertical arrows). The polymorphism labeled GSK1 is also designated rs13312998 in the NCBI SNP database; The polymorphism labeled GSK1 is also designated rs6438552 in the NCBI SNP database; and polymorphism labeled GSK3 is also designated rs7633279 in the NCBI SNP database.

FIG. 1B is a diagrammatic representation of a transcript map showing known genes adjacent to GSK-3β. These genes encode the following proteins Cytochrome c oxidase assembly protein homolog (COX17); nuclear receptor subfamily 1, group I, member 2 (NR1I2); GABAB-related G-protein coupled receptor (GABABL); and Follistatin-like 1 (FSTL1). Relative positions of the SNPs used for linkage disequilibrium analysis are indicated (vertical arrows).

FIG. 1C is a graphical representation showing the D′ values (i.e., the degree of linkage disequilibrium between two markers) for pair-wise comparisons between the GSK2 SNP (rs6438552) (shown in FIG. 1A) and adjacent polymorphisms (Shown in FIG. 1B).

FIG. 1D is a diagrammatic representation showing D′ values and r² values for pair-wise comparisons between the GSK-3β SNPs (shown in FIG. 1A) and adjacent polymorphisms (shown in FIG. 1B).

FIG. 2A is a diagrammatic representation of the exon trap pSPL3 vector with the region spanning GSK-3β exon 6 and flanking intronic sequence. A chimeric exon is generated by the fusion of a cryptic splice donor site from pSPL3 (CSD) and two alternative cryptic splice acceptor sites. Exon trap transcripts are generated by splicing of the vector's exon boundaries (TAT) with splice donor and acceptor sites within the subcloned genomic fragment.

FIG. 2B is a copy of a photographic representation showing of electrophoresis of exon trap products on a 2% agarose gel. Exon trapping was performed in two cell lines, HEK293 (upper panel) and SK-N-MC (lower panel), transfected with the parental pSPL3 vector (vec), or constructs carrying genomic fragments with the each allele of the three intronic polymorphisms of GSK-3β (shown in FIG. 1A). Schematic diagrams of the three exon trap products associated with the utilization of three possible splice acceptor sites A (native splice acceptor site), B (IVS 6+212), and C (IVS 6+181) are shown for the exon trap analysis of GSK3 SNP. The relevant nucleotide at the site of the SNP is indicated at the top of the gel in the upper panel.

FIG. 2C is a graphical representation of semi-quantitative analysis of exon trap products isolated from HEK-293 cells transfected with either the C allele (open bars) or T allele (black bars) of the GSK2 SNP (rs6438552) (i.e. the SNP located in intron 5 of GSK-3β, shown in FIG. 1A). Mean values±SD obtained from four separate transfections. Pairwise Student's t test comparisons were performed between the T and C allele exon trap products. Statistical significance is indicated (*=p<0.05).

2D is a graphical representation of semi-quantitative analysis of exon trap products isolated from SK-N-MC cells transfected with either the C allele (open bars) or T allele (black bars) of the GSK2 SNP (rs6438552) (i.e. the SNP located in intron 5 of GSK-3β, shown in FIG. 1A). Mean values±SD obtained from four separate transfections. Pairwise Student's t test comparisons were performed between the T and C allele exon trap products. Statistical significance is indicated (*=p<0.05).

FIG. 3A is a copy of a photographic representation showing electrophoresis of ³³P labelled RT-PCR products from GSK-3β transcripts produced using RNA isolated from patient lymphocytes on a 6% PAGE gel; The major bands correspond to GSKΔexon9 and GSKΔexon9+11 transcripts. The identity of each of the bands is indicated on the right hand side of the figure.

FIG. 3B is a graphical representation showing semi-quantitative analysis of GSK-3β splice isoform transcripts from RNA isolated from patient lymphocytes. The relative level of each splice isoform is indicated for C/C genotype (open bars) or T/T genotype (black bars). Pairwise comparisons were made between two genotype groups (n=22) using Student's t test. Significance is indicated (*=p<0.05, **=p<0.005).

FIG. 3C is a copy of a photographic representation showing a Western blot of COS-7 cells transfected with GSK-3β cDNAs. Lane 1 GSK.Full length cDNA; lane 2=GSKΔexon9 cDNA; lane 3=GSKΔexon11 cDNA; lane 4=GSKΔexon9+11 cDNA; and lane 5=Lymphocyte control. The results obtained with the lymphocyte sample indicate that the two major protein bands which to GSKΔexon9 and GSKΔexon9+11.

FIG. 4A is a graphical representation showing quantification of Tau [pSER396] levels in COS-7 cells transfected with a GSK-3β cDNA. Results were normalized for GSK-3β levels by Western blot from three separate transfections. Significant differences were observed for the GSKΔexon9+11 isoform (***=p<0.0005) following ANOVA and post-hoc analysis.

FIG. 4B is a graphical representation showing the correlation between GSKΔexon9+11 transcript levels and Tau [pSER396] levels. Lymphocyte cells were analyzed for relative de novo GSKΔexon9+11 transcript levels and Tau [pSER396] levels in individuals with T/T (black circles) and C/C (open circles) genotypes for the GSK2 SNP (rs6438552). Significance for the coefficient of determination (r²) was calculated using linear regression analysis with the null hypothesis that there is no correlation within the population (r²=0).

FIG. 4C is a graphical representation showing relative levels of Tau [pSER396] phosphorylation when cells expressing GSKΔexon9 or GSKΔexon9+11 were incubated in the presence of increasing concentrations of lithium chloride. Values were normalized against the level of Tau [p396] phosphorylation detected in the absence of inhibitor. Dose response curves and mean values (±SEM) are plotted against a semi-logarithmic axis.

FIG. 4D is a graphical representation showing relative levels of Tau [pSER396] phosphorylation when cells expressing GSKΔexon9 or GSKΔexon9+11 were incubated in the presence of increasing concentrations of TDZD8. Values were normalized against the level of Tau [p396] phosphorylation detected in the absence of inhibitor. Dose response curves and mean values (±SEM) are plotted against a semi-logarithmic axis.

FIG. 5A is a schematic representation showing the putative binding sites for transcription factors in the promoter region of GSK-3β. The binding sites of the following transcription factors were analyzed M2F (open circle), SP1 (black circle), AP4 (open square), AP1 (black square), CREL (open diamond), NFY (black diamond), c-myb (open triangle) and v-myb (black triangle). The site of the rs334558 SNP is indicated by the vertical arrow with the relevant nucleotide present also indicated.

FIG. 5B is a graphical representation showing the level of detected light emitted in a luciferase reporter gene assay for GSK-3β promoter activity in HEK293 cells. The relevant nucleotide at the site of the rs334558 SNP is indicated. Mean values A SEM were derived from four separate transfections into either 293 or SK-N-MC cells. Fluorescence units for each promoter haplotype were normalized to the values for the pGL3 vector (vec) for each transfection. Significance is indicated (**=p<0.005).

FIG. 5C is a graphical representation showing the level of detected light emitted in a luciferase reporter gene assay for GSK-3β promoter activity in SK-N-MC cells. The relevant nucleotide at the site of the rs334558 SNP (located at a position equivalent to nucleotide number 231 of SEQ ID NO: 1) is indicated. Mean values±SEM were derived from four separate transfections into either 293 or SK-N-MC cells. Fluorescence units for each promoter haplotype were normalized to the values for the pGL3 vector (vec) for each transfection. Significance is indicated (*=p<0.05).

FIG. 6 is a graphical representation showing the level of phosphorylation of tau polypeptide at serine 396 in samples from brains of control subjects (left-hand column) and samples from brains of subjects suffering from Parkinson's disease (right-hand column).

FIG. 7 is a graphical representation showing the level of expression of GSK-3 in subjects comprising either an AA or AT genotype at the site of the rs3755557 SNP (located at a position corresponding to nucleotide position 1679 of SEQ ID NO: 49). Genotypes are indicated at the base of the graph.

### DETAILED DESCRIPTION OF THE PREFERRED EMBODIMENTS

Diseases or Disorders Associated with Aberrant GSK-3β Expression/Activity

In a preferred embodiment, the method of the invention is useful for determining a subject that suffers from or is predisposed to a neurodegenerative disease. As used herein, the term “neurodegenerative disease” shall be taken to mean a disease that is characterized by neuronal cell death. The neuronal cell death observed in a neurodegenerative disease is often preceded by neuronal dysfunction, sometimes by several years. Accordingly, the term “neurodegenerative disease” includes a disease or disorder that is characterized by neuronal dysfunction and eventually neuronal cell death. Often neurodegenerative diseases are also characterized by increased gliosis (e.g., astrocytosis or microgliosis) in the region/s of neuronal death.

The cellular events observed in a neurodegenerative disease often manifest as a behavioral change (e.g., deterioration of thinking and/or memory) and/or a movement change (e.g., tremor, ataxia, postural change and/or rigidity). Examples of neurodegenerative disease include, for example, Alzheimer's disease, amyotrophic lateral sclerosis, ataxia (e.g., spinocerebellar ataxia or Friedreich's Ataxia), Creutzfeldt-Jakob Disease, a polyglutamine disease (e.g., Huntington's disease or spinal bulbar muscular atrophy), Hallervorden-Spatz disease, idiopathic torsion disease, Lewy body disease, multiple system atrophy, neuroanthocytosis syndrome, olivopontocerebellar atrophy, Parkinson's disease, Pelizaeus-Merzbacher disease, Pick's disease, progressive supranuclear palsy, syringomyelia, torticollis, spinal muscular atophy or a trinucleotide repeat disease (e.g., Fragile X Syndrome).

In one embodiment, the neurodegenerative disease is associated with aberrant deposition or tau and/or hyperphosphorylation of tau. For example, the neurodegenerative disease is selected from the group consisting of frontotemporal dementia, corticobasal degeneration, progressive supranuclear palsy, a Parkinson's disease or an Alzheimer's disease. Preferably, the method of the invention is useful for diagnosing or determining a predisposition to a neurodegenerative disease selected from the group consisting of a Parkinson's disease and an Alzheimer's disease.

By “Parkinson's disease” is meant a chronic progressive nerve disease characterized by muscle tremors, weakness, rigid movements, halting gait, drooping posture and expressionless facial appearance. The pathology of a Parkinson's disease is characterized by loss of neuromelanin-containing monoamine neurons, particularly dopamine (DA) neurons in the substantia nigra pars compacta. A pathologic hallmark is the presence of cytoplasmic eosinophilic inclusions (Lewy bodies) in monoamine neurons. The term “a Parkinson's disease” shall be understood to encompass early onset Parkinson's disease and late onset Parkinson's disease, juvenile onset Parkinson's disease, idiopathic Parkinson's disease and monogenic Parkinson's disease.

In another embodiment, a neurodegenerative disease is a dementing neurodegenerative disease. By “dementing neurodegenerative disease” is meant a disease that is characterized by chronic loss of mental capacity, particularly progressive deterioration of thinking, memory, behavior, personality and motor function, and may also be associated with psychological symptoms such as depression and apathy. Preferably, a dementing neurodegenerative disease is not caused by, for example, a stroke, an infection or a head trauma. Examples of a dementing neurodegenerative disease include, for example, an Alzheimer's disease, vascular dementia, dementia with Lewy bodies, frontotemporal dementia and prion disease, amongst others.

Preferably, the dementing neurodegenerative disease is an Alzheimer's disease. By “an Alzheimer's disease” is meant a neurodegenerative disease characterized by progressive impairments in memory, behavior, language and/or visuo-spatial skills. Pathologically, an Alzheimer's disease is characterized by neuronal loss, gliosis, neurofibrillary tangles, senile plaques, Hirano bodies, granulovacuolar degeneration of neurons, amyloid angiopathy and/or acetylcholine deficiency. The term “an Alzheimer's disease” shall be taken to include early onset Alzheimer's disease (e.g., with an onset earlier than the sixth decade of life), a late onset Alzheimer's disease (e.g., with an onset later then, or in, the sixth decade of life) and a juvenile onset Alzheimer's disease.

In another preferred embodiment, the invention is useful for diagnosing a psychiatric disorder or determining a predisposition to a psychiatric disorder. By “psychiatric disorder” is meant a disorder that is associated with behavioral changes in an individual without neurodegeneration (or without obvious neurodegeneration). A suitable psychiatric disorder includes, for example, a somatoform disorder, an anxiety disorder, a dissociative disorder, a mood disorder, a personality disorder, a psycho-sexual disorder and a schizophrenia. For example, a psychiatric disorder is a disorder such as, for example, a bipolar affective disorder, a cyclothymic disorder, schizophrenia, a schizoaffective disorder, a schizophreniform disorder and a brief psychotic disorder.

In a preferred embodiment, the psychiatric disorder is a bipolar affective disorder. As used herein, the term “a bipolar affective disorder” shall be taken to include all forms of bipolar affective disorder, including bipolar I disorder (severe bipolar affective (mood) disorder), schizoaffective disorder, bipolar II disorder or unipolar disorder.

In another preferred embodiment, the method of the invention is useful for diagnosing or determining a predisposition to a disease or disorder associated with aberrant glucose metabolism. By “disease or disorder associated with aberrant glucose metabolism” is meant a disease or disorder that is caused by or associated with increased or reduced glucose metabolism in a subject compared to a normal and/or healthy subject. Suitable diseases and/or disorders associated with aberrant sugar metabolism include, for example, type I diabetes, type II diabetes, insulin resistance, reduced insulin sensitivity, hyperinsulinemia, hypoinsulinemia, hyperglycemia, hypoglycemia and obesity. Preferably, the disease or disorder associated with aberrant sugar metabolism is insulin resistance, reduced insulin sensitivity or type II diabetes.

In another preferred embodiment, the method of the invention is useful for determining a subject that suffers from or is predisposed to an inflammatory disease, more preferably, a chronic inflammatory disease. A chronic inflammatory disease is caused by an autoimmune response against a self-antigen in a subject, wherein the antigen is non-limiting and persists until, for example, a tissue is destroyed or exposure to the antigen eliminated. Examples of a chronic inflammatory disease include, for example, inflammatory bowel disease, rheumatoid arthritis and multiple sclerosis.

In a still further embodiment, the method of the invention is useful for determining a subject that suffers from or is predisposed to a cancer. As used herein, the term “cancer” shall be taken to mean any disease or disorder characterized by hyperproliferation of a cell in a subject. The term cancer includes a primary cancer or tumor, a metastasis of a cancer or tumor, or a recurrence of a cancer or tumor. Preferred cancers include, for example, a glioma, a lymphoma, a pancreatic cancer, thyroid carcinoma and a hepatoma.

### GSK-3β Gene and Expression Products

In one embodiment, the marker that is associated with a disease or disorder associated with aberrant GSK-3β expression and/or activity comprises, consists of or is located within a GSK-3β genomic gene. Preferably, a GSK-3β genomic gene comprises a nucleotide sequence at least about 80% identical to the nucleotide sequence set forth in SEQ ID NO: 1 or the complement thereof. More preferably, the degree of identity is at least about 85% to about 90%, more preferably, about 90% to about 95%, even more preferably about 95% to about 99%.

A genomic gene of GSK-3β shall be understood to include the coding region of a GSK-3β protein (e.g., codons required to encode various isozymes of GSK-3β in addition to intervening intronic sequences in addition to regulatory regions that control the expression of said gene, e.g., a promoter or fragment thereof.

In another embodiment, a marker that is associated with a disease or disorder associated with aberrant GSK-3β expression and/or activity comprises or is within an expression product of a GSK-3β gene. For example, the marker comprises, consists of or is located within a nucleic acid that comprises a nucleotide sequence at least about 80% identical to a GSK-3β cDNA or GSK-3β mRNA. For example, the nucleotide sequence of isoforms of a GSK-3β cDNA are set forth in SEQ ID NOs: 2, 4, 6, and 8. Preferably, the degree of identity is at least about 85% to about 90%, more preferably, about 90% to about 95%, even more preferably about 95% to about 99%.

In another embodiment, a marker that is associated with a disease or disorder associated with aberrant GSK-3β expression and/or activity comprises, consists of or is located within a nucleic acid that is capable of encoding a GSK-3β polypeptide. Preferably, the marker comprises, consists of or is located within a nucleic acid capable of encoding a polypeptide comprising an amino acid sequence at least about 80% identical to the amino acid sequence set forth in SEQ ID NOs: 3, 5, 7, or 9. Preferably, the degree of identity is at least about 85% to about 90%, more preferably, about 90% to about 95%, even more preferably about 95% to about 99%.

In a still further embodiment, a marker that is associated with a disease or disorder associated with aberrant GSK-3β expression and/or activity comprises, consists of or is located within a GSK-3β polypeptide. Preferably, the marker comprises, consists of or is located within a polypeptide comprising an amino acid sequence at least about 80% identical to the amino acid sequence set forth in SEQ ID NOs: 3, 5, 7, or 9. Preferably, the degree of identity is at least about 85% to about 90%, more preferably, about 90% to about 95%, even more preferably about 95% to about 99%.

In determining whether or not two amino acid sequences fall within the defined percentage identity limits supra, those skilled in the art will be aware that it is possible to conduct a side-by-side comparison of the amino acid sequences. In such comparisons or alignments, differences will arise in the positioning of non-identical residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using software of the Computer Genetics Group, Inc., University Research Park, Maddison, Wis., United States of America, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximize the number of identical/similar residues and to minimize the number and/or length of sequence gaps in the alignment.

Alternatively, a suite of commonly used and freely available sequence comparison algorithms is provided by the National Center for Biotechnology Information (NCBI) Basic Local Alignment Search Tool (BLAST) (Altschul et al. J. Mol. Biol. 215: 403-410, 1990), which is available from several sources, including the NCBI, Bethesda, Md. The BLAST software suite includes various sequence analysis programs including “blastn,” that is used to align a known nucleotide sequence with other polynucleotide sequences from a variety of databases and “blastp” used to align a known amino acid sequence with one or more sequences from one or more databases. Also available is a tool called “BLAST 2 Sequences” that is used for direct pairwise comparison of two nucleotide sequences.

As used herein the term “NCBI” shall be taken to mean the database of the National Center for Biotechnology Information at the National Library of Medicine at the National Institutes of Health of the Government of the United States of America, Bethesda, Md., 20894.

In determining whether or not two nucleotide sequences fall within a particular percentage identity limitation recited herein, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more nucleotide sequences shall be taken to refer to the number of identical residues between said sequences as determined using any standard algorithm known to those skilled in the art. For example, nucleotide sequences may be aligned and their identity calculated using the BESTFIT program or other appropriate program of the Computer Genetics Group, Inc., University Research Park, Madison, Wis., United States of America (Devereaux et al, Nucl. Acids Res. 12, 387-395, 1984). As discussed supra BLAST is also useful for aligning nucleotide sequences and determining percentage identity.

Markers Associated with a Disease or Disorder

In one embodiment, a marker associated with a disease or disorder associated with aberrant GSK-3β expression and/or activity is a nucleic acid marker. Preferably, the marker comprises or consists of a nucleotide sequence at least about 80% identical to at least about 20 nucleotides in length, more preferably at least about 30 nucleotides in length, of a sequence selected from the group consisting of:
(i) a sequence at least about 80% identical to a sequence selected from the group consisting of SEQ ID NO: 1, SEQ ID NO: 2, SEQ ID NO: 4, SEQ ID NO: 6 and SEQ ID NO: 8;
(ii) a sequence capable of encoding an amino acid sequence at least 80% identical to the sequence set forth in SEQ ID NO: 3, SEQ ID NO: 5, SEQ ID NO: 7 and SEQ ID NO: 9; and
(iii) a sequence complementary to a sequence set forth in (i) or (ii).

Such a nucleic acid marker may be or comprise, for example, a polyorphism, an insertion into a GSK-3β gene, a deletion from a GSK-3β gene, a transcript of a GSK-3β gene or a fragment thereof or an alternatively spliced transcript of a GSK-3β gene or a fragment thereof.

In one embodiment, a polymorphism associated with a disease or disorder is a simple nucleotide polymorphism. As used herein, the term “simple nucleotide polymorphism” shall be taken to mean a polymorphism that comprises or consists of a small change (e.g., an insertion, a deletion, a transition or a transversion) in the genome of a subject or an expression product thereof compared to the nucleotide sequence observed in a normal subject or the majority of a normal population of subjects. For example, a simple nucleotide polymorphism comprises or consists of, a single nucleotide-insertion or deletion, insertion or deletion of two, three or four or more nucleotides, transition of one or more nucleotides, or transversion of one or more nucleotides.

Preferably, the marker that is associated with a disease or disorder associated with aberrant GSK-3β activity and/or expression or a predisposition to the disease or disorder is a single nucleotide polymorphism in its homozygous state.

In another preferred embodiment, a polymorphism associated with a disease or disorder is additionally associated with or causes alternative splicing of a GSK-3β mRNA. As used herein, the term “alternative splicing” shall be taken to mean the insertion or removal of exons into/from a GSK-3β mRNA. Accordingly, an alternatively spliced GSK-3β mRNA comprises additional exons, or lack exons (e.g., nucleotides) compared, to the sequence of a GSK-3β cDNA set forth in SEQ ID NO: 2.

In one embodiment, the presence of a polymorphism that is associated with alternative splicing of a GSK-3β mRNA is correlated with modulated levels of alternatively spliced GSK-3β mRNA. Accordingly, the level of a specific splice form of GSK-3β is increased or decreased when the specific polymorphism is present and is useful for detecting a marker associated with a disease or disorder.

Preferably, an alternatively spliced GSK-3β transcript comprises a nucleotide sequence at least about 80% identical to a nucleotide sequence selected from the group consisting of SEQ ID NO: 4, SEQ ID NO: 6 and SEQ ID NO: 8. Preferably, the degree of sequence identity is at least about 90% or 95% or 98% or 99%.

In another embodiment, an alternatively spliced GSK-3β transcript comprises a nucleotide sequence that encodes a polypeptide comprising an amino acid sequence at least about 80% identical to an amino acid sequence selected from the group consisting of SEQ ID NO: 5, SEQ ID NO: 7 and SEQ ID NO: 9 Preferably, the degree of sequence identity is at least about 90% or 95% or 98% or 99%.

In one embodiment, the polymorphism is associated with or causes the splicing of exon 9 in a GSK-3β mRNA (e.g., produces a cDNA that comprises a nucleotide sequence at least about 80% identical to the nucleotide sequence set forth in SEQ ID NO: 5.

In another embodiment, the polymorphism is associated with or causes the splicing of exon 11 in a GSK-3β mRNA (e.g., produces a cDNA that comprises a nucleotide sequence at least about 80% identical to the nucleotide sequence set forth in SEQ ID NO:7.

In one embodiment, the polymorphism is associated with or causes the splicing of exon 9 and exon 11 in a GSK-3β mRNA (e.g., produces a cDNA that comprises a nucleotide sequence at least about 80% identical to the nucleotide sequence set forth in SEQ ID NO:9.

In one embodiment, the polymorphism comprises, consists of or is located within intron 5 of the GSK-3β gene. In this context, the term “intron 5” shall be taken to mean the intron occurring between exons 5 and 6 of the cDNA sequence set forth in SEQ ID NO: 2. Preferably, the term “intron 5” shall be taken to mean a nucleic acid comprising a nucleotide sequence at least abut 80% identical or 90% identical or 98% identical to the nucleotides in the region spanning from nucleotide position 178,624 to nucleotide position 181,858 of SEQ ID NO: 1.

Preferably, a marker that is associated with a disease or disorder associated with aberrant GSK-3β expression and/or activity comprises a thymidine at a position corresponding to nucleotide position 181,700 of SEQ ID NO: 1. Preferably, the polymorphism is in a homozygous form.

Alternatively, a marker that is associated with a disease or disorder associated with aberrant GSK-3β expression and/or activity comprises a cytosine at a position corresponding to nucleotide position 181,700 of SEQ ID NO: 1. Preferably, the polymorphism is in a homozygous form.

In a preferred embodiment, the marker within intron 5 of the GSK-3β gene is used to diagnose or determine a predisposition to any disease or disorder associated with aberrant GSK-3β activity and/or expression. Preferably, the marker within intron 5 of the GSK-3β gene is used to diagnose or determine a predisposition to a neurodegenerative disease (e.g., an Alzheimer's disease or a Parkinson's disease).

In another preferred embodiment, the marker within intron 5 of the GSK-3β gene is used to diagnose or determine a predisposition to is used to determine a disease or disorder or a predisposition to a disease or disorder in a Caucasian subject and/or an Asian subject. Preferably, the Asian subject is a Chinese subject, more preferably, a Hong Kong Chinese subject or a Singaporean Chinese subject. Preferably, the marker is in a homozygous form. In a preferred embodiment, the subject does not express the ε4 isoform of the apolipoprotein E gene.

The present inventors have additionally shown association of a polymorphism in the promoter region of a GSK-3β gene and the development of a disease or disorder associated with aberrant GSK-3β activity and/or expression. Accordingly, in another embodiment of the invention, the polymorphism comprises, consists of or is located within the promoter region of GSK-3β. As used herein the term “promoter region of GSK-3β” shall be taken to mean a nucleic acid that comprises one or more elements that are associated with control of expression of one or more isoforms of GSK-3β.

Preferably, a polymorphism located within the promoter region of a GSK-3β gene comprises, consists of or is located within a nucleic acid that comprises a nucleotide sequence corresponding to the region spanning from approximately nucleotide position 1 to nucleotide position 1232 of SEQ ID NO: 1 or that is set forth in SEQ ID NO: 49.

In a preferred embodiment a polymorphism located within the promoter region of a GSK-3β gene is also associated with or causes increased expression of a GSK-3β expression product. Methods for determining expression of a GSK-3β mRNA or polypeptide are known in the art and/or described herein.

In a preferred embodiment, the polymorphism comprises or consists of a thymidine or a cytosine at a position corresponding to nucleotide position 232 of SEQ ID NO: 1.

In another embodiment, the polymorphism comprises or consists of a cytosine at a position corresponding to nucleotides position 3356 of SEQ ID NO: 49. In another embodiment, the polymorphism comprises or consists of a thymidine at a position corresponding to nucleotides position 3356 of SEQ ID NO: 49.

Preferably, this marker within a promoter region of a GSK-3β gene is used to diagnose or determine a predisposition to a disease or disorder associated with aberrant GSK-3β expression other than a psychiatric disorder (e.g., a bipolar affective disorder or a schizophrenia). More preferably, a marker within a promoter region of a GSK-3β gene is used to diagnose or determine a predisposition to a neurodegenerative disease (e.g., a Parkinson's disease or an Alzheimer's disease).

In another preferred embodiment, the polymorphism comprises an adenosine or a cytosine at a position corresponding to nucleotide position 1679 of SEQ ID NO: 49. Such a marker is preferably of use in diagnosing any disease or disorder associated with aberrant GSK-3β expression and/or activity (for example, a psychiatric disorder, e.g., a bipolar affective disorder).

In one embodiment, the method of the invention comprises detecting or determining the presence of a plurality of markers associated with a disease or disorder. For example, the present inventors have shown that the detection of a thymidine at a position corresponding to position 232 of SEQ ID NO: 1 and a cytosine at a position corresponding to nucleotide position 181,700 of SEQ ID NO: 1 indicates' that the subject suffers from or has an increased predisposition to a disease or disorder associated with aberrant GSK-3β expression and/or activity. Preferably, both of the polymorphisms are in a homozygous form.

Accordingly, in one embodiment, the method of the invention comprises:
(i) detecting a first polymorphism comprising a cytosine or a thymidine in homozygous form at a position corresponding to nucleotide position 181,700 of SEQ ID NO: 1; and
(ii) detecting a second polymorphism comprising a cytosine or a thymidine in homozygous form at a position corresponding to nucleotide position 232 of SEQ ID NO: 1,
   wherein detection of the first and the second polymorphisms is ‘indicative of the disease’ or disorder or a predisposition to the disease or disorder in the subject.

Preferably, the method of the invention comprises:
(i) detecting a first polymorphism comprising a cytosine or a thymidine in homozygous form at a position corresponding to nucleotide position 181,700 of SEQ ID NO: 1;
(ii) detecting a second polymorphism comprising a cytosine or a thymidine in homozygous form at a position corresponding to nucleotide position 232 of SEQ ID NO: 1; and
(iii) detecting a third polymorphism comprising an adenosine and/or a thymidine at a position corresponding to nucleotide position 1679 of SEQ ID NO: 49,
   wherein detection of the first, second and third polymorphisms is indicative of the disease or disorder or a predisposition to the disease or disorder in the subject.

Preferably, the third polymorphism is an adenosine at a position corresponding to nucleotide position 1679 of SEQ ID NO: 49.

### Detection of Nucleic Acid Changes in a GSK-3β Gene or Expression Product

In one embodiment the method for determining the predisposition of a subject to a disease or disorder associated with aberrant GSK-3β expression and/or activity or diagnosing the disease or disorder disease comprises determining the presence of the marker within a GSK-3β gene or expression product thereof that is associated with the disease or disorder in a test sample derived from a subject, wherein the presence of the marker indicates that the subject being tested is predisposed to or suffers from the disease or disorder.

As will be apparent to the skilled artisan, a marker that is associated with a disease or disorder is preferably detectable by standard procedures, for example by nucleic acid hybridization. Accordingly, a nucleic acid marker is preferably at least about 8 nucleotides in length (for example, for detection using a locked nucleic acid (LNA) probe). To provide more specific hybridization, a marker is preferably at least about 15 nucleotides in length or more preferably at least 20 to 30 nucleotides in length. Such markers are particularly amenable to detection by nucleic acid hybridization-based detection means assays, such as, for example any known format of PCR or ligase chain reaction.

Generally, a method for detecting a nucleic acid marker comprises hybridizing an oligonucleotide to the marker linked to nucleic acid in a sample from a subject under moderate to high stringency conditions and detecting hybridization of the oligonucleotide using a detection means, such as for example, an amplification reaction or a hybridization reaction.

For the purposes of defining the level of stringency to be used in these diagnostic assays, a low stringency is defined herein as being a hybridization and/or a wash carried out in 6×SSC buffer, 0.1% (w/v) SDS at 28° C., or equivalent conditions. A moderate stringency is defined herein as being a hybridization and/or washing carried out in 2×SSC buffer, 0.1% (w/v) SDS at a temperature in the range 45° C. to 65° C., or equivalent conditions. A high stringency is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample DNA, and/or the type of hybridization probe used.

In another embodiment, stringency is determined based upon the temperature at which a probe or primer dissociates from a target sequence (i.e., the probe or primers melting temperature or Tm). Such a temperature may be determined using, for example, an equation or by empirical means. Several methods for the determination of the Tm of a nucleic acid are known in the art. For example the Wallace Rule determines the G+C and the T+A concentrations in the oligonucleotide and uses this information to calculate a theoretical Tm (Wallace et al., Nucleic Acids Res. 6, 3543, 1979). Alternative methods, such as, for example, the nearest neighbour method are known in the art, and described, for example, in Howley, et al., J. Biol. Chem. 254, 4876, Santa Lucia, Proc. Natl. Acad. Sci. USA, 95: 1460-1465, 1995 or Bresslauer et al., Proc. Natl. Acad. Sci. USA, 83: 3746-3750, 1986. A temperature that is similar to (e.g., within 5° C. or within 10° C.) or equal to the proposed denaturing temperature of a probe or primer is considered to be high stringency. Medium stringency is to be considered to be within 10° C. to 20° C. or 10° C. to 15° C. of the calculated Tm of the probe or primer.

As will be apparent to the skilled artisan a probe or primer capable of specifically detecting a marker that is associated with a disease or disorder associated with aberrant GSK-3β activity and/or expression is any probe or primer that is capable of selectively hybridizing to the region of the genome that comprises said marker, or an expression product thereof. As used herein, the term “selectively hybridizes” means that the nucleic acid used as a probe hybridizes to a target nucleic acid (e.g., a nucleic acid comprising the marker) at a level significantly above background. The background hybridization may be due to other nucleic acids present, for example, in the sample being screening. Accordingly, background hybridization is a level of hybridization that occurs between the probe and a non-specific nucleic acid in the sample being assayed. Preferably, the a background interaction occurs less than 10 fold, preferably less than 100 fold as often as the specific (target) interaction observed with the target nucleic acid. The degree of interaction is measured, for example, by labeling the probe with a detectable marker, e.g. with ³²P or by performing an amplification reaction and determining the approximate level of amplification product produced, e.g., using gel electrophoresis.

In one embodiment, a preferred probe or primer comprises, consists of a nucleic acid comprising a nucleotide sequence at least about 80% identical to a sequence selected from the group consisting of:
(i) a sequence at least about 80% identical to a sequence selected from the group consisting of SEQ ID NO: 1, SEQ ID NO: 2, SEQ ID NO: 4, SEQ ID NO: 6, SEQ ID NO: 8 and SEQ ID NO: 10;
(ii) a sequence capable of encoding an amino acid sequence at least 80% identical to the sequence set forth in SEQ ID NO: 3, SEQ ID NO: 5, SEQ ID NO: 7 and SEQ ID NO: 9; and
(iii) a sequence complementary to a sequence set forth in (i) or (ii).

Probe/Primer Design and Production

As will be apparent to the skilled artisan, the specific probe or primer used in an assay of the present invention will depend upon the assay format used. Clearly, a probe or primer that is capable of specifically hybridizing to or detecting the marker of interest is preferred. Methods for designing probes and/or primers for, for example, PCR or hybridization are known in the art and described, for example, in Dieffenbach and Dveksler (Eds) (In: PCR Primer: A Laboratory Manual, Cold Spring Harbour Laboratories, NY, 1995). Furthermore, several software packages are publicly available that design optimal probes and/or primers for a variety of assays, e.g. Primer 3 available from the Center for Genome Research, Cambridge, Mass., USA. Probes and/or primers useful for detection of a marker associated with a disease or disorder associated with aberrant GSK-3β activity and/or expression are assessed to determine those that do not form hairpins, self-prime or form primer dimers (e.g. with another probe or primer used in a detection assay).

Furthermore, a probe or primer (or the sequence thereof) is assessed to determine the temperature at which it denatures from a target nucleic acid (i.e. the melting temperature of the probe or primer, or Tm). Methods of determining Tm are known in the art and described, for example, in Santa Lucia, Proc. Natl. Acad. Sci. USA, 95: 1460-1465, 1995 or Bresslauer et al., Proc. Natl. Acad. Sci. USA, 83: 3746-3750, 1986. A primer or probe useful for detecting a SNP in an allele specific PCR assay or a ligase chain reaction assay is designed such that the 3′ terminal nucleotide hybridizes to the site of the SNP. The 3′ terminal nucleotide may be any of the nucleotides known to be present at the site of the SNP. When complementary nucleotides occur in the probe or primer and at the site of the polymorphism the 3′ end of the probe or primer hybridizes completely to the marker of interest and facilitates, for example, PCR amplification or ligation to another nucleic acid. Accordingly, a probe or primer that completely hybridizes to the target nucleic acid produces a positive result in an assay.

In another embodiment, a primer useful for a primer extension reaction is designed such that it specifically hybridizes to a region adjacent to a specific nucleotide of interest, e.g. a SNP. While the specific hybridization of a probe or primer may be estimated by determining the degree of homology of the probe or primer to any nucleic acid using software, such as, for example, BLAST, the specificity of a probe or primer can only be determined empirically using methods known in the art.

A locked nucleic acid (LNA) or protein-nucleic acid (PNA) probe or a molecular beacon useful, for example, for detection of a SNP or microsatellite by hybridization is at least about 8 to 12 nucleotides in length. Preferably, the nucleic acid, or derivative thereof, that hybridizes to the site of the SNP or microsatellite is positioned at approximately the centre of the probe, thereby facilitating selective hybridization and accurate detection.

Methods for producing/synthesizing a probe or primer of the present invention are known in the art. For example, oligonucleotide synthesis is described, in Gait (Ed) (In: Oligonucleotide Synthesis: A Practical Approach, IRL Press, Oxford, 1984). For example, a probe or primer may be obtained by biological synthesis (eg. by digestion of a nucleic acid with a restriction endonuclease) or by chemical synthesis. For short sequences (up to about 100 nucleotides) chemical synthesis is preferable.

For longer sequences standard replication methods employed in molecular biology are useful, such as, for example, the use of M13 for single stranded DNA as described by J. Messing (1983) Methods Enzymol, 101, 20-78.

Other methods for oligonucleotide synthesis include, for example, phosphotriester and phosphodiester methods (Narang, et al. Meth. Enzymol 68: 90, 1979) and synthesis on a support (Beaucage, et al Tetrahedron Letters 22: 1859-1862, 1981) as well as phosphoramidate technique, Caruthers, M. H., et al., “Methods in Enzymology,” Vol. 154, pp. 287-314 (1988), and others described in “Synthesis and Applications of DNA and RNA,” S. A. Narang, editor, Academic Press, New York, 1987, and the references contained therein.

LNA synthesis is described, for example, in Nielsen et al, J. Chem. Soc. Perkin Trans., 1: 3423, 1997; Singh and Wengel, Chem. Commun. 1247, 1998. While, PNA synthesis is described, for example, in Egholm et al., Am. Chem. Soc., 114: 1895, 1992; Egholm et al., Nature, 365: 566, 1993; and Orum et al., Nucl. Acids Res., 21: 5332, 1993.

In one embodiment, a probe or primer useful for performance of the method of the invention comprises a nucleotide sequence set forth in SEQ ID-NO: 12, SEQ ID NO: 13, SEQ ID NO: 50, SEQ ID NO: 51, SEQ ID NO: 54 or SEQ ID NO: 55.

In one embodiment, the probe or primer comprises one or more detectable markers. For example, the probe or primer comprises a fluorescent label such as, for example, fluorescein (FITC), 5,6-carboxymethyl fluorescein, Texas red, nitrobenz-2-oxa-1,3-diazol4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, fluorescein (5-carboxyfluorescein-N-hydroxysuccinimide ester), rhodamine (5,6-tetramethyl rhodamine). The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm).

Alternatively, the probe or primer is labeled with, for example, a fluorescent semiconductor nanocrystal (as described, for example, in U.S. Pat. No. 6,306,610), a radiolabel or an enzyme (e.g. horseradish peroxidase (HRP), alkaline phosphatase (AP) or β-galactosidase).

Such detectable labels facilitate the detection of a probe or primer, for example, the hybridization of the probe or primer or an amplification product produced using the probe or primer. Methods for producing such a labeled probe or primer are known in the art. Furthermore, commercial sources for the production of a labeled probe or primer will be known to the skilled artisan, e.g., Sigma-Genosys, Sydney, Australia.

Clearly, the present invention encompasses a probe or primer comprising at least 20 nucleotides that is capable of selectively hybridizing to the nucleotide sequence set forth in SEQ ID NO: 1 and detecting a marker that is associated with α-disease or disorder associated with aberrant GSK-3β activity (preferably, a neurodegenerative disease or a psychiatric disease). Preferably, the probe or primer comprises a nucleotide sequence selected from the group consisting of SEQ ID NO: 10, SEQ ID NO: 11, SEQ ID NO: 50, SEQ ID NO: 51, SEQ ID NO: 54 and SEQ ID NO: 55.

The present invention additionally contemplates the use a probe or primer produced according to the methods described herein in the manufacture of a diagnostic reagent for diagnosing or determining a predisposition to a disease or disorder associated with aberrant GSK-3β expression and/or activity (e.g., a neurodegenerative disease or a psychiatric disease).

Detection Methods

Methods for detecting nucleic acids are known in the art and include for example, hybridization based assays, amplification based assays and restriction endonuclease based assays. For example, a change in the sequence of a region of the genome or an expression product thereof, such as, for example, an, insertion, a deletion, a transversion, a transition, alternative splicing or a change in the preference of or occurrence of a splice form of a gene is detected using a method, such as, polymerase chain reaction (PCR) strand displacement amplification, ligase chain reaction, cycling probe technology or a DNA microarray chip amongst others.

Methods of PCR are known in the art and described, for example, in Dieffenbach (Ed) and Dveksler (Ed) (In: PCR Primer: A Laboratory Manual, Cold Spring Harbour Laboratories, NY, 1995). Generally, for PCR two non-complementary nucleic acid primer molecules comprising at least about 20 nucleotides, and more preferably at least nucleotides are hybridized to different strands of a nucleic acid template molecule, and specific nucleic acid molecule copies of the template are amplified enzymatically. PCR products may be detected using electrophoresis and detection with a detectable marker that binds nucleic acids. Alternatively, one or more of the oligonucleotides are labeled with a detectable marker (e.g. a fluorophore) and the amplification product detected using, for example, a lightcycler (Perkin Elmer, Wellesley, Mass., USA). Alternatively, PCR products are detected, for example, using mass spectrometry. Clearly, the present invention also encompasses quantitative forms of PCR, such as, for example, a Taqman assay.

Strand displacement amplification (SDA) utilizes oligonucleotides, a DNA polymerase and a restriction endonuclease to amplify a target sequence. The oligonucleotides are hybridized to a target nucleic acid and the polymerase used to produce a copy of this region. The duplexes of copied nucleic acid and target nucleic acid are then nicked with an endonuclease that specifically recognizes a sequence of nucleotides at the beginning of the copied nucleic acid. The DNA polymerase recognizes the nicked DNA and produces another copy of the target region at the same time displacing the previously generated nucleic acid. The advantage of SDA is that it occurs in an isothermal format, thereby facilitating high-throughput automated analysis.

Ligase chain reaction (described in, for example, EU 320,308 and U.S. Pat. No. 4,883,750) uses two or more oligonucleotides that hybridize to adjacent target nucleic acids. A ligase enzyme is then used to link the oligonucleotides. In the presence of a nucleotide that is not complementary to the nucleotide at an end of one of the primers that is adjacent to the other primer, the ligase is unable to link the primers, thereby failing to produce a detectable amplification product. Using thermocycling the ligated oligonucleotides then become a target for further oligonucleotides. The ligated fragments are then detected, for example, using electrophoresis, or MA/DI-TOF. Alternatively, or in addition, one or more of the probes is labeled with a detectable marker, thereby facilitating rapid detection.

Cycling Probe Technology uses chimeric synthetic probe that comprises DNA-RNA-DNA that is capable of hybridizing to a target sequence. Upon hybridization to a target sequence the RNA-DNA duplex formed is a target for RNase H that cleaves the probe. The cleaved probe is then detected using, for example, electrophoresis or MALDI-TOF.

In a preferred embodiment, a detection method detects the presence or absence of a specific allele at the site of a SNP. Methods for detecting SNPs are known in the art, and reviewed, for example, in Landegren et al, Genome Research 8: 769-776, 1998.

For example, a SNP that introduces or alters a sequence that is a recognition sequence for a restriction endonuclease is detected by digesting DNA with the endonuclease and detecting the fragment of interest using, for example, Southern blotting (described in Ausubel et al (In: Current Protocols in Molecular Biology. Wiley Interscience, ISBN 047 150338, 1987) and Sambrook et al (In: Molecular Cloning: Molecular Cloning: A Laboratory Manual, Cold Spring Harbor Laboratories, New York, Third Edition 2001)). Alternatively, a nucleic acid amplification method described supra, is used to amplify the region surrounding the SNP. The amplification product is then incubated with the endonuclease and any resulting fragments detected, for example, by electrophoresis, MALDI-TOF or PCR.

The direct analysis of the sequence of polymorphisms of the present invention can be accomplished using either the dideoxy-chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)). As will be apparent to the skilled artisan, the presence of a polymorphism in a homozygous or heterozygous form may be detected using standard sequencing methods.

Alternatively, a SNP is detected using single stranded conformational polymorphism (SSCP) analysis. SSCP analysis relies upon the formation of secondary structures in nucleic acids and the sequence dependent nature of these secondary structures. In one form of this analysis an amplification method, such as, for example, a method described herein, is used to amplify a nucleic acid that comprises a SNP. The amplified nucleic acids are then denatured, cooled and analyzed using, for example, non-denaturing polyacrylamide gel electrophoresis, mass spectrometry, or liquid chromatography (e.g. HPLC or dHPLC). Regions that comprise different sequences form different secondary structures, and as a consequence migrate at different rates through, for example, a gel and/or a charged field. Accordingly, both homozygous forms of a SNP and a heterozygous form of the SNP may be detected using such analysis. Clearly, a detectable marker may be incorporated into a probe/primer useful in SSCP analysis to facilitate rapid marker detection. SSCP analysis is also particularly suited to determining whether a SNP is in a homozygous or a heterozygous form, as it detects and differentiates between both forms of the polymorphism.

Alternatively, any nucleotide changes are detected using, for example, mass spectrometry or capillary electrophoresis. For example, amplified products of a region of DNA comprising a SNP from a test sample are mixed with amplified products from a normal/healthy individual. The products are denatured and allowed to re-anneal. Clearly, those samples that comprise a different nucleotide at the position of the SNP will not completely anneal to a nucleic acid molecule from a normal/healthy individual thereby changing the charge and/or conformation of the nucleic acid, when compared to a completely annealed nucleic acid. Such incorrect base pairing is detectable using, for example, mass spectrometry. As with SSCP, methods for detecting a polymorphism using, for example, mass spectrometry or capillary electrophoresis are particularly suited for determining whether a polymorphism is in a homozygous form or a heterozygous form.

Mass spectrometry is also useful for detecting the molecular weight of a short amplified product, wherein a nucleotide change causes a change in molecular weight of the nucleic acid molecule (such a method is also described, for example, in U.S. Pat. No. 6,574,700).

Allele specific PCR (as described, for example, In Liu et al, Genome Research, 7: 389-398, 1997) is also useful for determining the presence of one or other allele of a SNP. An oligonucleotide is designed, in which the most 3′ base of the oligonucleotide hybridizes with the SNP. During a PCR reaction, if the 3′ end of the oligonucleotide does not hybridize to a target sequence, little or no PCR product is produced, indicating that a base other than that present in the oligonucleotide is present at the site of SNP in the sample. PCR products are then detected using, for example, gel or capillary electrophoresis or mass spectrometry.

Primer extension methods (described, for example, in Dieffenbach (Ed) and Dveksler (Ed) (In: PCR Primer: A Laboratory Manual, Cold Spring Harbour Laboratories, NY, 1995)) are also useful for the detection of a SNP. An oligonucleotide is used that hybridizes to the region of a nucleic acid adjacent to the SNP. This oligonucleotide is then used in a primer extension protocol with a polymerase and a free nucleotide diphosphate or dideoxynucleotide triphosphate that corresponds to either or any of the possible bases that occur at the SNP. Preferably the nucleotide-diphosphate is labeled with a detectable marker (e.g. a fluorophore). Following primer extension, unbound labeled nucleotide diphosphates are removed, e.g. using size exclusion chromatography or electrophoresis, or hydrolyzed, using for example, alkaline phosphatase, and the incorporation of the labeled nucleotide into the oligonucleotide is detected, indicating the base that is present at the site of the SNP. Alternatively, or in addition, as exemplified herein primer extension products are detected using mass spectrometry (e.g. MALDI-TOF).

By using a plurality of different free nucleotide diphosphates, wherein each of the free nucleotide diphosphates is labeled with a different detectable marker, the heterozygosity or homozygosity of a polymorphism is determined.

Clearly, the present invention extends to high-throughput forms primer extension analysis, such as, for example, minisequencing (Sy Vämen et. al., Genomics 9: 341-342, 1995). In such a method, a probe or primer (or multiple probes or primers) are immobilized on a solid support (e.g. a glass-slide). A biological sample comprising nucleic acid is then brought into direct contact with the probe/s or primer/s, and a primer extension protocol performed with each of the free nucleotide bases labeled with a different detectable marker. The nucleotide present at a SNP or a number of SNPs is then determined by determining the detectable marker bound to each probe and/or primer.

Fluorescently labeled locked nucleic acid (LNA) molecules or fluorescently labeled protein-nucleic acid (PNA) molecules are useful for the detection of SNPs (as described in Simeonov and Nikiforov, Nucleic Acids Research, 30(17): 1-5, 2002). LNA and PNA molecules bind, with high affinity, to nucleic acid, in particular, DNA. Fluorophores (in particular, rhodomine or hexachlorofluorescein) conjugated to the LNA or PNA probe fluoresce at a significantly greater level upon hybridization of the probe to target nucleic acid. However, the level of increase of fluorescence is not enhanced to the same level when even a single nucleotide mismatch occurs. Accordingly, the degree of fluorescence detected in a sample is indicative of the presence of a mismatch between the LNA or PNA probe and the target nucleic acid, such as, in the presence of a SNP. Preferably, fluorescently labeled LNA or PNA technology is used to detect a single base change in a nucleic acid that has been previously amplified using, for example, an amplification method known in the art and/or described herein.

As will be apparent to the skilled artisan, LNA or PNA detection technology is amenable to a high-throughput detection of one or more markers by immobilizing an LNA or PNA probe to a solid support, as described in Orum et al., Clin. Chem. 45: 1898-1905, 1999.

Molecular Beacons™ are also useful for detecting SNPs directly in a sample or in an amplified product (see, for example, Mhlang and Malmberg, Methods 25: 463-471, 2001). Molecular Beacons™ are single stranded nucleic acid molecules with a stem-and-loop structure. The loop structure is complementary to the region surrounding the SNP of interest. The stem structure is formed by annealing two “arms” complementary to each other that are on either side of the probe (loop). A fluorescent moiety is bound to one arm and the other arm comprises a quenching moiety that suppresses any detectable fluorescence when the molecular beacon is not bound to a target sequence. Upon binding of the loop region to its target nucleic acid the arms are separated and fluorescence is detectable. However, even a single base mismatch significantly alters the level of fluorescence detected in a sample. Accordingly, the presence or absence of a particular base at the site of a SNP is determined by the level of fluorescence detected.

A single nucleotide polymorphism can also be identified by hybridization to nucleic acid arrays, an example of which is described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

Clearly, the present invention encompasses other methods of detecting a SNP that is within a GSK-3β gene and associated with a disease or disorder associated with aberrant GSK-3β activity and/or expression, such as, for example, a SNP microarray (for example, as commercially available from Affymetrix, and/or described, for example, in U.S. Pat. No. 6,468,743 or Hacia et al, Nature Genetics, 14: 441, 1996), a Taqman assay (as described, for example, in Livak et al, Nature Genetics, 9: 341-342, 1995), solid phase minisequencing (as described in Syvämen et al, Geonomics, 13: 1008-1017, 1992), minisequencing with FRET (as described in Chen and Kwok, Nucleic Acids Res. 25: 347-353, 1997) or pyrominisequencing (as reviewed in Landegren et al., Genome Res., 8(8): 769-776, 1998).

In a preferred embodiment, the disease or disorder is a neurodegenerative disease and the neurodegenerative disease or a predisposition to the neurodegenerative disease is determined by performing a method comprising:
(i) amplifying nucleic acid from the subject using an amplification reaction, wherein the amplification reaction is performed using a pair of primers selected from the group consisting of:
   (a) a primer comprising a nucleotide sequence set forth in SEQ ID NO: 10 and a primer comprising a nucleotide sequence set forth in SEQ ID NO: 11; and
   (b) a primer comprising a nucleotide sequence set forth in SEQ ID NO: 50 and a primer comprising a nucleotide sequence set forth in SEQ ID NO: 51; and
(ii) detecting a polymorphism in the amplified nucleic acid from (i), wherein said polymorphism is indicative of a neurodegenerative disease or a predisposition to a neurodegenerative disease.

Preferably, the polymorphism is detected by determining the nucleotide sequence of the amplified nucleic acid.

### Detection of Altered Splicing of a GSK-3β Gene—Positive Assays

As exemplified herein, a SNP in a GSK-3β gene that is associated with a disease or disorder associated with aberrant GSK-3β expression and/or activity is also associated and/or causes a change in splicing of a GSK-3β mRNA. Accordingly, such an alternate splice form provides a marker associated with a disease or disorder. Alternatively, a marker associated with a disease or disorder is detected by detecting the alternate splice form of a GSK-3β transcript.

In one embodiment, an alternatively spliced GSK-3β transcript comprises additional nucleic acid, e.g., an additional exon. Preferably, the alternatively spliced GSK-3β transcript comprises additional nucleic acid compared to a transcript that consists of the nucleotide sequence set forth in SEQ ID NO: 2.

Such alternate splice forms may be determined using a positive read-out assay. In the present context, the term “positive read-out assay” shall be taken to mean that a positive result in an assay indicates that a biological sample comprises an alternatively spliced GSK-3β transcript.

A positive read-out assay that detects such a marker associated with a disease or disorder or a predisposition to a disease or disorder comprises detecting the presence of an additional exon in the transcript.

In one embodiment, the marker is detected by hybridizing a nucleic acid probe or primer comprising, consisting of or located within the region of alternate splicing of a GSK-3β transcript to a nucleic acid that is linked to the marker in a biological sample derived from a subject and detecting the hybridization by a detection means, wherein hybridization of the probe or primer indicates that the subject being tested is predisposed to or suffers from a disease or disorder disease. Preferably, the detection means is an amplification reaction, or a nucleic acid hybridization reaction, such as, for example, as described herein.

In an alternative embodiment, the marker is detected by amplifying (e.g., using PCR, RT-PCR, NASBA, TMA or ligase chain reaction amongst other methods) an alternatively spliced region of a GSK-3β transcript. In accordance with this embodiment, a probe or primer (or two or more probes or primers) that flank or abut the region of a GSK-3β transcript that is alternatively spliced are hybridized to a nucleic acid linked to the marker in a biological sample. The marker is then detected using an amplification or primer extension protocol. Clearly, such a method is useful for detecting insertion of additional nucleic acid or removal of nucleic acid (i.e., splicing-out of nucleic acid e.g., an exon). Detection of an alternatively spliced region of a GSK-3β transcript indicates that a subject is at risk of developing a disease or disorder associated with aberrant GSK-3β expression and/or activity or suffers from such a disease or disorder.

As will be apparent to the skilled artisan, an alternatively spliced region of a GSK-3β transcript may be detected using a probe or primer that hybridizes to a region that flanks or is adjacent to a region of alternative splicing of a GSK-3β transcript and a probe or primer that hybridizes to a region of the GSK-3β transcript that is alternatively spliced. Furthermore, a probe or primer may hybridize to an alternative splice site (i.e., a region of the probe or primer hybridizes to the region adjacent to the alternative splice site and another region of the probe or primer hybridizes to the region of the GSK-3β transcript that is alternatively spliced).

As alternatively spliced forms of GSK-3β are detected using mRNA or cDNA derived therefrom, assays that detect changes in mRNA are particular preferred (e.g. RT-PCR, NASBA, TMA or ligase chain reaction).

Methods of RT-PCR are known in the art and described, for example, in Dieffenbach (ed) and Dveksler (ed) (In: PCR Primer: A Laboratory Manual, Cold Spring Harbour Laboratories, NY, 1995).

Methods of TMA or self-sustained sequence replication (3SR) use two or more oligonucleotides that flank a target sequence, a RNA polymerase, RNase H and a reverse transcriptase. One oligonucleotide (that also comprises a RNA polymerase binding site) hybridizes to an RNA molecule that comprises the target sequence and the reverse transcriptase produces cDNA copy of this region. RNase H is used to digest the RNA in the RNA-DNA complex, and the second oligonucleotide used to produce a copy of the cDNA. The RNA polymerase is then used to produce a RNA copy of the cDNA, and the process repeated.

NASBA systems relies on the simultaneous activity of three enzymes (a reverse transcriptase, RNase H and RNA polymerase) to selectively amplify target mRNA sequences. The mRNA template is transcribed to cDNA by reverse transcription using an oligonucleotide that hybridizes to the target sequence and comprises a RNA polymerase binding site at its 5′ end. The template RNA is digested with RNase H and double stranded DNA is synthesized. The RNA polymerase then produces multiple RNA copies of the cDNA and the process is repeated.

Clearly, the hybridization to and/or amplification of a marker associated with a disease or disorder associated with aberrant GSK-3β activity and/or expression using any of these methods is detectable using, for example, electrophoresis and/or mass spectrometry. In this regard, one or more of the probes/primers and/or one or more of the nucleotides used in an amplification reactions may be labeled with a detectable marker to facilitate rapid detection of a marker, for example, a fluorescent label (e.g. Cy5 or Cy3) or a radioisotope (e.g. ³²P).

Alternatively, amplification of a nucleic acid may be continuously monitored using a melting curve analysis method, such as that described in, for example, U.S. Pat. No. 6,174,670. Such methods are suited to determining the level of an alternative splice form in a biological sample.

Clearly, the present invention also encompasses the use of any nucleic acid detection method to detect alternative splicing in a GSK-3β mRNA or cDNA derived therefrom.

In a preferred embodiment, an alternatively spliced GSK-3β transcript comprises fewer exons that a wild-type GSK-3β (i.e. a GSK-3β transcript comprising the sequence set forth in SEQ ID NO: 2), e.g., a nucleic acid comprising or consisting of the nucleotide sequence set forth in SEQ ID NO: 4, SEQ ID NO: 6 or SEQ ID NO: 8. Preferably, an alternatively spliced GSK-3β lack exon 9 or exon 11 or both exon 9 and exon 11.

A positive read-out assay that detects an alternate splice-form of a GSK-3β transcript that lacks an exon comprises hybridizing a probe or primer to a region of a GSK-3β transcript adjacent to the splice site and determining whether or not the exon is present.

For example a PCR primer is hybridized to a region 5′ to the splice site, and another. PCR primer is hybridized to a region 3′ of the splice site. Following amplification of the intervening sequence using a DNA polymerase, the size of the PCR product indicates whether or not the exon is present. Clearly, any amplification assay, e.g. RT-PCR, NASBA, TMA or ligase chain reaction is amenable to this form of analysis.

Alternatively, a probe or primer hybridizes to the region of the GSK-3β transcript comprising the splice site of interest. Accordingly, such a probe or primer will only hybridize to a sample that has been alternatively spliced. A Molecular Probe and/or a LNA probe and/or a PNA probe is particularly amenable to this form of analysis.

### Detection of Altered Splicing of a GSK-3βGene—Negative Assays

As will be apparent the skilled artisan, an alternate splice form of a GSK-3β transcript is also detectable using a negative read-out assay. As used herein, the term “negative read-out assay” shall be taken to mean that a negative result in an assay indicates that a biological sample comprises an alternatively spliced GSK-3β transcript.

For example, a negative read-out assay for the detection of an alternative splice-form of a GSK-3β transcript comprises hybridizing a probe or primer to a region of a GSK-3β transcript that is “spliced-out” or absent in the alternative splice form of interest. In one embodiment, such a probe or primer hybridizes to exon 9 or exon 11 of a GSK-3β transcript. As will be apparent to the skilled artisan, such a probe or primer will only hybridize to a form of a GSK-3β transcript that includes or comprises the region that is spliced. Accordingly, an assay that fails to detect hybridization of the probe or primer to nucleic acid in a biological sample (or detects a reduced amount of hybridization compared to a suitable control) indicates the presence of an alternative splice form of a GSK-3β transcript.

The detection of the hybridization of a probe or primer to nucleic acid in a biological sample is detected using any method known in the art, such as, for example, a method described supra. For example, a PCR primer is designed such that it hybridizes to exon 9 of GSK-3β. A second PCR primer is designed that hybridizes to exon 10 of GSK-3β. Use of such primers in a PCR reaction facilitates detection of a GSK-3β transcript that has spliced out exon 9. As will be apparent to the skilled artisan, the first oligonucleotide will not hybridize to nucleic acid in a biological sample in which there is a GSK-3β transcript that lacks exon 9. Accordingly, a reduced level of PCR product will be produced. Such a method may also be developed for the detection of alternate splicing of exon 11 and/or both exon 9 and exon 11 (e.g. using an oligonucleotides that hybridizes to exon 9 and an oligonucleotides that hybridizes to exon 11).

As will be apparent to the skilled artisan a negative read-out assay for the detection of an alternative splice-form of a GSK-3β transcript may also include a positive control Such a control is useful as failure to detect the alternative splice-form of a GSK-3β transcript may indicate that the detection reaction has failed. Such a positive control may involve, for example, using a probe or primer that hybridizes to a region of GSK-3β that is not alternatively spliced to determine the presence of GSK-3β encoding nucleic acid in a biological sample. Alternatively, a positive control may comprise a probe or primer that hybridizes to a transcript of a gene other than GSK-3β (that is known to be expressed in the biological sample) to determine the presence of nucleic acid in a biological sample. Examples of such genes include, actin, glyceraldehyde 3-phosphate dehydrogenase (GAPDH), β2 microglobulin, hydroxy-methylbilane synthase, hypoxanthine phosphoribosyl-transferase 1 (HPRT), ribosomal protein L13c, succinate dehydrogenase complex subunit A and TATA box binding protein (TBP) amongst others.

### Detection of Amino Acid Changes in a GSK-3β Polypeptide

In one embodiment, a marker associated with a disease or disorder associated with aberrant GSK-3β activity and/or expression is within a GSK-3β polypeptide. Such a marker is, for example, a single amino acid change, or the addition of or deletion of regions of a GSK-3β polypeptide (e.g. as a result of alternate splicing of a GSK-3β transcript.

Preferably, a protein marker encoded by nucleic acid that is associated with a disease or disorder associated with aberrant GSK-3β expression or activity is suitable for antigen-based detection. As will be apparent to the skilled artisan, even a single amino acid change in a polypeptide is detectable in an antigen-based assay, using, for example a monoclonal antibody, a single chain antibody, or an F′ab antibody fragment. However, for an antibody or ligand to detect such a change, a marker is preferably at least about 6 amino acids in length, more preferably at least about 8 to 10 amino acids in length, even more preferably at least about 14 amino acids in length. A protein marker may also be an entire protein, e.g. wherein the protein that is associated with a disease or disorder is, for example, a conformation different to the protein in a normal or healthy individual.

Generally, a polypeptide marker is detected in an assay that utilizes a ligand and/or antibody capable of binding to the marker.

As used herein the term “ligand” shall be taken in its broadest context to include any chemical compound, polynucleotide, peptide, protein, lipid, carbohydrate, small molecule, natural product, polymer, etc. that is capable of selectively binding, whether covalently or not, to one or more specific sites on a GSK-3β polypeptide. The ligand may bind to its target via any means including hydrophobic interactions, hydrogen bonding, electrostatic interactions, van der Waals interactions, pi stacking, covalent bonding, or magnetic interactions amongst others. It is preferred that a ligand is capable of specifically binding to a particular form of a GSK-3β polypeptide (e.g., a polypeptide encoded by an alternatively spliced GSK-3β mRNA).

As used herein the term “antibody” refers to intact monoclonal or polyclonal antibodies, immunoglobulin (IgA, IgD, IgG, IgM, IgE) fractions, humanized antibodies, or recombinant single chain antibodies, as well as fragments thereof, such as, for example Fab, F(ab)₂, and Fv fragments.

Antibodies referred to herein are obtained from a commercial source, or alternatively, produced by conventional means. For example, a polyclonal anti-GSK-3β antibody is available from Genex Bioscience Inc., Hayward, Calif., USA.

High titer antibodies are preferred, as these are more useful commercially in kits for analytical, diagnostic and/or therapeutic applications. By “high titer” is meant a titer of at least about 1:103 or 1:104 or 1:105. Methods of determining the titer of an antibody will be apparent to the skilled artisan. For example, the titer of an antibody in purified antiserum may be determined using an ELISA assay to determine the amount of IgG in a sample. Typically an anti-IgG antibody or Protein G is used in such an assay. The amount detected in a sample is compared to a control sample of a known amount of purified and/or recombinant IgG. Alternatively, a kit for determining antibody may be used, e.g. the Easy TITER kit from Pierce (Rockford, Ill., USA).

Antibodies may be prepared by any of a variety of techniques known to those of ordinary skill in the art, and described, for example in, Harlow and Lane (In: Antibodies: A Laboratory Manual, Cold Spring Harbor Laboratory, 1988). In one such technique, an immunogen comprising the antigenic polypeptide is initially injected into any one of a wide variety of animals (e.g., mice, rats, rabbits, sheep, humans, dogs, pigs, chickens and goats). The immunogen is derived from a natural source, produced by recombinant expression means, or artificially generated, such as by chemical synthesis (e.g., BOC chemistry or FMOC chemistry). In this step, the polypeptides or fragments thereof of this invention may serve as the immunogen.

A peptide, polypeptide or protein is joined to a carrier protein, such as bovine serum albumin or keyhole limpet hemocyanin. The immunogen and optionally a carrier for the protein is injected into the animal host, preferably according to a predetermined schedule incorporating one or more booster immunizations, and blood collected from said the animals periodically. Optionally, the immunogen may be injected in the presence of an adjuvant, such as, for example Freund's complete or incomplete adjuvant, lysolecithin and dinitrophenol to enhance the immune response to the immunogen. Monoclonal or polyclonal antibodies specific for the polypeptide may then be purified from the blood isolated from an animal by, for example, affinity chromatography using the polypeptide coupled to a suitable solid support.

Monoclonal antibodies specific for the antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described supra. The spleen cells are immortalized by, for example, fusion with a myeloma cell fusion partner, preferably one that is syngenic with the immunized animal. A variety of fusion techniques may be employed, for example, the spleen cells and myeloma cells may be combined with a nonionic detergent or electrofused and then grown in a selective medium that supports the growth of hybrid cells, but not myeloma cells. A preferred selection technique uses HAT (hypoxanthine, aminopterin, and thymidine) selection. After a sufficient time, usually about 1 to 2 weeks, colonies of hybrids are observed. Single colonies are selected and growth media in which the cells have been grown is tested for the presence of binding activity against the polypeptide (immunogen). Hybridomas having high reactivity and specificity are preferred.

Monoclonal antibodies are isolated from the supernatants of growing hybridoma colonies using methods such as, for example, affinity purification as described supra. In addition, various techniques may be employed to enhance the yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction. The marker associated with neurodegeneration of this invention may be used in the purification process in, for example, an affinity chromatography step.

It is preferable that an immunogen used in the production of an antibody is one which is sufficiently antigenic to stimulate the production of antibodies that will bind to the immunogen and is preferably, a high titer antibody. In one embodiment, an immunogen may be an entire protein.

In another embodiment, an immunogen consists of a peptide representing a fragment of a polypeptide, for example a region of a GSK-3β polypeptide that is alternatively spliced. Preferably an antibody raised to such an immunogen also recognizes the full-length protein from which the immunogen was derived, such as, for example, in its native state or having native conformation.

Alternatively, or in addition, an antibody raised against a peptide immunogen will recognize the full-length protein from which the immunogen was derived when the protein is denatured. By “denatured” is meant that conformational epitopes of the protein are disrupted under conditions that retain linear B cell epitopes of the protein. As will be known to a skilled artisan linear epitopes and conformational epitopes may overlap.

Alternatively, a monoclonal antibody capable of binding to a form of a GSK-3β polypeptide of interest or a fragment thereof is produced using a method such as, for example, a human B-cell hybridoma technique (Kozbar et al., Immunol. Today 4:72, 1983), a EBV-hybridoma technique to produce human monoclonal antibodies (Cole et al. Monoclonal Antibodies in Cancer Therapy, 1985 Allen R. Bliss, Inc., pages 77-96), or screening of combinatorial antibody libraries (Huse et al., Science 246:1275, 1989).

Such an antibody is then particularly useful in detecting the presence of a marker of a disease or disorder associated with aberrant GSK-3β activity and/or expression.

Positive Read-Out Assay

In one embodiment, the method of the invention detects the presence of a marker in a polypeptide that is associated with a disease or disorder associated with aberrant GSK-3β expression and/or activity. Such a marker may comprise, for example, an amino acid change encoded by a polymorphism.

In another embodiment, the marker associated with a disease or disorder associated with aberrant GSK-3β activity and/or expression comprises consists of or is within a GSK-3β polypeptide that is encoded by an alternatively spliced GSK-3β transcript. For example, a GSK-3β polypeptide comprising the amino acid sequence selected from the group consisting of SEQ ID NO: 5, SEQ ID NO: 7 and SEQ ID NO: 9. Clearly, a positive read-out assay is useful for the detection of a GSK-3β polypeptide that includes additional amino acids compared to the native GSK-3β (e.g., a polypeptide comprising the amino acid sequence set forth in SEQ ID NO: 3). Such a GSK-3β polypeptide may be encoded, for example by a transcript that comprises an additional exon compared to native GSK-3β.

A positive read-out assay is also amenable to the detection of a GSK-3β that comprises fewer amino acids than a native GSK-3β polypeptide (e.g., a polypeptide encoded by a GSK-3β transcript that has spliced out an exon). For example, a ligand or antibody that is capable of specifically binding to the region of the GSK-3β polypeptide that overlie the amino acids encoded by nucleic acids on either side of the splice site only binds those forms of the polypeptide that lack the alternatively spliced exon.

Alternatively, a conformational specific ligand/antibody is useful for detecting a change in a GSK-3β polypeptide that causes a change in the conformation of the polypeptide. Such a ligand/antibody preferably only detects the GSK-3β polypeptide encoded by the alternatively spliced transcript.

The amount, level or presence of a polypeptide is determined using any of a variety of techniques known to the skilled artisan such as, for example, a technique selected from the group consisting of, immunohistochemistry, immunofluorescence, an immunoblot, a Western blot, a dot blot, an enzyme linked immunosorbent assay (ELISA), radioimmunoassay (RIA), enzyme immunoassay, fluorescence resonance energy transfer (FRET), matrix-assisted laser desorption/ionization time of flight (MALDI-TOF), electrospray ionization (ESI), mass spectrometry (including tandem mass spectrometry, e.g. LC MS/MS), biosensor technology, evanescent fiber-optics technology or protein chip technology.

In one embodiment the assay used to determine the amount or level of a protein is a semi-quantitative assay.

In another embodiment the assay used to determine the amount or level of a protein in a quantitative assay.

Immunoassays

Preferably, the amount of antibody or ligand bound to a marker of a disease or disorder within a GSK-3β polypeptide is determined using an immunoassay. Preferably, using an assay selected from the group consisting of, immunohistochemistry, immunofluorescence, enzyme linked immunosorbent assay (ELISA), fluorescence linked immunosorbent assay (FLISA) Western blotting, RIA, a biosensor assay, a protein chip assay, a mass spectrometry assay, a fluorescence resonance energy transfer assay and an immunostaining assay (e.g. immunofluorescence).

Standard solid-phase ELISA or FLISA formats are particularly useful in determining the concentration of a protein from a variety of samples.

In one form such an assay involves immobilizing a biological sample onto a solid matrix, such as, for example a polystyrene or polycarbonate microwell or dipstick, a membrane, or a glass support (e.g. a glass slide).

An antibody that specifically binds to a marker of a disease or disorder within a GSK-3β polypeptide is brought into direct contact with the immobilized biological sample, and forms a direct bond with any of its target protein present in said sample. This antibody is generally labeled with a detectable reporter molecule, such as for example, a fluorescent label (e.g. FITC or Texas Red) or a fluorescent semiconductor nanocrystal (as described in U.S. Pat. No. 6,306,610) in the case of a FLISA or an enzyme (e.g. horseradish peroxidase (HRP), alkaline phosphatase (AP) or β-galactosidase) in the case of an ELISA, or alternatively a second labeled antibody can be used that binds to the first antibody. Following washing to remove any unbound antibody the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal) in the case of an enzymatic label.

Such ELISA or FLISA based systems are particularly suitable for quantification of the amount of a protein in a sample, by calibrating the detection system against known amounts of a protein standard to which the antibody binds, such as for example, an isolated and/or recombinant GSK-3β polypeptide or immunogenic fragment thereof or epitope thereof.

In another form, an ELISA consists of immobilizing an antibody or ligand that specifically binds a marker of a disease or disorder within a GSK-3β polypeptide on a solid matrix, such as, for example, a membrane, a polystyrene or polycarbonate microwell, a polystyrene or polycarbonate dipstick or a glass support. A sample is then brought into physical relation with said antibody, and said marker within a GSK-3β polypeptide is bound or ‘captured’. The bound protein is then detected using a labeled antibody. For example, if the marker is captured from a human sample, a labeled anti-human GSK-3β antibody that binds to an epitope that is distinct from the first (capture) antibody is used to detect the captured protein. Alternatively, a third labeled antibody can be used that binds the second (detecting) antibody.

It will be apparent to the skilled person that the assay formats described herein are amenable to high throughput formats, such as, for example automation of screening processes or a microarray format as described in Mendoza et al., Biotechniques 27(4): 778-788, 1999. Furthermore, variations of the above-described assay will be apparent to those skilled in the art, such as, for example, a competitive ELISA.

Alternatively, the presence of a marker of a disease or disorder within a GSK-3β polypeptide is detected using a radioimmunoassay (RIA). The basic principle of the assay is the use of a radiolabeled antibody or antigen to detect antibody-antigen interactions. An antibody or ligand that specifically binds to the marker within a GSK-3β polypeptide is bound to a solid support and a sample brought into direct contact with said antibody. To detect the level of bound antigen, an isolated and/or recombinant form of the antigen is radiolabeled and brought into contact with the same antibody. Following washing, the level of bound radioactivity is detected. As any antigen in the biological sample inhibits binding of the radiolabeled antigen the level of radioactivity detected is inversely proportional to the level of antigen in the sample. Such an assay may be quantitated by using a standard curve using increasing known concentrations of the isolated antigen.

As will be apparent to the skilled artisan, such an assay may be modified to use any reporter molecule, such as, for example, an enzyme or a fluorescent molecule, in place of a radioactive label.

In another embodiment, Western blotting is used to determine the level of a marker of a disease or disorder within a GSK-3β polypeptide in a sample. In such an assay protein from a sample is separated using sodium doedecyl sulphate polyacrylamide gel electrophoresis (SDS-PAGE) using techniques known in the art and described in, for example, Scopes (In: Protein Purification: Principles and Practice, Third Edition, Springer Verlag, 1994). Separated proteins are then transferred to a solid support, such as, for example, a membrane (e.g., a PVDF membrane), using methods known in the art, for example, electrotransfer. This membrane is then blocked and probed with a labeled antibody or ligand that specifically binds to a marker of a disease or disorder within a GSK-3β polypeptide. Alternatively, a labeled secondary, or even tertiary, antibody or ligand is used to detect the binding of a specific primary antibody. The level of label is then determined using an assay appropriate for the label used. An appropriate assay will be apparent to the skilled artisan.

For example, the level or presence a marker of a disease or disorder within a GSK-3β polypeptide is determined using methods known in the art, such as, for example, densitometry. In one embodiment, the intensity of a protein band or spot is normalized against the total amount of protein loaded on a SDS-PAGE gel using methods known in the art. Alternatively, the level of the marker detected is normalized against the level of a control/reference protein. Such control proteins are known in the art, and include, for example, actin, glyceraldehyde 3-phosphate dehydrogenase (GAPDH), β2 microglobulin, hydroxy-methylbilane synthase, hypoxanthine phosphoribosyl-transferase 1 (HPRT), ribosomal protein L13c, succinate dehydrogenase complex subunit A and TATA box binding protein (TBP).

In an alternative embodiment, a marker of a disease or disorder within a GSK-3β polypeptide is detected within a cell, using methods known in the art, such as, for example, immunohistochemistry or immunofluorescence.

For example, a cell or tissue section that is to be analyzed to determine the presence of a marker of a disease or disorder within a GSK-3β polypeptide is fixed to stabilize and protect both the cell and the proteins contained within the cell. Preferably, the method of fixation does not disrupt or destroy the antigenicity of the marker, thus rendering it undetectable. Methods of fixing a cell are known in the art and include for example, treatment with paraformaldehyde, treatment with alcohol, treatment with acetone, treatment with methanol, treatment with Bouin's fixative and treatment with glutaraldehyde. Following fixation a cell is incubated with a ligand or antibody capable of binding the marker. The ligand or antibody is, for example, labeled with a detectable marker, such as, for example, a fluorescent label (e.g. FITC or Texas Red), a fluorescent semiconductor nanocrystal (as described in U.S. Pat. No. 6,306,610) or an enzyme (e.g. horseradish peroxidase (HRP)), alkaline phosphatase (AP) or β-galactosidase. Alternatively, a second labeled antibody that binds to the first antibody is used to detect the first antibody. Following washing to remove any unbound antibody, the level of the bound to said labeled antibody is detected using the relevant detection means. Means for detecting a fluorescent label will vary depending upon the type of label used and will be apparent to the skilled artisan.

Optionally, a method of detecting a marker of a disease or disorder within a GSK-3β polypeptide using immunofluorescence or immunohistochemistry will comprise additional steps such as, for example, cell permeabilization (using, for example, n-octyl-BD-glucopyranoside, deoxycholate, a non-ionic detergent such as Triton X-100 NP-40, low concentrations of ionic detergents, such as, for example SDS or saponin) and/or antigen retrieval (using, for example, heat).

Methods using immunofluorescence are preferable, as they are quantitative or at least semi-quantitative. Methods of quantitating the degree of fluorescence of a stained cell are known in the art and described, for example, in Immunohistochemistry (Cuello, 1984 John Wiley and Sons, ASIN 0471900524).

Biosensor devices generally employ an electrode surface in combination with current or impedance measuring elements to be integrated into a device in combination with the assay substrate (such as that described in U.S. Pat. No. 5,567,301). An antibody/ligand that specifically binds to a marker of a disease or disorder associated with aberrant GSK-3β activity and/or expression is preferably incorporated onto the surface of a biosensor device and a biological sample contacted to said device. A change in the detected current or impedance by the biosensor device indicates protein binding to said antibody. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several proteins or peptides in a small amount of body fluids.

Evanescent biosensors are also preferred as they do not require the pretreatment of a biological sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the target polypeptide to the antibody or ligand.

Micro- or nano-cantilever biosensors are also preferred as they do not require the use of a detectable label. A cantilever biosensor utilizes a ligand and/or antibody capable of specifically detecting the analyte of interest that is bound to the surface of a deflectable arm of a micro- or nano-cantilever. Upon binding of the analyte of interest (e.g. a marker within a GSK-3β polypeptide) the deflectable arm of the cantilever is deflected in a vertical direction (i.e. upwards or downwards). The change in the deflection of the deflectable arm is then detected by any of a variety of methods, such as, for example, atomic force microscopy, a change in oscillation of the deflectable arm or a change in pizoresistivity. Exemplary micro-cantilever sensors are described in USSN 20030010097.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods of generating a protein chip are known in the art and are described in for example U.S. Patent Application No. 20020136821, 20020192654, 20020102617 and U.S. Pat. No. 6,391,625. To bind a protein to a solid support it is often necessary to treat the solid support so as to create chemically reactive groups on the surface, such as, for example, with an aldehyde-containing silane reagent. Alternatively, an antibody or ligand may be captured on a microfabricated polyacrylamide gel pad and accelerated into the gel using microelectrophoresis as described in, Arenkov et al. Anal. Biochem. 278:123-131, 2000.

A protein chip may comprise only one protein, ligand or antibody, and be used to screen one or more patient samples for the presence of one polypeptide of interest. Such a chip may also be used to simultaneously screen an array of patient samples for a polypeptide of interest.

Preferably, a protein sample to be analyzed using a protein chip is attached to a reporter molecule, such as, for example, a fluorescent molecule, a radioactive molecule, an enzyme, or an antibody that is detectable using methods known in the art. Accordingly, by contacting a protein chip with a labeled sample and subsequent washing to remove any unbound proteins the presence of a bound protein is detected using methods known in the art, such as, for example, using a DNA microarray reader.

Alternatively, biomolecular interaction analysis-mass spectrometry (BIA-MS) is used to rapidly detect and characterize a protein present in complex biological samples at the low- to sub-fmole level (Nelson et al. Electrophoresis 21: 1155-1163, 2000). One technique useful in the analysis of a protein chip is surface enhanced laser desorption/ionization-time of flight-mass spectrometry (SELDI-TOF-MS) technology, to characterize a protein bound to the protein chip. Alternatively, the protein chip is analyzed using ESI as described in U.S. Patent Application 20020139751.

As will be apparent from the preceding discussion, it is particularly preferred to employ a detection system that is antibody or ligand based as such assays are amenable to the detection of a marker of a disease or disorder within a GSK-3β polypeptide. Immunoassay formats are even more particularly preferred.

Negative Read-Out Assays

In one embodiment, a marker of a disease or disorder associated with aberrant GSK-3β activity and/or expression within a GSK-3β polypeptide is detected by contacting a biological sample derived from a subject with an antibody or ligand capable of specifically binding to said marker for a time and under conditions sufficient for an antibody/ligand complex to form and then detecting the complex wherein lack of detection of the complex indicates that the subject being tested is predisposed to or suffers from the disease or disorder.

In one embodiment, the marker associated with a disease or disorder comprises, consists of or is within a GSK-3β polypeptide that is encoded by an alternatively spliced GSK-3β transcript. For example, such a GSK-3β polypeptide comprising the amino acid sequence set forth in SEQ ID NO: 5, SEQ ID NO: 7 or SEQ ID NO: 9. A negative read-out assay is useful for the detection of a GSK-3β polypeptide that has fewer amino acids compared to the native GSK-3β (e.g., a polypeptide comprising the amino acid sequence set forth in SEQ ID NO: 3). Such a GSK-3β polypeptide may be encoded, for example by a transcript that comprises has fewer exons (e.g. has an exon spliced out) compared to a native GSK-3β.

A negative read-out assay is also amenable to the detection of a GSK-3β polypeptide that comprises additional amino acids compared to a native GSK-3 polypeptide (e.g., a polypeptide encoded by a GSK-3β transcript that has spliced out an exon). For example, a ligand or antibody that is capable of specifically binding to the region of the GSK-3β polypeptide that flank the splice site only binds those forms of the polypeptide that are encoded by a GSK-3β transcript that includes an alternatively spliced exon.

In accordance with this embodiment of the invention such a negative read-out assay will not produce a detectable result or will detect a reduced level of detection of a marker associated with a disease or disorder in a GSK-3β polypeptide. For example, an assay that uses an antibody capable of specifically binding an epitope within exon 11 of a GSK-3β polypeptide will not detect a form of GSK-3β that is encoded by a transcript that lack exon 11. Accordingly, a negative result indicates that the subject suffers from a disease or disorder and/or is at risk of developing a disease or disorder.

As will be apparent to the skilled artisan a negative read-out assay for the detection of a polypeptide encoded by an alternative splice-form of a GSK-3β transcript may also include a positive control Such a control is useful as failure to detect the polypeptide may indicate that the detection reaction has failed. Such a positive control may involve, for example, using an antibody or ligand that binds to a region of GSK-3β that is not alternatively spliced, to determine the presence of GSK-3β encoding nucleic acid in a biological sample. Alternatively, a positive control may comprise using an antibody or ligand that binds to a polypeptide other than GSK-3β (that is known to be expressed in the biological sample) to determine the presence of polypeptide in a biological sample. Suitable proteins are described supra.

Suitable methods for detecting a polypeptide are known in the art and/or described supra.

### Detection of an Enhanced Level of a GSK-3β Transcript

The present inventors have also shown that polymorphisms in the GSK-3β gene are associated with increased expression of a transcript of the GSK-3β gene in a subject suffering from a disease or disorder associated with aberrant GSK-3β activity and/or expression.

Accordingly, in one embodiment, a marker that is associated with a disease or disorder is detected by determining an enhanced or reduced level of a GSK-3β transcript in a sample derived from the subject, wherein said enhanced or reduced level of the GSK-3β transcript is indicative of a disease or disorder and/or a predisposition to a disease or disorder.

In one embodiment, the GSK-3β transcript comprises the amino acid sequence set forth in SEQ ID NO: 2. Alternatively, the GSK-3β transcript is an alternatively spliced GSK-3β transcript, for example, a nucleic acid comprising a nucleotide sequence at least 80% identical one or more nucleotide sequences selected from the group selected from SEQ ID NO: 4, SEQ ID NO: 6 and SEQ ID NO: 8.

Methods for detecting a transcript of a GSK-3β gene are described supra and are to be taken to apply mutatis mutandis to the present aspect of the invention. For example, the level of the GSK-3β transcript is determined by performing a process comprising hybridizing a nucleic acid probe that selectively hybridizes to the GSK-3β transcript with altered splicing to nucleic acid in a sample derived from the subject under moderate to high stringency hybridization conditions and detecting, the hybridization using a detection means, wherein the level of hybridization of the probe to the sample nucleic acid is indicative of the level of the GSK-3β transcript in the sample.

In one embodiment, an enhanced or reduced level of a GSK-3β transcript is detected by performing a process comprising:

(i) determining the level of the GSK-3β transcript in a sample derived from the subject;
(ii) determining the level of the GSK-3β transcript in a suitable control sample,
wherein an enhanced or reduced level of the GSK-3β transcript at (i) compared to (ii) is indicative of a disease or disorder and/or a predisposition to a disease or disorder. A suitable control sample is described herein.

Preferably, the level of a transcript comprising a nucleotide sequence set forth in SEQ ID NO: 6 or SEQ ID NO: 8 is enhanced in a subject suffering from a disease or disorder.

In another embodiment, the level of a transcript comprising a nucleotide sequence set forth in SEQ ID NO: 4 is reduced in a subject suffering from a disease or disorder.

### Detection of an Enhanced Level of a GSK-3β Polypeptide

The present inventors have also demonstrated that the level of expression of a GSK-3β polypeptide is associated with development of a disease or disorder associated with aberrant GSK-3β expression and/or activity.

Accordingly, in one embodiment, a marker that is associated with a disease or disorder is detected by determining an enhanced or reduced level of a GSK-3β polypeptide in a sample derived from the subject, wherein said enhanced or reduced level of the GSK-3β polypeptide is indicative of a disease or disorder and/or a predisposition to a disease or disorder.

Preferably, the polypeptide is encoded by an alternatively spliced GSK-3β transcript, such as, for example, a GSK-3β transcript comprising the nucleotide sequence set forth in SEQ ID NO: 4, SEQ ID NO: 6 or SEQ ID NO: 8. Accordingly, in one embodiment, the method comprises determining the level of a polypeptide comprising an amino acid sequence set forth in SEQ ID NO: 5, SEQ ID NO: 7 or SEQ ID NO: 9.

Methods for determining the level of expression of a polypeptide are described supra and are to be taken to apply mutatis mutandis to the present aspect of the invention. For example, the level of the GSK-3β polypeptide is detected by performing a process comprising contacting a biological sample derived from the subject with an antibody or ligand capable of selectively binding to the GSK-3β polypeptide for a time and under conditions sufficient for an antibody/ligand complex to form and then detecting the complex wherein the level of the complex is indicative of the level of the GSK-3β polypeptide in the subject.

Preferably, a method for detecting or determining an enhanced or reduced level of the GSK-3β polypeptide in a sample comprises performing a process comprising:

(i) determining the level of the GSK-3β polypeptide in the sample;
(ii) determining the level of the GSK-3β polypeptide in a suitable control sample,
wherein an enhanced or reduced level of the GSK-3β polypeptide at (i) compared to (ii) is indicative of a disease or disorder and/or a predisposition to a disease or disorder. A suitable control sample will be apparent to the skilled artisan and/or are described herein.

In a preferred embodiment, the level of a polypeptide comprising an amino acid sequence set forth in SEQ ID NO: 7 or SEQ ID NO: 9 is enhanced in a subject suffering from a disease or disorder.

Alternatively, or in addition, the level of a polypeptide comprising an amino acid sequence set forth in SEQ ID NO: 5 is reduced or undetectable in a subject suffering from a disease or disorder.

Determining a Subject with a Reduced Risk of Developing a Disease or Disorder

As will be apparent to the skilled person, the present invention additionally provides a method for determining a subject that has a reduced risk of developing a disease or disorder associated with aberrant GSK-3β expression and/or activity. Such a method comprises detecting a marker that is associated with a reduced risk of developing the disease or disorder in a subject.

In one embodiment, the marker that is associated with a reduced risk of developing the disease or disorder comprises or consists of a polymorphism within a GSK-3 gene that is in heterozygous form. In a particularly preferred embodiment, the marker comprises or consists of a single nucleotide polymorphism within a GSK-3β gene.

As used herein “heterozygous form” shall be taken to mean that a different form of the marker associated with a reduced risk of a disease or disorder associated with aberrant GSK-3β activity and/or expression occurs at the same locus on homologous chromosomes. For example, a different nucleotide is found at the site of a SNP located within a GSK-3β gene on each copy of chromosome 3.

Suitable polymorphisms will be apparent to the skilled person based on the description supra.

In a preferred embodiment, the marker associated with a reduced risk of developing a disease or disorder associated with aberrant GSK-3β expression and/or activity comprises or consists of a nucleotide located at a position corresponding to nucleotide position 181,700 of SEQ ID NO: 1. Preferably, the polymorphism is in a heterozygous form.

In a particularly preferred embodiment, the marker that is associated with a reduced risk of developing a disease or disorder associated with aberrant GSK-3β expression and/or activity comprises a cytosine at a position corresponding to nucleotide position 181,700 of SEQ ID NO: 1 within one copy of a GSK-3β genomic, gene and a thymidine at a position corresponding to nucleotide position 181,700 of SEQ ID NO: 1 within another copy of a GSK-3β genomic gene.

### Monitoring the Efficacy of Treatment

The methods described herein are also to be taken to apply mutatis mutandis to a method for monitoring the efficacy of treatment of a disease or disorder associated with aberrant GSK-3β activity and/or expression (e.g., a neurodegenerative disease).

In one embodiment, the present invention provides a method for monitoring the efficacy of treatment of a subject undergoing treatment for a disease or disorder comprising:
(i) determining the level of expression of a GSK-3β expression product in a sample derived from the subject; and
(ii) determining the level of expression of the GSK-3β expression product in a suitable control sample,
   wherein a similar level of expression of the GSK-3β expression product at (i) compared to (ii) indicates that the treatment is effective for the treatment of the disease or disorder.

Preferably, the GSK-3β expression product is a nucleic acid comprising a nucleotide sequence selected from the group consisting of SEQ ID NO: 4, SEQ ID NO: 6 and SEQ ID NO: 8.

Alternatively, the GSK-3β expression product is a polypeptide comprising an amino acid sequence selected from the group consisting of SEQ ID NO: 5, SEQ ID NO: 7 and SEQ ID NO: 9.

### Determining a Suitable Treatment

The present inventors have also shown that subjects that comprise or express a specific marker are more likely to respond positively to treatment with a specific therapeutic compound. Accordingly, in one embodiment, the present invention provides method for determining a subject likely to respond to a treatment for a disease or disorder associated with aberrant GSK-3β expression and/or activity, said method comprising detecting a marker within a GSK-3β gene or an expression product thereof that is associated with the disease or disorder in a sample derived from a subject, wherein the detection (or detected marker) is indicative of the disease or disorder or a predisposition to the disease or disorder in the subject.

In one embodiment, the disease or disorder is a neurodegenerative disease and the therapy is, for example, L-dopa, lithium or TDZD8.

In another embodiment, the disease or disorder is a psychiatric disorder and the therapy is, for example, lithium, carbamazepine, valproate, gabapentin, an antidepressant (e.g., bupropion) a neuroleptic or a benzodiazepine.

In a further embodiment, the disease or disorder is a disorder associated with aberrant glucose metabolism and the therapy is, for example, treatment with a biguanide, such as, for example, Metformin; treatment with an alpha-glucosidase inhibitor, such as, for example, Acarbose; or treatment with a compound such as, for example, troglitazone.

Suitable markers are described supra and are to be taken to apply to this embodiment of the invention. Preferably, the marker is a SNP comprising adenosine at a position corresponding to nucleotide position 1679 of SEQ ID NO: 49.

In a preferred embodiment, the disease or disorder is a bipolar affective disorder, the treatment is administration of lithium and the marker is a SNP comprising adenosine at a position corresponding to nucleotide position 1679 of SEQ ID NO: 49.

### Biological Samples

As embodiments of the present invention are based upon detection of a marker in genomic DNA any cell or sample that comprises genomic DNA is useful for determining a disease or disorder and/or a predisposition to a disease or disorder. Preferably, the cell or sample is derived from a human. Preferably, comprises a nucleated cell.

Preferred biological samples include, for example, whole blood, serum, plasma, peripheral blood mononuclear cells (PBMC), a buffy coat fraction, saliva, urine, a buccal cell, urine, fecal material, sweat or a skin cell.

In a particularly preferred embodiment, a biological sample comprises a white blood cell, more preferably, a lymphocyte cell.

Furthermore, as GSK-3β is almost ubiquitously expressed, any cell or sample comprising a cell may be used to determine a subject's predisposition to a disease or disorder associated with aberrant GSK-3β expression and/or activity or to diagnose the disease or disorder on the basis of a GSK-3β expression product provided that the cell expresses GSK-3β.

Alternatively, the biological sample is a cell isolated using a method selected from the group consisting of amniocentesis, chorionic villus sampling, fetal blood sampling (e.g. cordocentesis or percutaneous umbilical blood sampling) and other fetal tissue sampling (e.g. fetal skin biopsy). Such biological samples are useful for determining the predisposition of a developing embryo to a disease or disorder.

As will be apparent to the skilled artisan, the size of a biological sample will depend upon the detection means used. For example, an assay, such as, for example, PCR or single nucleotide primer extension may be performed on a sample comprising a single cell, although greater numbers of cells are preferred. Alternative forms of nucleic acid detection may require significantly more cells than a single cell. Furthermore, protein-based assays require sufficient cells to provide sufficient protein for an antigen based assay.

Preferably, the biological sample has been derived previously from the subject. Accordingly, in one embodiment, the method of the invention additionally comprises providing the biological sample.

In one embodiment, the method is performed using an extract from a biological sample, such as, for example, genomic DNA, mRNA, cDNA or protein.

As the present invention also includes detection of a marker in a GSK-3β gene that is associated with a disease or disorder in a cell (e.g. using immunofluorescence), the term “biological sample” also includes samples that comprise a cell or several cells, whether processed for analysis or not.

As will be apparent from the preceding description, such an assay may require the use of a suitable control, e.g. a normal individual or a typical population, e.g., for quantification.

As used herein, the term “normal individual” shall be taken to mean that the subject is selected on the basis that they do not comprise or express a marker that comprises, consists of or is within a GSK-3β gene or expression product thereof and that is associated with a disease or disorder, nor do they suffer from the disease or disorder.

For example, the normal subject has not been diagnosed with any form of neurodegenerative disease, using, for example, clinical analysis. For example, a subject may be tested for a neurodegenerative disease using a neuropsycbological test (e.g. a Wechsler Adult Intelligence Scale test, MDRS or GDS), an EEG, a CAT scan or a MRI scan.

Alternatively, the subject has not been diagnosed as suffering from or is not at risk of developing a disorder associated with aberrant glucose metabolism, e.g., type II diabetes or insulin resistance.

Alternatively, or in addition, a suitable control sample is a control data set comprising measurements of the marker being assayed for a typical population of subjects known not to suffer from a specific disease or disorder associated with aberrant GSK-3β activity and/or expression. Preferably the subject is not at risk of developing such a disease or disorder, and, in particular, the subject does not have a family history of the disease or disorder.

In the present context, the term “typical population” with respect to subjects known not to suffer from a disease or disorder and/or comprise or express a marker of a disease or disorder shall be taken to refer to a population or sample of subjects tested using, for example, known methods for determining the disease or disorder and determined not to suffer from the disease or disorder and/or tested to determine the presence or absence of a marker of the disease or disorder, wherein said subjects are representative of the spectrum of healthy subjects or subjects known not to suffer from the disease or disorder.

Given that many diseases or disorders are quantitative traits, a subject may suffer from the disease or disorder and not comprise or express a marker of the disease or disorder described herein. Alternatively, a subject may not suffer from the disease or disorder, yet comprise or express a marker of as described herein. However, a suitable control sample for the instant invention is a sample derived from a subject that does not suffer from the disease or disorder and does not comprise or express a marker of the disease or disorder (e.g., as described herein).

In one embodiment, a reference sample is not included in an assay. Instead, a suitable reference sample is derived from an established data set previously generated from a typical population. Data derived from processing, analyzing and/or assaying a test sample is then compared to data obtained for the sample population.

Data obtained from a sufficiently large number of reference samples so as to be representative of a population allows the generation of a data set for determining the average level of a particular parameter. Accordingly, the amount of a protein that is diagnostic of a disease or disorder or a predisposition to a disease or disorder can be determined for any population of individuals, and for any sample derived from said individual, for subsequent comparison to levels of the expression product determined for a sample being assayed. Where such normalized data sets are relied upon, internal-controls are preferably included in each assay conducted to control for variation.

Methods for Determining a Marker Associated with a Disease or Disorder

In one embodiment, the method of the invention additionally comprises determining an association between a marker and a disease or disorder associated with aberrant GSK-3β expression and/or activity.

Furthermore, given the tight association of the human GSK-3β gene to a disease or disorder associated with aberrant GSK-3β activity and/or expression, and the provision of several markers associated with a number of diseases or disorders, the present invention further provides methods for identifying new markers for a disease or disorder.

Accordingly, the present invention additionally provides a method for identifying a marker that is associated with a disease or disorder associated with aberrant GSK-3β expression and/or activity, said method comprising:

(i) identifying a polymorphism or allele within a GSK-3β gene or an expression product thereof;
(ii) analyzing a panel of subjects to determine those that suffer from the disease or disorder, wherein not all members of the panel comprise the polymorphism or allele; and
(iii) determining the variation in the development of the disease or disorder wherein said variation indicates that the polymorphism or allele is associated with a subject's predisposition to the disease or disorder.

Methods for determining the association between a marker and a disease, disorder and/or a phenotype are known in the art and reviewed, for example, in King (Ed) Rotter (Ed) and Motulski (Ed), The Genetic Basis of Common Disease, Oxford University Press, 2nd Edition, ISBN 0195125827, and Miller and Cronin (Eds), Genetic Polymorphisms and Susceptibility to Disease, Taylor and Francis, 1st Edition, ISBN 0748408223.

Generally, determining an association between a marker (e.g. a polymorphism and/or allele and/or a splice form) and a disease, disorder or phenotype involves comparing the frequency of a polymorphism, allele or splice form at a specific locus between a sample of unrelated affected individuals (i.e., they comprise the phenotype of interest and/or suffer from the disease/disorder of interest) and an appropriate control that is representative of the allelic distribution in the normal population. Accordingly, association studies are performed at the population level rather than within families, as is the case for linkage studies.

Several methods are useful for determining an association between a marker and a disease, disorder and/or phenotype of interest. However, any such study should consider several parameters to avoid difficulties, such as, for example, population stratification, that may produce false positive results.

Population stratification occurs when there are multiple subgroups with different allele frequencies present within a population. The different underlying allele frequencies in the sampled subgroups may be independent of the disease, disorder and/or phenotype within each group, and, as a consequence, may produce erroneous conclusions of linkage disequilibrium or association.

Generally, problems of population stratification are avoided by using appropriate control samples. For example, case-comparison based design may be used in which a comparison between a group of unrelated probands with the disease, disorder and/or phenotype and a group of control (comparison) individuals who are unrelated to each other or to the probands, but who have been matched to the proband group on relevant variable (other than affection status) that may influence genotype (e.g. sex, ethnicity and/or age).

Alternatively, controls are screened to exclude those subjects that have a personal history of the disease, disorder and/or phenotype of interest (and/or a family history of the disease, disorder and/or phenotype of interest). Such a “supernormal” control group is representative of the allele distribution of individuals unaffected by a disease, disorder and/or phenotype of interest.

Alternatively, a family-based association method may be used, in which non-transmitted alleles of the parents of a singly, ascertained proband are used as a random sample of alleles from which the proband was sampled. Such non-transmitted alleles are used to construct a matched control sample.

One extension of a family-based association method, the transmission disequilibrium test (TDT) uses a McNemar statistic to test for excess transmission of a marker allele to affected individuals above that expected by chance (Spielman et al., Am. J. Hum. Genet., 52: 506-516, 1993). Essentially, TDT considers parents who are heterozygous for an allele and/or polymorphism and/or splice variant associated with a disease, disorder or phenotype and evaluates the frequency with which the allele and/or polymorphism and/or splice variant or its alternate is transmitted to affected offspring. By only studying heterozygous parental genotypes TDT provides a test of association between linked loci and, as a consequence, avoids false associations between unlinked loci in the presence of population stratification.

The TDT method has been further refined to account for, for example multiallelic markers (Sham and Curtis Ann. Hum. Genet., 59: 323-326, 1995), multiple siblings in a family (Spielman and Ewens Am. J. Hum. Genet., 62:450-458, 1998), missing parental data (Curtis, Ann. Hum. Genet., 61: 319-333, 1997) and quantitative traits (Allison, Am. J. Hum. Genet., 60: 676-690, 1997 and Martin et al., Am. J. Hum. Genet., 67: 146-154, 2000).

In general, analysis of association is a test to detect non-random distribution of one or more alleles and/or polymorphisms and/or splice variants within subjects affected by a disease/disorder and/or phenotype of interest. The comparison between the test population and a suitable control population is made under the null hypothesis assumption that the locus to which the alleles and/or polymorphisms are linked has no influence on phenotype, and from this a nominal p-value is produced. For analysis of a biallelic polymorphism (e.g. a SNP) using a case control study, a chi-square analysis (or equivalent test) of a 2×2-contingency table (for analysis of alleles) or a 3×2 contingency table (for analysis of genotypes) is used.

For analysis using a family-based association study, marker data from members of the family of each proband are used to estimate the expected null distributions and an appropriate statistical test performed that compares observed data with that expected under the null hypothesis.

Another method useful in the analysis of association of a marker with a disease, disorder and/or phenotype is the genomic control method (Devlin and Roeder, Biometrics, 55: 997-1004, 1999). For a case-control analysis of candidate allele/polymorphism the genetic control method computes chi-square test statistics for both null and candidate loci. The variability and/or magnitude of the test statistics observed for the null loci are increased if population stratification and/or unmeasured genetic relationships among the subjects exist. This data is then used to derive a multiplier that is used to adjust the critical value for significance test for candidate loci. In this manner, genetic control permits analysis of stratified case-control data without an increased rate of false positives.

A structured association approach (Pritchard et al., Am. J. Hum. Genet., 67: 170-181, 2000) uses marker loci unlinked to a candidate marker to infer subpopulation membership. Latent class analysis is used to control for the effect of population substructure. Essentially, null loci are used to estimate the number of subpopulations and the probability of a subject's membership to each subpopulation. This method is then capable of accounting for a change in allele/polymorphism frequency as a result of population substructure.

Alternatively, or in addition, should a particular gene or gene product be likely to be involved in a disease, disorder or phenotype of interest a Bayesian statistical approach may be used to determine the significance of an association between an allele and/or polymorphism of that gene and the disease, disorder or phenotype of interest. Such an approach takes account of the prior probability that the locus under examination is involved in the disease, disorder or phenotype of interest (e.g., Morris et al., Am. J. Hum. Genet., 67:155-169, 2001).

Publicly available software is used to determine an association between an allele and/or polymorphism and/or a splice form and a disease or disorder or a predisposition to a disease or disorder. Such software include, for example, the following:
(i) Analysis of Complex Traits (ACT), which includes methods for multivariate analysis of complex traits. ACT is based on the research reported in Amos, et al., Ann. Hum. Genet. 60:143-160, 1996 and Amos, Am. J. Hun. Genet., 54:535-543, 1994;
(ii) ADMIXMAP, a general-purpose program for modeling admixture using marker genotypes and trait data of individuals from an admixed population; useful for estimate individual and population level admixture, test for a relationship between disease risk and individual admixture in case-control, cross-sectional or cohort studies, localize genes underlying ethnic differences in disease, risk by admixture mapping and control for population structure (variation in individual admixture) in genetic association studies so as to eliminate associations with unlinked genes;
(iii) ANALYZE, an accessory program for the LINKAGE program that facilitates both parametric and non-parametric tests for association;
(iii) BAMA (Bayesian analysis of multilocus association), useful for selecting a trait-associated subset of markers among many candidates; and,
(iv) CLUMP, a Monte Carlo method for assessing significance of a case-control association study with multi-allelic marker;
(v) ET-TDT (evolutionary tree-transmission disequilibrium test) and ETTDT (extended transmission disequilibrium test), extensions of the TDT test; and
(vi) FBAT (family based association test), useful for testing for association/linkage between disease phenotypes and haplotypes by utilizing family-based controls

Preferably, a marker that is determined using any of the methods described supra is within a GSK-3β gene and is associated with a disease or disorder associated with aberrant GSK-3β expression and/or activity (more preferably, a neurodegenerative disease).

As will be apparent to the skilled person, the methods described supra are also useful for determining a marker whether or not a marker that is associated with a disease or disorder associated with aberrant GSK-3β expression and/or activity is associated with another disease or disorder associated with aberrant GSK-3β expression and/or activity. Accordingly, the present invention additionally provides a method for determining a marker within a GSK-3β gene or expression product that is associated with a disease or disorder associated with aberrant GSK-3β expression and/or activity, said method-comprising:
(i) identifying a marker within a GSK-3β gene or expression product that is associated with a neurodegenerative disease and/or a psychiatric disease; and
(ii) determining a marker from (i) that is additionally associated with the disease or disorder associated with aberrant GSK-3β expression and/or activity.

In a preferred embodiment, the marker at (i) is associated with a neurodegenerative disease. For example, a suitable marker is described herein.

The present invention is also useful for determining a disease or disorder that is associated with a marker in a GSK-3 gene. For example, a marker described herein and/or identified using a method described herein. Accordingly, another embodiment of the invention provides a method for the identification of a disease or disorder associated with aberrant GSK-3β expression and/or activity, said method comprising:
(i) identifying a marker within a GSK-3β gene or expression product that is associated with a neurodegenerative disease and/or a psychiatric disease; and
(ii) determining a disease or disorder that is associated with the marker from (i).

Preferably, the marker is a marker described herein. Accordingly, in another embodiment, the method of the invention comprises determining a disease or disorder that is associated with a marker selected from the group consisting of a thymidine at a position corresponding to nucleotide position 181,700 of SEQ ID NO: 1, a cytosine at a position corresponding to nucleotide position 181,700 of SEQ ID NO: 1, a thymidine at a position corresponding to nucleotide position 232 of SEQ ID NO: 1, a cytosine at a position corresponding to nucleotide position 232 of SEQ ID NO: 1, an adenosine at a position corresponding to nucleotide position 1679 of SEQ ID NO: 49, a thymidine at a position corresponding to nucleotide position 1679 of SEQ ID NO: 49 and mixtures thereof.

Suitable methods for determining the association between a marker and a disease or disorder are described supra and are to be taken to apply mutatis mutandis to the present embodiment of the invention.

### Compounds for the Treatment of a Disease or Disorder

As exemplified herein, the level of alternative splicing of GSK-3β is modulated by a polymorphism associated with a disease or disorder. Accordingly, the present invention also provides a method of determining a candidate compound for the treatment of a disease or disorder associated with aberrant GSK-3β expression and/or, activity comprising:
(i) administering a candidate compound to an animal or cell comprising or expressing a marker within a GSK-3β gene that is associated with a disease or disorder associated with aberrant GSK-3β expression and/or activity and determining the level of alternative splicing of GSK-3β mRNA in said cell or animal;
(ii) administering a candidate compound to an animal or cell that does not comprise or express the marker within a GSK-3β gene and determining the level of alternative splicing of GSK-3β mRNA in said cell or animal; and
(iii) comparing the level of alternative splicing at (i) and (ii), wherein a decreased level of alternate splicing at (i) relative to (ii) indicates that the compound is a candidate compound for the treatment of a disease or disorder associated with aberrant GSK-3β expression and/or activity.

Preferably, the marker is associated with a neurodegenerative disease.

Preferably, the level of alternative splicing is determined by determining the level of different forms of a GSK-3β polypeptide in a cell or animal. Methods for determining the level of a GSK-3β polypeptide in a cell or animal are known in the art and/or described herein.

For example, standard solid-phase ELISA or FLISA assay formats are useful for identifying the level of expression of a GSK-3β polypeptide encoded by an alternatively spliced GSK-3β mRNA. In accordance with this embodiment, an antibody that specifically binds to a form of a GSK-3β polypeptide of interest (e.g. a polypeptide encoded by an alternate splice form of a GSK-3β gene that is associated with a disease or disorder) is immobilized on a solid matrix, such as, for example an array of polymeric pins or a glass support. Lysate from a cell that has been incubated in the presence of a candidate compound or a cell that has not been incubated in the presence of a candidate compound (a negative control) is brought into physical relation with the immobilized antibody. Alternatively, or in addition, the control cell is a positive control cell, e.g., the cell has been incubated in the presence of a compound that is a known therapeutic of the disease or disorder (e.g., lithium and/or L-DOPA in the case of a neurodegenerative disease), as a positive control. The level of captured GSK-3β polypeptide is then detected with a suitable antibody, e.g. another anti-GSK-3β antibody that binds to an epitope different to that of the capture antibody, or, for example, another anti-GSK-3β antibody. The antibody is generally labeled with fluorescent molecules or conjugated to an enzyme (e.g. horseradish peroxidase), or alternatively, a second labeled antibody can be used that binds to the first antibody. The level of GSK-3β polypeptide is then determined using, for example, a standard curve determined using known amounts of, for example, recombinant GSK-3β polypeptide, e.g., known amounts of GSK-3β encoded by an alternatively spliced transcript or cDNA derived therefrom. Alternatively, the level of a GSK-3β polypeptide detected in the sample comprising a candidate compound may be directly compared to the level of GSK-3β detected in a sample comprising no compound. Clearly, such an assay will require some degree of normalization, e.g., for total protein concentration.

In a preferred embodiment, the level of alternative splicing is determined by determining the level of forms of a GSK-3β mRNA in a cell or animal, for example, using PCR. Methods for determining the level of a specific isoform of GSK-3β are known in the art and/or described herein. For example, as exemplified herein, a PCR reaction that uses oligonucleotide primers that flank the region of GSK-3β spanning from exon 6 to exon 12 are useful for determining the level of an isoform that comprise exons 6 through 12, an isoform that lacks exon 9, an exon that lacks exon 11 and/or an exon that lacks both exon 9 and exon 11.

In another preferred embodiment, the level of alternate splicing is determined by an exon-trapping experiment. Methods of exon-tapping are known in the art and kits for such experiments are commercially available, for example from Stratagene (La Jolla, Calif., USA). Generally, an exon trapping experiment comprises cloning a region of a gene of interest into a vector such as, for example, pSPL1 or pSPL3 (Stratagene). Such a vector contains an artificial mini gene consisting of:
(i) a segment of the simian virus 40 (SV40) genome that contains an origin of replication and a promoter sequence;
(ii) two splicing-competent exons flanking an intron sequence which contains a multiple cloning site (MCS); and
(iii) an SV40 polyadenylation site.

The recombinant DNA is inserted into a restriction site in the MCS and the vector is transfected into a mammalian cell line e.g. COS7 cells or HEK293 cells. Transcription is driven by the SV40 promoter and the RNA undergoes splicing under the control of the host cell's RNA splicing machinery. The result is that any exon contained in the genomic fragment becomes attached between the upstream and downstream minigene exons. RT-PCR with primers specific for the minigene exons produces a PCR product containing the exons which can be detected using, for example agarose gel electrophoresis. Products containing additional exons are detected as larger bands when compared to PCR products produced using the vector alone. As the minigene sequence is already known, the nucleotide positions at which the inserted exon starts can be determined by, for example, sequencing of the RT-PCR product.

Depending on the size and number of fragments obtained after RT-PCR, several further experiments are performed. Sequencing, followed by database searches is useful for obtaining sequence information from the trapped sequences. Trapped exons are also putative cDNAs and can be used as probes for screening a cDNA library or as hybridization probes in a Southern blot or a Northern blot.

The present inventors have also shown that a polypeptide encoded by an alternatively spliced GSK-3β mRNA increases tau phosphorylation compared to wild-type GSK-3β. Accordingly, the present invention also provides a method of determining a candidate compound for the treatment of a disease or disorder associated with aberrant GSK-3β expression and/or activity comprising:
(i) administering a candidate compound to an animal or cell comprising or expressing a marker within a GSK-3β gene that is associated with a disease or disorder associated with aberrant GSK-3β expression and/or activity and determining the level of tau phosphorylation in said cell or animal;
(ii) administering a candidate compound to an animal or cell that does not comprise or express the marker within a GSK-3β gene and determining the level of determining the level of tau phosphorylation in said cell or animal; and
(iii) comparing the level of tau phosphorylation at (i) and (ii) wherein a decreased level of tau phosphorylation at (i) relative to (ii) indicates that the compound is a candidate compound for the treatment of a disease or disorder associated with aberrant GSK-3β expression and/or activity.

Preferably, the marker is associated with a neurodegenerative disease. Such an assay is useful for, for example, determining a candidate compound for the treatment of a neurodegenerative disease.

Preferably, the marker is an alternatively-spliced GSK-3β mRNA (or protein encoded thereby) that enhances tau phosphorylation in an animal or human subject or a cell derived therefrom.

Methods for determining tau phosphorylation are known in the art and/or described herein.

For example, an antibody that specifically binds phosphorylated tau is useful for an assay such as, for example, an ELISA. Both monoclonal and polyclonal anti-phosphorylated tau antibodies are commercially available, for example, from Research Diagnostics, Flanders, N.J., USA. Such an assay is described, for example, in Herrmann et al., European Neurology, 42: 205-210, 1999.

Hu et al., Am. J. Pathol., 160(4): 1269-1278, 2002 describe a bienzyme-substrate-recycle enzyme-linked immunosorbent assay for the detection of phosphorylated tau such an assay uses a primary antibody that binds to phosphorylated tan and a secondary antibody that is conjugated to alkaline phosphatase. Alkaline phosphatase dephosphorylates NADP⁺ to NAD⁺. The NAD⁺ then enters a highly NAD⁺-specific redox cycle, in which NAD⁺ is reduced to NADH by alcohol dehydrogenase, and the NADH produced is oxidized back to NAD⁺ by diaphorase with the concomitant reduction of resazurin (a nonfluorescent substrate) to resorufin (a fluorescent product). The resorufin accumulates with each cycle of NAD⁺-NADH-NAD⁺ and the fluorescence of resorufin is detected.

The present inventors have developed a cellular system with cells expressing wild type GSK-3β (encoded by a cDNA comprising the sequence set forth in SEQ ID NO: 2); or GSK-3β lacking exon 9 (encoded by a cDNA comprising the sequence set forth in SEQ ID NO: 4); or GSK-3β lacking exon 11 (encoded by a cDNA comprising the sequence set forth in SEQ ID NO: 6); or GSK-3 lacking exon 9 and exon 11 (encoded by a cDNA comprising the sequence set forth in SEQ ID NO: 2). The inventors showed that GSK-3β lacking exon 9 and exon 11 phosphorylated significantly more tau than wild type GSK-3β or GSK-3β lacking exon 9 or exon 11.

Using the cells expressing GSK-3β lacking exon 9 and exon 11 or GSK-3β lacking exon 9, the present inventors have shown that lithium is capable of suppressing the ability of GSK-3β lacking exon 9 and exon 11 to phosphorylate tau 3.7 times more efficiently than GSK-3β lacking exon 9. The inventors also found that the heterocyclic thiadiazolidinone competitive inhibitor of GSK-3β, TZD8 was also capable of suppressing the level of tau phosphorylation in a cell expressing GSK-3β lacking exon 9 and exon 11 at a level greater than observed in a cell expressing GSK-3β lacking exon 9.

Accordingly, in one embodiment, the present invention provides a method of determining a candidate compound for the treatment of a disease or disorder associated with aberrant GSK-3β expression and/or activity comprising:
(i) administering a candidate compound to an animal or cell comprising or expressing a marker within a GSK-3β gene that is associated with a disease or disorder associated with aberrant GSK-3β expression and/or activity- and increased tau phosphorylation and determining the level of tau phosphorylation in said cell or animal;
(ii) administering a candidate compound to an animal or cell that does not comprise or express the marker and determining the level of tau phosphorylation in said cell or animal; and
(iii) comparing the level of tau phosphorylation at (i) and (ii)
   wherein a decreased level of tau phosphorylation at (i) relative to (ii) indicates that the compound is a candidate compound for the treatment of a disease or disorder associated with aberrant GSK-3β expression and/or activity.

In one embodiment, the method additionally comprises normalizing the level of tau phosphorylation detected. For example, the level of phosphorylated tau detected is normalized relative to the number of cells in an assay.

In a preferred embodiment, the level of tau phosphorylation detected is normalized relative to the level of tau phosphorylation detected in an animal or cell comprising or expressing the marker within a GSK-3β gene that is associated with a disease or disorder associated with aberrant GSK-3β expression and/or activity that has not had the compound administered.

Accordingly, in one embodiment, the method comprises:
(i) administering a candidate compound to an animal or cell comprising or expressing a marker within a GSK-3β gene that is associated with a disease or disorder associated with aberrant GSK-3β expression and/or activity and determining the level of tau phosphorylation in said cell or animal;
(ii) determining the level of tau phosphorylation in a cell or animal comprising or expressing the marker that has not had the compound administered; and
(iii) comparing the level of tau phosphorylation at (i) and (ii), wherein a decreased level of tau phosphorylation at (i) relative to (ii) indicates that the compound is a candidate compound for the treatment of the disease or disorder.

In a preferred embodiment, the assay is performed in a cell, i.e., the assay is performed in vitro.

Methods for producing a cell expressing a particular form of GSK-3β will be apparent to the skilled artisan. For example, an expression conectruct is produced by placing a nucleic acid encoding the isoform of GSK-3β in operable connection with a promoter. The expression construct is then introduced into a cell and the cell incubated under conditions sufficient for expression to occur.

The term “promoter” is to be taken in its broadest context and includes the transcriptional regulatory sequences of a genomic gene, including the TATA box or initiator element, which is required for accurate transcription initiation, with or without additional regulatory elements (i.e. upstream activating sequences, transcription factor binding sites, enhancers and silencers) which alter gene expression in response to developmental and/or external stimuli, or in a tissue specific manner. In the present context, the term “promoter” is also used to describe a recombinant, synthetic or fusion molecule, or derivative which confers, activates or enhances the expression of a nucleic acid molecule to which it is operably linked, and which encodes the peptide or protein. Preferred promoters can contain additional copies of one or more specific regulatory elements to further enhance expression and/or alter the spatial expression and/or temporal expression of said nucleic acid molecule.

Placing a nucleic acid molecule under the regulatory control of, i.e., “in operable connection with”, a promoter sequence means positioning said molecule such that expression is controlled by the promoter sequence. Promoters are generally positioned 5′ (upstream) to the coding sequence that they control. To construct heterologous promoter/structural gene combinations, it is generally preferred to position the promoter at a distance from the gene transcription start site that is approximately the same as the distance between that promoter and the gene it controls in its natural setting, i.e., the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the gene from which it is derived. Again, as is known in the art, some variation in this distance can also occur.

Typical promoters suitable for expression in a mammalian cell, mammalian tissue or intact mammal include, for example, a promoter selected from the group consisting of, retroviral LTR elements, a SV40 early promoter, a SV40 late promoter, a cytomegalovirus (CMV) promoter, a CMV IE (cytomegalovirus immediate early) promoter, an EF_{1α} promoter (from human elongation factor 1α), an EM7 promoter, an UbC promoter (from human ubiquitin C).

Methods for producing expression constructs are known in the art and are described, for example, in Ausubel et al (In: Current Protocols in Molecular Biology. Wiley Interscience, ISBN 047 150338, 1987) or Sambrook et al (In: Molecular Cloning: Molecular Cloning: A Laboratory Manual, Cold Spring Harbor Laboratories, New York, Third Edition 2001).

Expression vectors that contain a suitable promoter for expression in a mammalian cell or a mammal include, but are not limited to, the pcDNA vector suite supplied by Invitrogen, the pCI vector suite (Promega), the pCMV vector suite (Clontech), the pM vector (Clontech), the pSI vector (Promega), the VP16 vector (Clontech) and the pDISPLAY-vectors (Invitrogen).

Preferred mammalian cells for expression of the nucleic acid fragments include, for example, a cell from a cell line selected from the group consisting of COS, CHO, murine 10T, MEF, NIH3T3, MDA-MB-231, MDCK, HeLa, K562, HEK 293′ and 293T. The use of a cell from another cell line or a primary cell is also contemplated herein.

Preferably, the cell expresses tau to facilitate determining the level of tau phosphorylation. Accordingly, a bacterial or a yeast cell may be used provided that the cell expresses tau and any other proteins required for tau phosphorylation by GSK-3β.

Following production of a suitable gene construct, said construct is introduced into the relevant cell: Methods of introducing the gene constructs into a cell or organism for expression are known to those skilled in the art and are described for example, in Ausubel et al (In: Current Protocols in Molecular Biology. Wiley Interscience, ISBN 047 150338, 1987) and (Sambrook et al (In: Molecular Cloning: Molecular Cloning: A Laboratory Manual, Cold Spring Harbor Laboratories, New York, Third Edition 2001). The method chosen to introduce the gene construct in depends upon the cell type in which the gene construct is to be expressed. Means for introducing recombinant DNA into cells include, but are not limited to electroporation or chemical transformation into-cells previously treated to allow for said transformation, PEG mediated transformation, microinjection, transfection mediated by DEAE-dextran, transfection mediated by calcium phosphate, transfection mediated by liposomes such as by using Lipofectamine (Invitrogen) and/or cellfectin (Invitrogen), transduction by Adenoviruses, Herpesviruses, Togaviruses or Retroviruses and microparticle bombardment such as by using DNA-coated tungsten or gold particles (Agacetus Inc., WI, USA).

### Suitable Compounds

In one embodiment, the compound administered comprises nucleic, acid, for example, the nucleic acid is an antagonist of GSK-3β expression, such as, for example, an antisense nucleic acid, peptide nucleic acid (PNA), ribozyme, or interfering RNA, which is complementary, in whole or in part, to a target molecule comprising a sense strand, and can hybridize with the target molecule. Preferably, the nucleic acid compound specifically binds to an alternatively spliced form of a GSK-3β (more preferably a form of a GSK-3β that lacks exon 9 or exon 11 or both exon 9 and 11). When introduced into a cell using suitable methods, such a nucleic acid inhibits the expression of the GSK-3β gene or a specific isoform thereof encoded by the sense strand. Antisense nucleic acid, ribozymes (e.g. Cech et al., U.S. Pat. No. 4,987,071; Cech et al, U.S. Pat. No. 5,116,742; Bartel and Szostak, Science 261, 1411-1418, 1993), nucleic acid capable of forming a triple helix (e.g. Helene, Anticancer Drug Res. 6, 569-584, 1991), PNAs (Hyrup et al., Bioorganic & Med. Chem. 4, 5-23, 1996; O'Keefe et al., Proc. Natl. Acad. Sci. USA 93, 14670-14675, 1996), interfering RNAs (Elbashir et al., Nature 411, 494-498, 2001; Sharp, Genes Devel. 15, 485-490, 2001; Lipardi et al., Cell 107, 297-307, 2001; Nishikura, Cell 107, 415-418, 2001) or small interfering RNAs (siRNA) may be produced by standard techniques known to the skilled artisan, based upon the sequences disclosed herein.

Accordingly, the antisense nucleic acid comprises a nucleic acid that is complementary to at least about 15-20 contiguous nucleotides of a sequence having at least about 80% identity to SEQ ID NOs: 1, 2, 4, 6 or 8. Ribozyme, PNA, interfering RNA or siRNA comprises a sequence that is complementary to at least about 15-20 contiguous nucleotides of a sequence having at least about 80% identity to SEQ ID NOs: 2, 4, 6 or 8 or a sequence transcribed from the sequence set forth in SEQ ID NO: 1 and can hybridize thereto. Preferably, an antisense ribozyme, PNA, interfering RNA or siRNA is capable of specifically hybridizing to a GSK-3β gene that comprises a marker that is associated with a disease or disorder associated with aberrant GSK-3β expression and/or activity.

Longer molecules, comprising a sequence that is complementary to at least about 25, or 30, or 35, or 40, or 45, or 50 contiguous nucleotides of mRNA encoding a human GSK-3β polypeptide are also encompassed by the present invention.

In one embodiment, the antisense molecule, ribozyme, PNA, RNAi or siRNA specifically hybridize to a splice form of GSK-3β. For example the antisense molecule, ribozyme, PNA, RNAi or siRNA comprises or consists of a sequence that is complementary to a region of a splice form of GSK-3β that spans a relevant splice site (e.g. a RNAi molecule comprises a sequence spans includes sequence from Exon 10 and Exon 12 of GSK-3β and, as a consequence only binds to those mRNA that do not comprise Exon 11).

In one embodiment, a construct comprising an antisense nucleic acid, ribozyme, PNA, interfering RNA or siRNA, is introduced into a suitable cell to inhibit expression of GSK-3β. In another embodiment, such a construct is introduced into some or all of the cells of a mammal. The antisense nucleic acid, ribozyme, PNA, or interfering RNA, inhibits expression of the target gene.

The use of antibodies that can inhibit one or more functions characteristic of a GSK-3β protein, such as a binding activity (e.g. GSK-3β binding to tau), is also encompassed by the present invention. Suitable methods for producing an antibody are known in the art and/or described herein.

Furthermore, the use of ligands, such as, for example, peptides that modulate interaction of a GSK-3β polypeptide with a tau polypeptide is also encompassed by the present invention. Preferably, such a peptide is expressed within the cell, although the peptide may also be introduced into the cell using methods known in the art.

The present inventors have also shown that modulation of the level of an alternative splice form of GSK-3β is associated with a disease or disorder associated with aberrant GSK-3β expression and/or activity. Accordingly, another compound useful for the treatment of a disease or disorder associated with aberrant GSK-3β expression and/or activity is an alternative splice form of a GSK-3β or a nucleic acid encoding same. In one embodiment, the alternative splice form of a GSK-3β comprises or consists of an amino acid sequence selected from the group consisting of SEQ ID NO: 5, SEQ ID NO: 7, SEQ ID NO: 7 and SEQ ID NO: 9.

In another embodiment, the compound administered is a chemical molecule, such as, for example lithium or a compound related to lithium. Such a compound also represents a useful control in the assays of the present invention.

In another embodiment, a compound administered is a small-molecule or is related in structure to a small molecule, such as, for example hymenialdisine (Meijer et al., Chem. Biol., 7: 51-63, 2000), 5-chloro-indirubin (Lau et al., J. Pept. Res., 54: 85-91, 1999), indirubin-3′-monoxime (Yoganathan et al., Biochem. Pharmacol. 60: 1115-1119, 2000), staurosporine, GF 109203× (Davies et al, Int. J. Oncol., 18: 843-847, 2001), Ro-82220, M1 muscarininc agonists (e.g. AF102B (Ter Haar et al., Nat. Struct. Biol. 8: 593-596, 2001) and AF150 (Dajani et al., Cell, 105: 721-732, 2001)) or a thiadiazolinone (Martinez et al, J. Med. Chem., 2002). Such compounds also represent suitable controls in an assay of the present invention.

The modulators identified using the methods described herein are useful for the therapeutic or prophylactic treatment of a neurodegenerative disease.

The present invention clearly encompasses the use of any in silico analytical method and/or industrial process for carrying the screening methods described herein into a pilot scale production or industrial scale production of an inhibitory compound identified in such screens. This invention also provides for the provision of information for any such production. Accordingly, a further aspect of the present invention provides a process for identifying or determining a compound or modulator supra, said method comprising:
(i) performing a method as described herein to thereby identify or determine a compound for the treatment of a disease or disorder associated with aberrant GSK-3β expression and/or activity;
(ii) optionally, determining the structure of the compound; and
(iii) providing the compound or the name or structure of the compound such as, for example, in a paper form, machine-readable form, or computer-readable form.

Preferably, the method provides a compound for the treatment of a neurodegenerative disease.

Naturally, for compounds that are known albeit not previously tested for their function using a screen provided by the present invention, determination of the structure of the compound is implicit in step (i) supra. This is because the skilled artisan will be aware of the name and/or structure of the compound at the time of performing the screen.

As used herein, the term “providing the compound” shall be taken to include any chemical or recombinant synthetic means for producing said compound or alternatively, the provision of a compound that has been previously synthesized by any person or means.

In a preferred embodiment, the compound or the name or structure of the compound is provided with an indication as to its use e.g., as determined by a screen described herein.

A further aspect of the present invention provides a process for producing a compound supra, said method comprising:

a process for identifying or determining a compound or modulator supra, said method comprising:
(i) performing a method as described herein to thereby identify or determine a compound for the treatment of a disease or disorder associated with aberrant GSK-3β expression and/or activity;
(ii) optionally, determining the structure of the compound;
(iii) optionally, providing the name or structure of the compound such as, for example, in a paper form, machine-readable form, or computer-readable form; and
(iv) providing the compound.

In a preferred embodiment, the synthesized compound or the name or structure of the compound is provided with an indication as to its use e.g., as determined by a screen described herein.

A further aspect of the present invention provides a method for manufacturing a compound for the treatment of a disease or disorder associated with aberrant GSK-3β expression and/or activity comprising:
(i) determining a candidate compound for the treatment of a disease or disorder associated with aberrant GSK-3β expression and/or activity; and
(ii) using the compound in the manufacture of a therapeutic or prophylactic for the treatment of the disease or disorder.

Preferably, the method is for manufacturing a compound for the treatment of a neurodegenerative disease

In one embodiment, the method comprises the additional step of isolating the candidate compound. Alternatively, a compound is identified and is produced for use in the manufacture of a compound for the treatment of a disease or disorder associated with aberrant GSK-3β activity.

Formulation of a compound to be administered will depend upon the route of administration selected (e.g. solution, emulsion, capsule). An appropriate composition or medicament comprising the compound can be prepared in a physiological carrier or vehicle (see, generally Remington's Pharmaceutical Sciences, 17th Edition, Mack Publishing Co., Pa., 1985).

Alternatively, or in addition the nucleic acid encoding the protein/peptide can be introduced into a host cell capable of expressing said protein/peptide for delivery. In accordance with this embodiment, the cells can be implanted (either-alone or in a barrier device), injected or introduced by any other method known in the art.

Preferably, the compound is a pharmaceutical compound.

Formulation of a pharmaceutical compound will vary according to the route of administration selected (e.g., solution, emulsion, capsule). An appropriate composition comprising the identified modulator to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluid, nutrient or electrolyte replenishers and the like (See, generally, Remington's Pharmaceutical Sciences, 17th Edition, Mack Publishing Co., Pa., 1985). For inhalation, the agent can be solubilized and loaded into a suitable dispenser for administration (e.g., an atomizer, nebulizer or pressurized aerosol dispenser).

Furthermore, where the agent is a protein or peptide, the agent can be administered via in vivo expression of the recombinant protein. In vivo expression can be accomplished via somatic cell expression according to suitable methods (see, e.g. U.S. Pat. No. 5,399,346). In this embodiment, nucleic acid encoding the protein can be incorporated into a retroviral, adenoviral or other suitable vector (preferably, a replication deficient infectious vector) for delivery, or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

As will be apparent to a skilled artisan, a compound that is active in vivo is particular preferred. A compound that is active in a human subject is even more preferred. Accordingly, when manufacturing a compound that is for the treatment of a disease or disorder associated with aberrant GSK-3β expression and/or activity it is preferable to ensure that any components added to the compound do not inhibit or modify the activity of said compound.

The present invention is further described in the following non-limiting examples.

### Example 1

### Association of a SNP Within GSK-3β with Neurodegenerative Diseases

### 1.1 Samples

Clinical data and DNA samples from the UK case/control cohort were collected from 546 individuals (74% females, 26% males) with Alzheimer's disease and 546 control subjects (74% females, 26% males). Age of onset ranged from 60 to 92 years (mean=75.45 years, SD=6.485). Controls were matched for age (mean=76.30, SD=6.257), gender and ethnicity. The sample was recruited from both community and hospital settings and an assessment battery was used to form a diagnosis of probable AD according to NINCDS-ADRDA criteria (McKhann, G. et al. Neurology 34: 939-944, 1984). Controls were selected in two ways. Spouse controls were selected on the basis of the participation of their AD partner. Controls were also collected from general practices in the same areas as the AD patients. The Sydney Older Person Study (SOPS) cohort is a population-based cohort of community living elderly people (>75 years old) within a specific geographical region of Sydney. Senile dementia status was determined using both DSM-III-R and NINCDS-ADRDA criteria (Waite et al., Australasian J. Aging 20: 67-72, 2001). The late-onset Caucasian PD patient group was recruited from movement disorders clinics as described previously (Buchanan et al., Neurosci Lett; 327: 91-94 2001). Probable PD was diagnosed by a specialist neurologist in accordance with standard criteria (Buchanan et al., Neurosci Lett; 327: 91-94 2001). The Hong Kong Chinese cohort was recruited from two major hospitals in Hong Kong (Chan et al., Neurology 60: 1002-1005, 2003). PD cases were diagnosed by consultant neurologists or geriatricians based on diagnostic criteria with supportive evidence of a good response to levodopa (Chan et al., Neurology 60: 1002-1005, 2003). Patients from the two PD cohorts had an average age of 71-72 years. Unaffected control subjects were matched to PD cases for age, gender and locality for both cohorts.

1.2 Single Nucleotide Polymorphism Detection within GSK-3β

All coding exons and flanking intronic sequences of GSK-3β were amplified by PCR from genomic DNA and nucleotide variants were detected by direct sequencing of PCR products. The three polymorphisms detected in the screen were amplified by PCR using oligonucleotides comprising the sequences set forth in Table 1

TABLE 1

sequence of oligonucleotides used in PCR
amplification and sequencing of SNPs in GSK-3β

| | | Region | SEQ ID |
|---|---|---|---|
| Name | Sequence | amplified | NO. |
| GSKEx3F | TATAAAAGCTCTAAACACTCTCAA | Exon 3 | 10 |
| GSKEx3R | AATGCTTTCCTGATATAACTAATG | Exon 3 | 11 |
| GSKEx6F | GCTTTTGGTGCCTTCTTAGGTGAC | Exon 6 | 12 |
| GSKEx6R | CGAAACATTGGGTTCTCCTCG | Exon 6 | 13 |
| GSKEx9F | GCCATGTCTGTGGATGATGAATC | Exon 9 | 14 |
| GSKEx9R | CAATGAATCACCCAAGAGGCTG | Exon 9 | 15 |

Each SNP was detected by direct sequencing of PCR products, except for the GSK2 SNP (rs6438552) in the UK AD cohort which was genotyped by a restriction length polymorphism assay using the enzyme Hpy188I (New England Biolabs, MA) which cleaves the T allele. The rs334558 SNP was detected by the restriction enzyme Alu I (Promega, Wis.) (essentially using the method described by Russ et al., Mol. Psychiatry, 6, 320-324, 2001).

### 1.3 Linkage Disequilibrium Analysis

The Celera database (available from Celera Genomics Rockville, Rockville, Md., USA) and the UCSC Genome Bioinformatics Site (described in Kent et al., Genome Res. 12:996-1006, 2002) were used to identify genes adjacent to GSK-3β. Each flanking gene was examined for known SNPs using the CHIP Bioinformatics Tool (available from the Children's Hospital Informatics Program, Children's Hospital Boston, Boston, Mass., USA). For each gene, two verified SNPs were amplified by PCR and sequenced with oligonucleotides comprising the sequences set forth in Table 2:

TABLE 2

Oligonucleotides for use in determining linkage
disequilibrium between GSK-3β polymorphisms
and polymorphisms in surrounding genes

| SNP | Oligonucleotide | | | SEQ ID |
|---|---|---|---|---|
| analyzed | name | Sequence | Gene | NO: |
| rs3732363 | GABABL.SNP1F | TCTGATGGCTTCTCTGGTTCCC | GABAL | 16 |
| rs3732363 | GABABL.SNP1R | TCTTGTGCTGTGTGTGTGCTGTC | GABAL | 17 |
| rs3732362 | GABABL.SNP2F | TTGAGGGAGTGTTTCTGGGATAAG | GABAL | 18 |
| rs3732362 | GABABL.SNP2R | TGTGTGTGTGCTGTCCAAGTGG | GABAL | 19 |
| rs916509 | FSTL1.SNP1F | GCGTCTCAAGGACAGTGAAACATC | FSTL1 | 20 |
| rs916509 | FSTL1.SNP1R | ACAGGGGCTGAAATCCAAACTAC | FSTL1 | 21 |
| rs1123897 | FSTL1.SNP2F | GGGTCCACGCAATAGTTTCAGAC | ESTL1 | 22 |
| rs1123897 | FSTL1.SNP2R | AAGCCAACCGCTCAACAACACTGG | FSTL1 | 23 |
| rs1523127 | NR1I2.SNP1F | CCTTTACTTCAGTGGGAATCTCGG | NR1I2 | 24 |
| rs1523127 | NR1I2.SNP1R | TCACACACATCTTTTGGCTGGAG | NR1I2 | 25 |
| rs2276706 | NR1I2.SNP2F | TGTTTGAGGTCAGCATCATAGTGG | NR1I2 | 26 |
| rs2276706 | NR1I2.SNP2R | CCAGAGAGCATCAGTAATGGGG | NR1I2 | 27 |
| rs1880041 | COX17.SNP1F | CAGGACATATTCAAGCAGAGTT | COX17 | 28 |
| rs1880041 | COX17.SNP1R | TTTAGCACTGTTATTAGCATTC | COX17 | 29 |
| rs523476 | COX17.SNP2F | CTGTGTCCTACTGTCTCTAAAT | COX17 | 30 |
| rs523476 | COX17.SNP2R | GCTTCAACCACTCATTCCTTC | COX17 | 31 |

### 1.3 Results

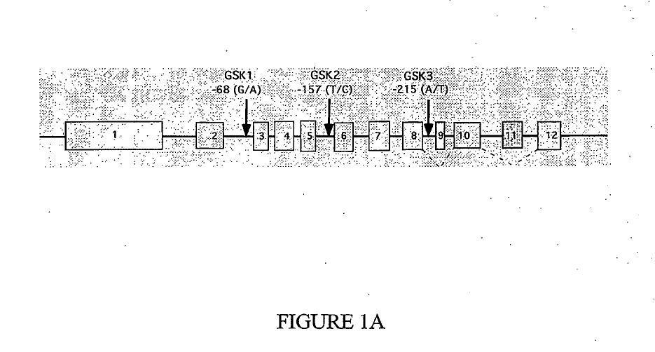

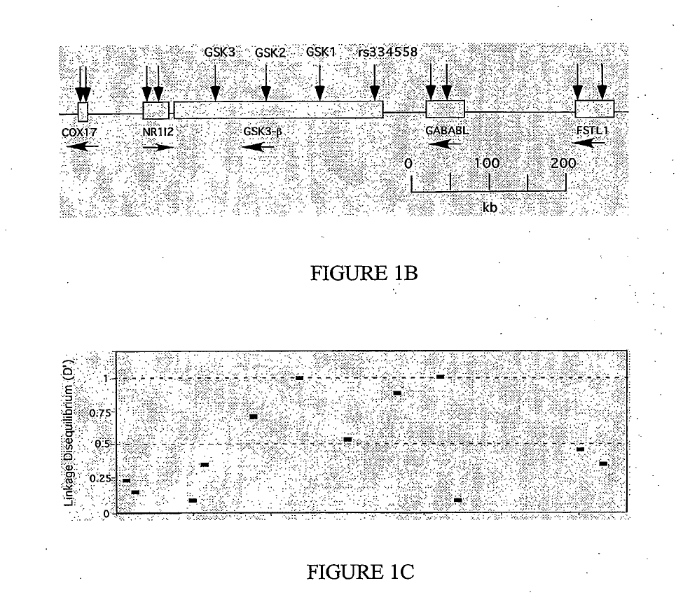

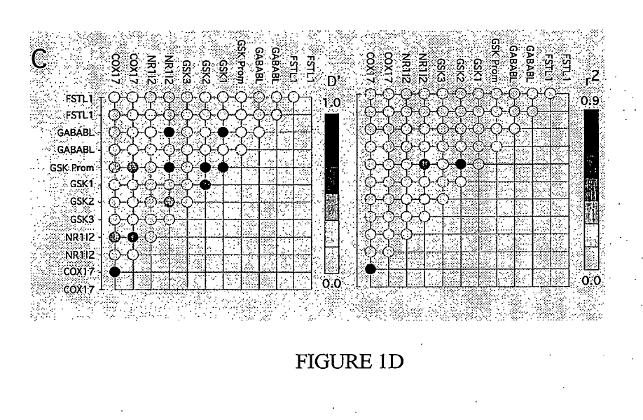

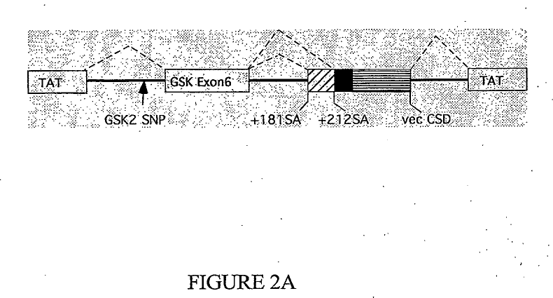

The entire coding region and flanking intronic sequences of the GSK-3β gene was analyzed for potential regulatory polymorphisms. Three potential regulatory SNPs located within intronic sequences 2, 5 and 8 (IVS 2, acceptor −68 G/A=GSK1 (also designated rs13312998 in the NCBI SNP database at May 6, 2005); IVS 5 acceptor −157 T/C=GSK2 (also designated rs6438552 in the NCBI SNP database at May 6, 2005 and/or located at a position corresponding to nucleotide position 181,700 of SEQ ID NO: 1); and IVS 8, acceptor −215 A/T=GSK3 (also designated rs7633279 in the NCBI SNP database at May 6, 2005)) were detected. These SNPs have not been previously reported (FIG. 1a). Initial analysis of association between late-onset PD and the GSK3B promoter polymorphism (rs334558) (Russ, supra) as well as the three intronic variants showed that the GSK2 SNP (rs6438552) showed association with a PD cohort. Accordingly, the association of the GSK2 (rs6438552) genotype and allele frequencies with late-onset AD and PD in four independent cohorts derived from two ethnic groups (n=1090 all affected, n=1046 all controls, Table 3) was determined. There was a significantly increased frequency of the homozygote genotypes, T/T or C/C, in the disease groups compared to control groups with an odds ratio of 1.23 (95% CI 1.04-1.46, p=0.016). Within each independent cohort, stratified either by ethnicity for the PD cohorts or ApoE 4 genotype for the dementia cohorts, there is a skewing of allele frequencies towards either the T or C allele. In the Caucasian PD case/control cohort (n=254 PD, n=186 controls), the T allele was significantly increased in PD patients compared to controls with an odds ratio of 1.34 (95% CI 1.02-1.76, p=0.033). Similarly, in the ApoE4 negative group within a community-based AD cohort (n=81 AD, n=127 control), the T allele was significantly increased in PD patients compared to controls with an odds ratio of 1.62 (1.08-2.43, p=0.021). On the other hand, in the Chinese PD cohort (n=177 PD, n=164 control), there was a significantly increased frequency of the C allele rather than the T allele. The odds ratio for this was 1.40 (0.71 if calculated using T-allele as the risk value in the same way as the other cohorts, Table 3), with a 95% CI of 1.01-1.93 (p=0.034).

Overall, the pooled results from all four cohorts found a significant association with the GSK2 SNP (rs6438552) (p=0.016). A correlation between the homozygote genotypes of GSK2 SNP (rs6438552) and the ApoE4 allele was observed in the UK dementia cohort (p=0.018). Given a strong association between ApoE4 allele and disease in the UK cohort (odds ratio 4.93, p<0.0001), and a weaker effect in the Australian cohort (odds ratio of 1.85, p=0.023), further analysis was carried out using ApoE4 allele as a confounder. When ApoE allele status was taken into account in the pooled analysis by stratification, the overall p-value for the four cohorts was raised to 0.061 (Table 1). Logistic regression was used to examine the interaction between ApoE 4 allele and GSK2 SNP (rs6438552) in the two AD cohorts. Using individual data, no significant interaction was found between ApoE4 and the homozygote genotypes in either the UK (0.25≦p≦0.5) and Australian AD cohort (0.25≦p≦0.5).

TABLE 3
Association of GSK3B IVS5-157T/C (GSK2) SNP (rs6438552) in four independent cohorts of neurodegenerative patients.

| | | | | Homozygote | | | |
|---|---|---|---|---|---|---|---|
| | | | | vs. | | | T-allele vs. C-allele |
| | | | | heterozygote | | | Chi-squared p-value |
| | GENOTYPE | | | Chi-squared | ALLELE | | (Odds |
| DISEASE COHORT (Ethnicity)^{a} | TT | TC | CC | p-value | T | C | Ratio, 95% CI) |
| I) Parkinson (Caucasian/Aus) | | | | | | | |
| PD (n = 254) | 102 (40%) | 111 (44%) | 41 (16%) | 0.598 | 315 (62%) | 193 (38%) | 0.033* |
| Control (n = 186) | 59 (32%) | 86 (46%) | 41 (22%) | | 204 (55%) | 168 (45%) | (1.34, 1.02-1.76) |
| II) Parkinson (Chinese/HK) | | | | | | | |
| PD (n = 177) | 22 (13%) | 75 (42%) | 80 (45%) | 0.102 | 119 (34%) | 235 (66%) | 0.034* |
| Control (n = 164) | 26 (16%) | 84 (51%) | 54 (33%) | | 136 (41%) | 192 (59%) | (0.71, 0.52-0.98) |
| III) Alzheimer (Caucasian/Aus) | | | | | | | |
| All AD (n = 128) | 57 (45%) | 51 (40%) | 20 (16%) | 0.398 | 165 (64%) | 91 (36%) | 0.083 |
| All Control (n = 163) | 57 (35%) | 73 (45%) | 33 (20%) | | 187 (57%) | 139 (43%) | |
| AD ApoE4− (n = 82) | 39 (48%) | 30 (37%) | 13 (16%) | 0.242 | 108 (66%) | 56 (34%) | 0.021* |
| Control ApoE4− (n = 125) | 40 (32%) | 56 (45%) | 29 (23%) | | 136 (54%) | 114 (46%) | (1.62, 1.08-2.43) |
| AD ApoE4+ (n = 46) | 18 (39%) | 21 (46%) | 7 (15%) | 0.934 | 57 (62%) | 35 (38%) | 0.490 |
| Control ApoE4+ (n = 38) | 17 (45%) | 17 (45%) | 4 (11%) | | 51 (67%) | 25 (33%) | |
| IV) Alzheimer (Caucasian/UK) | | | | | | | |
| All AD (n = 531) | 211 (40%) | 231 (44%) | 89 (17%) | 0.092 | 653 (61%) | 409 (39%) | 0.730 |
| All Control (n = 528) | 200 (38%) | 257 (49%) | 71 (13%) | | 657 (62%) | 399 (38%) | |
| AD ApoE4− (n = 196) | 65 (33%) | 96 (49%) | 35 (18%) | 0.907 | 226 (58%) | 166 (42%) | 0.239 |
| Control ApoE4− (n = 392) | 143 (36%) | 194 (50%) | 55 (14%) | | 480 (61%) | 304 (39%) | |
| AD ApoE4+ (n = 335) | 146 (44%) | 135 (40%) | 54 (16%) | 0.231 | 427 (64%) | 243 (36%) | 0.697 |
| Control ApoE4+ (n = 136) | 57 (42%) | 63 (46%) | 16 (12%) | | 177 (65%) | 95 (35%) | |
| POOLED DATA | | | | | | | |
| Overall Disease (n = 1090) | 392 (36%) | 468 (43%) | 230 (21%) | | | | |
| Overall Control (n = 1041) | 342 (33%) | 500 (48%) | 199 (19%) | | | | |

| | | | | |
|---|---|---|---|---|
| Mantel-Haenzsel Homozygote vs Heterozygote | p-value: | 0.016* | Odds Ratio (95% CI): | 1.23 (1.04-1.46) |
| | | | Heterogeneity p-value: | 0.859 |
| Mantel-Haenzsel Homozygote vs. Heterozygote | p-value: | 0.061 | Odds Ratio (95% CI): | 1.19 (0.99-1.42) |
| (stratified for ApoE4 genotype) | | | Heterogeneity p-value: | 0.809 |

^{a}Australian (Aus), Hong Kong (HK), United Kingdom (UK)
*Statistically significant at p < 0.05 level;
# All control groups in Hardy-Weinberg equilibrium

To determine the extent of linkage disequilibrium (LD) around the GSK2 SNP (rs6438552), 32 individuals were genotyped for the three other GSK3β SNPs as well as validated SNPs from four flanking genes (FIG. 1B). Lewontin's LD coefficient (D′) (Devlin and Risch Genomics 29: 311-322, 1995) was calculated and the square of the correlation coefficient between loci (r²) (Devlin and Risch Genomics 29: 311-322, 1995) using from Expectation-Maximisaton derived haplotype frequencies (Fallin and Schork, Am. J. Hum. Genet. 67, 947-959, 2000). These results indicate the GSK3B SNPs did not form a contiguous haplotype block, with D′ values ranging from 0.1 to 1, although GSK2 (rs6438552) and rs334558 SNPs were in LD with each other (D′>0.9) to form a discernible haplotype. Linkage disequilibrium between GSK2 SNP (rs6438552) and rs334558 was still apparent using the more stringent measure r², with a value of >0.5 (FIGS. 1C and 1D). The flanking genes did not appear to be in LD, with most D′ values below 0.2 (FIGS. 1C and 1D). Thus, the GSK2 SNP (rs6438552) in GSK3β fulfils a key criterion required for a regulatory SNP as predicted in the common disease-common variant hypothesis; namely that the pathogenic polymorphism is ancient and LD around it is low (Weiss and Clarke, Trends Genet. 18, 19-24, 2002).

### Example 2

### Effect of the Intron 5 SNP on Splicing of GSK-3β

### 2.1 Exon Trap Assay

Each allele from the three intronic polymorphisms described in Example 1 was subcloned in the exon trap vector pSPL3 (Invitrogen, Carlsbad, Calif., USA). For the exon 3 SNP, a 506 bp genomic fragment with 239 bp 5′ and 183 bp 3′ intronic sequences was used. For the exon 6 SNP, a 600 bp genomic fragment with 250 bp 5′ and 243 bp 3′ intronic sequences was used. For the exon 9 SNP, a 549 bp genomic fragment with 406 bp 5′ and 206 bp 3′ intronic sequences was used. Each recombinant vector was transfected into the neuroblastoma cell line, SK-N-MC (ATCC HTB 10) and embryonic kidney 293 cells (ATCC CRL 1573) using Lipofectamine 2000 (Invitrogen, Carlsbad, Calif.). Cells were left for 48 hours before total RNA was extracted and the exon trap products detected by RT-PCR essentially as described previously (Teare et al., Ann. Hum. Genet. 66: 223-233 2002).

### 2.2 Results

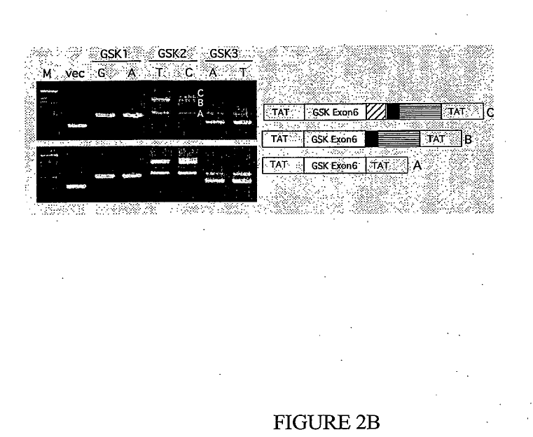

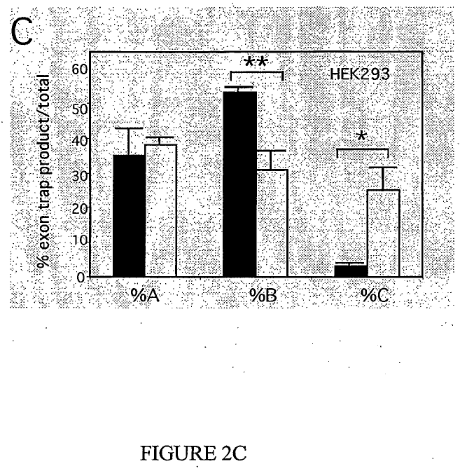

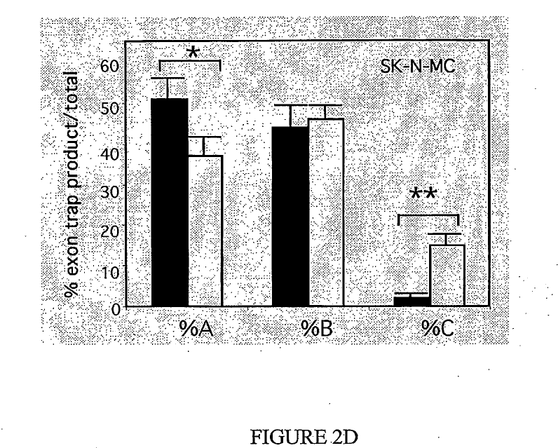

The three intronic GSK-3β SNPs described in Example 1 were examined by exon trapping (essentially as described by Stanford et al., Brain; 123: 880-893, 2000) to determine whether they would have an effect on splicing of their cognate exons in two human cell lines. A diagrammatic representation of the exon 6 gene construct is shown in FIG. 2A. In both the embryonic kidney HEK293 and the neuroblastoma SK-N-MC cell lines, the GSK2 polymorphism (rs6438552) was associated with altered ratios of exon trap products when each allele was analyzed, indicating that the GSK2 variant (rs6438552) has an effect on splicing (as shown in FIG. 2B). The exon trap products associated with GSK2 SNP (rs6438552) were sequenced and it was shown that these products correspond to differential use of three splice acceptor sites; the de novo exon 6 splice acceptor site and two cryptic splice acceptor sites in intron 6 (IVS 6+181 and UVS 6+212) (as shown in FIG. 2B). In both cell lines, there was an 8-fold decrease in utilization of the IVS VI+181 cryptic splice acceptor site (p=0.013 and p=0.003 for HEK293 and SK-N-MC respectively, Student's t test) for the T allele compared with the C allele (as shown in FIGS. 2C and 2D). The exon trap assays demonstrate that the intron 5 SNP can modulate the utilization of splice acceptor sites in downstream intronic sequences.

### Example 3

### Analysis of Splicing of GSK-3β in Patient Samples

### 3.1 Analysis of GSK-3β Transcripts

Total RNA was extracted from lymphocyte cells derived from subjects homozygous for either the T or C allele at the SNP in intron 5 of GSK-3β. RNA was extracted using the SV Total RNA Isolation System (Promega). 2 μg of RNA was reverse transcribed using the Superscript II RT enzyme (GibcoBRL) and a random hexamer primer (GibcoBRL), followed by PCR amplification using the primers GSKRT-2F (5′-TGTTGGAGTTCCCAGGACCTTG-3′) (SEQ ID NO: 32) and GSKRT-R (5′-AGTAACTGGTGGTTTTTCCTGTGC-3′) (SEQ ID NO: 33).

The relative ratio of PCR products with or without exon 9 and exon 11 sequences was determined semi-quantitatively by PCR amplification essentially as described by Stanford et al., Brain, 123, 880-893, 2000) using 0.2 μg of cDNA template and ³³P end-labeled GSKRT-F primer.

### 3.2 Western Blot Analysis of GSK-3β

Approximately 25 μg of protein lysates from lymphocytes were heated to 95° C. for 10 minutes prior to electrophoresis on a 7.5% SDS-PAGE gel and transfer to a nitrocellulose membrane (Trans-blot transfer medium, Biorad). A mouse monoclonal antibody raised against the amino terminus of GSK-3β (BD Bioscience, Calif., USA) was used to detect GSK-3β protein.

### 3.2 Results

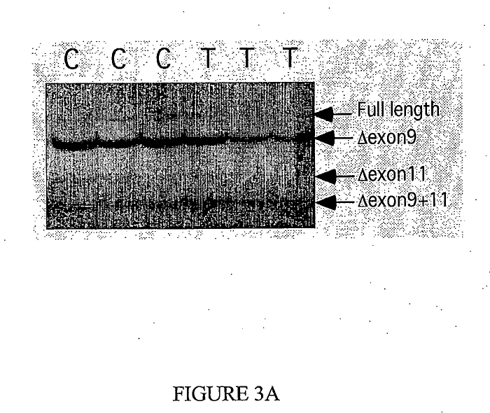

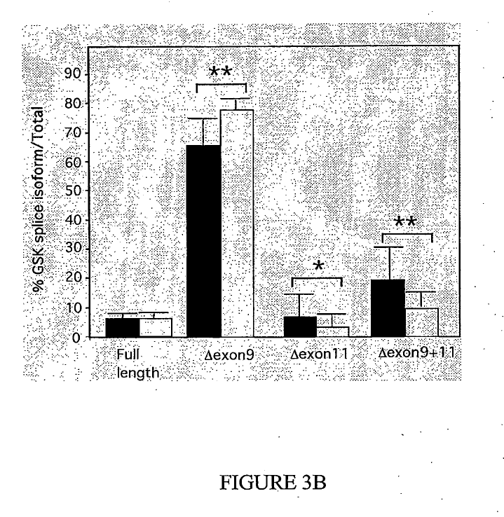

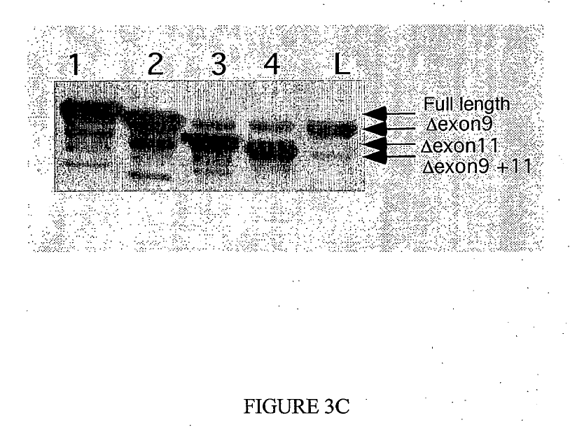

The effect of GSK2 SNP (rs6438552) was examined in a panel of 22 lymphocyte cell lines derived from individuals homozygous for either the T or C allele. The relative levels of all four GSK-3β splice isoforms was measured by RT-PCR of total RNA using primers that spanned exons 6 to 12. This allowed detection of the full length GSK-3β transcript (GSK.full) and transcripts lacking the alternatively spliced exons 9 (GSKΔexon9), 11 (GSKΔexon11), or both (GSKΔexon9+11) (FIG. 3A). When the levels of the GSK-3β transcripts were stratified according to genotype, there was a significant 1.8-fold increase in GSKΔexon11 (p=0.006, Student's t test) and 1.9-fold increase in GSKΔexon9+11 (p=0.003) when comparing individuals with T/T versus C/C genotypes (FIG. 3B). Conversely, there was a 1.2-fold decrease in GSKΔexon9 transcripts (p=0.002) between the T/T and C/C genotypes (FIG. 3B).

When GSK-3β protein levels were examined by Western blotting, only two bands were detected in lymphocytes corresponding to GSKΔexon9 and GSKΔexon9+11 (FIG. 3C). Quantification of the relative levels of the two isoforms revealed a 1.4-fold increase in the GSKΔexon9+11 isoform (p=0.111) relative to total GSK-3β protein when individuals with T/T genotypes were compared with C/C genotypes (data not shown).

### Example 4

### Effect of Alternate Splicing on Tau Phosphorylation

### 4.1 Construction of Expression Constructs

The four GSK-3β splice isoforms cDNAs amplified by PCR using lymphocyte cDNA (produced as described in Example 3) using the primer GSKRT-1F (5′-CGAGGGACACTAAATACAGTTCAA-3′) (SEQ ID NO: 34) and GSKRT-R (SEQ ID NO: 33), and each product was subcloned into the mammalian expression vector pcDNA3.1 (Invitrogen).

COS-7 cells were then transfected with the gene constructs.

### 4.2 Detection of Tau Species

Lymphocytes (as described in Example 3) or transfected cells were lysed in 1× Lysis buffer (50 mM Tris.HCl (pH 7.4), 150 mM NaCl, 1 mM PMSF, 1× complete cocktail protease inhibitor (Boehringer Mannheim) and 0.05% Triton X-100. Approximately 2-25 μg of total protein was used to assay for total Tau or Tau phosphorylated at serine residue 396 using the Human Tau or Human Tau [pS396] ELISA kit respectively (Biosource International, CA, USA).

### 4.3 Results

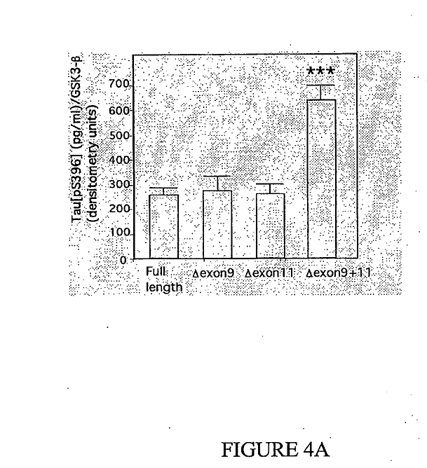

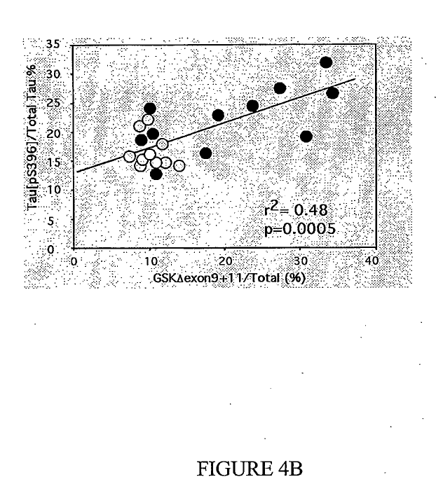

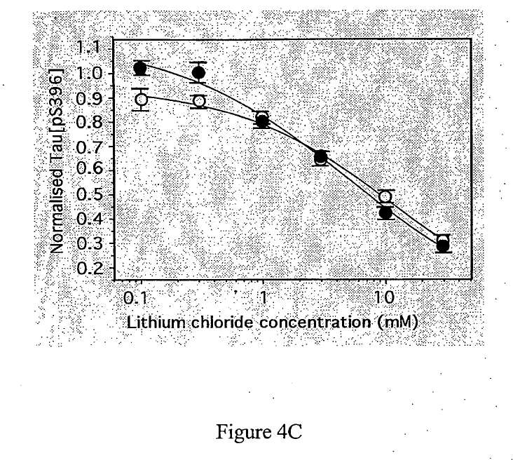

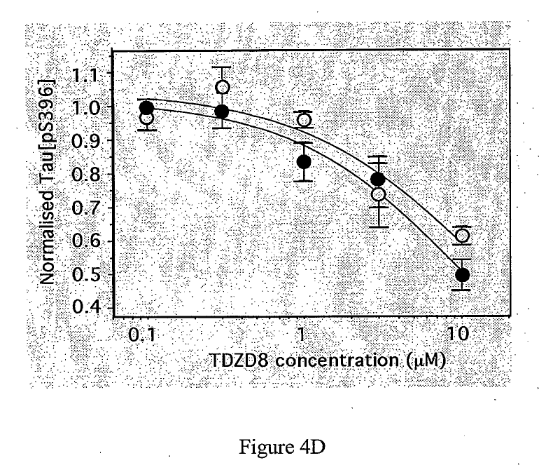

The ability of each GSK-3β splice isoform to phosphorylate Tau at a serine residue 396 (Tau [pSer396]) was examined. COS-7 cells were transfected with each cDNA and endogenous Tau phosphorylation was measured by ELISA (FIG. 4A). GSKΔexon9+11 showed the highest Tau phosphorylation, resulting in a 2.5-fold increase in levels of Tau [pSer396] compared with the other cDNAs (p<0.0001, ANOVA and post-hoc analysis). This increase in the ability of GSKΔexon9+11 to phosphorylate Tau explains the correlation (r²=0.48, p=0.0005) between the relative levels of endogenous GSKΔexon9+11 and Tau [pSer396] in lymphocyte cell lines (FIG. 4B). A 2.7-fold increase in Tau [pSer396] (p=0.021, Student's t test) protein was also observed in lymphocytes from individuals with the T/T genotype compared to the C/C genotype (data not shown).

Thus, the data suggests that the inheritance of the T allele of the GSK2 SNP (rs6438552) results in altered splicing to give rise to increased levels of GSK3Δexon9+11, which in turn leads to increased phosphorylation of Tau. The converse decrease in Tau phosphorylation is seen for the C allele.

### Example 5

### GSK-3β Isoforms Differ in their Sensitivity to Inhibitors

### 5.1 Production of Cells Expressing Specific GSK-3β Isoforms

COS-7 cells plated onto 12 well plates at concentration of 1×10⁵ cells/well and allowed to recover for 24 hours. Each well was transfected with either the GSKΔexon9 or GSKΔexon9+11 cDNA constructs (as described in Example 4) using Lipofectamine 2000. After 48 hours, growth media were removed and cells exposed to each inhibitor, Lithium (Sigma Chemical Co., MO) and TDZD8 (Calbiochem, CA) serially diluted growth medium. Media were removed (after 3 hours for lithium and 4 hours for TDZD8), cells lysed in situ, and the level of endogenous Tau [pS396] phosphorylation measured as described above.

### 5.2 Results

To gain biologically relevant insights into the actions of GSK-3β inhibitors, the ability of lithium and TDZD8 to inhibit phosphorylation of endogenous Tau protein was examined in living COS-7 cells that overexpress either of the two major splice isoforms, GSK3exon9 and GSKΔexon9+11.

Lithium acts in a complex manner involving either direct competition for its active site with Mg²⁺ or indirectly by preventing the phosphorylation of GSK-3β (Jope et al., Trends Biochem Sci. 29, 95-102, 2004.). Furthermore, the sequence encoded by exon 9 is close to the region (Zhang et al., J. Biol. Chem. 278, 33067-33077, 2003) that binds to lithium (Pilcher, Nature 425, 118-120, 2003).

TDZD8 is a heterocyclic thiadiazolidinones that is a non-ATP competitive inhibitor of GSK-3β (Martinez et al., J. Med. Chem. 45, 1292-1299, 2002).

As shown in FIG. 4C, the GSKΔexon9+11 (IC50=4.7 mM) was 3.7-fold more sensitive to lithium (p=0.039, n=5, Student's t test) at the therapeutically effective millimolar range (Jope, supra) than GSKΔexon9 (IC50=17.3 mM). A smaller increase (2-fold) in sensitivity to TDZD8 was observed for GSKΔexon9+11 (IC50=5.2 μM) at the micromolar range (p=0.181, n 4, Student's t test) compared with GSKΔexon9 (IC50=11.2 μM) (FIG. 4D).

### Example 6

### Transcriptional Activity of the GSK-3β Promoter is Influenced by the rs334558 SNP

### 6.1 Production of an Expression Construct Containing Luciferase Under Control of the GSK-3β Promoter

A 1510 bp DNA fragment comprising the promoter and transcription start site of GSK3β was amplified by PCR using primers comprising the sequences AAAAGATCCAAAGCTACCACGGC (SEQ ID NO: 50) and TCCTTCCTTCCTTTGTCACTTGGC (SEQ ID NO: 51). The amplification product was subcloned into the pGL3-Basic Luciferase vector (Promega). Each promoter haplotype was assayed for transcriptional efficiency. Each recombinant vector was transfected into the SK-N-MC (ATCC HTB 10) and 293 cells as described supra. Cells were lysed with 1× Lysis buffer (Promega) after 48 hours and assayed for luciferase activity using the Bright-Glo Luciferase assay system (Promega).

### 6.2 Results

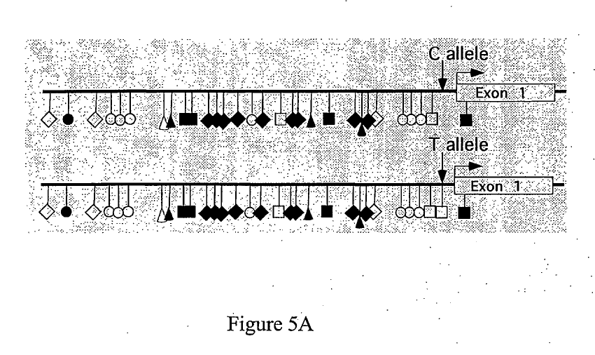

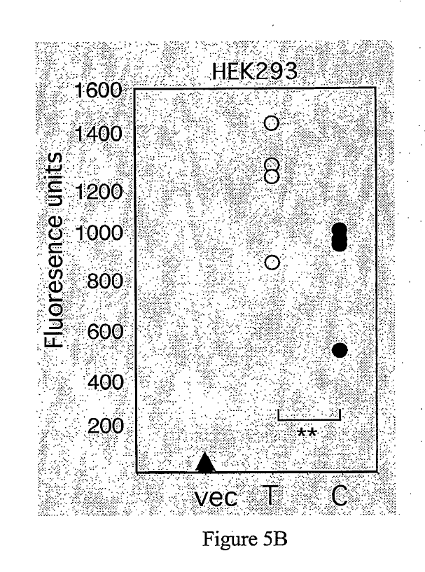

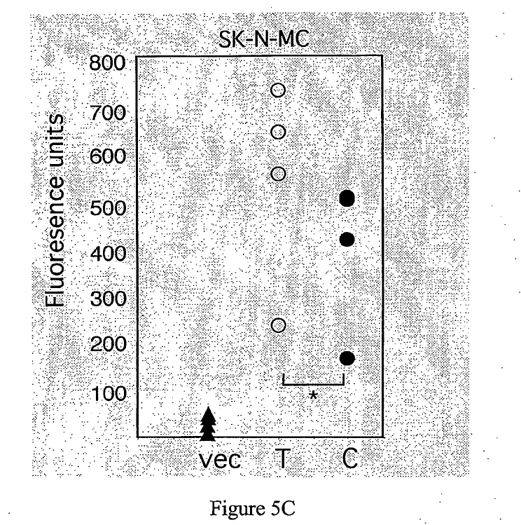

The promoter sequence of GSK3B was analyzed to determine possible binding sites of transcription factors using the MatInspector v2.2 software and the TRANSFAC 4.0 database (Quandt et al., Nucleic Acids Res. 23, 4878-48840, 1995). Using a high stringency of selection (maximal ‘Core similarity’ setting of 1 and ‘Matrix similarity’ of 0.85), a series of binding sites were detected for common transcription factors including AP-1 and SP-1 (FIG. 5A). The rs334558 SNP (located at a site corresponding to nucleotide position 232 of SEQ ID NO: 1) is located within a binding site for the AP4 transcription factor (FIG. 5A). The C allele was predicted by to abrogate binding of the transcription factor to the site.

The ability of each promoter haplotype to drive the expression of a luciferase reporter gene was examined. The T allele was associated with a significant 1.4 fold increase in transcriptional efficiency relative to the C allele in 293 (p=0.002, Student t test) and SK-N-MC (p=0.015, Student t test) respectively (FIGS. 5B and 5C, respectively).

### Example 7

### Haplotype Analysis of Two Functional SNPs of GSK-3β in Neurodegenerative Cohorts

As discussed in Example 6 the rs334558 SNP (located at position 231 of SEQ ID NO: 1) has an effect on expression of GSK-3β. Accordingly, haplotypes derived from the two functional SNPs were examined to determine whether or not they were more informative in the three neurodegenerative cohorts that showed the strongest association with GSK2 SNP (rs6438552) (located at a position corresponding to nucleotide position 181,700 of SEQ ID NO: 1). As shown in Table 4, rs334558 has an association in the AD (Aus) cohort when stratified for ApoE (odds ratio 1.7, p=0.015). When the haplotype frequencies for each cohort was estimated using the expectation-maximization algorithm, a significant association of GSK3β haplotypes in all three cohorts was observed, either as a global difference in haplotypes (PD/Chinese p=0.002 and AD/Aus/ApoE4-p=0.025), or in specific haplotype (Table 4). The strongest association was observed for the rs334558 T allele—GSK2 (is 6438552) C allele haplotype in the two PD cohorts (odds ratio 0.5-2.9, p=0.019-0.002).

TABLE 4
Association of GSK3B Promoter SNP (rs 334558) in four neurodegenerative cohorts

| | | | | Chi- | | | Chi-square |
|---|---|---|---|---|---|---|---|
| | GENOTYPE | | | square | ALLELE | | p value |
| DISEASE COHORT (Ethnicity)^{a} | TT | TC | CC | p value | T | C | (odds ratio) |
| I) Parkinson (Caucasian/Aus) | | | | | | | |
| PD (n = 220) | 90 (41%) | 99 (45%) | 31 (14%) | 0.772 | 279 (63%) | 161 (37%) | 0.469 |
| Control (n = 229) | 100 (44%) | 101 (44%) | 28 (12%) | | 301 (66%) | 157 (34%) | |
| II) Parkinson (Chinese/HK) | | | | | | | |
| PD (n = 177) | 21 (13%) | 73 (42%) | 83 (45%) | 0.098 | 115 (32%) | 239 (68%) | 0.087 |
| Control (n = 184) | 24 (16%) | 94 (51%) | 66 (33%) | | 142 (39%) | 226 (61%) | |
| III) Parkinson (Chinese/Singapore) | | | | | | | |
| PD (n = 169) | 27 (16%) | 79 (47%) | 63 (37%) | 0.917 | 133 (39%) | 205 (61%) | 0.752 |
| Control (n = 164) | 29 (18%) | 75 (46%) | 60 (36%) | | 133 (41%) | 195 (49%) | |
| III) Alzheimer (Caucasian/Aus) | | | | | | | |
| All AD (n = 129) | 61 (47%) | 51 (40%) | 17 (13%) | 0.227 | 173 (67%) | 85 (33%) | 0.139 |
| All Control (n = 166) | 62 (37%) | 79 (48%) | 25 (15%) | | 203 (61%) | 129 (39%) | |
| AD ApoE4− (n = 81) | 44 (54%) | 26 (32%) | 11 (14%) | 0.016* | 114 (70%) | 48 (30%) | 0.015* (1.7) |
| Control ApoE4− (n = 128) | 44 (34%) | 62 (49%) | 22 (17%) | | 150 (59%) | 106 (41%) | |
| AD ApoE4+ (n = 48) | 19 (39%) | 21 (44%) | 8 (17%) | 0.454 | 59 (61%) | 37 (39%) | 0.258 |
| Control ApoE4+ (n = 38) | 18 (47%) | 17 (45%) | 3 (8%) | | 53 (70%) | 23 (30%) | |
| IV) Alzheimer (Caucasian/UK) | | | | | | | |
| All AD (n = 249) | 119 (48%) | 105 (42%) | 25 (10%) | 0.940 | 343 (69%) | 155 (31%) | 0.845 |
| All Control (n = 252) | 121 (48%) | 108 (43%) | 23 (9%) | | 350 (69%) | 154 (31%) | |
| AD ApoE4− (n = 96) | 45 (47%) | 43 (45%) | 8 (8%) | 0.980 | 133 (69%) | 59 (31%) | 0.848 |
| Control ApoE4− (n = 184) | 84 (46%) | 84 (46%) | 16 (9%) | | 252 (68%) | 116 (32%) | |
| AD ApoE4+ (n = 153) | 74 (48%) | 62 (41%) | 17 (11%) | 0.704 | 210 (69%) | 96 (31%) | 0.469 |
| Control ApoE4+ (n = 68) | 37 (54%) | 24 (35%) | 7 (10%) | | 98 (72%) | 38 (23%) | |

| HAPLOTYPE ANALYSIS | | | | | |
|---|---|---|---|---|---|
| (rs334558 (T/C) - GSK3B IVS 5- | | | | | |
| 157 T/C) - Using EM Method | GENOTYPE | | | | Chi-square |
| (Arlequin Program) | T-T | T-C | C-T | C-C | p value |
| I) Parkinson (Caucasian/Aus) | | | | | |
| PD (n = 410) | 232 (57%) | 25 (6%) | 19 (5%) | 134 (33%) | P = 0.059 |
| Control (n = 342) | 177 (52%) | 37 (11%) | 10 (3%) | 118 (34%) | |
| P value (odds ratio)* | p = 0.185 (1.2) | p = 0.019* (0.5) | p = 0.225 (1.6) | p = 0.599 (0.9) | |
| II) Parkinson (Chinese/HK) | | | | | |
| PD (n = 272) | 61 (23%) | 28 (10%) | 31 (11%) | 152 (56%) | P = 0.002* |
| Control (n = 292) | 95 (32%) | 11 (4%) | 26 (9%) | 160 (55%) | |
| P value (odds ratio) | p = 0.007* (0.6) | p = 0.002* (2.9) | p = 0.326(1.3) | p = 0.795 (1.0) | |
| II) Parkinson | | | | | |
| (Chinese/Singapore) | 124 (38%) | 6 (2%) | 15 (5%) | 179 (55%) | P = 0.836 |
| PD (n = 324) | 116 (37%) | 9 (3%) | 13 (4%) | 174 (56%) | |
| Control (n = 312) | p = 0.776 (1.0) | p = 0.391 (0.6) | p = 0.776 (1.1) | p = 0.895 (1.0) | |
| P value (odds ratio) | | | | | |
| III) Alzheimer (Caucasian/Aus) | | | | | |
| All AD (n = 252) | 166 (66%) | 11 (4%) | 2 (1%) | 73 (29%) | P = 0.076 |
| All Control (n = 318) | 178 (56%) | 15 (5%) | 7 (2%) | 118 (37%) | |
| P value (odds ratio) | p = 0.016* (1.5) | p = 0.841 (0.9) | p = 0.187 (0.4) | p = 0.041* (0.7) | |
| AD ApoE4− (n = 160) | 104 (65%) | 10 (6%) | 0 (0%) | 46 (29%) | |
| Control ApoE4− (n = 246) | 131 (53%) | 11 (5%) | 4 (2%) | 100 (40%) | P = 0.025* |
| P value (odds ratio) | p = 0.019* (1.6) | p = 0.429 (1.4) | p = 0.105 (—) | p = 0.015* (0.6) | |
| AD ApoE4+ (n = 92) | 55 (60%) | 2 (2%) | 2 (2%) | 32 (36%) | |
| Control ApoE4+ (n = 72) | 47 (65%) | 4 (6%) | 3 (4%) | 17 (25%) | P = 0.356 |
| P value (odds ratio) | p = 0.471 (0.8) | p = 0.252 (0.4) | p = 0.461 (0.5) | p = 0.082 (1.8) | |
| IV) Alzheimer (Caucasian/UK) | | | | | |
| All AD (n = 496) | 287 (58%) | 47 (10%) | 12 (2%) | 150 (30%) | P = 0.804 |
| All Control (n = 498) | 303 (61%) | 44 (9%) | 10 (2%) | 141 (28%) | |
| P value (odds ratio) | p = 0.339 (0.9) | p = 0.726 (1.1) | p = 0.659 (1.2) | p = 0.621 (1.1) | |
| AD ApoE4− (n = 192) | 110 (57%) | 25 (13%) | 2 (1%) | 55 (29%) | |
| Control ApoE4− (n = 362) | 217 (60%) | 31 (8%) | 7 (2%) | 107 (30%) | P = 0.352 |
| P value (odds ratio) | p = 0.546 (0.9) | p = 0.098 (1.6) | p = 0.429 (0.5) | p = 0.822 (1.0) | |
| AD ApoE4+ (n = 304) | 177 (58%) | 22 (7%) | 10 (3%) | 95 (32%) | |
| Control ApoE4+ (n = 136) | 86 (63%) | 13 (10%) | 3 (2%) | 34 (25%) | P = 0.448 |
| P value (odds ratio) | p = 0.322 (0.8) | p = 0.455 (0.7) | p = 0.534 (1.5) | p = 0.183 (1.4) | |

^{a}Australian (Aus), Hong Kong (HK).
*2 × 2 contigency tables p values and odds ratio calculated for each haplotype vs. all other haplotypes.

### Example 8

### Analysis of SNPs Using Fluorescent Single Nucleotide Primer Extension Analysis

### 8.1 PCR

In the first round of PCR, a fragment is amplified from genomic DNA using 0.5 μM each two primers comprising the sequences TTCTGCCCTTGCTTCTTTGT (SEQ ID NO: 35) or TGTTAGTGCAAAGCAGCTGG (SEQ ID NO: 36) and PCR supermix high fidelity (Life Technologies, Rockville, Md., USA) in a final volume of 50 μL containing 10 U/mL DNA polymerase in 60 mM Tris-SO₄ (pH 9.1), 18 mM (NH₄)₂SO₄, 2 mM MgSO₄, and 200 μM each of the four dNTPs. Thermocycling conditions: 3 min initial denaturation at 96° C. and 25 cycles of 1 min 96° C., 30 s 58° C., 3 min 72° C.

### 8.2 Primer Extension

PCR products are purified using Centri-sep columns (Princeton Separations, Adelphia, N.J., USA); 25-500 ng of this 422 bp fragment then serves as a template for subsequent primer extension using Cy5 labeled primer comprising the sequence CATATATGAAAAAGAAAATGTAAACTGTAACTATCTCT (SEQ ID NO: 37) (300 nM) and 4 U of Thermo Sequenase, 0.2 mM of the three dNTPs (dATP, dCTP and dGTP) and 0.2 mM of ddTTP in a buffer of 26 mM Tris-HCl (pH 9.5) and 6.5 mM MgCl₂ in a final volume of 20 μL. Thermocycling conditions: 3 min initial denaturation at 94° C. and 40 cycles of 30 s 94° C., 30 s 60° C., 30 s 72° C., followed by a 2 min final extension at 72° C. dNTPs are from Life Technologies, ddTTP are from Sigma (St. Louis, Mo., USA), Thermo Sequenase and Sequenase reaction buffer: are from Amersham (Cleveland, Ohio, USA). 10 μL formamide is added to 2 μL primer extension product and incubated at 95° C. for 2 min prior to CE analysis for denaturation.

8.3 Capillary electrophoresis

For all experiments the P/ACE-MDQ (Beckman Coulter Inc., Fullerton, Calif., USA) instrument is used in normal polarity separations mode (cathode at the detection site). The separation is monitored on-column by laser-induced fluorescence detection (LIF) using a red diode laser (635 nm) and a narrow band pass emission filter (670 nm). The temperature of the cartridge holding the separation capillary column is thermostated at 30±0.1° C. by the active liquid cooling of the device. A 10 cm effective separation length capillary (30 cm total) with an ID of 75 μm is used for separation of the denatured primer extension products. 10% PVP (Mᵣ 1 300 000 from Aldrich) solution is used as sieving and wall coating matrix dissolved in 1×TBE buffer (89 mM Tris, 89 mM boric acid, 2 mM EDTA·Na₂, pH 8.4). Bodipy FL Hydrazide is used as neutral marker for electroosmotic flow (EOF) measurement (Molecular Probes, Eugene, Oreg., USA). The primer extension samples are electrokinetically injected at 10 kV for 30 s and separated at 20 kV. The data is acquired and evaluated by the P/ACE System MDQ software.

### Example 9

### Analysis of SNPs Using MALDI-TOF Mass Spectrometry

### 9.1 Amplification of Regions Containing a Single Nucleotide Polymorphism

The SNP associated with a neurodegenerative disorder in intron 5 of GSK-3β is amplified under universal conditions in a 25-μL reaction containing 50-100 ng DNA, 0.2-0.4 μM each primer (TTCTGCCCTTGCTTCTTTGT (SEQ ID NO: 38) or TGTTAGTGCAAAGCAGCTGG (SEQ ID NO: 39)), 2.5 mM MgCl₂, 200 μM each dNTP and 1 U HotStarTaq DNA Polymerase (Qiagen) in reaction buffer supplied by the manufacturer. PCR thermal cycling conditions are: 15 min at 95° C. followed by 15 cycles of 94° C. for 30 s, 58° C. for 1 min and 72° C. for 1 min, then 30 cycles of 94° C. for 30 s, 58° C. for 1 min and 72° C. for 1 min, with a final 10 min incubation at 72° C.

### 9.2 Single Nucleotide Primer Extension Assay

Following PCR, 1 U of shrimp alkaline phosphatase (SAP; Roche Molecular Biochemicals) is added to 20 μL of PCR product to hydrolyze residual dNTPs. The mixture is incubated for 60 min at 37° C. followed by enzyme inactivation for 20 min at 95° C. Primer extension reactions are performed in 10-μL volumes and contained 5 μL of ‘SAP-treated’ PCR product, 30 μM each ddNTP, 1-3 μM genotyping primer (comprising the sequence CATATATGAAAAAGAAAATGTAAACTGTAACTATCTCT, SEQ ID NO: 40) and 1-2 U ThermoSequenase DNA polymerase (Amersham Pharmacia Biotech). Thermal cycling conditions consist of denaturation at 94° C. for 2 min, followed by 50 cycles of 94° C. for 30 s and 37° C. for 90 s. Primer concentrations are balanced empirically to ensure that some unextended primer remained to act as an internal-mass standard.

### 9.3 Sample Preparation and MALDI-TOFMS Analysis

Prior to analysis, samples are desalted by dialysis through 96-well membrane filter plates with 0.05 μm pore size (MAVM NO₅, Millipore) which are floated on deionized water for approximately 2 h. The MALDI plate is spotted with 1 μL of 3-hydroxypicolinic acid (3-HPA) matrix (50 mg/mL in 50% acetonitrile and 25 mM diammonium citrate) and allowed to dry before deposition of 1 μL of desalted primer extension sample. Mass spectra are collected using a Voyager-DE™ PRO MALDI-TOF instrument (PerSeptive Biosystems, Framingham, Mass., USA) equipped with a 337 nm nitrogen laser. Measurements are taken manually in linear, positive ion mode at a 20-24 kV acceleration voltage and 200 ns delayed ion extraction. To avoid saturation of the detector from matrix ions, masses <1000 Da are suppressed by an ion deflector. Unextended primers are used as internal standards for mass calibration. The nucleotide incorporated at the variant site is determined manually by calculating the mass difference between the unextended primer and the extension product. In the case of a heterozygote, two mass-resolved extension products are produced.

### Example 10

### Detection of SNPs Using Fluorescence-Adapted SSCP

### 10.1 PCR

The primers described in Examples 8 and 9 are adapted for fluorescence-adapted SSCP analysis. The primers are prepared as follows: the sequence-specific forward primer (TTCTGCCCTTGCTTCTTTGT, SEQ ID NO: 41) is conjugated with 5′-TGACCGGCAGCAAAATTG-3′ (SEQ ID NO: 42) tail at its 5′ end (to form an oligonucleotide that comprises the nucleotide sequence TGACCGGCAGCAAAATTGTTCTGCCCTTGCTTCTTTGT, SEQ ID NO: 43); the sequence-specific reverse primer (TGTTAGTGCAAAGCAGCTGG, SEQ ID NO: 44) is conjugated with 5′-TGTAAAACGACGGCCAGT-3′ (SEQ ID NO: 45) tail at its 3′ end to form an oligonucleotide comprising the nucleotide sequence TGTTAGTGCAAAGCAGCTGGTGTAAAACGACGGCCAGT; (SEQ ID NO: 46) the Cy-5 labeled 5′-TGACCGGCAGCAAAATTG-3′ (SEQ ID NO: 47) primer is obtained from Amersham Bioscience, NJ, USA as is the Cy-5 labeled 5′-TGTAAAACGACGGCCAGT-3′ (SEQ ID NO: 48) primer.

PCR is performed using a PCR mix containing 50-100 ng of Genomic DNA (from normal or subjects suspected of suffering from a neurodegenerative disease, or a subject being tested). 2 μmol of the conjugated sequence-specific forward primer, 2 μmol of the conjugated sequence-specific reverse primer, 10 μmol of Cy-5 labeled primers, 200 μM of dNTP, 2 μL of 10× reaction buffer, 1 U of Taq polymerase (Takara Bio, Shiga, Japan) in a final 20 μL reaction volume. The PCR amplification procedure entails an initial denaturation cycle (94° C., 4 min); 25 cycles of 1 min each at 94° C., 58° C. and 72° C., and 10 cycles of 1 min each at 94° C., 58° C. and 72° C. This is followed by 7 min at 72° C. for final extension in a GeneAmp PCR System 9600 (Applied Biosystems, CA, USA).

### 7.2 Gel Electrophoresis

The fluorescence-labeled DNA fragments are diluted 2-5 times with a loading buffer consisting of formamide and EDTA (100% formamide:50 mM EDTA=5:1). The DNA samples are heated at 94° C. for 5 min and cooled immediately on ice. This solution (1 to 5 μL) is applied to a non-denaturing polyacrylamide gel using 0.5×MDE gel solution-(bioWhittaker Molecular Applications, ME, USA) with 1×TBE buffer (100 mM Tris-borate, pH 8.3, 1 mM EDTA). The short gel plate is applied to the Amersham Biosciences Model ALF Express DNA sequencer. The gel temperature is maintained at 15° C. Electrophoresis is performed in 0.5×TBE buffer at 30 W for 5 h, and the data is collected and analyzed using Fragment Manager software (Amersham Biosciences).

Samples are then analyzed to determine those that migrate at a different rate compared to PCR products generated from a control sample, suggesting the presence of a polymorphism that causes a change in the conformation of the amplified nucleic acid.

### Example 11

### Preparation of a Monoclonal Antibody that Recognizes a Polypeptide Encoded by Exon 11 of GSK-3β

A monoclonal antibody that specifically binds a region of GSK-3β encoded by exon 11 is produced using methods known in the art. Briefly, a peptide antigen that corresponds to the region of GSK-3β is synthesized essentially using the methods described in Bodanszky, M. (1984) Principles of Peptide Synthesis, Springer-Verlag, Heidelberg and Bodanszky, M. & Bodanszky, A. (1984) The Practice of Peptide Synthesis, Springer-Verlag, Heidelberg.

Peptides are purified using HPLC and purity assessed by amino acid analysis.

Female BalB/c mice are immunized with a purified form of the peptide. Initially mice are sensitized by intraperitoneal injection of Hunter's Titermax adjuvant (CytRx Corp., Norcross, Ga.). Three boosts of the peptide are administered at 2, 5.5 and 6.5 months post initial sensitization. The first of these boosts is a subcutaneous injection while the remaining are administered by intraperitoneal injection. The final boost is administered 3 days prior to fusion.

The splenocytes of one of the immunized BALB/c mice is fused to X63-Ag8.653 mouse myeloma cells using PEG 1500. Following exposure to the PEG 1500 cells are incubated at 37° C. for 1 hour in heat inactivated fetal bovine serum. Fused cells are then transferred to RPMI 1640 medium and incubated overnight at 37° C. with 10% CO₂. The following day cells are plated using RPMI 1640 media that has been supplemented with macrophage culture supernatants.

Two weeks after fusion, hybridoma cells are screened for antibody production by solid phase ELISA assay. Standard microtitre plates are coated with recombinant GSK-3β (produced essentially as described by Woodgett EMBO J, 9: 2431-2438, 1990) in a carbonate based buffer. Plates are then blocked with BSA, washed and then the test samples (i.e. supernatant from the fused cells) is added, in addition to control samples, (i.e. supernatant from an unfused cell). Antigen-antibody binding is detected by incubating the plates with goat-anti-mouse HRP conjugate (Jackson ImmunoResearch Laboratories) and ABTS peroxidase substrate system (Vector Laboratories, Burlingame, Calif. 94010, USA). Absorbance is read on an automatic plate reader at a wavelength of 405 nm.

Any colonies that are identified as positive by these screens continue to be grown and screened for several further weeks. Stable colonies are then isolated and stored at 80° C.

Positive stable hybridomas are then cloned by growing in culture for a short period of time and diluting the cells to a final concentration of 0.1 cells/well of a 96 well tissue culture plate. These clones are then screened using the previously described assay. This procedure is then repeated in order to ensure the purity of the clone.

Four different dilutions, 5 cells/well, 2 cells/well, 1 cell/well, 0.5 cells/well of the primary clone are prepared in 96-wells microtiter plates to start the secondary cloning. Cells are diluted in IMDM tissue culture media containing the following additives: 20% fetal bovine serum (FBS), 2 mM L-glutamine, 100 units/ml of penicillin, 100 μg/ml of streptomycin, 1% GMS-S, 0.075% NaHCO₃. To determine clones that secrete anti-human GSK-3β antibody, supernatants from individual wells of the 0.2 cells/well microtiter plate are withdrawn after two weeks of growth and tested for the presence of antibody by ELISA assay as described above.

All positive clones are then adapted and expanded in RPMI media containing the following additives: 10% FBS, 2 mM L-glutamine, 100 units/ml of penicillin, 100 μg/ml of streptomycin, 1% GMS-S, 0.075% NaHCO₃, and 0.013 mg/ml of oxalaacetic acid. A specific antibody is purified by Protein A affinity chromatography from the supernatant of cell culture.

The titer of the antibodies produced using this method are determined using the Easy Titer kit available from Pierce (Rockford, Ill., USA). This kit utilizes beads that specifically bind mouse antibodies, and following binding of such an antibody these beads aggregate and no longer absorb light to the same degree as unassociated beads. Accordingly, the amount of an antibody in the supernatant of a hybridoma is assessed by comparing the OD measurement obtained from this sample to the amount detected in a standard, such as for example mouse IgG.

The specificity of the monoclonal antibody is then determined using a Western blot essentially as described in Example 3.2. As a control the antibody that binds to the N-terminus of GSK-3β is also used. The monoclonal antibody that specifically binds to the region of GSK-3β encoded by exon 11 detects all forms of GSK-3β but GSKΔexon9+11 and GSKΔexon11.

### Example 12

### Determining the Level of GSK-3β Comprising Exon 11 in a Biological Sample

A monoclonal antibody that binds to the region of GSK-3β encoded by exon 11 as described in Example 8 is used in the production of a two-site ELISA to determine the level of GSK-3β comprising exon 11 in a biological sample.

A polyclonal antibody that binds to the N-terminal region of GSK-3β (BD Biosciences) is absorbed to a microtitre plate at 20° C. for 16 hours. Plates are then washed and blocked for 1 hour. Recombinant GSK-3β is serially diluted, added to wells of the microtitre plate and incubated for 1 hour.

The monoclonal antibody capable of specifically binding, the region of GSK-3β encoded by exon 11 described in Example 11 is conjugated to horseradish peroxidase (HERP) using a HRP conjugation kit (Alpha Diagnostics International, Inc., San Antonio, Tex., USA).

Following washing of the microtitre plates, the HRP conjugated monoclonal antibody is added to each well of the plate and incubated. Plates are then washed and ABTS (Sigma Aldrich, Sydney, Australia) is added to each well. Reactions are stopped after approximately 20 minutes and absorbance values measured at 415 nm.

The amount of absorbance detected in negative control wells (no GSK-3β added) is subtracted from the absorbance of each other well to determine the amount of antibody bound to GSK-3β.

To assess the amount of GSK-3 encoded by exon 11 in a patient sample, the total amount of GSK is also determined using a fluorescently conjugated mouse anti-human GSK-3β monoclonal antibody (Sigma Aldrich, the antibody is labeled using Fluorotag FITC conjugation kit also available from Sigma Aldrich). The level of GSK-3β encoded by exon 11 is then determined relative to total GSK-3β.

### Example 13

### Production of a Transgenic Mouse Expressing GSK-3β Comprising a SNP Associated with a Neurodegenerative Disease

To produce a transgenic mouse expressing a form of GSK-3β that is associated with a neurodegenerative disease a BAC (RP11-359H3) is mutated to contain the T allele at the site of the SNP in intron 5 of GSK-3β. The point mutation is produced essentially as described by Lalioti and Heath (Nucl. Acids Res. 29: e14, 2001). Essentially, this method involves homologous recombination between the original BAG and a shuttle vector providing the point mutation. Each recombination step is monitored using positive and negative selection markers, which are the Kanamycin-resistance gene, the sacB gene and temperature-sensitive replication, all conferred by the shuttle plasmid.

The mutant BAC is then microinjected into the pronucleus of a fertilized mouse oocyte essentially as described in Hogan et al (In: Manipulating the Mouse Embryo. A Laboratory Manual, 2^{nd} Edition. Cold Spring Harbour Laboratory. ISBN: 0879693843, 1994). Microinjected oocytes are then injected into a pseudopreganant mouse and offspring screened for the presence of the mutant GSK-3β gene using PCR. Positive mice are then screened using Western blotting with the monoclonal antibody described in Example 8 to determine those that express human GSK-3β.

### Example 14

### Production of Transgenic Mice that Express GSK-3β, GSK-3β(Δexon 9), GSK-3β(Δexon 11) or GSK-3β(Δexon 9+11)

The expression constructs encoding the four different forms of GSK-3β identified (described in Example 4) are linearized and used in the production of transgenic mice essentially as described in Example 10.

Offspring are screened using PCR with oligonucleotides specific to the CMV promoter in the pcDNA3.1 vector. Those that carry the transgene are then further screened for transgene expression using oligonucleotides that specifically detect expression of human GSK-3β and with Western blotting using the monoclonal antibody described in Example 8 to determine those that express human GSK-3β.

The BAC transgenic mice (Example 10) and the GSK-3β, GSK-3β(Δexon 9), GSK-3β(Δexon 11) or GSK-3β(Δexon 9+11) transgenic mice are all then crossed with heterozygous GSK-3β knockout mice (mGSK-3β^{+/-}) (Hoeflich et al., Nature 406: 86-90, 2000). These mice are then bred to produce mice that are heterozygous for a specific transgene and homozygous knockouts (mGSK-3β^{-/-}). These mice now only express the forms of human GSK-3β expressed by the relevant transgene.

All transgenic mice are then assessed for aberrant tau phosphorylation, essentially as described in Example 4. In particular, brain tissue and CSF fluid are assessed for aberrant tau phosphorylation compared to wild-type mice and transgenic mice expressing GSK-3β (i.e. mGSK-3β^{-/-}hGSK-3β+/−).

Furthermore, transgenic mice are assessed for neurodegenerative phenotypes. Initially mice are assessed using the SHIPA protocol (Rogers et al. Mamm. Genome, 8: 711-713, 1997). This screening strategy includes a behavior observation protocol; screening of locomoter activity, food and water intake, balance and co-ordination, analgesia, histology and biochemistry of mice; and measures of anxiety, learning and memory, prepulse inhibition, EEG, nerve conduction and MRI.

The brains of all mice are studied to determine the presence of Lewy bodies and/or neurofibrillary tangles.

### Example 15

### Analysis of Intron 5 SNP in GSK-3β in type II Diabetes Subjects

DNA is isolated from whole blood of subjects who suffer from a type II diabetes or age and sex matched controls. Suitable samples are described, for example, in Ghoussaini et al., BMC Med. Gen., 6: 11-19, 2005. Briefly, studies are performed with at least about 195 unrelated lean children (only those that have no obese siblings are included) and a set of at least about 395 unrelated obese children. The pool of obese children used in association studies comprises unrelated obese children.

Children with a BMI greater than the 97^{th} percentile of BMI for age and sex reported on the tables of Rolland-Cachera et al. Eur. J. Clin. Nutr. 45:13-21, 1991 are defined as obese as recommended by the European Childhood Obesity Group (ECOG) (Poskitt Acta. Paediatr. 84:961-963, 1995).

The Body Mass Index (BMI) is calculated as weight (Kg) divided by height (m) squared. The Z score of BMI is calculated using Cole's last mean square method (Cole et al., Arch. Dis. Child. 73:25-29, 1995). Quantitative measurements of plasma insulin are performed using double-antibody radio immunoassays. Serum glucose concentrations are measured using a glucose oxidase procedure. HOMA-IR and HOMA-B are calculated according to Matthews et al Diabetologia 28:412-419, 1985.

DNA samples are extracted from EDTA whole-blood samples using Puregene Kit (Gentra, Minneapolis, Minn., USA). Samples are then genotyped essentially as described in Example 1.2.

Results showing a significant association between any of the alleles or genotypes (determined using methods described in Example 1) indicate that the Intron 5 SNP is associated with the development of type II diabetes.

### Example 16

### Tau Phosphorylation is Enhanced in Parkinson's Disease Subjects

A cohort of 12 Parkinson's disease subjects and 8 control subject were recruited Subjects were age and sex matched. Soluble protein was isolated from cortical tissue from each subject using Trizol reagent (Sigma) essentially according to manufacturer's instructions. Approximately 1 μg of total protein was used to assay for total Tau or Tau phosphorylated at serine residue 396 using the Human Tau or Human Tau [pSER396] ELISA kit, respectively (Biosource International, CA) essentially according to manufacturer's instructions.

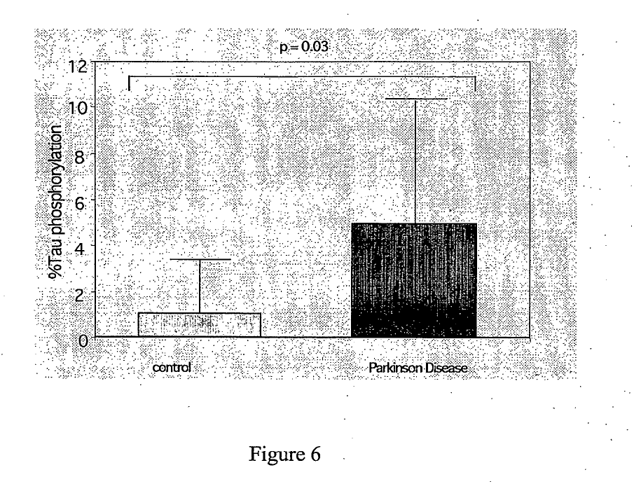

As shown in FIG. 6 there was a significant (5-fold) increase in level of Tau phosphorylation in protein isolated from the brain of a Parkinson's disease subject than the level detected in a control sample (p=0.03, two tailed Student's T test). These results suggest a role for Tau phosphorylation in Parkinson's disease.

### Example 17

### Transcriptional Activity of the GSK-3β Promoter is Influenced by a Promoter SNP (rs3755557)

Total RNA was isolated from 30 brain tissue samples (isolated from the cerebellum) using either Trizol reagent (Sigma) or SV Total RNA Isolation kit (Promega) essentially according to the relevant manufacturer's instructions.

For each sample, approximately 1 μg of RNA was reverse transcribed using an oligo dT primer, followed by Dnase 1 digestion. 0.1 μg of brain cDNA was then used to amplify GSK3β transcripts using GSK3B-RT-2F: CCAGTGGTGAGAAGAAAGATGAGG (SEQ ID NO: 53) and GSK3B-RT-2R: TGTTGGAGTTCCCAGGACCTG (SEQ ID NO: 54). The absolute level of GSK3β transcripts in each sample was quantified by real-time PCR (SYBR-Green chemistry). The difference in total RNA levels for each sample was determined by calculating the relative level of GSK-3β RNA (normalized to the level of GAPDH RNA) in each sample and comparing this level between samples.

The genotype of each individual at the site of the rs3755557 SNP (either an adenosine or a thymidine at a position corresponding to position 1679 of SEQ ID NO: 49) was determined using the following method:

Genomic DNA from each brain sample supra was used as template to amplify a 170 bp product using the primers rs3755557-F (5′-GCCATCCTGATTGTAATCCAGTGG-3′) (SEQ ID NO: 54) and rs3755557-R (5′-GCTTACTTTGTTCTGTCCCAGGTCC-3′) (SEQ ID NO: 55). The PCR product was then digested with the restriction enzyme MseI. Upon electrophoresis in a 4% agarose gel, the presence of the T allele is indicated by a 170 bp band, while the A allele is indicated by the presence of a 90 bp and 80 bp band.

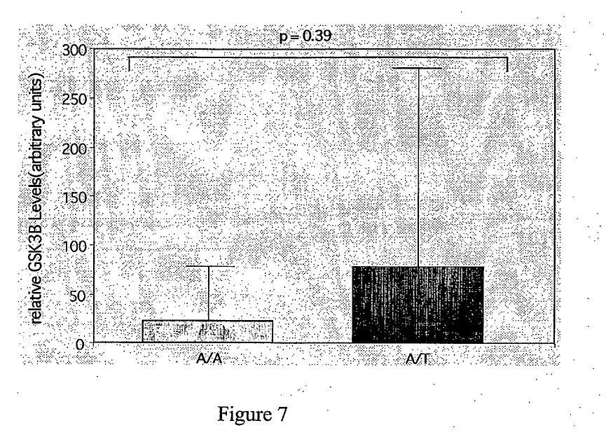

As shown in FIG. 7 there was a 3.3-fold difference (p=0.39, two tailed Student's T test) in GSK30 mRNA levels between the two genotype groups (i.e.; AA or AT). As shown in this figure the T allele is associated with increased expression of GSK3β.

### Example 18

### The Genotype at SNP rs3755557 Affects the Response to Lithium

Samples from 38 patients suffering from a bipolar affective disorder were analyzed to determine the genotype present at the site of the rs3755557 SNP (either an adenosine or a thymidine at a position corresponding to position 1679 of SEQ ID NO: 49). Genotype was determined essentially as described in Example 17.

The medical records from these 38 bipolar patients were also examined to determine the effectiveness of treatment with lithium (ranked as highly responsive to lithium; variably responsive to lithium or poorly responsive to lithium).

As shown in Table 5 subjects that responded well to treatment with lithium were significantly more likely to have the AA homozygous genotype than the AT heterozygous genotype (Odds ratio=15, p=0.01).

Accordingly, the genotype of the subject is associated with the response of the subject to therapy with lithium.

TABLE 5
Genotype of subjects that responded to treatment with lithium

| | | Variably | |
|---|---|---|---|
| | Highly Responsive | Responsive | Poorly Responsive |
| AA | 11 | 4 | 4 |
| AT | 1 | 0 | 4 |

### Example 19

### Haplotype Analysis of Three Functional SNPs of GSK-3β in Neurodegenerative Cohorts

As previously discussed the rs334558 SNP (located at position 231 of SEQ ID NO: 1) and the rs6438552 (located at position 181,700 of SEQ ID NO: 1) are useful for determining a subject that is at risk of developing a neurodegenerative disease.

To determine the ability of these SNPs in conjunction with the rs3755557 SNP to diagnose a bipolar affective disease a case/control cohort of bipolar patients were genotyped for each SNP.

As shown in Table 6 there is a significant difference in the distribution of haplotype frequencies between the two groups (i.e., bipolar versus control) (p=0.000*). In particular, there significant differences in frequencies of the following genotypes A-C-C; A-C-T; A-T-C; or A-T-T (the alleles listed are for the following SNPs: rs3755557, rs334558 and rs6438552, respectively) in control and bipolar groups.

TABLE 6
Haplotype analysis of bipolar affective subjects

| | Haplotype | | | | | | | |
|---|---|---|---|---|---|---|---|---|
| | A-C-C | A-C-T | A-T-C | A-T-T | T-C-C | T-C-T | T-T-C | T-T-T |
| Bipolar Disorder (n = 240) | 11% | 11% | 32% | 35% | 7% | 4% | 0% | 0% |
| Control (n = 228) | 22% | 5% | 6% | 52% | 12% | 2% | 0.5% | 0.5% |
| P value for each haplotype | 0.003* | 0.042* | 0.000* | 0.000* | NS# | NS | NS | NS |
| (odds ratio) | (0.451) | (2.187) | (7.664) | (0.493) | | | | |

*significant at <0.01 level
#NS = not significant

1. A method for determining a disease or disorder associated with aberrant glycogen synthase kinase-3β (GSK-3β) expression or activity or a predisposition to the disease or disorder, said method comprising detecting a marker within a GSK-3β gene or an expression product thereof that is associated with the disease or disorder in a sample derived from a subject, wherein the detection is indicative of the disease or disorder or a predisposition to the disease or disorder in the subject.

2. The method according to claim 1 wherein the disease or disorder is selected from the group consisting of a neurodegenerative disease, a psychiatric disorder, a disorder associated with aberrant glucose metabolism, a stroke, a stroke induced ischemia, muscle hypertrophy, a cancer and mixtures thereof.

3. The method according to claim 1 wherein the disorder is a disorder associated with aberrant glucose metabolism selected from the group consisting of insulin resistance, type II diabetes and mixtures thereof

4. The method according to claim 1 wherein the disease or disorder is a neurodegenerative disease selected from the group consisting of, an Alzheimer's disease, a Parkinson's disease and mixtures thereof.

5. The method according to claim 1 wherein the marker is within a GSK-3β genomic gene comprising a nucleotide sequence at least 80% identical to the sequence set forth in SEQ ID NO: 1 or the complement thereof.

6. The method according to claim 1 wherein the marker is within a GSK-3β mRNA that comprises a nucleotide sequence at least 80% identical to a nucleotide sequence selected from the group consisting of SEQ ID NO: 2, SEQ ID NO: 4, SEQ ID NO: 6 and SEQ ID NO: 8.

7. The method according to claim 1 wherein the marker comprises or consists of a nucleotide sequence at least 80% identical to a region at least 20 nucleotides in length of a sequence selected from the group consisting of:
(i) a sequence at least about 80% identical to a sequence selected from the group consisting of SEQ ID NO: 1, SEQ ID NO: 2, SEQ ID NO: 4, SEQ ID NO: 6 and SEQ ID NO: 8;
(ii) a sequence capable of encoding an amino acid sequence at least 80% identical to the sequence set forth in SEQ ID NO: 3, SEQ ID NO: 5, SEQ ID NO: 7 and SEQ ID NO: 9; and
(iii) a sequence complementary to a sequence set forth in (i) or (ii).

8. The method according to claim 1 wherein the marker is within a GSK-3β polypeptide that comprises an amino acid sequence at least 80% identical to an amino acid sequence selected from the group consisting of SEQ ID NO: 3, SEQ ID NO: 5, SEQ ID NO: 7 and SEQ ID NO: 9.

9. The method according to claim 1 wherein the marker comprises a polymorphism in the GSK-3β gene or an expression product thereof.

10. The method according to claim 9 wherein the polymorphism is in homozygous form.

11. The method according to claim 9 the polymorphism is associated with or causes alternative splicing of a GSK-3β mRNA.

12. The method according to claim 11 wherein the alternative splicing of the GSK-3β mRNA causes increased expression of a nucleic acid that comprises a nucleotide sequence at least 80% identical one or more nucleotide sequences selected from the group selected from SEQ ID NO: 4, SEQ ID NO: 6 and SEQ ID NO: 8.

13. The method according to claim 9 wherein the polymorphism is associated with or causes increased expression of a GSK-3β gene.

14. The method according to claim 9 wherein the polymorphism is located within intron 5 of the GSK-3β gene.

15. The method according to claim 14 wherein intron 5 of the GSK-3β gene comprises or consists of the nucleotides in the region spanning from nucleotide position 178,624 to nucleotide position 181,858 of SEQ ID NO: 1.

16. The method according to claim 9 wherein the polymorphism is located within the promoter region of the GSK-3β gene.

17. The method according to claim 16 wherein the promoter region of the GSK-3β gene comprises a nucleotide sequence corresponding to the region spanning from nucleotide position 1 to nucleotide position 1232 of SEQ ID NO: 1 and/or the nucleotide sequence set forth in SEQ ID NO: 49.

18. The method according to claim 9 wherein the polymorphism comprises a single nucleotide polymorphism (SNP).

19. The method according to claim 18 wherein the SNP is selected from the group consisting of a thymidine at a position corresponding to nucleotide position 181,700 of SEQ ID NO: 1, a cytosine at a position corresponding to nucleotide position 181,700 of SEQ ID NO: 1, a thymidine at a position corresponding to nucleotide position 232 of SEQ ID NO: 1, a cytosine at a position corresponding to nucleotide position 232 of SEQ ID NO: 1, an adenosine at a position corresponding to nucleotide position 1679 of SEQ ID NO: 49, a thymidine at a position corresponding to nucleotide position 1679 of SEQ ID NO: 49 and mixtures thereof.

20. The method according to claim 1 wherein the marker is an alternatively spliced GSK-3β transcript comprising one or more nucleotide sequences selected from the group consisting of SEQ ID NO: 4, SEQ ID NO: 6 or SEQ ID NO: 8.

21. The method according to claim 1 wherein the marker is detected by performing a process comprising hybridizing a nucleic acid probe or primer comprising the sequence of the marker to a marker linked to nucleic acid in a biological sample derived from the subject under moderate to high stringency hybridization conditions and detecting the hybridization using a detection means, wherein hybridization of the probe to the sample nucleic acid indicates that the subject being tested is predisposed to or suffers from the disease or disorder.

22. The method according to claim 21 wherein the detection means is a nucleic acid hybridization or amplification reaction.

23. The method according to claim 21 wherein the detection means is a polymerase chain reaction (PCR).

24. The method according to claim 21 wherein the nucleic acid probe or primer comprises one or more nucleotide sequence selected from the group consisting of SEQ ID NO: 12, SEQ ID NO: 13, SEQ ID NO: 50, SEQ ID NO: 51, SEQ ID NO: 54 and SEQ ID NO: 55.

25. The method according to claim 1 wherein the marker is a polypeptide encoded by an alternatively spliced GSK-3β transcript, said polypeptide comprising one or more amino acid sequence selected from the group consisting of SEQ ID NO: 5, SEQ ID NO: 7 and SEQ ID NO: 9.

26. The method according to claim 1 wherein the marker is detected by performing a process comprising contacting a biological sample derived from the subject with an antibody or ligand capable of selectively binding to the marker for a time and under conditions sufficient for an antibody/ligand complex to form and then detecting the complex wherein detection of the complex is indicative of the disease or disorder and/or a predisposition to the disease or disorder in the subject.

27. The method according to claim 1 wherein the marker is detected by determining an enhanced or reduced level of a GSK-3β transcript in a sample derived from the subject, wherein said enhanced or reduced level of the GSK-3β transcript is indicative of the disease or disorder and/or a predisposition to the disease or disorder in the subject.

28. The method according to claim 27 wherein the GSK-3β transcript is an alternatively spliced GSK-3β transcript.

29. The method according to claim 28 wherein the alternatively spliced GSK-3β transcript comprises a nucleotide sequence at least 80% identical one or more nucleotide sequences selected from the group selected from SEQ ID NO: 4, SEQ ID NO: 6 and SEQ ID NO: 8.

30. The method according to claim 27 wherein an enhanced or reduced level of a GSK-3β transcript is detected by performing a process comprising:
(i) determining the level of the GSK-3β transcript in a sample derived from the subject;
(ii) determining the level of the GSK-3β transcript in a suitable control sample,
wherein an enhanced or reduced level of the GSK-3β transcript at (i) compared to (ii) is indicative of the disease or disorder and/or the predisposition to a disease or disorder in the subject.

31. The method according to claim 27 wherein the level of the GSK-3β transcript is determined by performing a process comprising hybridizing a nucleic acid probe that selectively hybridizes to the GSK-3β transcript with altered splicing to nucleic acid in a sample derived from the subject under moderate to high stringency hybridization conditions and detecting the hybridization using a detection means, wherein the level of hybridization of the probe to the sample nucleic acid is indicative of the level of the GSK-3β transcript in the sample.

32. The method according to claim 31 wherein the detection means is an amplification reaction or a hybridization reaction.

33. The method according to claim 31 wherein the detection means is a polymerase chain reaction (PCR).

34. The method according to claim 1 wherein the marker is detected by determining an enhanced or reduced level of a GSK-3β polypeptide in a sample derived from the subject, wherein said enhanced or reduced level of the GSK-3β polypeptide is indicative of the disease or disorder and/or a predisposition to the disease or disorder in the subject.

35. The method according to claim 34 wherein the GSK-3β polypeptide is encoded by an alternatively spliced GSK-3β transcript.

36. The method according to claim 35 wherein the GSK-3β polypeptide comprises an amino acid sequence at least 80% identical one or more amino acid sequences selected from the group selected from SEQ ID NO: 5, SEQ ID NO: 7 and SEQ ID NO: 9.

37. The method according to claim 34 wherein detecting an enhanced or reduced level of the GSK-3β polypeptide comprises performing a process comprising:
(i) determining the level of the GSK-3β polypeptide in a sample derived from the subject;
(ii) determining the level of the GSK-3β polypeptide in a suitable control sample,
wherein an enhanced or reduced level of the GSK-3β polypeptide at (i) compared to (ii) is indicative of the disease or disorder and/or a predisposition to the disease or disorder in the subject.

38. The method according to claim 34 wherein the level of the GSK-3β polypeptide is detected by performing a process comprising contacting a biological sample derived from the subject with an antibody or ligand capable of selectively binding to the GSK-3β polypeptide for a time and under conditions sufficient for an antibody/ligand complex to form and then detecting the complex wherein the level of the complex is indicative of the level of the GSK-3β polypeptide in the subject.

39. The method according to claim 1 further comprising determining an association between the marker and the disease or disorder.

40. The method according to claim 1 wherein the sample comprises a nucleated cell.

41. The method according to claim 40 wherein the sample is selected from the group consisting of whole blood, serum, plasma, a lymphocyte, saliva, urine, a buccal cell and a skin cell.

42. The method according to claim 40 wherein the sample is derived previously from the subject.

43. A method for determining a disease or disorder associated with aberrant glycogen synthase kinase-3β (GSK-3β) expression or activity or a predisposition to the disease or disorder in a subject, said method comprising:
(i) detecting a first polymorphism comprising a cytosine or a thymidine in homozygous form at a position corresponding to nucleotide position 181,700 of SEQ ID NO: 1; and
(ii) detecting a second polymorphism comprising a cytosine or a thymidine in homozygous form at a position corresponding to nucleotide position 232 of SEQ ID NO: 1,
wherein detection of the first and the second polymorphisms is indicative of the disease or disorder or a predisposition to the disease or disorder in the subject.

44. A method for determining a disease or disorder associated with aberrant glycogen synthase kinase-3β (GSK-3β) expression and/or activity or a predisposition to the disease or disorder in a subject, said method comprising:
(i) detecting a first polymorphism comprising a cytosine or a thymidine in homozygous form at a position corresponding to nucleotide position 181,700 of SEQ ID NO: 1;
(ii) detecting a second polymorphism comprising a cytosine or a thymidine in homozygous form at a position corresponding to nucleotide position 232 of SEQ ID NO: 1; and
(iii) detecting a third polymorphism comprising an adenosine and/or a thymidine at a position corresponding to nucleotide position 1679 of SEQ ID NO: 49,
wherein detection of the first, second and third polymorphisms is indicative of the disease or disorder or a predisposition to the disease or disorder in the subject.

45. The method according to claim 44 wherein the third polymorphism is an adenosine at a position corresponding to nucleotide position 1679 of SEQ ID NO: 49.

46. The method according to claim 45 wherein the disease or disorder associated with aberrant glycogen synthase kinase-3β (GSK-3β) expression and/or activity is a psychiatric disorder.

47. The method according to claim 46 wherein the psychiatric disorder is a bipolar affective disorder.

48. A method for determining a neurodegenerative disease or a predisposition to a neurodegenerative disease in a subject, said method comprising:
(i) amplifying nucleic acid from the subject using an amplification reaction, wherein the amplification reaction is performed using one or more pairs of primers selected from the group consisting of:
(a) a primer comprising a nucleotide sequence set forth in SEQ ID NO: 10 and a primer comprising a nucleotide sequence set forth in SEQ ID NO: 11; and
(b) a primer comprising a nucleotide sequence set forth in SEQ ID NO: 50 and a primer comprising a nucleotide sequence set forth in SEQ ID NO: 51; and
(ii) detecting a polymorphism in the amplified nucleic acid from (i),
wherein detection of said polymorphism is indicative of a neurodegenerative disease or a predisposition to a neurodegenerative disease.

49. The method according to claim 48 wherein the polymorphism is detected by determining the nucleotide sequence of the amplified nucleic acid.

50. A method for determining a subject likely to respond to a treatment for a disease or disorder associated with aberrant GSK-3β expression and/or activity, said method comprising detecting a marker within a GSK-3β gene or an expression product thereof that is associated with the disease or disorder in a sample derived from a subject, wherein the detection is indicative of the disease or disorder or a predisposition to the disease or disorder in the subject.

51. The method according to claim 50 is a SNP selected from the group consisting of a thymidine at a position corresponding to nucleotide position 181,700 of SEQ ID NO: 1, a cytosine at a position corresponding to nucleotide position 181,700 of SEQ ID NO: 1, a thymidine at a position corresponding to nucleotide position 232 of SEQ ID NO: 1, a cytosine at a position corresponding to nucleotide position 232 of SEQ ID NO: 1, an adenosine at a position corresponding to nucleotide position 1679 of SEQ ID NO: 49, and a thymidine at a position corresponding to nucleotide position 1679 of SEQ ID NO: 49.

52. The method according to claim 50 wherein the marker is a SNP comprising adenosine at a position corresponding to nucleotide position 1679 of SEQ ID NO: 49.

53. The method according to claim 50 wherein the disease or disorder is a bipolar affective disorder and the treatment is administration of lithium.

54. A method for determining a subject having a reduced risk of developing a disease or disorder associated with aberrant GSK-3β expression and/or activity comprising detecting a marker within a GSK-3β gene or an expression product thereof that is associated with reduced risk of developing the disease or disorder in a sample derived from a subject, wherein the detection is indicative of a reduced risk of developing the disease or disorder or a predisposition to the disease or disorder in the subject.

55. The method according to claim 54 wherein the marker is a polymorphism in heterozygous form.

56. The method according to claim 54 wherein the marker comprises a cytosine at a position corresponding to nucleotide position 181,700 of SEQ ID NO: 1 within one copy of a GSK-3β genomic gene and a thymidine at a position corresponding to nucleotide position 181,700 of SEQ ID NO: 1 within another copy of a GSK-3β genomic gene.

57. A probe or primer comprising at least 20 nucleotides that is capable of selectively hybridizing to the nucleotide sequence set forth in SEQ ID NO: 1 and detecting a marker that is associated with a disease or disorder associated with aberrant GSK-3β expression and/or activity.

58. The probe or primer according to claim 57 comprising a nucleotide sequence selected from the group consisting of SEQ ID NO: 10, SEQ ID NO: 11, SEQ ID NO: 50, SEQ ID NO: 51, SEQ ID NO: 54 and SEQ ID NO: 55.

59. (canceled)

60. A method for the treatment of a disease or disorder associated with aberrant GSK-3β expression and/or activity, said method comprising:
(i) detecting the disease or disorder or a predisposition to the disease or disorder by performing the method according to claim 1; and
(ii) administering or recommending a therapeutic for the treatment of the disease or disorder.

61. The method according to claim 60 wherein the disease or disorder is a neurodegenerative disease.

62. A method for monitoring the efficacy of treatment of a subject undergoing treatment for a disease or disorder associated with aberrant GSK-3β expression and/or activity, said method comprising:
(i) determining the level of expression of a GSK-3β expression product in a sample derived from the subject; and
(ii) determining the level of expression of the GSK-3β expression product in a suitable control sample,
wherein a similar level of expression of the GSK-3β expression product at (i) compared to (ii) indicates that the treatment is effective for the treatment of the disease or disorder.

63. The method according to claim 62 wherein the GSK-3β expression product is a nucleic acid comprising a nucleotide sequence selected from the group consisting of SEQ ID NO: 4, SEQ ID NO: 6 and SEQ ID NO: 8.

64. The method according to claim 63 wherein the GSK-3β expression product is a polypeptide comprising an amino acid sequence selected from the group consisting of SEQ ID NO: 5, SEQ ID NO: 7 and SEQ ID NO: 9.

65. A method for determining a subject that carries a gene or allele of a gene or a polymorphism that is associated with a disease or disorder associated with aberrant glycogen synthase kinase-3β (GSK-3β) expression or activity, said method comprising detecting a marker within a GSK-3β gene that is associated with the disease or disorder in a sample derived from the subject, wherein detection of said marker indicates that the subject is a carrier of a gene or allele of a gene or a polymorphism is associated with the disease or disorder.

66. A method for identifying a marker that is associated with a disease or disorder associated with aberrant glycogen synthase kinase-3β (GSK-3β) expression or activity, said method comprising:
(i) identifying a polymorphism or allele within a GSK-3β gene or an expression product thereof;
(ii) analyzing a panel of subjects to determine those that suffer from the disease or disorder associated with aberrant GSK-3β expression or activity, wherein not all members of the panel comprise the polymorphism or allele; and
(iii) determining the variation in the development of the disease or disorder wherein said variation indicates that the polymorphism or allele is associated with a subject's predisposition to the disease or disorder associated with aberrant GSK-3β expression or activity.

67. A method for determining a marker within a GSK-3β gene or expression product that is associated with a disease or disorder associated with aberrant GSK-3β expression and/or activity, said method comprising:
(i) identifying a marker within a GSK-3β gene or expression product that is associated with a neurodegenerative disease and/or a psychiatric disease; and
(ii) determining a marker from (i) that is additionally associated with the disease or disorder associated with aberrant GSK-3β expression and/or activity.

68. The method according to claim 67 wherein the marker is associated with a neurodegenerative disease.

69. The method according to claim 67 wherein the marker comprises a single nucleotide polymorphism selected from the group consisting of a thymidine at a position corresponding to nucleotide position 181,700 of SEQ ID NO: 1, a cytosine at a position corresponding to nucleotide position 181,700 of SEQ ID NO: 1, a thymidine at a position corresponding to nucleotide position 232 of SEQ ID NO: 1, a cytosine at a position corresponding to nucleotide position 232 of SEQ ID NO: 1, an adenosine at a position corresponding to nucleotide position 1679 of SEQ ID NO: 49, a thymidine at a position corresponding to nucleotide position 1679 of SEQ ID NO: 49 and mixtures thereof.

70. A method for identifying a disease or disorder associated with aberrant GSK-3β expression and/or activity, said method comprising:
(i) identifying a marker within a GSK-3β gene or expression product that is associated with a neurodegenerative disease and/or a psychiatric disease; and
(ii) determining a disease or disorder that is associated with the marker from (i), thereby identifying the disease or disorder associated with the marker.

71. A method for identifying a disease or disorder associated with aberrant GSK-3β expression and/or activity that is associated with a marker within a GSK-3β gene, said method comprising determining a disease or disorder that is associated with a marker selected from the group consisting of a thymidine at a position corresponding to nucleotide position 181,700 of SEQ ID NO: 1, a cytosine at a position corresponding to nucleotide position 181,700 of SEQ ID NO: 1, a thymidine at a position corresponding to nucleotide position 232 of SEQ ID NO: 1, a cytosine at a position corresponding to nucleotide position 232 of SEQ ID NO: 1, an adenosine at a position corresponding to nucleotide position 1679 of SEQ ID NO: 49, a thymidine at a position corresponding to nucleotide position 1679 of SEQ ID NO: 49 and mixtures thereof.

72. A method for determining a candidate compound for the treatment of a disease or disorder associated with aberrant glycogen synthase kinase-30 (GSK-3β) expression or activity, said method comprising:
(i) administering a candidate compound to an animal or cell comprising or expressing a marker within a GSK-3β gene that is associated with the disease or disorder associated with aberrant GSK-3β expression or activity and determining the level of alternative splicing of a GSK-3β transcript in said cell or animal;
(ii) administering the candidate compound to an animal or cell that does not comprise or express the marker and determining the level of alternative splicing of the GSK-3β transcript in said cell or animal; and
(iii) comparing the level of alternative splicing at (i) and (ii),
wherein a decreased level of alternate splicing at (i) relative to (ii) indicates that the compound is a candidate compound for the treatment of the disease or disorder.

73. A method of determining a candidate compound for the treatment of a disease or disorder associated with aberrant glycogen synthase kinase-30 (GSK-3β expression or activity, said method comprising:
(i) administering a candidate compound to an animal or cell comprising or expressing a marker within a GSK-3β gene that is associated with a disease or disorder associated with aberrant GSK-3β expression or activity and determining the level of tau phosphorylation in said cell or animal;
(ii) administering the candidate compound to an animal or cell that does not comprise or express the marker and determining the level of determining the level of tau phosphorylation in said cell or animal; and
(iii) comparing the level of tau phosphorylation at (i) and (ii)
wherein a decreased level of tau phosphorylation at (i) relative to (ii) indicates that the compound is a candidate compound for the treatment of the disease or disorder.

74. A process for identifying or determining a compound for the treatment of a disease or disorder associated with aberrant glycogen synthase kinase-3β (GSK-3β) expression or activity, said process comprising:
(i) performing the method according to claim 72 to thereby identify or determine a compound for the treatment of a disease or disorder associated with aberrant GSK-3β expression or activity;
(ii) optionally, determining the structure of the compound;
(iii) optionally, providing the name or structure of the compound; and
(iv) providing the compound.

75. A process of manufacturing a compound for the treatment of a disease or disorder associated with aberrant glycogen synthase kinase-3β (GSK-3β) expression or activity, said process comprising:
(i) determining a candidate compound for the treatment of a disease or disorder associated with aberrant GSK-3β expression or activity by performing the method according to claim 72; and
(ii) using the compound in the manufacture of a therapeutic or prophylactic for the treatment of a disease or disorder associated with aberrant GSK-3β expression or activity.

76. The method according to claim 62 wherein the disease or disorder is a neurodegenerative disease.
